(19) United States
(12) Patent Application Publication
(10) Pub. No.: US 2023/0202974 A1
(43) Pub. Date: Jun. 29, 2023

(54) CASPASE 6 INHIBITORS AND USES THEREOF

(71) Applicants: The Regents of the University of California, Oakland, CA (US); SHANGPHARMA INNOVATION INC., South San Francisco, CA (US)

(72) Inventors: Adam R. Renslo, Oakland, CA (US); Michelle R. Arkin, San Francisco, CA (US); R. Jeffrey Neitz, San Francisco, CA (US); Raymond Ng, South San Francisco, CA (US); Peter Lee, San Francisco, CA (US)

(21) Appl. No.: 17/777,832

(22) PCT Filed: Nov. 20, 2020
(86) PCT No.: PCT/US2020/061659

§ 371 Date: May 18, 2022

Related U.S. Application Data
(60) Provisional application No. 62/939,263, filed on Nov. 22, 2019.

(51) Int. Cl. C07C 311/08; C07D 209/04; C07D 401/06; C07D 401/12

(52) U.S. Cl. CPC C07C 311/08; C07D 209/04; C07D 401/06; C07D 401/12

(57) ABSTRACT

Disclosed herein, inter alia, are compounds and methods for inhibiting Caspase 6 and the treatment of diseases, pharmaceutical composition including a compound as described herein and a pharmaceutically acceptable excipient and methods of inhibiting human Caspase 6 protein activity, the method including: contacting the human Caspase 6 protein with a compound as described herein.

### CROSS-REFERENCES TO RELATED APPLICATIONS

This application claims the benefit of U.S. Provisional Application No. 62/939,263, filed Nov. 22, 2019, which is incorporated herein by reference in its entirety and for all purposes.

### REFERENCE TO A “SEQUENCE LISTING,” A TABLE, OR A COMPUTER PROGRAM LISTING APPENDIX SUBMITTED AS AN ASCII FILE

The Sequence Listing written in file 048536-664001WO_Sequence_Listing_ST25.txt, created Nov. 16, 2020, 9,466 bytes, machine format IBM-PC, MS Windows operating system, is hereby incorporated by reference.

### BACKGROUND

Caspase 6 is an enzyme that in humans is encoded by the CASP6 gene. Caspase 6 has known functions in apoptosis, early immune response and neurodegeneration in Huntington’s and Alzheimer’s disease. Identifying inhibitors of Caspase 6 has proven to be a challenge. Disclosed herein, inter alia, are solutions to these and other problems known in the art.

### BRIEF SUMMARY

In an aspect is provided a compound having the formula:

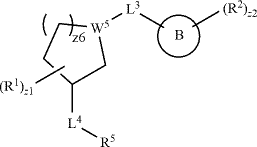

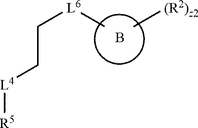

or

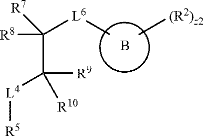

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O) NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents on adjacent carbons may optionally be joined to form a substituted or unsubstituted cycloalkyl, substituted or unsubstituted heterocycloalkyl, substituted or unsubstituted aryl, or substituted or unsubstituted heteroaryl or two R¹ substituents bonded to the same carbon atom may optionally be joined to form a substituted or unsubstituted alkyl or substituted or unsubstituted heterocycloalkyl.

z1 is an integer from 0 to 9.

R² is independently oxo, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O) NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R² substituents on adjacent carbons may optionally be joined to form a substituted or unsubstituted cycloalkyl, substituted or unsubstituted heterocycloalkyl, substituted or unsubstituted phenyl, or substituted or unsubstituted heteroaryl.

z2 is an integer from 0 to 6.

L³ is a bond, —S(O)₂—, —NR³—, —NH—, —O—, —S—, —C(O)—, —C(O)NR³—, —NR³C(O)—, —N(R³)CH₂—, —NR³C(O)NH —, —NHC(O)NR³—, —C(O)O—, —OC(O)—, substituted or unsubstituted alkylene, or substituted or unsubstituted heteroalkylene.

L⁴ is a bond, —NH—, —NR⁴—, or substituted or unsubstituted alkylene.

L⁶ is —N(R⁶)—L³— or —C(O)NH—.

W⁵ is CH or N.

z6 is 1 or 2.

R³, R⁴, and R⁶ are independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CO(C₁—C₆ alkyl), —CONH₂, —OCCl₃, —OCF₃, —OCBr₃, —OCI₃, —OCHCl₂, —OCHBr₂, —OCHI₂, —OCHF₂, —OCH₂ Cl, —OCH₂Br, —OCH₂I, —OCH₂F, unsubstituted alkyl, unsubstituted heteroalkyl, unsubstituted cycloalkyl, or unsubstituted heterocycloalkyl.

R⁷, R⁸, R⁹, and R¹⁰ are independently hydrogen or unsubstituted C₁-C₁₀ alkyl;

Ring B is aryl, or heteroaryl.

R⁵ is an electrophilic moiety.

R^{1A}, R^{1B}, R^{1C}, R^{1D}, R^{2A}, R^{2B}, R^{2C}, and R^{2D} are independently hydrogen, —CCl₃, —CBr₃, —CF₃, —CI₃, CHC1₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl.

X¹ and X² are independently —F, —Cl, —Br, or —I.

n1 and n2 are independently an integer from 0 to 4.

m1, m2, v1, and v2 are independently 1 or 2.

In an aspect is provided a pharmaceutical composition including a compound as described herein and a pharmaceutically acceptable excipient.

In an aspect is provided a method of inhibiting human Caspase 6 protein activity, the method including: contacting the human Caspase 6 protein with a compound as described herein.

In an aspect is provided a method of treating a neurodegenerative disease, the method including administering to a subject in need thereof an effective amount of a compound as disclosed herein.

In an aspect is provided a method of treating a memory loss, the method including administering to a subject in need thereof an effective amount of a compound as disclosed herein.

In an aspect is provided a method of treating axonal degradation, the method including administering to a subject in need thereof an effective amount of a compound as disclosed herein.

In an aspect is provided a method of treating an inflammatory disease, the method including administering to a subject in need thereof an effective amount of a compound as disclosed herein.

In an aspect is provided a method of treating neuroinflammation, the method including administering to a subject in need thereof an effective amount of a compound as disclosed herein.

In an aspect is provided a method of treating liver disease, the method including administering to a subject in need thereof an effective amount of a compound as disclosed herein.

In an aspect is provided a method of treating nonalcoholic steatohepatitis or nonalcoholic fatty liver disease, the method including administering to a subject in need thereof an effective amount of a compound as disclosed herein.

In an aspect is provided a method of treating a fibrotic disease, the method including administering to a subject in need thereof an effective amount of a compound as disclosed herein.

### BRIEF DESCRIPTION OF THE DRAWINGS

FIGS. 1A-1B. FIG. 1A: Compound SU20667-0134-01 inhibits VEIDase activity of purified caspase-6 (IC₅₀ = 10 nM). Assay method: 1 nM Caspase-6, 5 µM z-VEID-Glo, and various compound doses are mixed. After 30 min, luciferase detection reagent is added and the plate is read on a luminometer. In FIG. 1A, axis lable “0134-01” refers to compound SU20667-0134-01. FIG. 1B: compound SU20667-0134-01 inhibits caspase-6-mediated cleavage of laminA following staurosporine (STS) treatment in SKNAS cells (IC₅₀ =6 nM). For assay method, see Mintzer, et al. PLoS One, 2012, 7, e30376. In FIG. 1B, axis label “0134-01” refers to compound SU20667-0134-01.

FIG. 2. Compound SU20667-0134-01 inhibits z-VEID-ase activity (z-VEID-Glo) and LDHA release after treatment with staurosporine (STS) in iPSC-derived neurons from patient with frontotemporal dementia tau mutation (V337M) and wild-type tau (control). z-VAD-FMK, a pan-caspase inhibitor, also blocks VEID-ase activity and LDHA release. Assay methods: iPSC-derived induced neurons (iNs) with heterozygous V337MMAPT mutation and WT isogenic controls were generated and differentiated as previously described (Sohn et al., Neuron 2019, 104, 458-470; Wang et al., Stem Cell Reports 2017, 9, 1221-1233). After differentiation, cells were grown for twelve weeks and were then treated with 40 µM staurosporine (STS) for 48 hrs. Where indicated, 50 - 200 µM SU20667-0134-01 or z-VAD-FMK were added at the same time as STS. Following 6 h treatment, caspase-6 and caspase-3/7 levels were examined using Caspase-Glo 6 and 3/7, respectively (Promega), according to the manufacturer’s instructions. Cytotoxicity was measured using lactate dehydrogenase (LDH) release assay (Promega) following 48 h treatment, according to the manufacturer’s instructions. In FIG. 2, label “134” refers to compound SU20667-0134-01 and label “STS/134” refers to STS combined with compound SU20667-0134-01. In FIG. 2(cont.), label “134”, refers to compound SU20667-0134-01 and labels “STS/134_1” “STS/134_2”, and “STS/134_3” refers to STS combined with compound SU20667-0134-01.

FIG. 3. Compound SU20667-0134-01 inhibits spontaneous cell death in iPSC-derived mixed cortical cultures from a patient with frontotemporal dementia tau mutation (V337M). Levels of death are similar to those from cells with wild-type tau (V337V). Assay methods: cells were differentiated into post-mitotic neurons using previously reported methods (Karch, et al, Stem Cell Reports. 2019, 13, 930-55; van de Leemput J., et al. Neuron. 2014, 83, 51-68; Chambers S.M., et al. Nat Biotechnol. 2009, 27, 275-80). Ninety-day old V337M and control cortical cultures were treated with SU20667-0134-01 at the defined doses and cell death was assessed 48 hrs following treatment using an ethidium homodimer (EtHD) assay. Cells were incubated with EtHD (4 µM) for 30 min, rinsed with 1X PBS, incubated with Hoechst (1 µg/mL) for five minutes, and washed with PBS. Warm culture media was added to the plates, which were then imaged by microscopy. The fraction of dead cells was defined by the number of EtHD nuclei / number of Hoechst positive nuclei. In FIG. 3, label “134”, refers to compound SU20667-0134-01.

FIG. 4. Compound SU20667-0134-01 inhibits cell death phenotype following staurosporine (STS) treatment in iPSC-derived neurons from a patient with frontotemporal dementia tau mutation (V337M) and wild-type tau (control). z-VAD-FMK, a pan-caspase inhibitor, also inhibits the cell death phenotype. Cell methods as described for FIG. 2; cells stained for MAP2 and NeuN. In FIG. 4 (cont.), label “Inhibitor 134” refers to compound SU20667-0134-01.

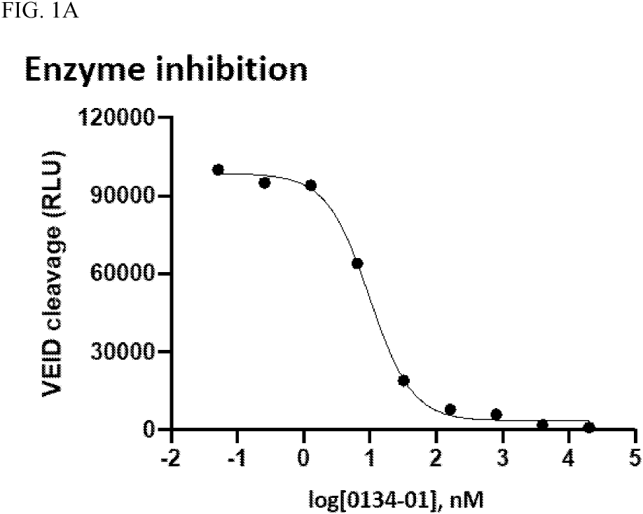

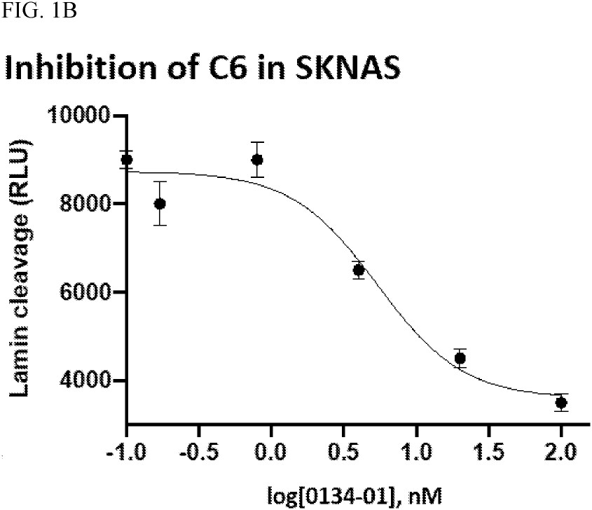

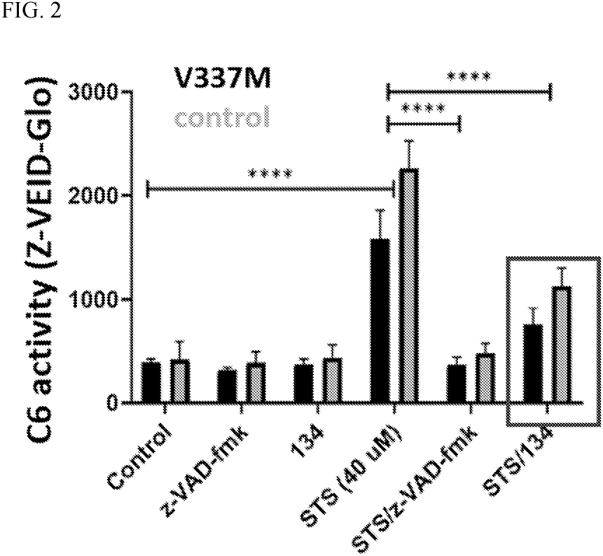

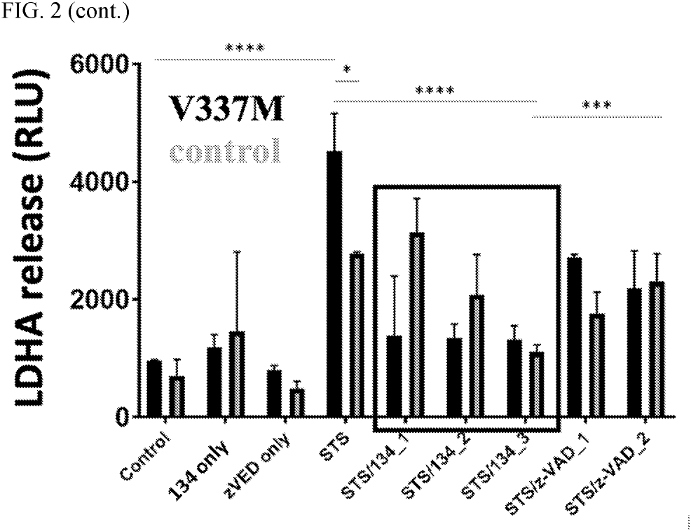

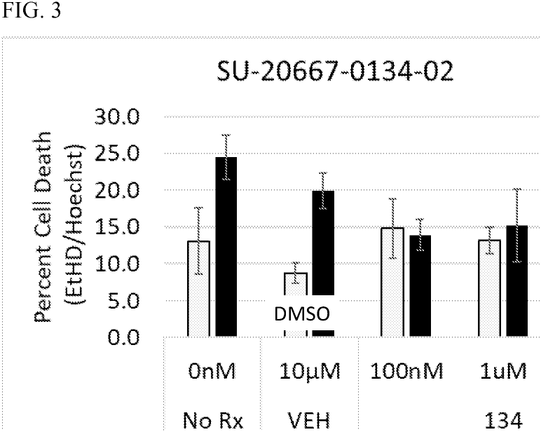

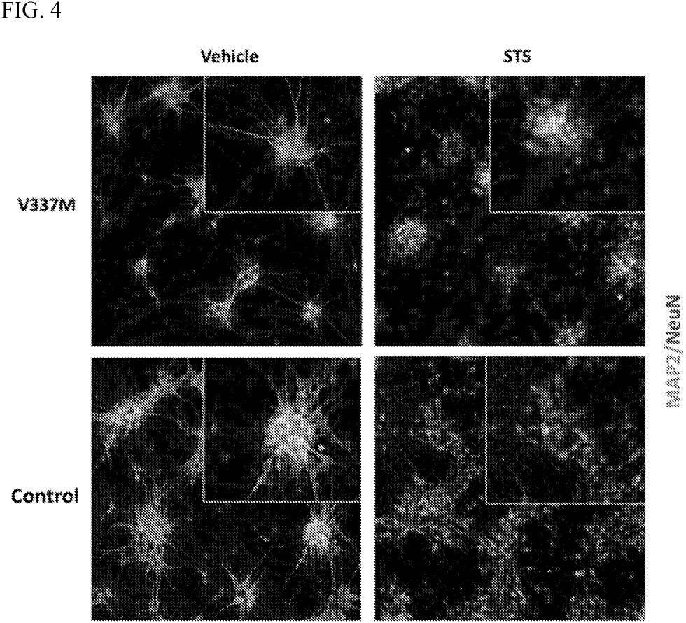

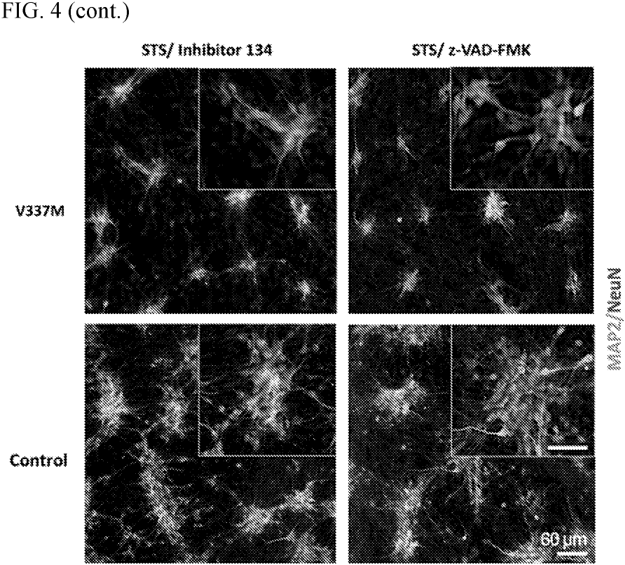

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1-and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds. In embodiments, the alkyl is fully saturated. In embodiments, the alkyl is monounsaturated. In embodiments, the alkyl is polyunsaturated.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. In embodiments, the alkylene is fully saturated. In embodiments, the alkylene is monounsaturated. In embodiments, the alkylene is polyunsaturated. In embodiments, an alkenylene includes one or more double bonds. In embodiments, an alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH—₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R’— represents both —C(O)₂R’— and —R’C(O)₂—. As described above, heteroalkyl groups, as used herein, include those groups that are attached to the remainder of the molecule through a heteroatom, such as —C(O)R’, —C(O)NR’, -NR’R”, -OR’, -SR’, and/or —SO₂R’. Where “heteroalkyl” is recited, followed by recitations of specific heteroalkyl groups, such as -NR’R” or the like, it will be understood that the terms heteroalkyl and -NR′R″ are not redundant or mutually exclusive. Rather, the specific heteroalkyl groups are recited to add clarity. Thus, the term “heteroalkyl” should not be interpreted herein as excluding specific heteroalkyl groups, such as -NR’R” or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. In embodiments, a heteroalkenylene includes one or more double bonds. In embodiments, a heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. In embodiments, a bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w} , where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In embodiments, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkyl is attached to the parent molecular moiety through any carbon atom contained within the base ring. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, a bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In embodiments, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, the term “heterocycloalkyl” means a monocyclic, bicyclic, or a multicyclic heterocycloalkyl ring system. In embodiments, heterocycloalkyl groups are fully saturated. In embodiments, a bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings. In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro- 1H-carbazol-9-yl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. In embodiments, a fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). In embodiments, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl, benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substitutents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “〰” denotes the point of attachment of a chemical moiety to the remainder of a molecule or chemical formula.

The term “oxo,” as used herein, means an oxygen that is double bonded to a carbon atom.

The term “alkylsulfonyl,” as used herein, means a moiety having the formula —S(O₂)—R’, where R’ is a substituted or unsubstituted alkyl group as defined above. R’ may have a specified number of carbons (e.g., “C₁-C₄ alkylsulfonyl”).

The term “alkylarylene” as an arylene moiety covalently bonded to an alkylene moiety (also referred to herein as an alkylene linker). In embodiments, the alkylarylene group has the formula:

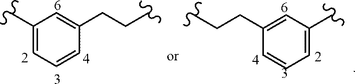

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃ —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR’, ═O, ═NR’, ═N—OR’, —NR’R”, —SR’, -halogen, —SiR’R”R’”, —OC(O)R’, —C(O)R’, —CO₂R’, —CONR’R”, —OC(O)NR’R”, —NR”C(O)R’, —NR’—C(O)NR”R’”, —NR”C(O)₂R’, —NR—C(NR’R”R’═”)═NR””, —NR—C(NR’R”)═NR’”, —S(O)R’, —S(O)₂R’, —S(O)2NR’R”, —NRSO₂R’, —NR’NR”R’”, —ONR’R”, —NR’C(O)NR”NR’”R””, —CN, —NO₂, —NR’SO₂R”, —NR’C(O)R”, —NR’C(O)—OR”, —NR’OR”, in a number ranging from zero to (2m’+1), where m’ is the total number of carbon atoms in such radical. R, R’, R”, R’”, and R”” each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R’, R”, R’”, and R”” group when more than one of these groups is present. When R’ and R” are attached to the same nitrogen atom, they can be combined with the nitrogen atom to form a 4-, 5-, 6-, or 7-membered ring. For example, —NR’R” includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR’, —NR’R”, —SR’, -halogen, —SiR’R”R’”, —OC(O)R’, —C(O)R’, —CO₂R’, —CONR’R”, —OC(O)NR’R”, —NR”C(O)R’, —NR’—C(O)NR”R’”, —N”C(O)₂R’, —NR—C(NR’R”R’”)═NR””, —NR—C(NR’R”)═NR’”, —S(O)R’, —S(O)₂R’, —S(O)₂NR’R” , —NRSO₂R’, —NR’NR”R’”, —ONR’R”, —NR’C(O)NRNR’”R””, —CN, —NO₂, —R’, —N₃, —CH(Ph)₂, fluoro(C₁-C₄)alkoxy, and fluoro(C₁-C₄)alkyl, —NR‘SO₂R”, —NR’C(O)R”, —NR’C(O)—OR”, —NR’OR”, in a number ranging from zero to the total number of open valences on the aromatic ring system; and where R’, R”, R’”, and R”” are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R’, R”, R’”, and R”” groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents on adjacent carbons may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula —T—C(O)—(CRR’)_{q}—U—, wherein T and U are independently —NR—, —O—, —CRR’—, or a single bond, and q is an integer of from 0 to 3. Alternatively, two of the substituents on adjacent atoms of the aryl or heteroaryl ring may optionally be replaced with a substituent of the formula —A—(CH₂)ᵣ—B—, wherein A and B are independently —CRR’—, —O—, —NR—, —S—, —S(O)—, —S(O)₂—, —S(O)₂NR’—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X’— (C”R”R’”)_{d}—, where s and d are independently integers of from 0 to 3, and X’ is —O—, —NR’—, —S—, —S(O)—, —S(O)₂—, or —S(O)₂NR’—. The substituents R, R’, R”, and R’” are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₂₀ alkyl, C₁-C₁₂ alkyl, C₁-C₈ alkyl, C₁-C₆ alkyl, C₁-C₄ alkyl, or C₁-C₂ alkyl), heteroalkyl (e.g., 2 to 20 membered heteroalkyl, 2 to 12 membered heteroalkyl, 2 to 8 membered heteroalkyl, 2 to 6 membered heteroalkyl, 4 to 6 membered heteroalkyl, 2 to 3 membered heteroalkyl, or 4 to 5 membered heteroalkyl), cycloalkyl (e.g., C₃-C₁₀ cycloalkyl, C₃-C₈ cycloalkyl, C₃-C₆ cycloalkyl, C₄-C₆ cycloalkyl, or C₅-C₆ cycloalkyl), heterocycloalkyl (e.g., 3 to 10 membered heterocycloalkyl, 3 to 8 membered heterocycloalkyl, 3 to 6 membered heterocycloalkyl, 4 to 6 membered heterocycloalkyl, 4 to 5 membered heterocycloalkyl, or 5 to 6 membered heterocycloalkyl), aryl (e.g., C₆-C₁₂ aryl, C₆-C₁₀ aryl, or phenyl), or heteroaryl (e.g., 5 to 12 membered heteroaryl, 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, -NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₂₀ alkyl, C₁-C₁₂ alkyl, C₁-C₈ alkyl, C₁-C₆ alkyl, C₁-C₄ alkyl, or C₁-C₂ alkyl), heteroalkyl (e.g., 2 to 20 membered heteroalkyl, 2 to 12 membered heteroalkyl, 2 to 8 membered heteroalkyl, 2 to 6 membered heteroalkyl, 4 to 6 membered heteroalkyl, 2 to 3 membered heteroalkyl, or 4 to 5 membered heteroalkyl), cycloalkyl (e.g., C₃-C₁₀ cycloalkyl, C₃-C₈ cycloalkyl, C₃-C₆ cycloalkyl, C₄-C₆ cycloalkyl, or C₅-C₆ cycloalkyl), heterocycloalkyl (e.g., 3 to 10 membered heterocycloalkyl, 3 to 8 membered heterocycloalkyl, 3 to 6 membered heterocycloalkyl, 4 to 6 membered heterocycloalkyl, 4 to 5 membered heterocycloalkyl, or 5 to 6 membered heterocycloalkyl), aryl (e.g., C₆-C₁₂ aryl, C₆-C₁₀ aryl, or phenyl), or heteroaryl (e.g., 5 to 12 membered heteroaryl, 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, -NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₂₀ alkyl, C₁-C₁₂ alkyl, C₁-C₈ alkyl, C₁-C₆ alkyl, C₁-C₄ alkyl, or C₁-C₂ alkyl), heteroalkyl (e.g., 2 to 20 membered heteroalkyl, 2 to 12 membered heteroalkyl, 2 to 8 membered heteroalkyl, 2 to 6 membered heteroalkyl, 4 to 6 membered heteroalkyl, 2 to 3 membered heteroalkyl, or 4 to 5 membered heteroalkyl), cycloalkyl (e.g., C₃-C₁₀ cycloalkyl, C₃-C₈ cycloalkyl, C₃-C₆ cycloalkyl, C₄-C₆ cycloalkyl, or C₅-C₆ cycloalkyl), heterocycloalkyl (e.g., 3 to 10 membered heterocycloalkyl, 3 to 8 membered heterocycloalkyl, 3 to 6 membered heterocycloalkyl, 4 to 6 membered heterocycloalkyl, 4 to 5 membered heterocycloalkyl, or 5 to 6 membered heterocycloalkyl), aryl (e.g., C₆-C₁₂ aryl, C₆-C₁₀ aryl, or phenyl), or heteroaryl (e.g., 5 to 12 membered heteroaryl, 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “ size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “ lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)-or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the term “bioconjugate” and “bioconjugate linker” refers to the resulting association between atoms or molecules of “bioconjugate reactive groups” or “bioconjugate reactive moieties”. The association can be direct or indirect. For example, a conjugate between a first bioconjugate reactive group (e.g., —NH2, —C(O)OH, —N—hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g. a first linker of second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g. a sulfhydryl). In embodiments, the first bioconjugate reactive group (e.g., haloacetyl moiety) is covalently attached to the second bioconjugate reactive group (e.g. a sulfhydryl). In embodiments, the first bioconjugate reactive group (e.g., pyridyl moiety) is covalently attached to the second bioconjugate reactive group (e.g. a sulfhydryl). In embodiments, the first bioconjugate reactive group (e.g., -N-hydroxysuccinimide moiety) is covalently attached to the second bioconjugate reactive group (e.g. an amine). In embodiments, the first bioconjugate reactive group (e.g., maleimide moiety) is covalently attached to the second bioconjugate reactive group (e.g. a sulfhydryl). In embodiments, the first bioconjugate reactive group (e.g., -sulfo-N-hydroxysuccinimide moiety) is covalently attached to the second bioconjugate reactive group (e.g. an amine).

Useful bioconjugate reactive moieties used for bioconjugate chemistries herein include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido or maleimide groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold, or react with maleimides;
(h) amine or sulfhydryl groups (e.g., present in cysteine), which can be, for example, acylated, alkylated or oxidized;
(i) alkenes, which can undergo, for example, cycloadditions, acylation, Michael addition, etc;
(j) epoxides, which can react with, for example, amines and hydroxyl compounds;
(k) phosphoramidites and other standard functional groups useful in nucleic acid synthesis;
(1) metal silicon oxide bonding;
(m) metal bonding to reactive phosphorus groups (e.g. phosphines) to form, for example, phosphate diester bonds;
(n) azides coupled to alkynes using copper catalyzed cycloaddition click chemistry; and
(o) biotin conjugate can react with avidin or strepavidin to form a avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13.1}, R^{13.2}, R ^{13.3}, R ^{13.4}, etc., wherein each of R ^{13.1}, R^{13.2}, R ^{13.3}, R ^{13.4}, etc. is defined within the scope of the definition of R¹³ and optionally differently.

A “detectable agent” or “detectable moiety” is a composition, substance, element, or compound; or moiety thereof; detectable by appropriate means such as spectroscopic, photochemical, biochemical, immunochemical, chemical, magnetic resonance imaging, or other physical means. For example, useful detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y. ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g. fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. A detectable moiety is a monovalent detectable agent or a detectable agent capable of forming a bond with another composition.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y. ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g. metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or -CH₃). Likewise, for a linker variable (e.g., L¹, L², or L³ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

The term “exogenous” refers to a molecule or substance (e.g., a compound, nucleic acid or protein) that originates from outside a given cell or organism. For example, an “exogenous promoter” as referred to herein is a promoter that does not originate from the plant it is expressed by. Conversely, the term “endogenous” or “endogenous promoter” refers to a molecule or substance that is native to, or originates within, a given cell or organism.

A charged moiety refers to a functional group possessing an abundance of electron density (i.e. electronegative) or is deficient in electron density (i.e. electropositive). Non-limiting examples of a charged moiety includes carboxylic acid, alcohol, phosphate, aldehyde, and sulfonamide. In embodiments, a charged moiety is capable of forming hydrogen bonds.

The terms “bind” and “bound” as used herein is used in accordance with its plain and ordinary meaning and refers to the association between atoms or molecules. The association can be direct or indirect. For example, bound atoms or molecules may be direct, e.g., by covalent bond or linker (e.g. a first linker or second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like).

The term “capable of binding” as used herein refers to a moiety (e.g. a compound as described herein) that is able to measurably bind to a target (e.g., a NF-κB, a Toll-like receptor protein). In embodiments, where a moiety is capable of binding a target, the moiety is capable of binding with a Kd of less than about 10 µM, 5 µM, 1 µM, 500 nM, 250 nM, 100 nM, 75 nM, 50 nM, 25 nM, 15 nM, 10 nM, 5 nM, 1 nM, or about 0.1 nM.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (-)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer’s, normal sucrose, normal glucose, binders, fillers, disintegrants, lubricants, coatings, sweeteners, flavors, salt solutions (such as Ringer’s solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/- 10% of the specified value. In embodiments, about includes the specified value.

A “synergistic amount” as used herein refers to the sum of a first amount (e.g., an amount of a Caspase 6 inhibitor) and a second amount (e.g., a therapeutic agent) that results in a synergistic effect (i.e. an effect greater than an additive effect). Therefore, the terms “synergy”, “synergism”, “synergistic”, “combined synergistic amount”, and “synergistic therapeutic effect” which are used herein interchangeably, refer to a measured effect of the Caspase 6 inhibitor in combination with a second agent (e.g., an anticancer agent) where the measured effect is greater than the sum of the individual effects of the Caspase 6 inhibitor provided herein and the second agent (e.g., anticancer agent) administered alone as a single agent.

In embodiments, a synergistic amount may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the Caspase 6 inhibitor provided herein when used separately from the therapeutic agent. In embodiments, a synergistic amount may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the therapeutic agent when used separately from the Caspase 6 inhibitor provided herein.

The term “EC50” or “half maximal effective concentration” as used herein refers to the concentration of a molecule (e.g., small molecule, antibody, chimeric antigen receptor or bispecific antibody) capable of inducing a response which is halfway between the baseline response and the maximum response after a specified exposure time. In embodiments, the EC50 is the concentration of a molecule (e.g., small molecule, antibody, chimeric antigen receptor or bispecific antibody) that produces 50% of the maximal possible effect of that molecule.

The term “IC₅₀” or “half maximal inhibitory concentration” as used herein refers to the concentration of an inhibitory molecule (e.g., small molecule, antibody, chimeric antigen receptor or bispecific antibody) that is required to inhibit a given biologal process or biological component by 50%.

The term “small molecule” is used in accordance with its well understood meaning and refers to a low molecular weight organic compound that may regulate a biological process. In embodiments, the small molecule is a compound that weighs less than 1000 daltons. In embodiments, the small molecule is a compound that weighs less than 900 daltons. In embodiments, the small molecule weighs less than 800 daltons. In embodiments, the small molecule weighs less than 700 daltons. In embodiments, the small molecule weighs less than 600 daltons. In embodiments, the small molecule weighs less than 500 daltons. In embodiments, the small molecule weighs less than 450 daltons. In embodiments, the small molecule weighs less than 400 daltons.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

As defined herein, the term “activation”, “activate”, “activating”, “activator” and the like in reference to a protein-inhibitor interaction means positively affecting (e.g. increasing) the activity or function of the protein relative to the activity or function of the protein in the absence of the activator. In embodiments activation means positively affecting (e.g. increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein which is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein

The terms “agonist,” “activator,” “upregulator,” etc. refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

A “Caspase 6 inhibitor” refers to a compound (e.g. a compound described herein) that reduces the activity of Caspase 6 when compared to a control, such as absence of the compound or a compound with known inactivity.

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “Caspase 6” or “Caspase-6” refers to a protein (including homologs, isoforms, and functional fragments thereof) that is a member of the cysteine-aspartic acid protease (caspase) family. Caspase 6 cleaves substrates (e.g., HTT in Huntington’s, APP in Alzheimer’s disease, tau in Alzheimer’s disease), which may result in protein aggregation of the fragments. In embodiments, caspase 6 cleaves substrates that lead to inflammation (e.g., neuroinflammation), and to cell death. In embodiments, cell death leads to cirrhosis and fibrosis (e.g., in liver or other organs). In embodiments, Caspase 6 is involved in axonal degradation. The term includes any recombinant or naturally-occurring form of Caspase 6 variants thereof that maintain Caspase 6 activity (e.g., within at least 30%, 40%, 50%, 60%, 70%, 80%, 90%, 95%, or 100% activity compared to wildtype Caspase 6). In embodiments, Caspase 6 is encoded by the CASP6 gene. In embodiments, Caspase 6 has the amino acid sequence set forth in or corresponding to Entrez 839, UniProt P55212, RefSeq (protein) NP_001217.2, or RefSeq (protein) NP_116787.1. In embodiments, Caspase 6 has the sequence:

MSSASGLRRGHPAGGEENMTETDAFYKREMFDPAEKYKMDHRRRGIALIF
NHERFFWHLTLPERRGTCADRDNLTRRFSDLGFEVKCFNDLKAEELLLKI
HEVSTVSHADADCFVCVFLSHGEGNHIYAYDAKIEIQTLTGLFKGDKCHS
LVGKPKIFIIQACRGNQHDVPVIPLDVVDNQTEKLDTNITEVDAASVYTL
PAGADFLMCYSVAEGYYSHRETVNGSWYIQDLCEMLGKYGSSLEFTELLT
LVNRKVSQRRVDFCKDPSAIGKKQVPCFASMLTKKLHFFPKSN (SEQ I
D NO:1)

The term “Caspase 3” or “Caspase-3” refers to a protein (including homologs, isoforms, and functional fragments thereof) that is a member of the cysteine-aspartic acid protease (caspase) family, and cleaves substrates following aspartic acid residues. The term includes any recombinant or naturally-occurring form of Caspase 3 variants thereof that maintain Caspase 3 activity (e.g., within at least 30%, 40%, 50%, 60%, 70%, 80%, 90%, 95%, or 100% activity compared to wildtype Caspase 3). In embodiments, Caspase 3 is encoded by the CASP3 gene. In embodiments, Caspase 3 has the amino acid sequence set forth in or corresponding to Entrez 836, UniProt P42574, RefSeq (protein) NP_004337.2, RefSeq (protein) NP_116786.1, RefSeq (protein) NP_001341706, RefSeq (protein) NP_001341707, RefSeq (protein) NP_001341708, or XP_011530603.1. In embodiments, Caspase 3 has the sequence:

MENTENSVDSKSIKNLEPKIIHGSESMDSGISLDNSYKMDYPEMGLCIII
NNKNFHKSTGMTSRSGTDVDAANLRETFRNLKYEVRNKNDLTREEIVELM
RDVSKEDHSKRSSFVCVLLSHGEEGIIFGTNGPVDLKKITNFFRGDRCRS
LTGKPKLFIIQACRGTELDCGIETDSGVDDDMACHKIPVEADFLYAYSTA
PGYYSWRNSKDGSWFIQSLCAMLKQYADKLEFMHILTRVNRKVATEFESF
SFDATFHAKKQIPCIVSMLTKELYFYH (SEQ ID NO:2)

The term “Caspase 2” or “Caspase-2” refers to a protein (including homologs, isoforms, and functional fragments thereof) that is a member of the cysteine-aspartic acid protease (caspase) family, and cleaves substrates following aspartic acid residues. The term includes any recombinant or naturally-occurring form of Caspase 2 variants thereof that maintain Caspase 2 activity (e.g., within at least 30%, 40%, 50%, 60%, 70%, 80%, 90%, 95%, or 100% activity compared to wildtype Caspase 2). In embodiments, Caspase 2 is encoded by the CASP2 gene. In embodiments, Caspase 2 has the amino acid sequence set forth in or corresponding to Entrez 835, UniProt P42575, RefSeq (protein) NP_001215.1, RefSeq (protein) NP_116764.2, or RefSeq (protein) NP_116765.2. In embodiments, Caspase 2 has the sequence:

MAAPSAGSWSTFQHKELMAADRGRRILGVCGMHPHHQETLKKNRVVLAKQ
LLLSELLEHLLEKDIITLEMRELIQAKVGSFSQNVELLNLLPKRGPQAFD
AFCEALRETKQGHLEDMLLTTLSGLQHVLPPLSCDYDLSLPFPVCESCPL
YKKLRLSTDTVEHSLDNKDGPVCLQVKPCTPEFYQTHFQLAYRLQSRPRG
LALVLSNVHFTGEKELEFRSGGDVDHSTLVTLFKLLGYDVHVLCDQTAQE
MQEKLQNFAQLPAHRVTDSCIVALLSHGVEGAIYGVDGKLLQLQEVFQLF
DNANCPSLQNKPKMFFIQACRGDETDRGVDQQDGKNHAGSPGCEESDAGK
EKLPKMRLPTRSDMICGYACLKGTAAMRNTKRGSWYIEALAQVFSERACD
MHVADMLVKVNALIKDREGYAPGTEFHRCKEMSEYCSTLCRHLYLFPGHP
PT (SEQ ID NO:3)

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulator” refers to a composition that increases or decreases the level of a target molecule or the function of a target molecule or the physical state of the target of the molecule relative to the absence of the modulator. In some embodiments, a Caspase 6 associated disease modulator is a compound that reduces the severity of one or more symptoms of a disease associated with Caspase 6 (e.g. neurodegenerative disease, liver disease, or cancer). A Caspase 6 modulator is a compound that increases or decreases the activity or function or level of activity or level of function of Caspase 6.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The term “associated” or “associated with” in the context of a substance or substance activity or function associated with a disease (e.g. a protein associated disease, a cancer associated with Caspase 6 activity, Caspase 6 associated cancer, Caspase 6 associated disease (e.g., neurodegenerative disease, liver disease, cancer, inflammatory disease, autoimmune disease, or infectious disease)) means that the disease (e.g. neurodegenerative disease, liver disease, cancer, inflammatory disease, autoimmune disease, or infectious disease) is caused by (in whole or in part), or a symptom of the disease is caused by (in whole or in part) the substance or substance activity or function. For example, a cancer associated with Caspase 6 activity or function may be a cancer that results (entirely or partially) from aberrant Caspase 6 function (e.g. enzyme activity, protein-protein interaction, signaling pathway) or a cancer wherein a particular symptom of the disease is caused (entirely or partially) by aberrant Caspase 6 activity or function. As used herein, what is described as being associated with a disease, if a causative agent, could be a target for treatment of the disease. For example, a cancer associated with Caspase 6 activity or function or a Caspase 6 associated disease (e.g., neurodegenerative disease, liver disease, cancer, inflammatory disease, autoimmune disease, or infectious disease), may be treated with a Caspase 6 modulator or Caspase 6 inhibitor, in the instance where increased Caspase 6 activity or function (e.g. signaling pathway activity) causes the disease (e.g., neurodegenerative disease, liver disease, cancer, inflammatory disease, autoimmune disease, or infectious disease).

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components. For example, binding of a Caspase 6 with a compound as described herein may reduce the level of a product of the Caspase 6 catalyzed reaction or the level of a downstream derivative of the product or binding may reduce the interactions between the Caspase 6 enzyme or a Caspase 6 reaction product and downstream effectors or signaling pathway components (e.g., epigenetic regulatory proteins MLL and the transcription factor (TF) IIA family of nuclear proteins), resulting in changes in cell growth, proliferation, or survival.

In this disclosure, “comprises,” “comprising,” “containing” and “having” and the like can have the meaning ascribed to them in U.S. Patent law and can mean “includes,” “including,” and the like. “Consisting essentially of or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

The terms “disease” or “condition” refer to a state of being or health status of a patient or subject capable of being treated with the compounds or methods provided herein. The disease may be a neurodegenerative disease. The disease may be a liver disease. The disease may be a cancer. The disease may be an autoimmune disease. The disease may be an inflammatory disease. The disease may be an infectious disease. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin’s lymphomas (e.g., Burkitt’s, Small Cell, and Large Cell lymphomas), Hodgkin’s lymphoma, leukemia (including AML, ALL, and CML), or multiple myeloma.

As used herein, the term “inflammatory disease” refers to a disease or condition characterized by aberrant inflammation (e.g. an increased level of inflammation compared to a control such as a healthy person not suffering from a disease). Examples of inflammatory diseases include autoimmune diseases, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto’s encephalitis, Hashimoto’s thyroiditis, ankylosing spondylitis, psoriasis, Sjogren’s syndrome, vasculitis, glomerulonephritis, auto-immune thyroiditis, Behcet’s disease, Crohn’s disease, ulcerative colitis, bullous pemphigoid, sarcoidosis, ichthyosis, Graves ophthalmopathy, inflammatory bowel disease, Addison’s disease, Vitiligo, asthma, allergic asthma, acne vulgaris, celiac disease, chronic prostatitis, inflammatory bowel disease, pelvic inflammatory disease, reperfusion injury, ischemia reperfusion injury, stroke, sarcoidosis, transplant rejection, interstitial cystitis, atherosclerosis, scleroderma, and atopic dermatitis.

As used herein, the term “cancer” refers to all types of cancer, neoplasm or malignant tumors found in mammals (e.g. humans), including leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin’s Disease, and Non-Hodgkin’s Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

As used herein, the term “autoimmune disease” refers to a disease or condition in which a subject’s immune system has an aberrant immune response against a substance that does not normally elicit an immune response in a healthy subject. Examples of autoimmune diseases that may be treated with a compound, pharmaceutical composition, or method described herein include Acute Disseminated Encephalomyelitis (ADEM), Acute necrotizing hemorrhagic leukoencephalitis, Addison’s disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet’s disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn’s disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic’s disease (neuromyelitis optica), Discoid lupus, Dressler’s syndrome, Endometriosis, Eosinophilic esophagitis, Eosinophilic fasciitis, Erythema nodosum, Experimental allergic encephalomyelitis, Evans syndrome, Fibromyalgia , Fibrosing alveolitis, Giant cell arteritis (temporal arteritis), Giant cell myocarditis, Glomerulonephritis, Goodpasture’s syndrome, Granulomatosis with Polyangiitis (GPA) (formerly called Wegener’s Granulomatosis), Graves’ disease, Guillain-Barre syndrome, Hashimoto’s encephalitis, Hashimoto’s thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere’s disease, Microscopic polyangiitis, Mixed connective tissue disease (MCTD), Mooren’s ulcer, Mucha-Habermann disease, Multiple sclerosis, Myasthenia gravis, Myositis, Narcolepsy, Neuromyelitis optica (Devic’s), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter’s syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren’s syndrome, Sperm & testicular autoimmunity, Stiff person syndrome, Subacute bacterial endocarditis (SBE), Susac’s syndrome, Sympathetic ophthalmia, Takayasu’s arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener’s granulomatosis (i.e., Granulomatosis with Polyangiitis (GPA).

As used herein, the term “inflammatory disease” refers to a disease or condition characterized by aberrant inflammation (e.g. an increased level of inflammation compared to a control such as a healthy person not suffering from a disease). Examples of inflammatory diseases include traumatic brain injury, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto’s encephalitis, Hashimoto’s thyroiditis, ankylosing spondylitis, psoriasis, Sjogren’s syndrome,vasculitis, glomerulonephritis, auto-immune thyroiditis, Behcet’s disease, Crohn’s disease, ulcerative colitis, bullous pemphigoid, sarcoidosis, ichthyosis, Graves ophthalmopathy, inflammatory bowel disease, Addison’s disease, Vitiligo, asthma, asthma, allergic asthma, acne vulgaris, celiac disease, chronic prostatitis, inflammatory bowel disease, pelvic inflammatory disease, reperfusion injury, sarcoidosis, transplant rejection, interstitial cystitis, atherosclerosis, and atopic dermatitis.

As used herein, the term “neurodegenerative disorder” or “neurodegenerative disease” refers to a disease or condition in which the function of a subject’s nervous system becomes impaired. Examples of neurodegenerative diseases that may be treated with a compound, pharmaceutical composition, or method described herein include Alexander’s disease, Alper’s disease, Alzheimer’s disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, chronic fatigue syndrome, Cockayne syndrome, Corticobasal degeneration, Creutzfeldt-Jakob disease, frontotemporal dementia, Gerstmann-Sträussler-Scheinker syndrome, Huntington’s disease, HIV-associated dementia, Kennedy’s disease, Krabbe’s disease, kuru, Lewy body disease, Lewy body dementia, Machado-Joseph disease (Spinocerebellar ataxia type 3), Multiple sclerosis, Multiple System Atrophy, myalgic encephalomyelitis, Narcolepsy, Neuroborreliosis, Parkinson’s disease, Pelizaeus-Merzbacher Disease, Pick’s disease, Primary lateral sclerosis, Prion diseases, Progressive Supranuclear Palsy, Refsum’s disease, Sandhoffs disease, Schilder’s disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease , progressive supranuclear palsy, or Tabes dorsalis.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient’s physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject’s condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease’s transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease’s spread; relieve the disease’s symptoms (e.g., ocular pain, seeing halos around lights, red eye, very high intraocular pressure), fully or partially remove the disease’s underlying cause, shorten a disease’s duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is not prophylactic treatment (e.g., the patient has a disease, the patient suffers from a disease).

The term “prevent” refers to a decrease in the occurrence of Caspase 6 disease symptoms in a patient. As indicated above, the prevention may be complete (no detectable symptoms) or partial, such that fewer symptoms are observed than would likely occur absent treatment.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual’s disease state.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

The term “irreversible covalent bond” is used in accordance with its plain ordinary meaning in the art and refers to the resulting association between atoms or molecules of (e.g., electrophilic chemical moiety and nucleophilic moiety) wherein the probability of dissociation is low. In embodiments, the irreversible covalent bond does not easily dissociate under normal biological conditions. In embodiments, the irreversible covalent bond is formed through a chemical reaction between two species (e.g., electrophilic chemical moiety and nucleophilic moiety).

The term “electrophilic moiety” is used in accordance with its plain ordinary chemical meaning and refers to a chemical group (e.g., monovalent chemical group) that is electrophilic. In embodiments, the electrophilic chemical moiety is referred to herein as a “warhead” or “E.” In embodiments, E is:

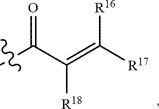

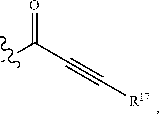

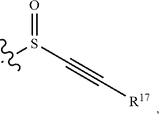

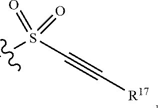

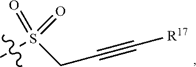

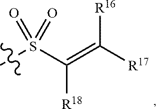

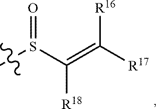

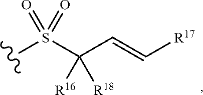

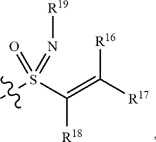

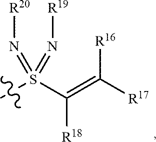

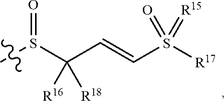

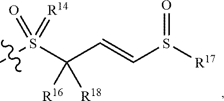

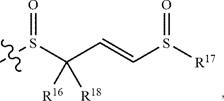

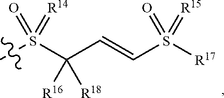

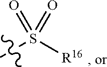

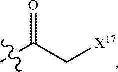

, wherein R¹⁴, R¹⁵, R¹⁶, R¹⁷, R¹⁸, R¹⁹, and R²⁰ are as described herein, including in embodiments. X¹⁷ is —F, C1, —Br, or —I. In embodiments, an electrophilic moiety is a covalent cysteine modifier moiety.

The term “covalent cysteine modifier moiety” as used herein refers to a monovalent electrophilic moiety that is able to measurably bind to a cysteine amino acid. In embodiments, the covalent cysteine modifier moiety binds via an irreversible covalent bond. In embodiments, the covalent cysteine modifier moiety is capable of binding with a Kd of less than about 10 µM, 5 µM, 1 µM, 500 nM, 250 nM, 100 nM, 75 nM, 50 nM, 25 nM, 15 nM, 10 nM, 5 nM, 1 nM, or about 0.1 nM. In embodiments, the covalent cysteine modifier moiety binds via a covalent bond.

The term “nucleophilic moiety” is used in accordance with its plain ordinary chemical meaning and refers to a chemical group (e.g., monovalent chemical group) that is nucleophilic.

An amino acid residue in a protein “corresponds” to a given residue when it occupies the same essential structural position within the protein as the given residue. Instead of a primary sequence alignment, a three-dimensional structural alignment can also be used, e.g., where the structure of the selected protein is aligned for maximum correspondence with the human protein and the overall structures compared. In this case, an amino acid that occupies the same essential position as a specified amino acid in the structural model is said to correspond to the specified residue. For example, a selected residue in a selected protein corresponds to C264 of a Caspase 6 protein (e.g., human Caspase 6 protein) when the selected residue occupies the same essential spatial or other structural relationship as C264 in the Caspase 6 protein (e.g., human Caspase 6 protein). In some embodiments, where a selected protein is aligned for maximum homology with the Caspase 6 protein (e.g., human Caspase 6 protein), the position in the aligned selected protein aligning with C264 is said to correspond to C264 of the Caspase 6 protein (e.g., human Caspase 6 protein). Instead of a primary sequence alignment, a three dimensional structural alignment can also be used, e.g., where the structure of the selected protein is aligned for maximum correspondence with the Caspase 6 protein (e.g., human Caspase 6 protein or SEQ ID NO: 1) and the overall structures compared. In this case, an amino acid that occupies the same essential position as C264 of a Caspase 6 protein (e.g., human Caspase 6 protein) in the structural model is said to correspond to the C264 residue. Another example is wherein a selected residue in a selected protein corresponds to C264 in a Caspase 6 protein (e.g., human Caspase 6 protein) when the selected residue (e.g., cysteine residue) occupies essential the same sequence, spatial, or other structural position within the protein as C264 in the Caspase 6 protein (e.g., human Caspase 6 protein).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide,” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

An amino acid or nucleotide base “position” is denoted by a number that sequentially identifies each amino acid (or nucleotide base) in the reference sequence based on its position relative to the N-terminus (or 5’-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

### II. Compounds

In an aspect is provided a compound having the formula:

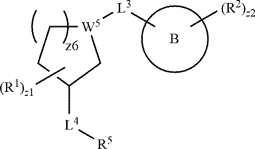

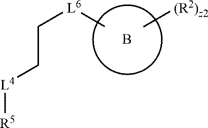

or

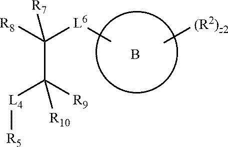

In an aspect, provided herein are compounds of formulae (I), (II) and/or (III), including embodiments thereof, or metabolites thereof.

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O) NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents on adjacent carbons may optionally be joined to form a substituted or unsubstituted cycloalkyl, substituted or unsubstituted heterocycloalkyl, substituted or unsubstituted aryl, or substituted or unsubstituted heteroaryl or two R¹ substituents bonded to the same carbon atom may optionally be joined to form a substituted or unsubstituted alkyl or substituted or unsubstituted heterocycloalkyl.

z1 is an integer from 0 to 9.

R² is independently oxo, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR²CNR^{2A}R^{2B},—NHC(O)NR^{2A}R^{2B}, —N(O)ₘ₂, —NR^{2A}R^{2B}, —C(O)R^{2C}, —C(O)—OR^{2C}, —C(O) NR2AR2B, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R² substituents on adjacent carbons may optionally be joined to form a substituted or unsubstituted cycloalkyl, substituted or unsubstituted heterocycloalkyl, substituted or unsubstituted phenyl, or substituted or unsubstituted heteroaryl.

z2 is an integer from 0 to 6.

L³ is a bond, —S(O)₂—, —NR³—, —NH—, —O—, —S—, —C(O)—, —C(O)NR³—, —NR³C(O)—, —N(R³)CH₂—, —NR³C(O)NH —, —NHC(O)NR³—, —C(O)O—, —OC(O)—, substituted or unsubstituted alkylene, or substituted or unsubstituted heteroalkylene.

L⁴ is a bond, —NH—, —NR⁴—, or substituted or unsubstituted alkylene.

L⁶ is —N(R⁶)—L³— or —C(O)NH—.

W⁵ is CH or N.

z6 is 1 or 2.

R³, R⁴, and R⁶ are independently hydrogen, —CC1₃, —CBr₃, —CF₃, —CI₃, CHC1₂, —CHBr₂, —CHF₂, —CHI₂, —CH₂C1, —CH₂Br, —CH₂F, —CH₂I, —CN, —OH, —NH₂, —COOH, —CO(C₁-C₆ alkyl), —CONH₂, —OCC1₃, —OCF₃, —OCBr₃, —OCI₃, —OCHC1₂, —OCHBr₂, —OCHI₂, —OCHF₂, —OCH₂ C1, —OCH₂Br, —OCH₂I, —OCH₂F, unsubstituted alkyl, unsubstituted heteroalkyl, unsubstituted cycloalkyl, or unsubstituted heterocycloalkyl.

R⁷, R⁸, R⁹, and R¹⁰ are independently hydrogen or unsubstituted C₁-C₁₀ alkyl.

Ring B is aryl or heteroaryl.

R⁵ is an electrophilic moiety.

R^{1A}, R^{1B}, R^{1C}, R^{1D}, R^{2A}, R^{2B}, R^{2C}, and R^{2D} are independently hydrogen, —CC1₃, —CBr₃, —CF₃, —CI₃, CHC1₂, —CHBr₂, —CHF₂, —CHI₂, —CH₂C1, —CH₂Br, —CH₂F, —CH₂I, —CN, —OH, —NH₂, —COOH, —CONH₂, —OCC1₃, —OCF₃, —OCBr₃, —O CI₃, —OCHC1₂, —OCHBr₂, —OCHI₂, —OCHF₂, —OCH₂C1, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl.

X¹ and X² are independently —F, —Cl, —Br, or —I.

n1 and n2 are independently an integer from 0 to 4.

m1, m2, v1, and v2 are independently 1 or 2.

In embodiments, is provided a compound having the formula:

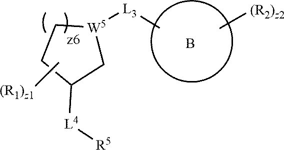

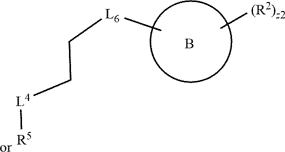

Ring B, R¹, z1, R², z2, R⁵, L³, L⁴, L⁶, z6, and W⁵ are as described herein, including in embodiments. In embodiments, is provided a compound having the formula:

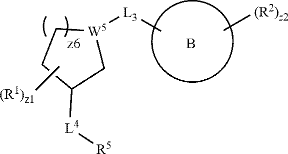

Ring B, R¹, z1, R², z2, R⁵, L³, L⁴, z6, and W⁵ are as described herein, including in embodiments. In embodiments, is provided a compound having the formula:

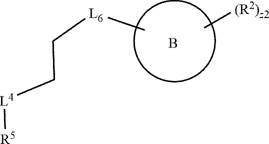

Ring B, R², z2, R⁵, L⁴, and L⁶ are as described herein, including in embodiments. In embodiments, is provided a compound having the formula:

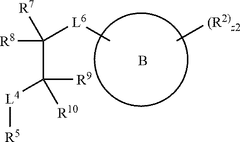

Ring B, R², z2, R⁵, R⁷, R⁸, R⁹, R¹⁰, L⁴, and L⁶ are as described herein, including in embodiments.

In embodiments, R³, R4, and R⁶ are independently hydrogen, —CC1₃, —CBr₃, —CF₃, —CI₃, CHC1₂, —CHBr₂, —CHF₂, —CHI₂, —CH₂C1, —CH₂Br, —CH₂F, —CH₂I, —CN, —OH, —NH₂, —COOH, —COCH₃, —CONH₂, —OCC1₃, —OCF₃, —OCBr₃, —OCI₃, —OCHC1₂, —OCHBr₂, —OCHI₂, —OCHF₂, —OC H₂C1, —OCH₂Br, —OCH₂I, —OCH₂F, unsubstituted alkyl, unsubstituted heteroalkyl, unsubstituted cycloalkyl, or unsubstituted heterocycloalkyl.

In embodiments, R³, R4, and R⁶ are independently hydrogen, —CC1₃, —CBr₃, —CF₃, —CI₃, CHC1₂, —CHBr₂, —CHF₂, —CHI₂, —CH₂C1, —CH₂Br, —CH₂F, —CH₂I, —CN, —OH, —NH₂, —COOH, —CONH₂, —OCC1₃, —OCF₃, —OCBr₃, —O CI₃, —OCHC1₂, —OCHBr₂, —OCHI₂, —OCHF₂, —OCH₂C1, —OCH₂Br, —OCH₂I, —OCH₂F, unsubstituted alkyl, or unsubstituted heteroalkyl.

In embodiments, the electrophilic moiety is a covalent cysteine modifier moiety. In embodiments, R⁵ is a covalent cysteine modifier moiety.

In embodiments, R⁵ is independently

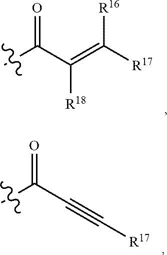

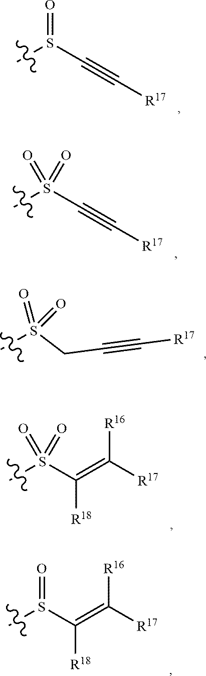

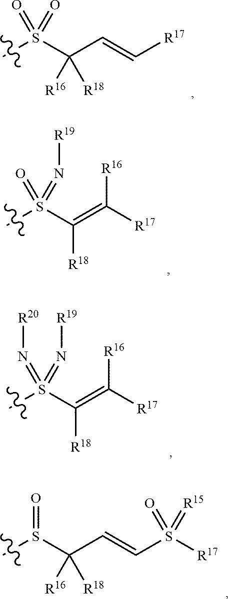

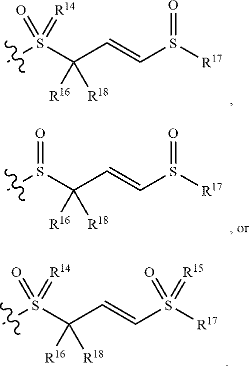

R¹⁴ is independently ═O or ═NR¹⁹.

R¹⁵ is independently ═O or ═NR²⁰.

R¹⁶, R¹⁷, and R¹⁸ are independently hydrogen, oxo, halogen, —CC1₃, —CBr₃, —CF₃, —CI₃, CHC1₂, —CHBr₂, —CHF₂, —CHI₂, —CH₂C1, —CH₂Br, —CH₂F, —CH₂I, —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCC1₃, —OCF₃, —OCBr₃, —OCI₃, —OCHC1₂, —OCHBr₂, —OCHI₂, —OCHF₂, —OCH₂C1, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R¹⁹ and R²⁰ are independently hydrogen, —CC1₃, —CBr₃, —CF₃, —CI₃, CHC1₂, —CHBr₂, —CHF₂, —CHI₂, —CH₂C1, —CH₂Br, —CH₂F, —CH₂I, —CN, —COOH, —CONH₂, —C(O)N(CH₃)₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments is provided a compound having the formula:

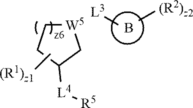

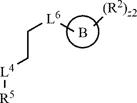

or

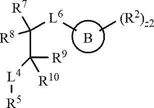

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O) NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents on adjacent carbons may optionally be joined to form a substituted or unsubstituted cycloalkyl, substituted or unsubstituted heterocycloalkyl, substituted or unsubstituted aryl, or substituted or unsubstituted heteroaryl or two R¹ substituents bonded to the same carbon atom may optionally be joined to form a substituted or unsubstituted alkyl or substituted or unsubstituted heterocycloalkyl; z1 is an integer from 0 to 9; R² is independently oxo, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B},—NHC(O)NR^{2A}R^{2B}, —N(O)ₘ₂, —NR^{2A}R^{2B}, —C(O)R^{2C}, —C(O)—OR^{2C}, —C(O) NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R² substituents on adjacent carbons may optionally be joined to form a substituted or unsubstituted cycloalkyl, substituted or unsubstituted heterocycloalkyl, substituted or unsubstituted phenyl, or substituted or unsubstituted heteroaryl; z2 is an integer from 0 to 6; L³ is a bond, —S(O)₂—, —NR³—, —NH—, —O—, —S—, —C(O)—, —C(O)NR³—, —NR³C(O)—, —N(R³)CH₂—, —NR³C(O)NH —, —NHC(O)NR³—, —C(O)O—, —OC(O)—, substituted or unsubstituted alkylene, or substituted or unsubstituted heteroalkylene; L⁴ is a bond, —NH—, —NR4—, or substituted or unsubstituted alkylene; L⁶ is —N(R⁶)—L³— or —C(O)NH—; W⁵ is CH or N; z6 is 1 or 2; R³, R⁴, and R⁶ are independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CO(C₁-C₆ alkyl), —CONH₂, —OCCl₃, —OCF₃, —OCBr₃, —OCI₃, —OCHCl₂, —OCHBr₂, —OCHI₂, —OCHF₂, —OCH₂ Cl, —OCH₂Br, —OCH₂I, —OCH₂F, unsubstituted alkyl, unsubstituted heteroalkyl, unsubstituted cycloalkyl, or unsubstituted heterocycloalkyl; R⁷, R⁸, R⁹, and R¹⁰ are independently hydrogen or unsubstituted C₁-C₁₀ alkyl; Ring B is aryl or heteroaryl; R⁵ is independently

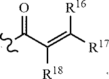

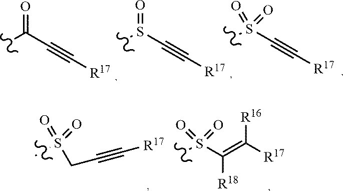

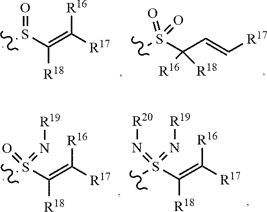

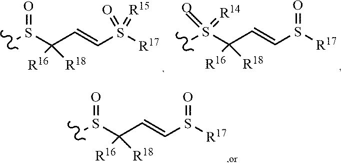

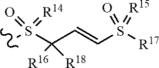

; wherein, R¹⁴ is independently =O or ═NR¹⁹; R¹⁵ is independently =O or =NR²⁰; R¹⁶, R¹⁷, and R¹⁸ are independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹⁹ and R²⁰ are independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —COOH, —CONH₂, —C(O)N(CH₃)₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A}, R^{1B}, R^{1C}, R^{1D}, R^{2A}, R^{2B}, R^{2C}, and R^{2D} are independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; X¹ and X² are independently —F, —Cl, —Br, or —I; n1 and n2 are independently an integer from 0 to 4; and m1, m2, v1, and v2 are independently 1 or 2.

In embodiments, R⁵ is independently

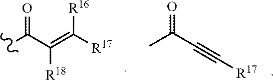

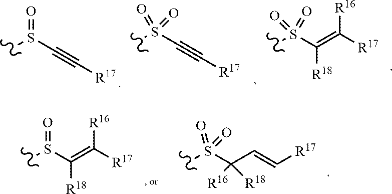

wherein R¹⁶, R¹⁷, and R¹⁸, are as described herein, including in embodiments.

In embodiments, R⁵ is independently

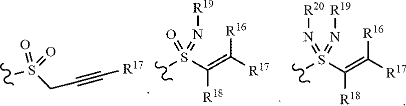

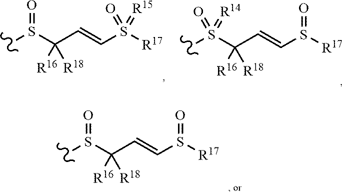

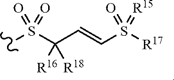

In embodiments is provided a compound having the formula:

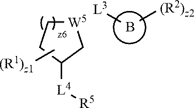

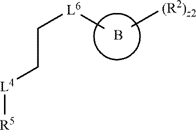

or

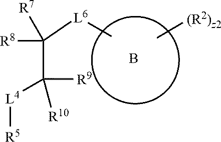

R¹ is independently halogen, -CX¹₃, -CHX¹₂, -CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O) NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl; two adjacent R¹ substituents on adjacent carbons may optionally be joined to form a substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl; or two R¹ substituents bonded to the same carbon atom may optionally be joined to form a substituted or unsubstituted C₃-C₆ cycloalkyl or substituted or unsubstituted 3 to 6 membered heterocycloalkyl; z1 is an integer from 0 to 9; R² is independently oxo, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B},—NHC(O)NR^{2A}R^{2B}, —N(O)ₘ₂, —NR^{2A}R^{2B}, —C(O)R^{2C}, —C(O)—OR^{2C}, —C(O) NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl; two adjacent R² substituents on adjacent carbons may optionally be joined to form a substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl; z2 is an integer from 0 to 6; L³ is a bond, —S(O)₂—, —NR³—, —NH—, —O—, —S—, —C(O)—, —C(O)NR³—, —NR³C(O)—, —N(R³)CH₂—, —NR³C(O)NH —, —NHC(O)NR³—, —C(O)O—, —OC(O)—, substituted or unsubstituted C₁-C₆ alkylene, or substituted or unsubstituted 2 to 6 membered heteroalkylene; L⁴ is a bond, —NH—, —NR4—, or substituted or unsubstituted C₁-C₂ alkylene; L⁶ is —N(R⁶)—L³— or —C(O)NH—; W⁵ is CH or N; z6 is 1 or 2; R³, R⁴, and R⁶ are independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —COCH₃, —CONH₂, —OCCl₃, —OCF₃, —OCBr₃, —OCI₃, —OCHCl₂, —OCHBr₂, —OCHI₂, —OCHF₂, —OC H₂Cl, —OCH₂Br, —OCH₂I, —OCH₂F, unsubstituted C₁-C₆ alkyl, unsubstituted 2 to 6 membered heteroalkyl, unsubstituted cycloalkyl, or unsubstituted heterocycloalkyl; R⁷, R⁸, R⁹, and R¹⁰ are independently hydrogen or unsubstituted C₁-C₁₀ alkyl; Ring B is C₆-C₁₀ aryl, or 5 to 10 membered heteroaryl; R⁵ is independently

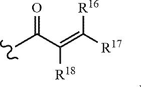

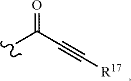

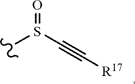

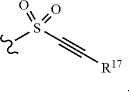

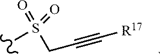

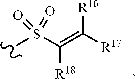

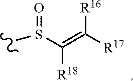

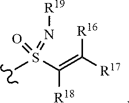

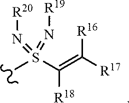

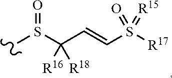

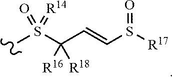

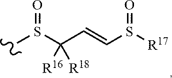

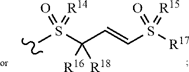

R¹⁴ is independently =O or ═NR¹⁹; R¹⁵ is independently =O or ═NR²⁰; R¹⁶, R¹⁷, and R¹⁸ are independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl; R¹⁹ and R²⁰ are independently hydrogen, —CCl₃, —CBr₃, —CF₃, —CI₃, CHCl₂, —CHBr₂, —CHF₂, —CHI₂, —CH₂Cl, —CH₂Br, —CH2F, —CH₂I, —CN, —COOH, —CONH₂, —C(O)N(CH₃)₂, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl; R^{1A}, R^{1B}, R^{1C}, R^{1D}, R^{2A}, R^{2B}, R^{2C}, and R^{2D} are independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be j oined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; X¹ and X² are independently —F, —Cl, —Br, or —I; n1 and n2 are independently an integer from 0 to 4; and m1, m2, v1, and v2 are independently 1 or 2.

In embodiments is provided a compound having the formula:

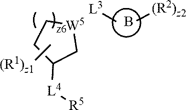

or

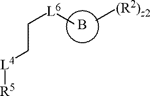

R^{1 is} independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O) NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl; two adjacent R¹ substituents on adjacent carbons may optionally be joined to form a substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl; or two R¹ substituents bonded to the same carbon atom may optionally be joined to form a substituted or unsubstituted C₃-C₆ cycloalkyl or substituted or unsubstituted 3 to 6 membered heterocycloalkyl; z1 is an integer from 0 to 9; R² is independently oxo, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR²CNR^{2A}R^{2B},—NHC(O)NR^{2A}R^{2B}, —N(O)ₘ₂, —NR^{2A}R^{2B}, —C(O)R^{2C}, —C(O)—OR^{2C}, —C(O) NR2AR2B, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl; two adjacent R² substituents on adjacent carbons may optionally be joined to form a substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl; z2 is an integer from 0 to 6; L³ is a bond, —S(O)₂—, —NR³—, —NH—, —O—, —S—, —C(O)—, —C(O)NR³—, —NR³C(O)—, —N(R³)CH₂—, —NR³C(O)NH —, —NHC(O)NR³—, —C(O)O—, —OC(O)—, substituted or unsubstituted C₁-C₆ alkylene, or substituted or unsubstituted 2 to 6 membered heteroalkylene; L⁴ is a bond, —NH—, —NR4—, or substituted or unsubstituted C₁-C₂ alkylene; L⁶ is —N(R⁶)—L³— or —C(O)NH—; W⁵ is CH or N; z6 is 1 or 2; R³, R⁴, and R⁶ are independently hydrogen, —CCl₃, —CBr₃, —CF₃, —CI₃, CHCl₂, —CHBr₂, —CHF₂, —CHI₂, CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, unsubstituted C₁-C₆ alkyl, or unsubstituted 2 to 6 membered heteroalkyl; Ring B is C₆-C₁₀ aryl, or 5 to 10 membered heteroaryl; R⁵ is independently

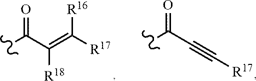

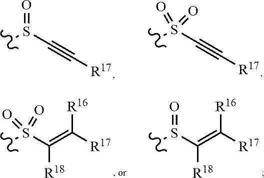

R¹⁶, R¹⁷, and R¹⁸ are independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl; R^{1A}, R^{1B}, R^{1C}, R^{1D}, R^{2A}, R^{2B}, R^{2C}, and R^{2D} are independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; X¹ and X² are independently —F, —Cl, —Br, or —I; n1 and n2 are independently an integer from 0 to 4; and m1, m2, v1, and v2 are independently 1 or 2.

In embodiments is provided a compound having the formula:

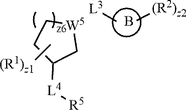

or

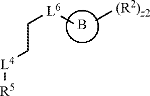

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O) NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl; two adjacent R¹ substituents on adjacent carbons may optionally be joined to form a substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl; or two R¹ substituents bonded to the same carbon atom may optionally be joined to form a substituted or unsubstituted C₃-C₆ alkyl or substituted or unsubstituted 3 to 6 membered heterocycloalkyl; z1 is an integer from 0 to 9; R² is independently oxo, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B},—NHC(O)NR^{2A}R^{2B}, —N(O)ₘ₂, —NR^{2A}R^{2B}, —C(O)R^{2C}, —C(O)—OR^{2C}, —C(O) NR2AR2B, —OR^{2D}, —NR^{2A}SO₂R^{2D}, —NR^{2A}C(O)R^{2C}, ₋NR^{2A}C(O)OR^{2C}, —NR^{2A}OR^{2C}, —SF₅, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl; two adjacent R² substituents on adjacent carbons may optionally be joined to form a substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl; z2 is an integer from 0 to 6; L³ is a bond, —S(O)₂—, —NR³—, —NH—, —O—, —S—, —C(O)—, —C(O)NR³—, —NR³C(O)—, —N(R³)CH₂—, —NR³C(O)NH —, —NHC(O)NR³—, —C(O)O—, —OC(O)—, substituted or unsubstituted C₁-C₆ alkylene, or substituted or unsubstituted 2 to 6 membered heteroalkylene; L⁴ is a bond, —NH—, —NR4—, or substituted or unsubstituted C₁-C₂ alkylene; L⁶ is —N(R⁶)—L³— or —C(O)NH—; W⁵ is CH or N; z6 is 1 or 2; R³, R⁴, and R⁶ are independently hydrogen, —CCl₃, —CBr₃, —CF₃, —CI₃, CHCl₂, —CHBr₂, —CHF₂, —CHI₂, CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, unsubstituted C₁-C₆ alkyl, or unsubstituted 2 to 6 membered heteroalkyl; Ring B is C₆-C₁₀ aryl, or 5 to 10 membered heteroaryl; R⁵ is independently

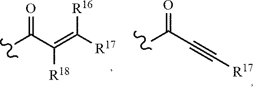

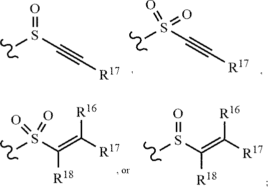

R¹⁶, R¹⁷, and R¹⁸ are independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl; R^{1A}, R^{1B}, R^{1C}, R^{1D}, R^{2A}, R^{2B}, R^{2C,} and R^{2D} are independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; X¹ and X² are independently —F, —Cl, —Br, or —I; n1 and n2 are independently an integer from 0 to 4; and m1, m2, v1, and v2 are independently 1 or 2.

In embodiments, the compound has the formula

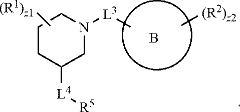

wherein R¹, R², R⁵, z1, z2, L³, L⁴, and Ring B are as described herein, including in embodiments.

In embodiments, the compound has the formula:

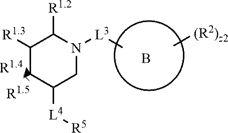

R^{1.2}, R^{1.3}, R^{1.4}, and R^{1.5} can be hydrogen or any value of R¹, as described herein, including embodiments..

In embodiments, R^{1.2} and R^{1.3} are independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, or substituted or unsubstituted heteroalkyl; or R^{1.2} and R^{1.3} substituents on adjacent carbons may optionally be joined to form a substituted or unsubstituted phenyl, or substituted or unsubstituted heteroaryl.

In embodiments, R^{1.2} and R^{1.3} are independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted C₁-C₆ alkyl, or substituted or unsubstituted 2 to 6 membered heteroalkyl; or R^{1.2} and R^{1.3} substituents on adjacent carbons may optionally be joined to form a substituted or unsubstituted phenyl, or substituted or unsubstituted 5 to 6 membered heteroaryl.

In embodiments, R^{1.4} and R^{1.5} are independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, or substituted or unsubstituted heteroalkyl.

In embodiments, R^{1.4} and R^{1.5} are independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted C₁-C₆ alkyl, or substituted or unsubstituted 2 to 6 membered heteroalkyl.

In embodiments, the compound has the formula:

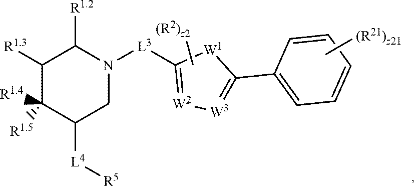

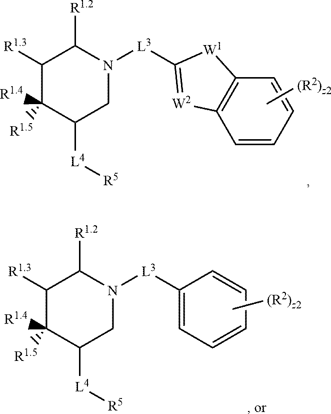

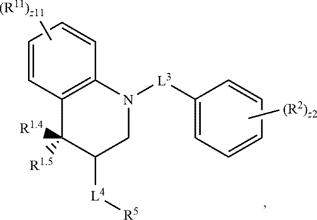

wherein R^{1.2}, R^{1.3}, R^{1.4}, R^{1.5}, R², z2, R⁵, L³, and L⁴are as described herein, including in embodiments.

W¹ is independently —O—, —NH—, or —NR²—.

W² and W³ are independently ═N—, ═CH—, or ═CR²—.

R¹¹ is independently oxo, halogen, —CX¹¹₃, —CHX¹¹₂, —CH₂X¹¹, —OCX¹¹₃, —OCH₂X¹¹, —OCHX¹¹₂, —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R²¹ is independently oxo, halogen, —CX²¹₃, —CHX²¹₂, —CH₂X²¹, —OCX²¹₃, —OCH₂X²¹, —OCHX²¹₂, —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

X¹¹ and X²¹ are independently —F, —Cl, —Br, or —I.

z11 is an integer from 0 to 4.

z21 is an integer from 0 to 5.

In embodiments, R² is independently oxo, halogen, —CX²₃, —CHX²₂, —CH₂X², —OCX²₃, —OCH₂X², —OCHX²₂, —CN, —OH, —NH₂, -COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, SF₅, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl.

In embodiments, R² is independently halogen, —OCX²₃, —OCH₂X², —OCHX²₂, unsubstituted C₁-C₃ alkyl, or unsubstituted 5 to 6 membered heteroaryl; R¹¹ is independently halogen, —OCX¹¹₃, —OCH₂X¹¹, -OCHX¹¹₂, unsubstituted C₁-C₃ alkyl, or unsubstituted 5 to 6 membered heteroaryl; and R²¹ is independently halogen, —OCX²¹₃, —OCH₂X²¹, —OCHX²¹₂, unsubstituted C₁-C₃ alkyl, or unsubstituted 5 to 6 membered heteroaryl.

In embodiments, R^{1.2}, R^{1.3}, R^{1.4} and R^{1.5} are independently hydrogen or halogen.

In embodiments, the compound has the formula:

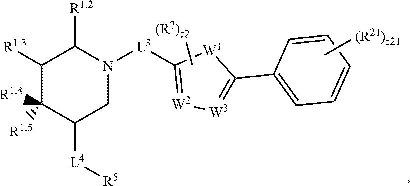

wherein R^{1.2}, R^{1.3} R^{1.4}, R^{1.5}, R², z2, R²¹, z21, R⁵, L³, L⁴, W¹, W², and W³ are as described herein, including in embodiments. In embodiments, the compound has the formula:

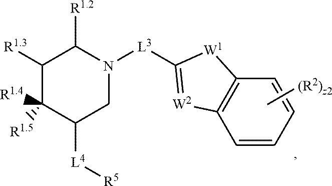

wherein R^{1.2}, R^{1.3}, R^{1.4}, R^{1.5}, R², z2, R⁵, L³, L⁴, W¹, and W², are as described herein, including in embodiments. In embodiments, the compound has the formula:

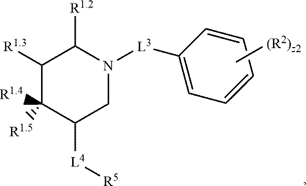

wherein R^{1.2}, R^{1.3}, R^{1.4}, R^{1.5}, R², z2, R⁵, L³, and L⁴ are as described herein, including in embodiments. In embodiments, the compound has the formula:

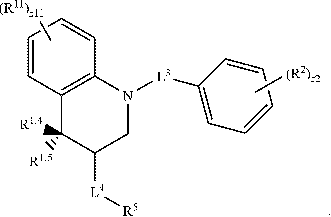

wherein R^{1.4}, R^{1.5}, R², z2, R⁵, R¹¹, z11, L³, and L⁴ are as described herein, including in embodiments.

In embodiments, L³ is —C(O)—, —CH₂—, —C(O)NH—, —NHC(O)—, —NHCH₂—, —CH₂CH₂NH—, —C(O)CH₂NH—, or —CH₂C(O)NH—.

In embodiments, L⁴ is a bond, —NH—, or —CH₂—. In embodiments, L⁴ is —NH—, —NR⁴—, or —CH₂—. In embodiments, L⁴ is a bond. In embodiments, L⁴ is —NH—. In embodiments, L⁴ is —NR⁴—. In embodiments, L⁴ is —CH₂—.

In embodiments, R⁵ is independently

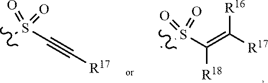

wherein R¹⁶, R¹⁷, and R¹⁸, are as described herein, including in embodiments.

In embodiments, R¹⁶, R¹⁷, and R¹⁸ are independently hydrogen, —C(O)N(CH₃)₂, or unsubstituted C₁-C₃ alkyl.

In embodiments, R⁵ is independently

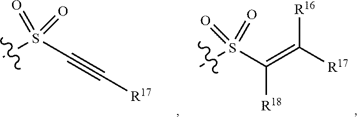

or

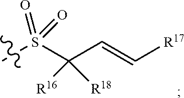

wherein R¹⁶, R¹⁷, and R¹⁸ are independently hydrogen, —C(O)N(CH₃)₂, or unsubstituted C₁-C₃ alkyl.

In embodiments, R⁵ is independently

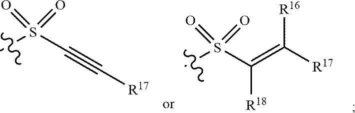

wherein R¹⁶, R¹⁷, and R¹⁸ are as described herein, including in embodiments.

In embodiments, the compound has the formula:

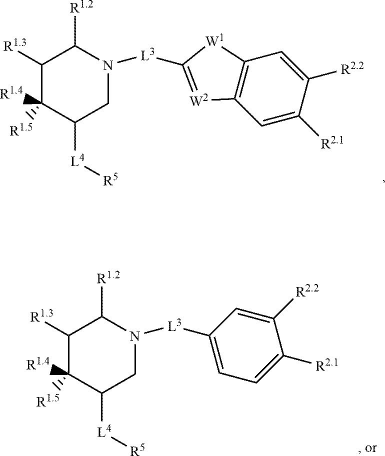

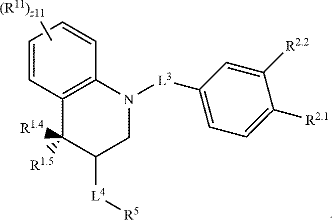

R^{1.2}, R^{1.3}, R^{1.4}, R^{1.5}, R⁵, R¹¹, z11, W¹, W², L³, and L⁴ are as described herein, including in embodiments. R^{2.1} and R^{2.2} are independently hydrogen or any value of R², as described herein, including in embodiments.

In embodiments, the compound has the formula:

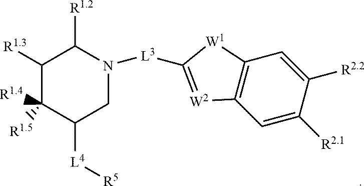

R^{1.2}, R^{1.3}, R^{1.4}, R^{1.5}, R^{2.1}, R^{2.2}, R⁵, W¹, W², L³, and L⁴ are as described herein, including in embodiments. In embodiments, the compound has the formula:

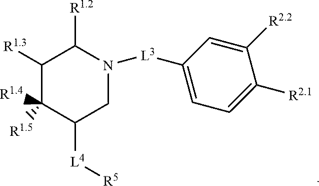

R^{1.2}, R^{1.3}, R^{1.4}, R^{1.5}, R^{2.1}, R^{2.2}, R⁵, L³, and L⁴ are as described herein, including in embodiments. In embodiments, the compound has the formula:

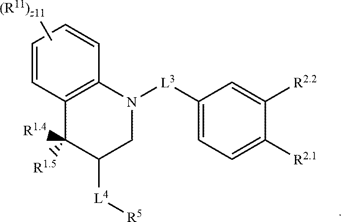

R^{1.4}, R^{1.5}, R^{2.1}, R^{2.2}, R⁵, R¹¹, z11, L³, and L⁴ are as described herein, including in embodiments.

In embodiments, R^{2.1} is independently hydrogen, —OCX²₃, or unsubstituted 5 to 6 membered heteroaryl.

In embodiments, R^{2.2} is independently hydrogen or halogen.

In embodiments, R¹¹ is independently halogen.

In embodiments, the compound has the formula:

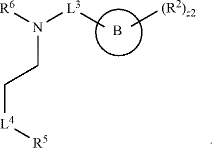

R², z2, R⁵, R⁶, L³, and L⁴ are as described herein, including in embodiments.

In embodiments, the compound has the formula:

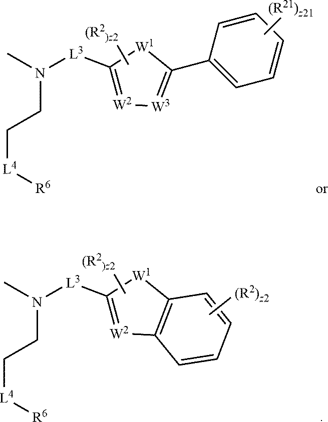

R², z2, R²¹, z21, R⁵, W¹, W², W³, L³, and L⁴ are as described herein, including in embodiments. In embodiments, the compound has the formula:

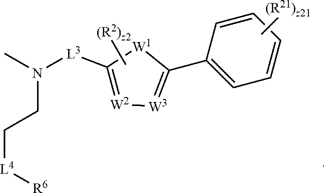

R², z2, R²¹, z21, R⁵, W¹, W², W³, L³, and L⁴ are as described herein, including in embodiments. In embodiments, the compound has the formula:

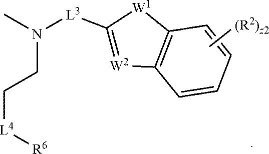

R², z2, R⁵, W¹, W², L³, and L⁴ are as described herein, including in embodiments.

In embodiments, W¹ is independently —O—, —NH—, or —NR²—.

In embodiments, R² is independently halogen, —OCX²₃, —OCH₂X², —OCHX²₂, unsubstituted C₁-C₃ alkyl, or unsubstituted 5 to 6 membered heteroaryl.

In embodiments, R²¹ is independently halogen, —OCX²₃, —OCH₂X, —OCHX², unsubstituted C₁-C₃ alkyl, or unsubstituted 5 to 6 membered heteroaryl.

In embodiments, L³ is —C(O)— or —CH₂—.

In embodiments, L⁴ is —NH—, —NR⁴—, or —CH₂—.

In embodiments, R¹⁶ is independently hydrogen, —C(O)N(CH₃)₂, or unsubstituted C₁-C₃ alkyl. In embodiments, R¹⁷ is independently hydrogen, —C(O)N(CH₃)₂, or unsubstituted C₁-C₃ alkyl. In embodiments, R¹⁸ is independently hydrogen, —C(O)N(CH₃)₂, or unsubstituted C₁-C₃ alkyl.

In embodiments, R⁵ is independently

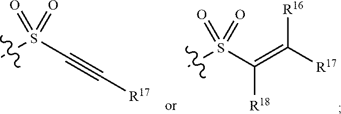

wherein R¹⁶, R¹⁷, and R¹⁸ are independently hydrogen, —C(O)N(CH₃)₂, or unsubstituted C₁-C₃ alkyl.

In embodiments, the compound has the formula

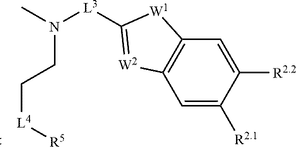

or

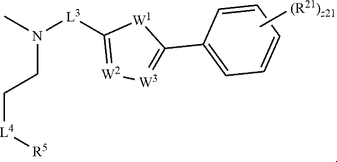

R^{2.1}, R^{2.2}, R⁵, R²¹, z21, W¹, W², W³, L³, and L⁴ are as described herein, including in embodiments.

In embodiments, the compound has the formula:

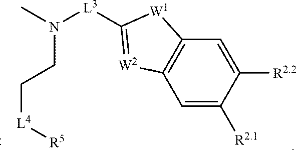

R^{2.1}, R^{2.2}, R⁵, W¹, W², L³, and L⁴ are as described herein, including in embodiments.

In embodiments, the compound has the formula:

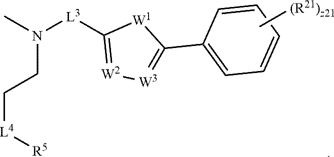

R⁵, R²¹, z21, W¹, W², W³, L³, and L⁴ are as described herein, including in embodiments.

In embodiments, the compound has the formula:

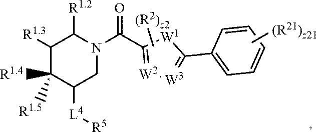

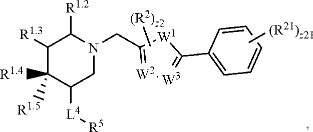

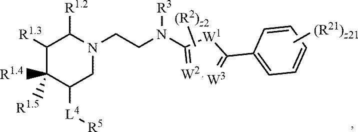

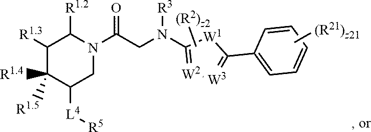

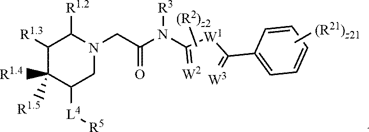

R^{1.2}, R^{1.3}, R^{1.4} , R^{1.5}, R², R³, R⁵, R²¹, L⁴, W¹, W², W³, z2 and z21 are as described herein, including in embodiments.

In embodiments, the compound has the formula:

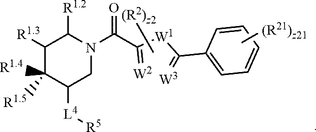

R^{1.2}, R^{1.3}, R^{1.4}, R^{1.5}, R², R⁵, R²¹, L⁴, W¹, W², W³, z2 and z21 are as described herein, including in embodiments. In embodiments, the compound has the formula:

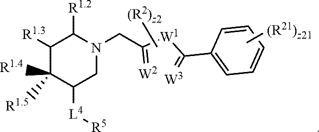

R^{1.2}, R^{1.3}, R^{1.4}, R^{1.5}, R², R⁵, R²¹, L⁴, W¹, W², W³, z2 and z21 are as described herein, including in embodiments. In embodiments, the compound has the formula:

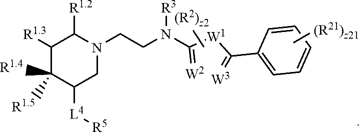

R^{1.2}, R^{1.3}, R^{1.4}, R^{1.5}, R², R³, R⁵, R²¹, L⁴, W¹, W², W³, z2 and z21 are as described herein, including in embodiments. In embodiments, the compound has the formula:

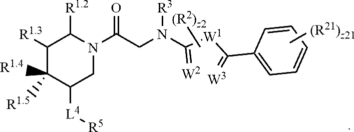

R^{1.2}, R^{1.3}, R^{1.4}, R^{1.5}, R², R³, R⁵, R²¹, L⁴, W¹, W², W³, z2 and z21 are as described herein, including in embodiments. In embodiments, the compound has the formula:

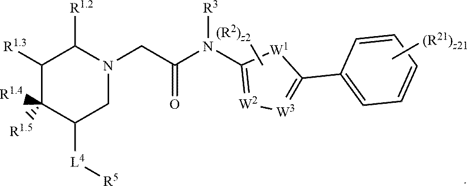

R^{1.2}, R^{1.3}, R^{1.4}, R^{1.5}, R², R³, R⁵, R²¹, L⁴, W¹, W², W³, z2 and z21 are as described herein, including in embodiments.

In embodiments, the compound has the formula:

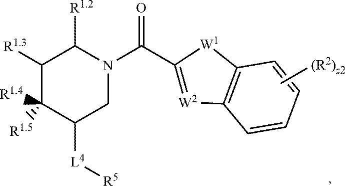

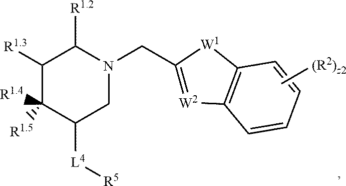

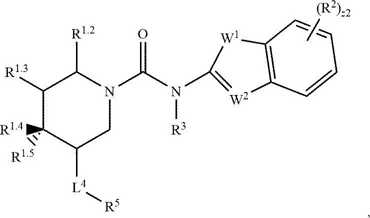

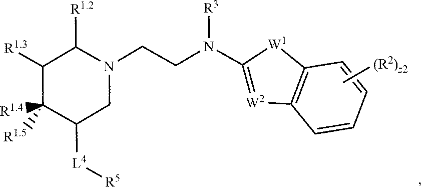

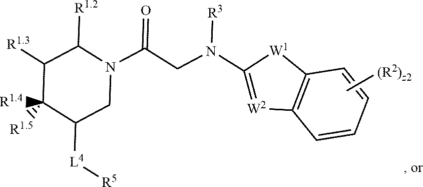

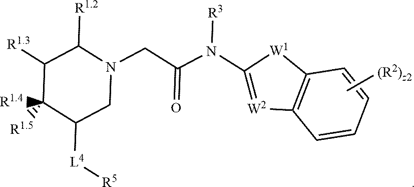

R^{1.2}, R^{1.3}, R^{1.4}, R^{1.5}, R², R³, R⁵, L⁴, W¹, W², and z2 are as described herein, including in embodiments.

In embodiments, the compound has the formula:

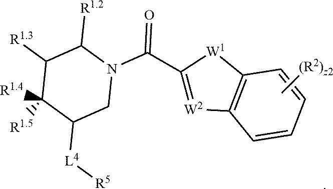

R^{1.2}, R^{1.3}, R^{1.4}, R^{1.5}, R², R⁵, L⁴, W¹, W², and z2 are as described herein, including in embodiments. In embodiments, the compound has the formula:

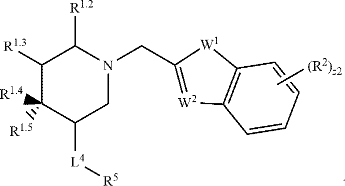

R^{1.2}, R^{1.3}, R^{1.4}, R^{1.5}, R², R⁵, L⁴, W¹, W², and z2 are as described herein, including in embodiments. In embodiments, the compound has the formula:

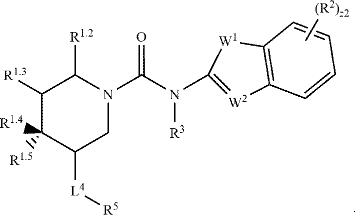

R^{1.2}, R^{1.3}, R^{1.4}, R^{1.5} , R², R³, R⁵, L⁴, W¹, W², and z2 are as described herein, including in embodiments. In embodiments, the compound has the formula:

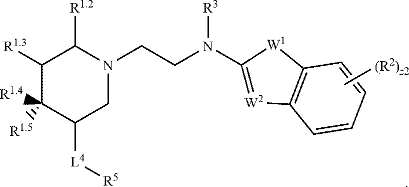

R^{1.2}, R^{1.3}, R^{1.4}, R^{1.5}, R², R³, R⁵, L⁴, W¹, W², and z2 are as described herein, including in embodiments. In embodiments, the compound has the formula:

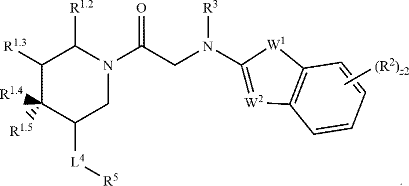

R^{1.2}, R^{1.3}, R^{1.4}, R^{1.5}, R², R³, R⁵, L⁴, W¹, W², and z2 are as described herein, including in embodiments. In embodiments, the compound has the formula:

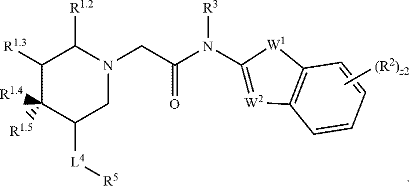

R^{1.2}, R^{1.3}, R^{1.4}, R^{1.5}, R², R³, R⁵, L⁴, W¹, W², and z2 are as described herein, including in embodiments.

In embodiments, the compound has the formula:

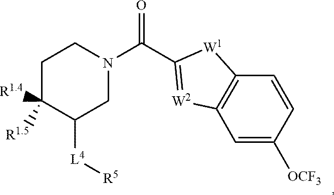

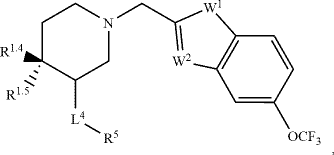

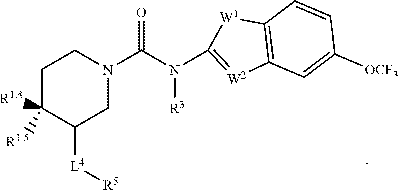

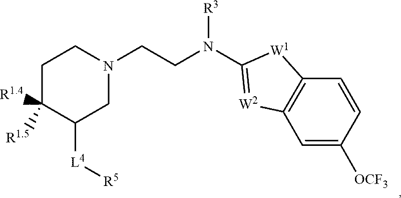

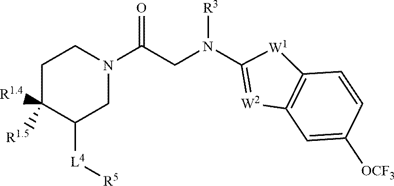

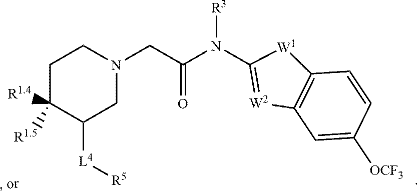

R^{1.4}, R^{1.5}, R³, R⁵, L⁴, W¹, and W² are as described herein, including in embodiments.

In embodiments, the compound has the formula:

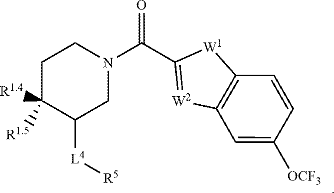

R^{1.4}, R^{1.5}, R⁵, L⁴, W¹, and W² are as described herein, including in embodiments. In embodiments, the compound has the formula:

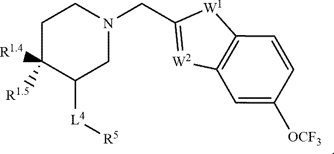

R^{1.4}, R^{1.5}, R⁵, L⁴, W¹, and W² are as described herein, including in embodiments. In embodiments, the compound has the formula:

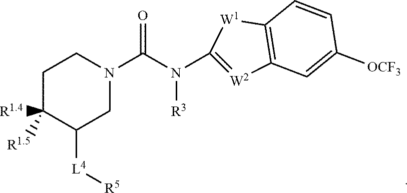

R^{1.4}, R^{1.5}, R³, R⁵, L⁴, W¹, and W² are as described herein, including in embodiments. In embodiments, the compound has the formula:

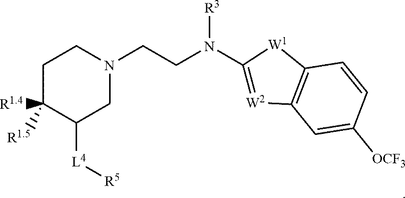

R^{1.4}, R^{1.5}, R³, R⁵, L⁴, W¹, and W² are as described herein, including in embodiments. In embodiments, the compound has the formula:

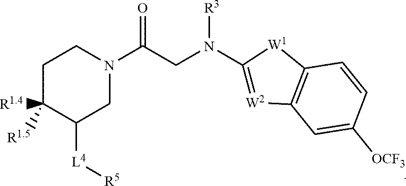

R^{1.4}, R^{1.5}, R³, R⁵, L⁴, W¹, and W² are as described herein, including in embodiments. In embodiments, the compound has the formula:

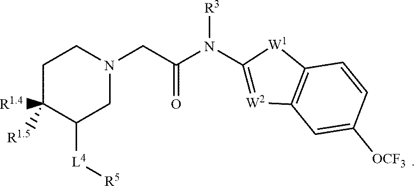

R^{1.4}, R^{1.5}, R³, R⁵, L⁴, W¹, and W² are as described herein, including in embodiments.

In embodiments, the compound has the formula:

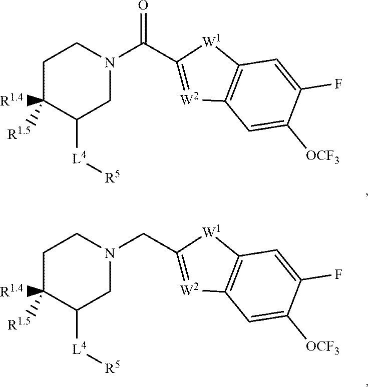

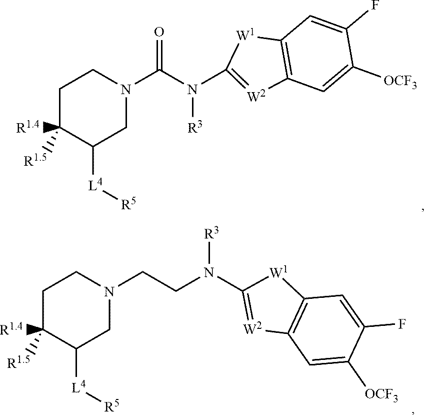

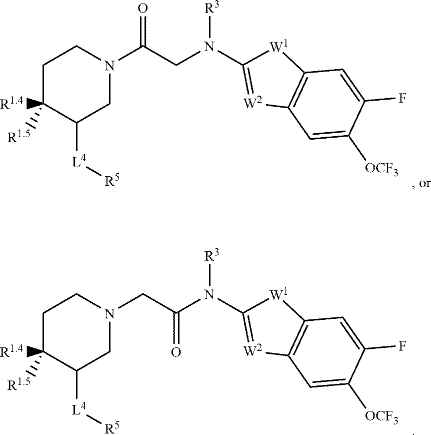

R^{1.4}, R^{1.5}, R³, R⁵, L⁴, W¹, and W² are as described herein, including in embodiments.

In embodiments, the compound has the formula:

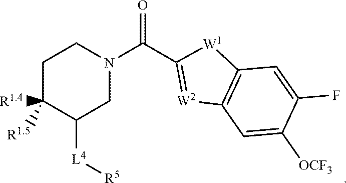

R^{1.4}, R^{1.5}, R⁵, L⁴, W¹, and W² are as described herein, including in embodiments. In embodiments, the compound has the formula:

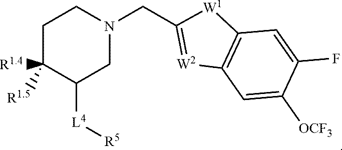

R^{1.4}, R^{1.5}, R⁵, L⁴, W¹, and W² are as described herein, including in embodiments. In embodiments, the compound has the formula:

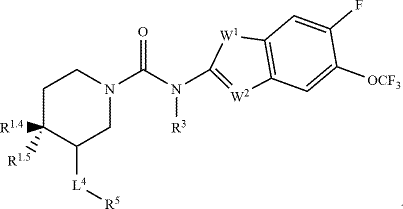

R^{1.4}, R^{1.5}, R³, R⁵, L⁴, W¹, and W² are as described herein, including in embodiments. In embodiments, the compound has the formula:

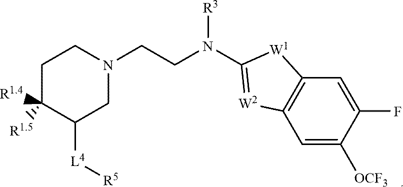

R^{1.4}, R^{1.5}, R³, R⁵, L⁴, W¹, and W² are as described herein, including in embodiments. In embodiments, the compound has the formula:

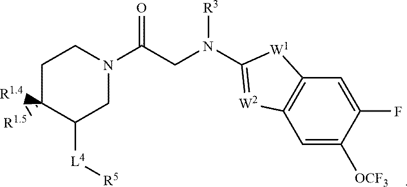

R^{1.4}, R^{1.5}, R³, R⁵, L⁴, W¹, and W² are as described herein, including in embodiments. In embodiments, the compound has the formula:

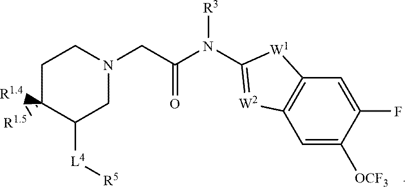

R^{1.4}, R^{1.5}, R³, R⁵, L⁴, W¹, and W² are as described herein, including in embodiments.

In embodiments, the compound has the formula:

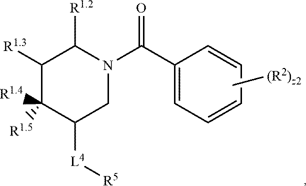

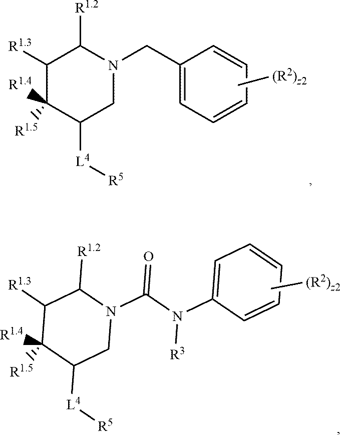

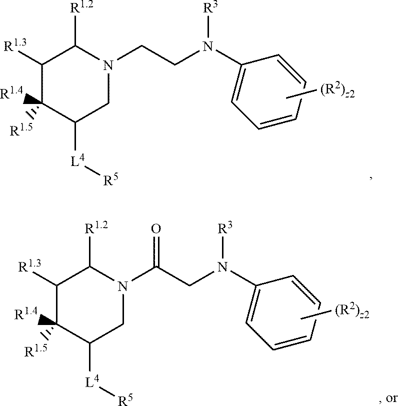

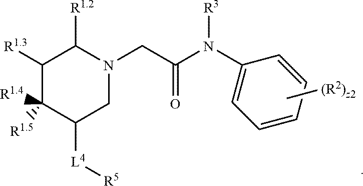

R^{1.2}, R^{1.3}, R^{1.4}, R^{1.5}, R², R³, R⁵, L⁴, and z2 are as described herein, including in embodiments.

In embodiments, the compound has the formula

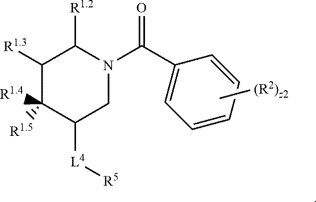

R^{1.2}, R^{1.3}, R^{1.4}, R^{1.5}, R², R⁵, L⁴, and z2 are as described herein, including in embodiments. In embodiments, the compound has the formula:

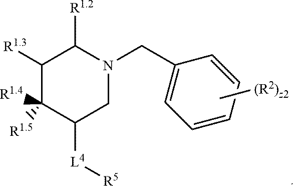

R^{1.2}, R^{1.3}, R^{1.4}, R^{1.5}, R², R⁵, L⁴, and z2 are as described herein, including in embodiments. In embodiments, the compound has the formula:

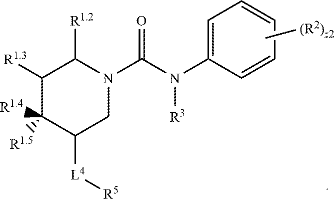

R^{1.2}, R^{1.3}, R^{1.4}, R^{1.5}, R², R³, R⁵, L⁴, and z2 are as described herein, including in embodiments. In embodiments, the compound has the formula:

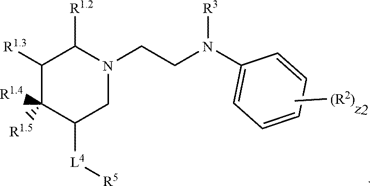

R^{1.2}, R^{1.3}, R^{1.4}, R^{1.5}, R², R³, R⁵, L⁴, and z2 are as described herein, including in embodiments. In embodiments, the compound has the formula:

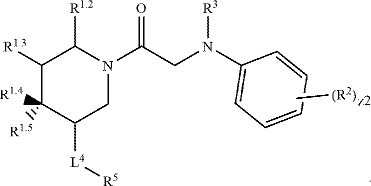

R^{1.2}, R^{1.3}, R^{1.4}, R^{1.5}, R², R³, R⁵, L⁴, and z2 are as described herein, including in embodiments. In embodiments, the compound has the formula:

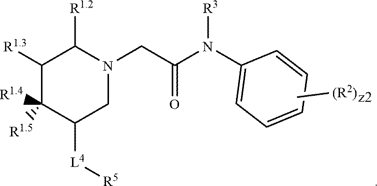

R^{1.2}, R^{1.3}, R^{1.4}, R^{1.5}, R², R³, R⁵, L⁴, and z2 are as described herein, including in embodiments.

In embodiments, the compound has the formula:

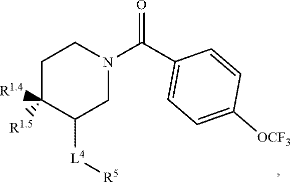

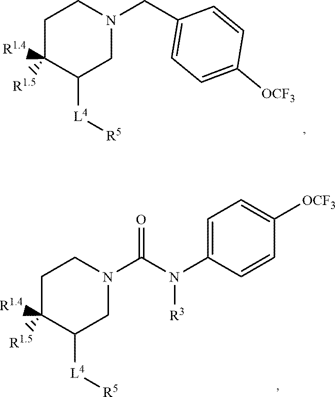

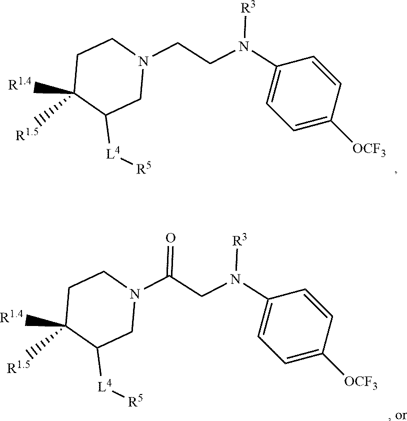

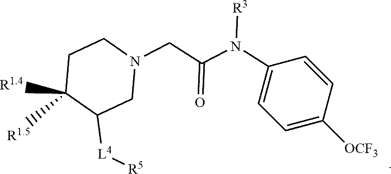

R^{1.4}, R^{1.5}, R³, R⁵, and L⁴ are as described herein, including in embodiments.

In embodiments, the compound has the formula:

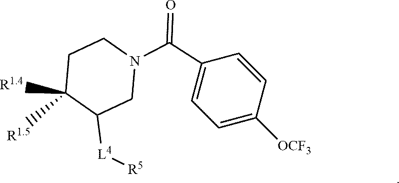

R^{1.4}, R^{1.5}, R⁵, and L⁴ are as described herein, including in embodiments. In embodiments, the compound has the formula:

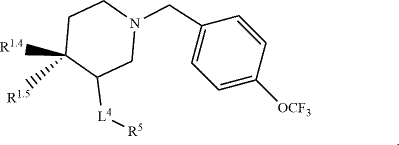

R^{1.4}, R^{1.5}, R³, R⁵, and L⁴ are as described herein, including in embodiments. In embodiments, the compound has the formula:

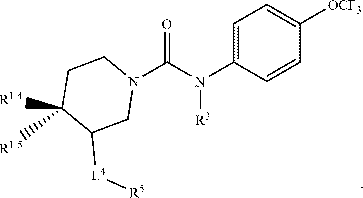

R^{1.4}, R^{1.5}, R³, R⁵, and L⁴ are as described herein, including in embodiments. In embodiments, the compound has the formula:

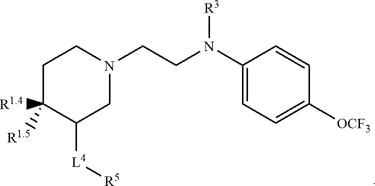

R^{1.4}, R^{1.5}, R³, R⁵, and L⁴ are as described herein, including in embodiments. In embodiments, the compound has the formula:

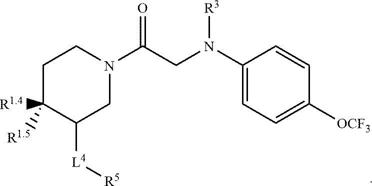

R^{1.4}, R^{1.5}, R³, R⁵, and L⁴ are as described herein, including in embodiments. In embodiments, the compound has the formula:

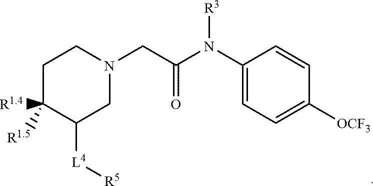

R^{1.4}, R^{1.5}, R³, R⁵, and L⁴ are as described herein, including in embodiments.

In embodiments, the compound has the formula:

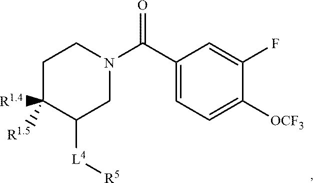

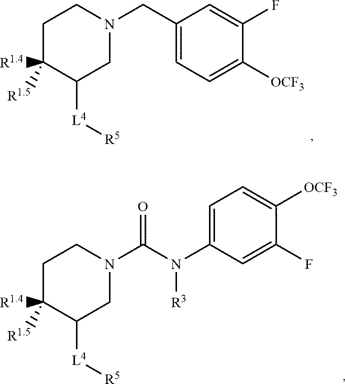

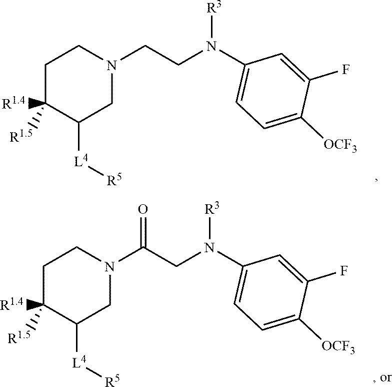

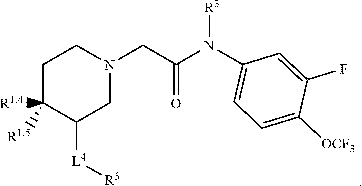

R^{1.4}, R^{1.5}, R³, R⁵, and L⁴ are as described herein, including in embodiments.

In embodiments, the compound has the formula:

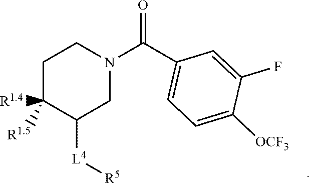

R^{1.4}, R^{1.5}, R⁵, and L⁴ are as described herein, including in embodiments. In embodiments, the compound has the formula:

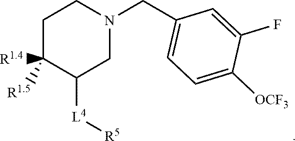

R^{1.4}, R^{1.5}, R⁵, and L⁴ are as described herein, including in embodiments. In embodiments, the compound has the formula:

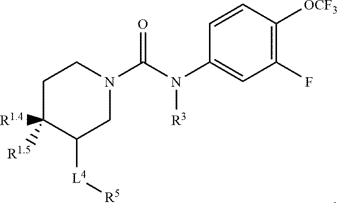

R^{1.4}, R^{1.5}, R³, R⁵, and L⁴ are as described herein, including in embodiments. In embodiments, the compound has the formula:

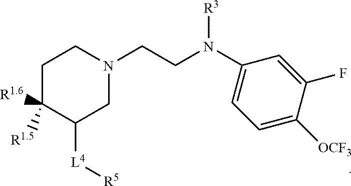

R^{1.4}, R^{1.5}, R³, R⁵, and L⁴ are as described herein, including in embodiments. In embodiments, the compound has the formula:

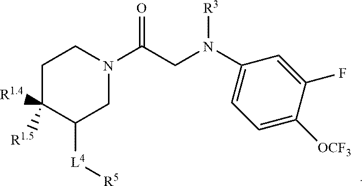

R^{1.4}, R^{1.5}, R³, R⁵, and L⁴ are as described herein, including in embodiments. In embodiments, the compound has the formula:

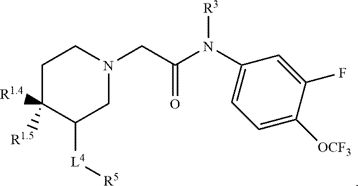

R^{1.4}, R^{1.5}, R³, R⁵, and L⁴ are as described herein, including in embodiments.

In embodiments, the compound has the formula:

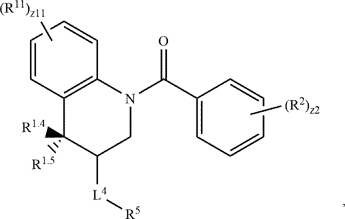

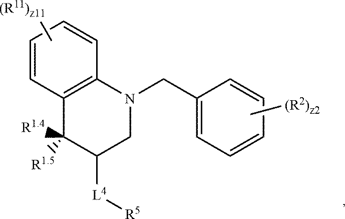

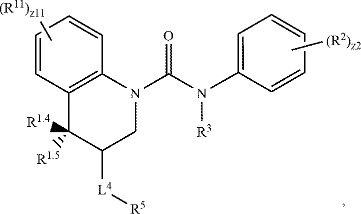

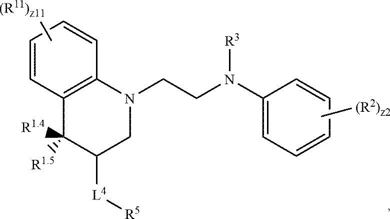

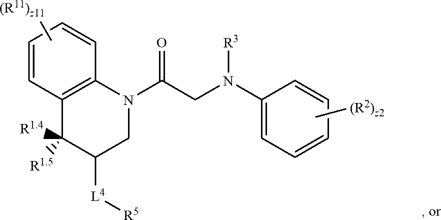

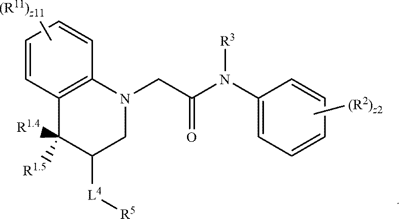

R^{1.4}, R^{1.5}, R², R³, R⁵, R¹¹, L⁴, z2, and z11 are as described herein, including in embodiments.

In embodiments, the compound has the formula:

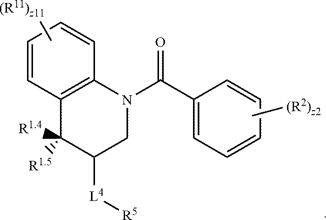

R^{1.4}, R^{1.5}, R², R⁵, R¹¹, L⁴, z2, and z11 are as described herein, including in embodiments. In embodiments, the compound has the formula:

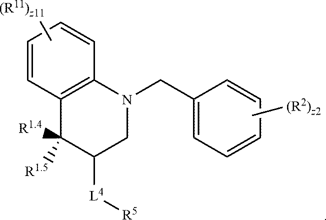

R^{1.4}, R^{1.5}, R², R⁵, R¹¹, L⁴, z2, and z11 are as described herein, including in embodiments. In embodiments, the compound has the formula:

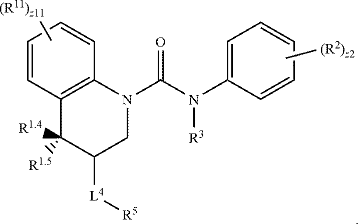

R^{1.4}, R^{1.5}, R², R³, R⁵, R¹¹, L⁴, z2, and z11 are as described herein, including in embodiments. In embodiments, the compound has the formula:

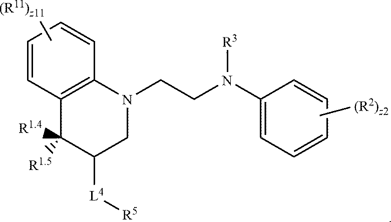

R^{1.4}, R^{1.5}, R², R³, R⁵, R¹¹, L⁴, z2, and z11 are as described herein, including in embodiments. In embodiments, the compound has the formula:

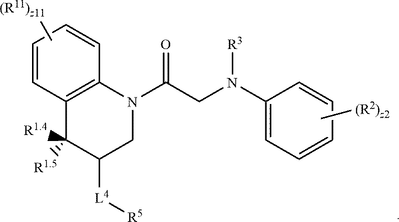

R^{1.4}, R^{1.5}, R², R³, R⁵, R¹¹, L⁴, z2, and z11 are as described herein, including in embodiments. In embodiments, the compound has the formula:

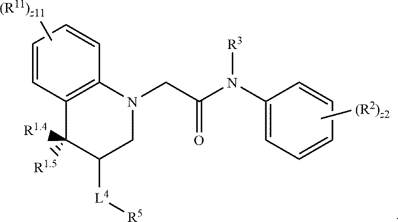

R^{1.4}, R^{1.5}, R², R³, R⁵, R¹¹, L⁴, z2, and z11 are as described herein, including in embodiments.

In embodiments, the compound has the formula:

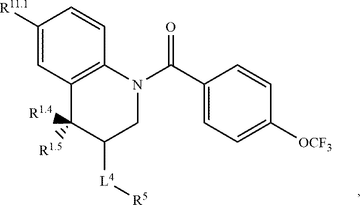

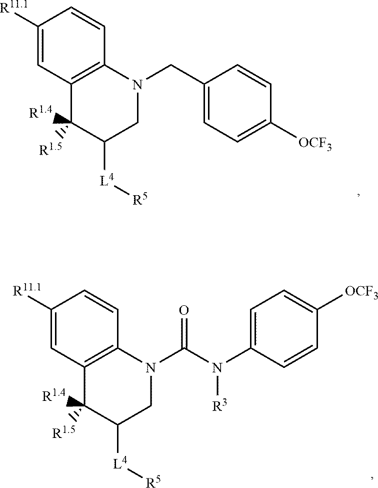

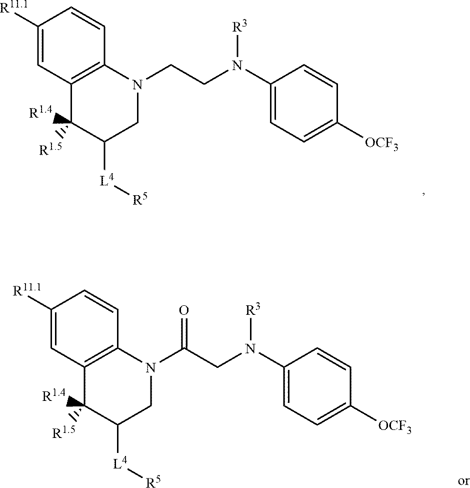

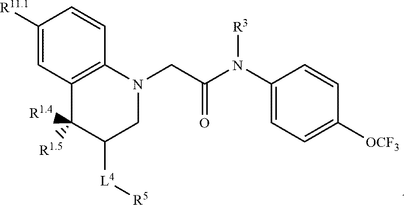

R^{1.4}, R^{1.5}, R³, R⁵, and L⁴ are as described herein, including in embodiments. R^{11.1} is independently hydrogen or any value of R¹¹, as described herein, including in embodiments.

In embodiments, the compound has the formula:

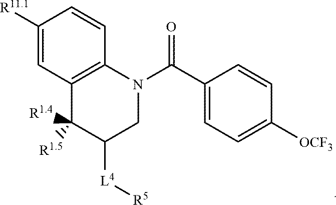

R^{1.4}, R^{1.5}, R⁵, R^{11.1}, and L⁴ are as described herein, including in embodiments. In embodiments, the compound has the formula:

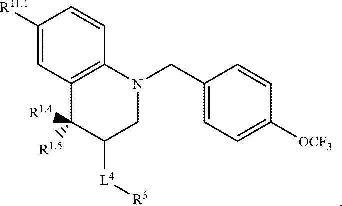

R^{1.4}, R^{1.5}, R⁵, R^{11.1}, and L⁴ are as described herein, including in embodiments. In embodiments, the compound has the formula:

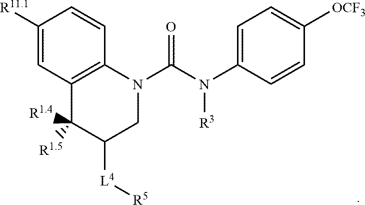

R^{1.4}, R^{1.5} , R³, R⁵, R^{11.1}, and L⁴ are as described herein, including in embodiments. In embodiments, the compound has the formula:

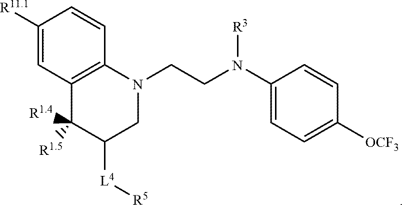

R^{1.4}, R^{1.5}, R³, R⁵, R^{11.1}, and L⁴ are as described herein, including in embodiments. In embodiments, the compound has the formula:

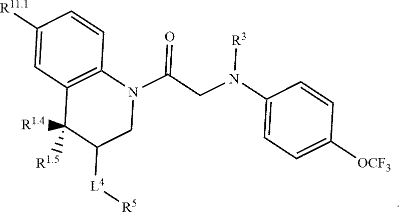

R^{1.4}, R^{1.5}, R³, R⁵, R^{11.1}, and L⁴ are as described herein, including in embodiments. In embodiments, the compound has the formula:

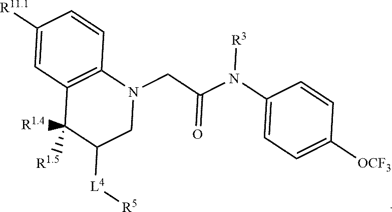

R^{1.4}, R^{1.5}, R³, R⁵, R^{11.1}, and L⁴ are as described herein, including in embodiments.

In embodiments, the compound has the formula:

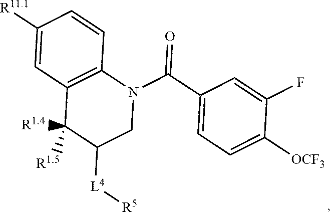

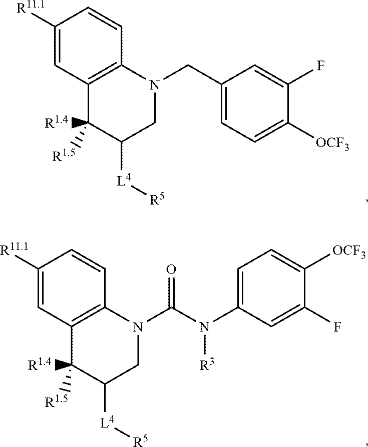

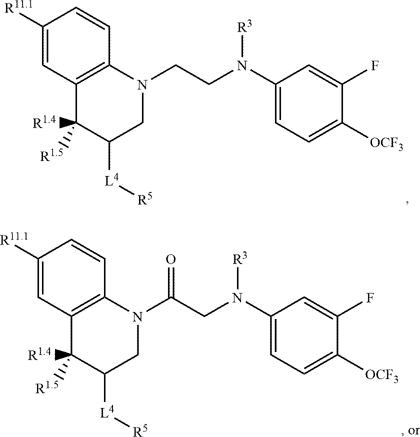

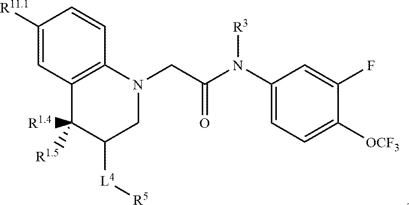

R^{1.4}, R^{1.5}, R³, R⁵, R^{11.1}, and L⁴ are as described herein, including in embodiments.

In embodiments, the compound has the formula:

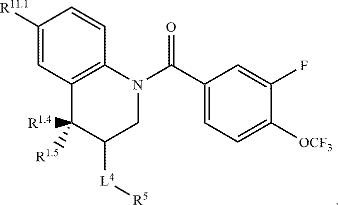

R^{1.4}, R^{1.5}, R⁵, R^{11.1}, and L⁴ are as described herein, including in embodiments. In embodiments, the compound has the formula:

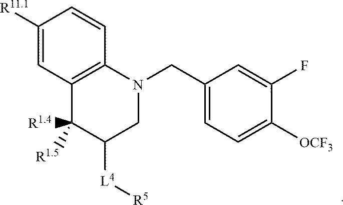

R^{1.4}, R^{1.5}, R⁵, R^{11.1}, and L⁴ are as described herein, including in embodiments. In embodiments, the compound has the formula:

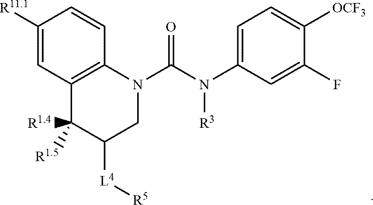

R^{1.4}, R^{1.5}, R³, R⁵, R^{11.1}, and L⁴ are as described herein, including in embodiments. In embodiments, the compound has the formula:

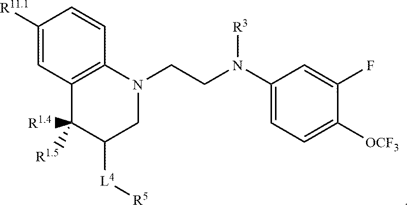

R^{1.4}, R^{1.5}, R³, R⁵, R^{11.1}, and L⁴ are as described herein, including in embodiments. In embodiments, the compound has the formula:

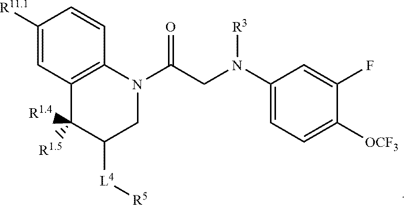

R^{1.4}, R^{1.5}, R³, R⁵, R^{11.1}, and L⁴ are as described herein, including in embodiments. In embodiments, the compound has the formula:

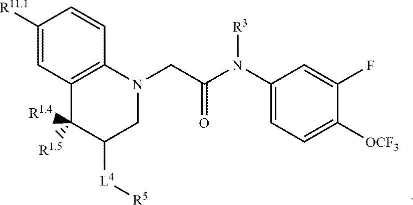

R^{1.4}, R^{1.5}, R³, R⁵, R^{11.1}, and L⁴ are as described herein, including in embodiments.

In embodiments, the compound has the formula:

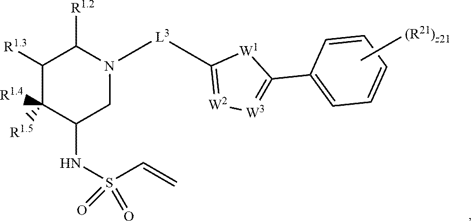

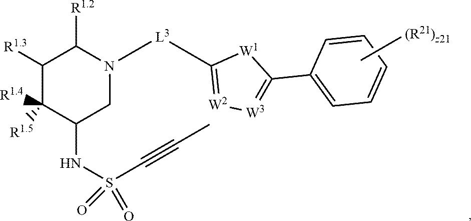

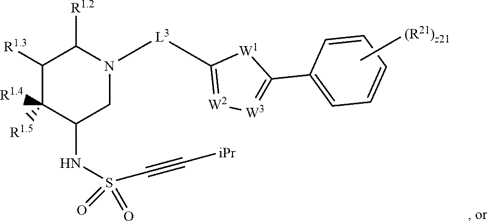

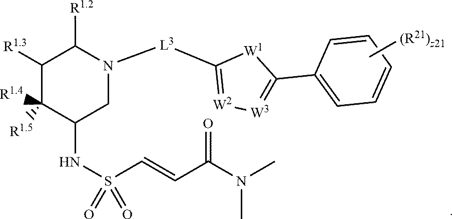

R^{1.2}, R^{1.3}, R^{1.4}, R^{1.5}, R²¹, L³, W¹, W², W³, and z21 are as described herein, including in embodiments.

In embodiments, the compound has the formula:

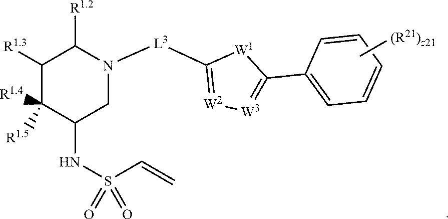

R^{1.2}, R^{1.3}, R^{1.4}, R^{1.5}, R²¹, L³, W¹, W², W³, and z21 are as described herein, including in embodiments. In embodiments, the compound has the formula:

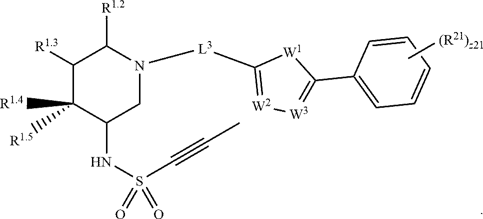

R^{1.2} , R^{1.3}, R^{1.4}, R^{1.5}, R²¹, L³, W¹, W², W³, and z21 are as described herein, including in embodiments. In embodiments, the compound has the formula:

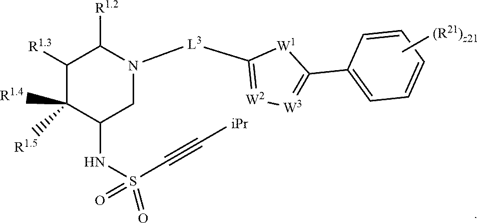

R^{1.2}, R^{1.3}, R^{1.4}, R^{1.5}, R²¹, L³, W¹, W², W³, and z21 are as described herein, including in embodiments. In embodiments, the compound has the formula:

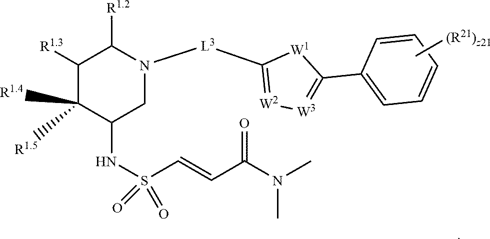

R^{1.2}, R^{1.3}, R^{1.4}, R^{1.5}, R²¹, L³, W¹, W², ³, and z21 are as described herein, including in embodiments.

In embodiments, the compound has the formula:

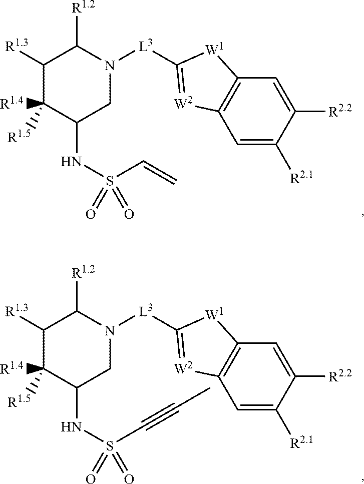

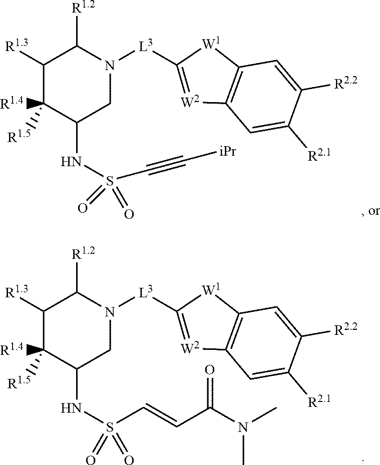

R^{1.2}, R^{1.3}, R^{1.4}, R^{1.5}, R^{2.1}, R^{2.2}, L³, W¹, and W² are as described herein, including in embodiments.

In embodiments, the compound has the formula:

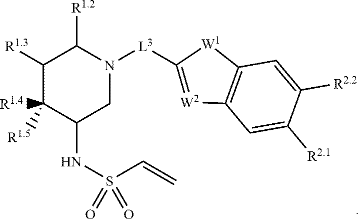

R^{1.2}, R^{1.3}, R^{1.4}, R^{1.5}, R^{2.1}, R^{2.2}, L³, W¹, and W² are as described herein, including in embodiments. In embodiments, the compound has the formula:

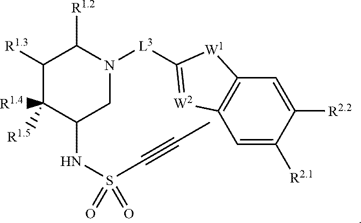

R^{1.2}, R^{1.3}, R^{1.4}, R^{1.5}, R^{2.1}, R^{2.2}, L³, W¹, and W² are as described herein, including in embodiments. In embodiments, the compound has the formula:

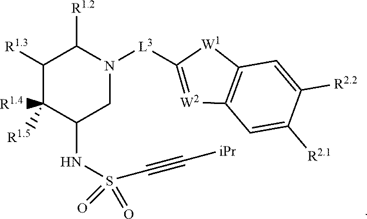

R^{1.2}, R^{1.3}, R^{1.4}, R^{1.5}, R^{2.1}, R^{2.2}, L³, W¹, and W² are as described herein, including in embodiments. In embodiments, the compound has the formula:

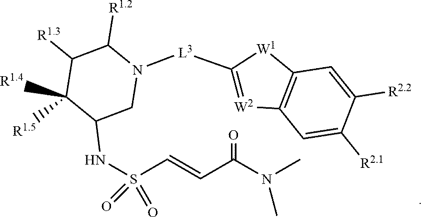

R^{1.2}, R^{1.3}, R^{1.4}, R^{1.5}, R^{2.1}, R^{2.2}, L³, W¹, and W² are as described herein, including in embodiments.

In embodiments, the compound has the formula:

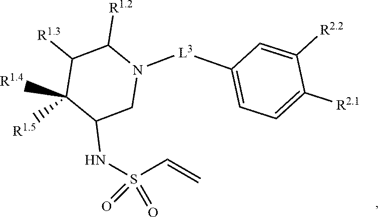

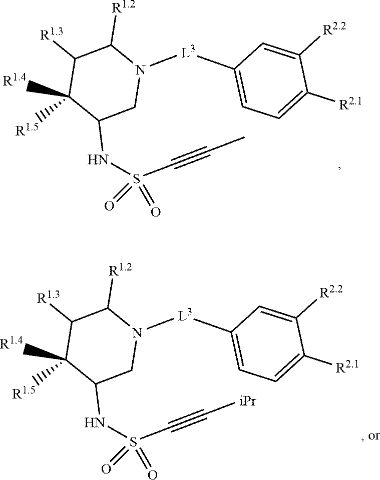

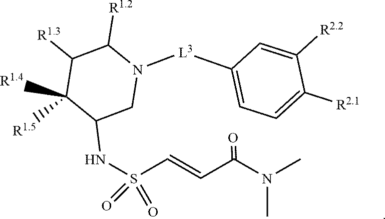

R^{1.2}, R^{1.3}, R^{1.4}, R^{1.5}, R^{2.1}, R^{2.2}, and L³ are as described herein, including in embodiments.

In embodiments, the compound has the formula:

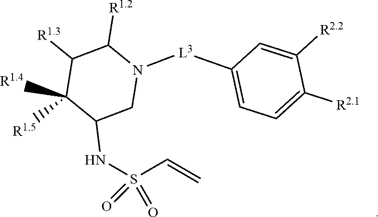

R^{1.2}, R^{1.3}, R^{1.4}, R^{1.5}, R^{2.1}, R^{2.2}, and L³ are as described herein, including in embodiments. In embodiments, the compound has the formula:

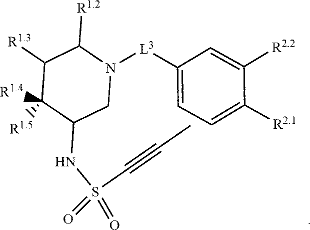

R^{1.2}, R^{1.3}, R^{1.4}, R^{1.5}, R^{2.1}, R^{2.2}, and L³ are as described herein, including in embodiments. In embodiments, the compound has the formula:

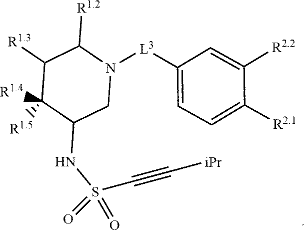

R^{1.2}, R^{1.3}, R^{1.4}, R^{1.5}, R^{2.1}, R^{2.2} , and L³ are as described herein, including in embodiments. In embodiments, the compound has the formula:

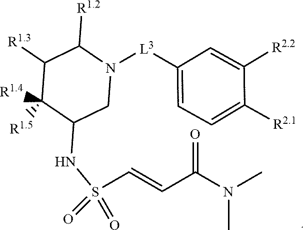

R^{1.2}, R^{1.3}, R^{1.4}, R^{1.5}, R^{2.1}, R^{2.2}, and L³ are as described herein, including in embodiments.

In embodiments, the compound has the formula:

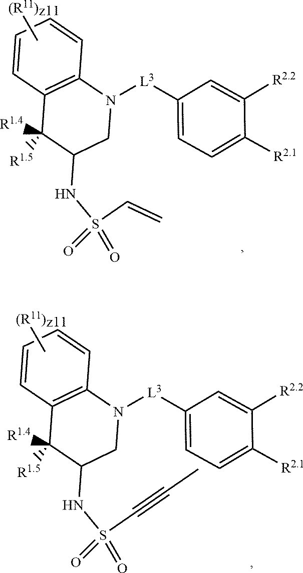

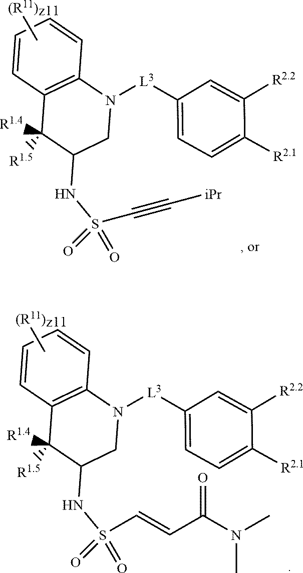

R^{1.4}, R^{1.5}, R¹¹, R^{2.1}, R^{2.2}, L³, and z11 are as described herein, including in embodiments.

In embodiments, the compound has the formula:

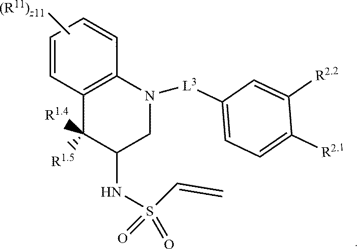

R^{1.4}, R^{1.5}, R¹¹, R^{2.1}, R^{2.2}, L³, and z11 are as described herein, including in embodiments. In embodiments, the compound has the formula:

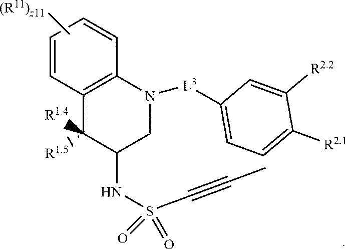

R^{1.4}, R^{1.5}, R¹¹, R^{2.1}, R^{2.2}, L³, and z11 are as described herein, including in embodiments. In embodiments, the compound has the formula:

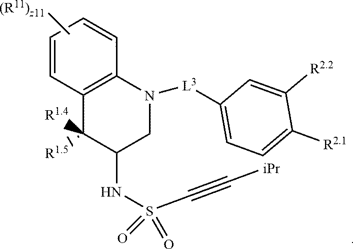

R^{1.4}, R^{1.5}, R¹¹, R^{2.1}, R^{2.2}, L³, and z11 are as described herein, including in embodiments. In embodiments, the compound has the formula:

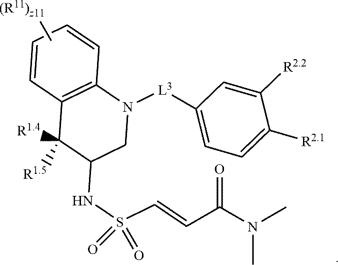

R^{1.4}, R^{1.5}, R¹¹, R^{2.1}, R^{2.2}, L³, and z11 are as described herein, including in embodiments.

In embodiments, the compound has the formula:

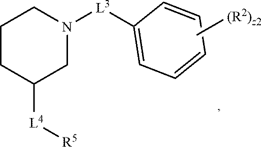

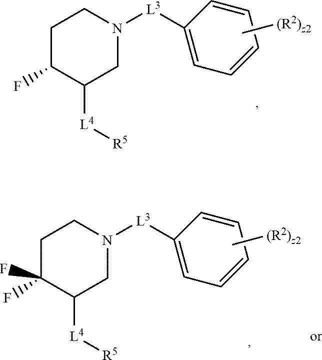

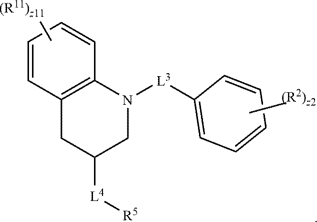

R², R⁵, R¹¹, L³, L⁴, and z11 are as described herein, including in embodiments.

In embodiments, the compound has the formula:

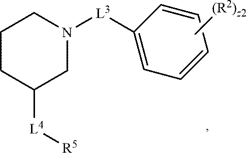

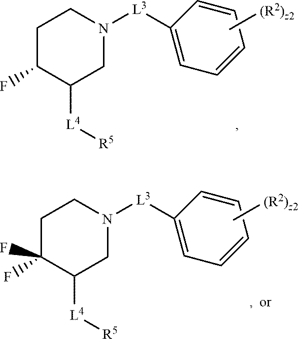

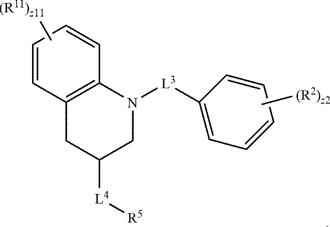

R², R⁵, R¹¹, L³, L⁴, z2, and z11 are as described herein, including in embodiments.

In embodiments, the compound has the formula:

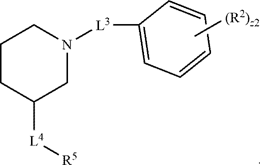

R², R⁵, L³, L⁴ and z2 are as described herein, including in embodiments. In embodiments, the compound has the formula:

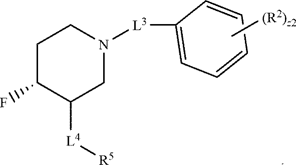

R², R⁵, L³, L⁴ and z2 are as described herein, including in embodiments. In embodiments, the compound has the formula:

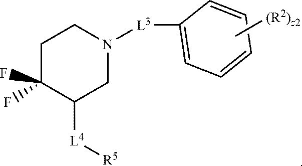

R², R⁵, L³, L⁴ and z2 are as described herein, including in embodiments. In embodiments, the compound has the formula:

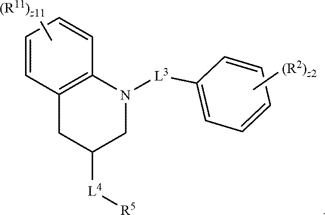

R², R⁵, R¹¹, L³, L⁴, z2, and z11 are as described herein, including in embodiments.

In embodiments, the compound has the formula:

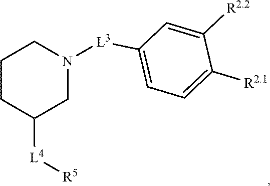

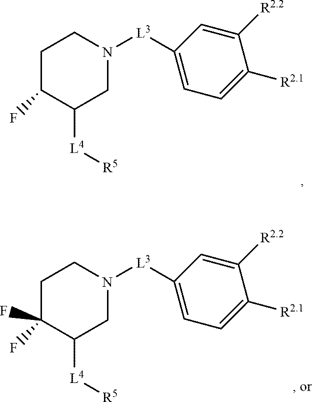

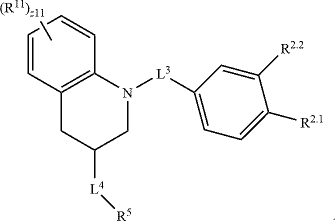

R⁵, R¹¹, L³, L⁴, R^{2.1}, R^{2.2}, and z11 are as described herein, including in embodiments.

In embodiments, the compound has the formula:

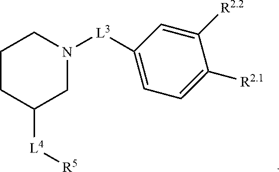

R⁵, L³, L⁴, R^{2.1},and R^{2.2} are as described herein, including in embodiments. In embodiments, the compound has the formula:

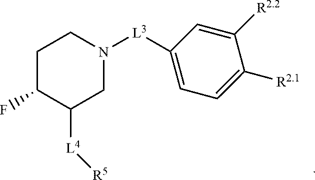

R⁵, L³, L⁴, R^{2.1}, and R^{2.2} are as described herein, including in embodiments. In embodiments, the compound has the formula:

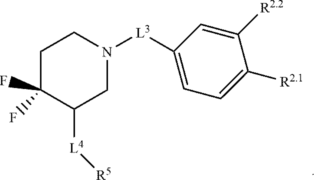

R⁵, L³, L⁴, R^{2.1}, and R^{2.2} are as described herein, including in embodiments. In embodiments, the compound has the formula:

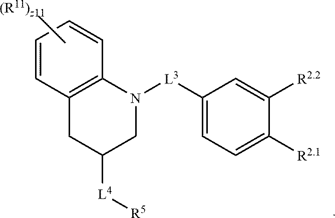

R⁵, R¹¹, L³, L⁴, R^{2.1}, R^{2.2}, and z11 are as described herein, including in embodiments.

In embodiments, the compound has the formula:

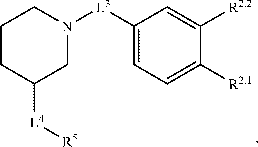

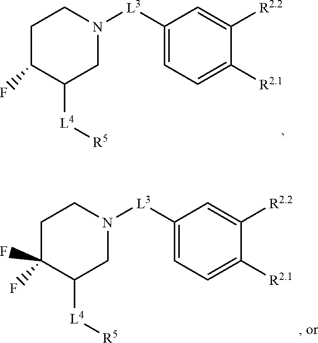

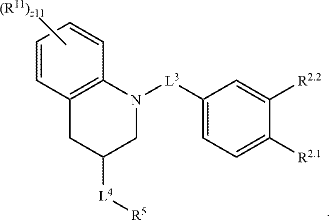

R⁵, R¹¹, L³, L⁴ and z11 are as described herein, including in embodiments. R^{2.1} and R^{2.2} are independently hydrogen, —F, or —OCF₃.

In embodiments, the compound has the formula:

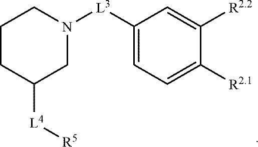

R⁵, L³, and L⁴ are as described herein, including in embodiments. R^{2.1} and R^{2.2} are independently hydrogen, —F, or —OCF₃. In embodiments, the compound has the formula:

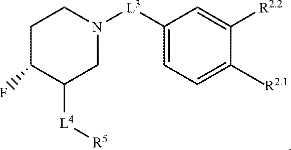

R⁵, L³, and L⁴ are as described herein, including in embodiments. R^{2.1} and R^{2.2} are independently hydrogen, —F, or —OCF₃. In embodiments, the compound has the formula:

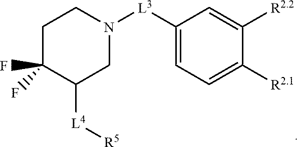

R⁵, L³, and L⁴ are as described herein, including in embodiments. R^{2.1} and R^{2.2} are independently hydrogen, —F, or —OCF₃. In embodiments, the compound has the formula:

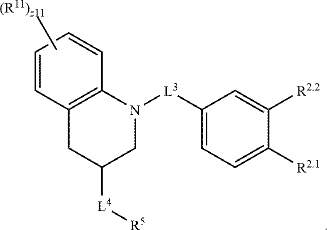

R⁵, R¹¹, L³, L⁴ and z11 are as described herein, including in embodiments. R^{2.1} and R^{2.2} are independently hydrogen, —F, or —OCF₃.

In embodiments, the compound has the formula:

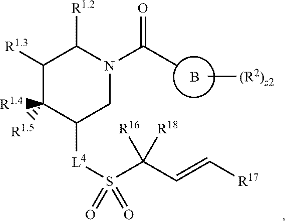

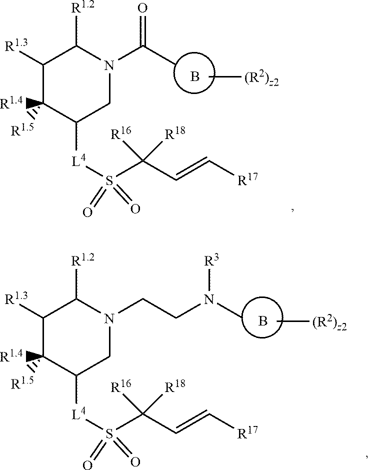

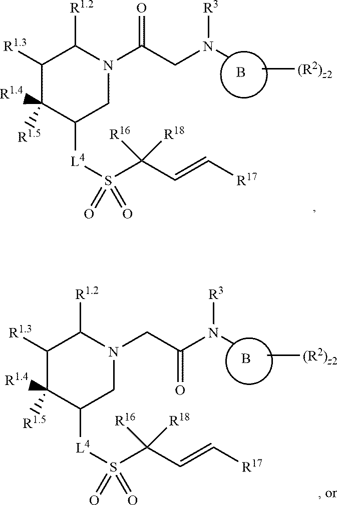

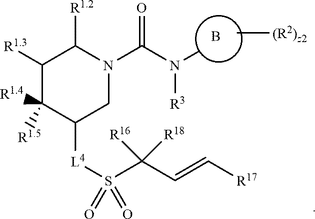

R^{1.2}, R^{1.3}, R^{1.4}, R^{1.5}, R², R³, R¹⁶, R¹⁷, R¹⁸, L⁴, Ring B, and z2 are as described herein, including in embodiments.

In embodiments, the compound has the formula:

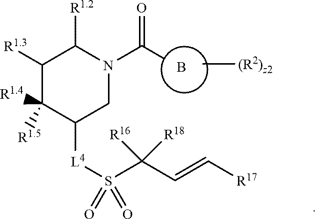

R^{1.2}, R^{1.3}, R^{1.4}, R^{1.5}, R², R¹⁶, R¹⁷, R¹⁸, L⁴, Ring B, and z2 are as described herein, including in embodiments. In embodiments, the compound has the formula:

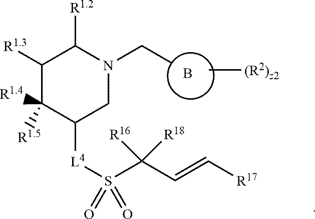

. R^{1.2}, R^{1.3}, R^{1.4}, R^{1.5}, R², R¹⁶, R¹⁷, R¹⁸, L⁴, Ring B, and z2 are as described herein, including in embodiments. In embodiments, the compound has the formula:

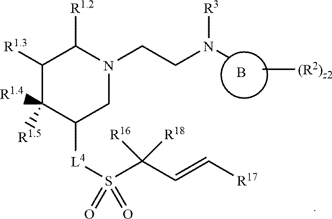

. R^{1.2}, R^{1.3}, R^{1.4}, R^{1.5}, R², R³, R¹⁶. R¹⁷, R¹⁸, L⁴, Ring B, and z2 are as described herein, including in embodiments. In embodiments, the compound has the formula:

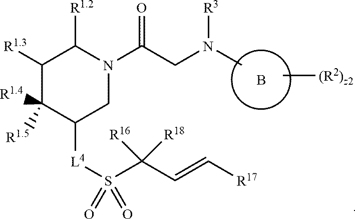

R^{1.2}, R^{1.3}, R^{1.4}, R^{1.5}, R², R³, R¹⁶, R¹⁷, R¹⁸, L⁴, Ring B, and z2 are as described herein, including in embodiments. In embodiments, the compound has the formula:

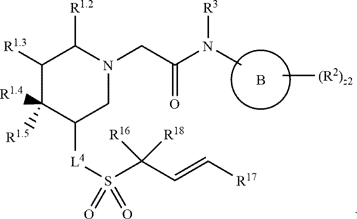

R^{1.2}, R^{1.3}, R^{1.4}, R^{1.5}, R², R³, R¹⁶, R¹⁷, R¹⁸, L⁴ Ring B, and z2 are as described herein, including in embodiments. In embodiments, the compound has the formula:

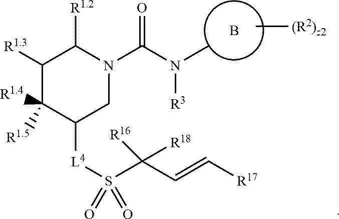

R^{1.2}, R^{1.3}, R^{1.4}, R^{1.5}, R², R³, R¹⁶, R¹⁷, R¹⁸, L⁴, Ring B, and z2 are as described herein, including in embodiments.

In embodiments, the compound has the formula:

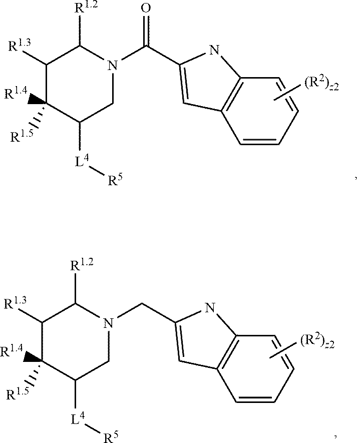

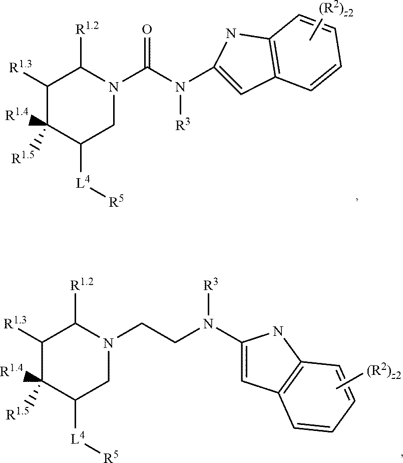

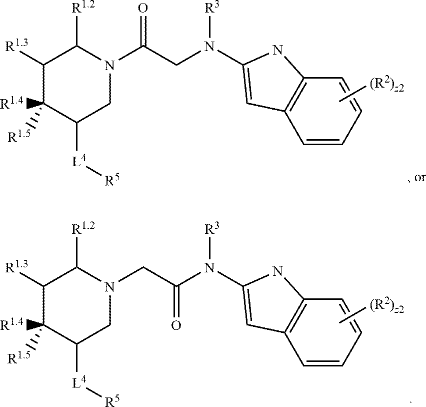

R^{1.2}, R^{1.3}, R^{1.4}, R^{1.5}, R², R³, R⁵, L⁴, and z2 are as described herein, including in embodiments.

In embodiments, the compound has the formula:

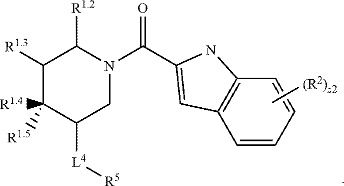

R^{1.2}, R^{1.3}, R^{1.4}, R^{1.5}, R², R⁵, L⁴, and z2 are as described herein, including in embodiments. In embodiments, the compound has the formula:

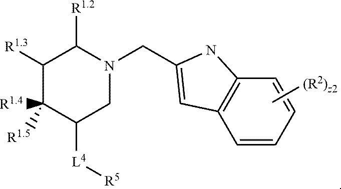

R^{1.2}, R^{1.3}, R^{1.4}, R^{1.5}, R², R⁵, L⁴, and z2 are as described herein, including in embodiments. In embodiments, the compound has the formula:

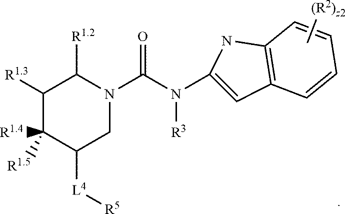

R^{1.2}, R^{1.3}, R^{1.4}, R^{1.5}, R², R³, R⁵, L⁴, and z2 are as described herein, including in embodiments. In embodiments, the compound has the formula:

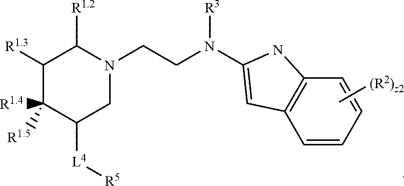

R^{1.2}, R^{1.3}, R^{1.4}, R^{1.5}, R², R³, R⁵, L⁴, and z2 are as described herein, including in embodiments. In embodiments, the compound has the formula:

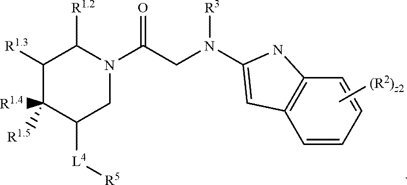

R^{1.2}, R^{1.3}, R^{1.4}, R^{1.5}, R², R³, R⁵, L⁴, and z2 are as described herein, including in embodiments. In embodiments, the compound has the formula:

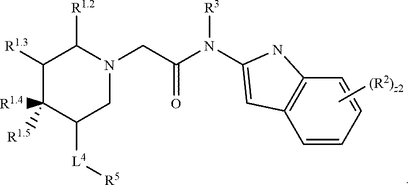

R^{1.2}, R^{1.3}, R^{1.4}, R^{1.5}, R², R³, R⁵, L⁴, and z2 are as described herein, including in embodiments.

In embodiments, the compound has the formula:

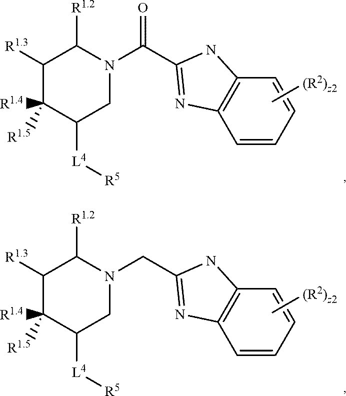

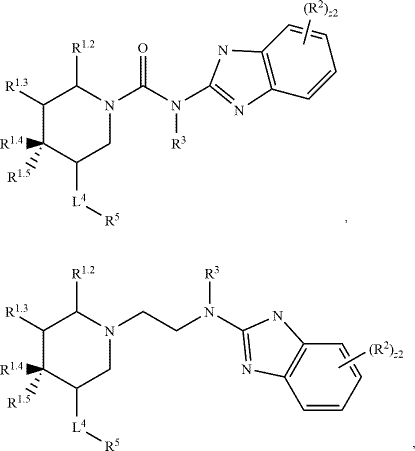

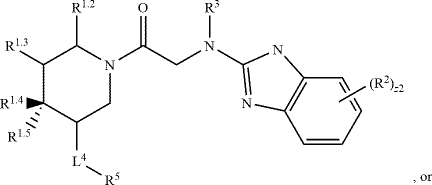

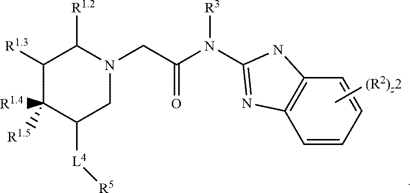

R^{1.2}, R^{1.3}, R^{1.4}, R^{1.5}, R², R³, R⁵, L⁴, and z2 are as described herein, including in embodiments.

In embodiments, the compound has the formula:

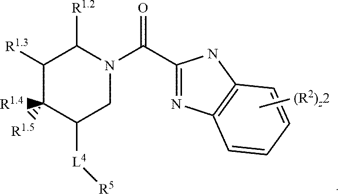

R^{1.2}, R^{1.3}, R^{1.4}, R^{1.5}, R², R⁵, L⁴, and z2 are as described herein, including in embodiments. In embodiments, the compound has the formula:

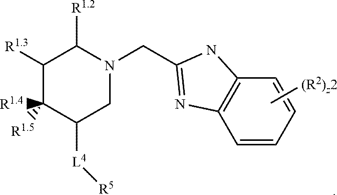

R^{1.2}, R^{1.3}, R^{1.4}, R^{1.5}, R², R⁵, L⁴, and z2 are as described herein, including in embodiments. In embodiments, the compound has the formula:

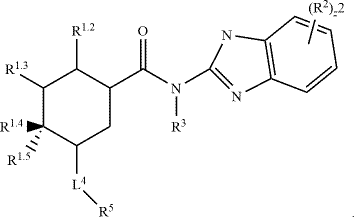

R^{1.2}, R^{1.5}, R^{1.4}, R^{1.5}, R², R³, R⁵, L⁴, and z2 are as described herein, including in embodiments. In embodiments, the compound has the formula:

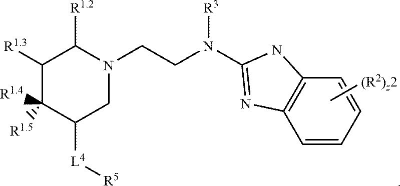

R^{1.2}, R^{1.3}, R^{1.4}, R^{1.5}, R², R³, R⁵, L⁴, and z2 are as described herein, including inembodiments. In embodiments, the compound has the formula:

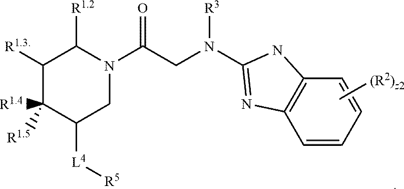

R^{1.2}, R^{1.3}, R^{1.4}, R^{1.5}, R², R³, R⁵, L⁴, and z2 are as described herein, including in embodiments. In embodiments, the compound has the formula:

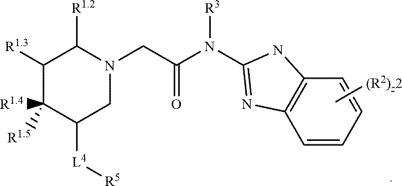

R^{1.2}, R^{1.3}, R^{1.4}, R^{1.5}, R², R³, R⁵, L⁴, and z2 are as described herein, including in embodiments.

In embodiments, the compound has the formula:

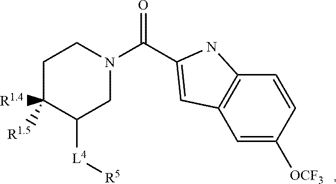

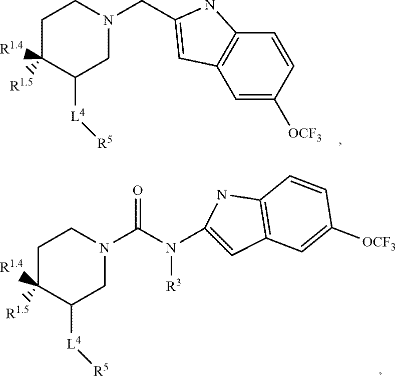

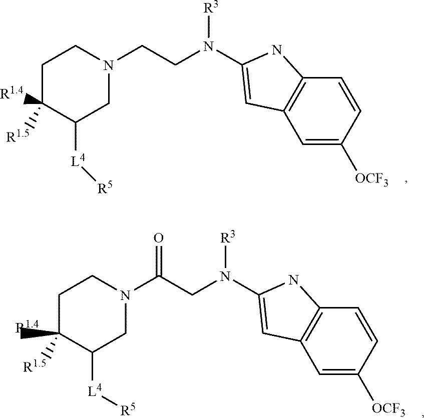

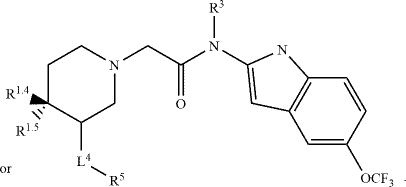

R^{1.4}, R^{1.5}, R³, R⁵, and L⁴ are as described herein, including in embodiments.

In embodiments, the compound has the formula:

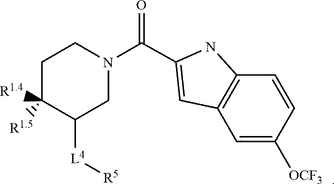

R^{1.4}, R^{1.5}, R⁵, and L⁴ are as described herein, including in embodiments. In embodiments, the compound has the formula:

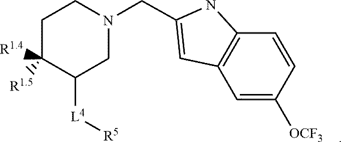

R^{1.4}, R^{1.5}, R⁵, and L⁴ are as described herein, including in embodiments. In embodiments, the compound has the formula:

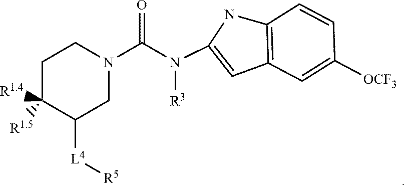

R^{1.4}, R^{1.5}, R³, R⁵, and L⁴ are as described herein, including in embodiments. In embodiments, the compound has the formula:

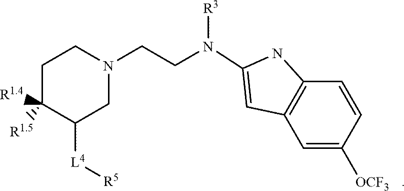

R^{1.4}, R^{1.5}, R³, R⁵, and L⁴ are as described herein, including in embodiments. In embodiments, the compound has the formula:

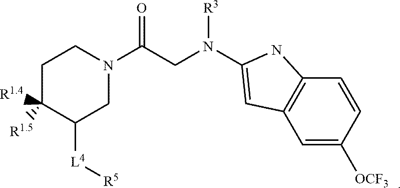

R^{1.4}, R^{1.5}, R³, R⁵, and L⁴ are as described herein, including in embodiments. In embodiments, the compound has the formula:

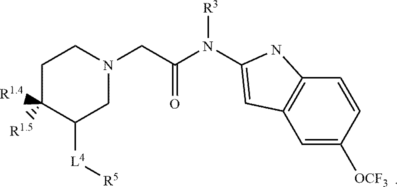

R^{1.4}, R^{1.5}, R³, R⁵, and L⁴ are as described herein, including in embodiments.

In embodiments, the compound has the formula:

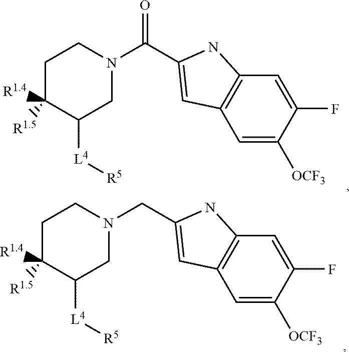

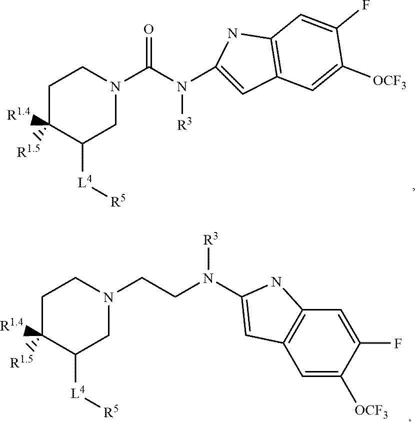

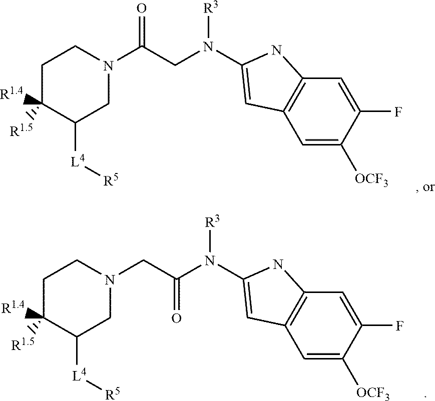

R^{1.4}, R^{1.5}, R³, R⁵, and L⁴ are as described herein, including in embodiments.

In embodiments, the compound has the formula:

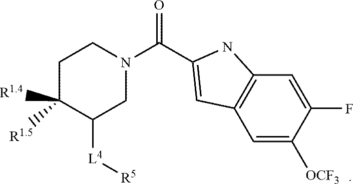

R^{1.4}, R^{1.5}, R⁵, and L⁴ are as described herein, including in embodiments. In embodiments, the compound has the formula:

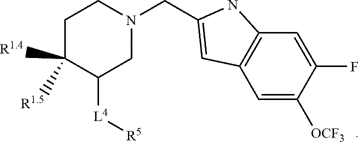

R^{1.4}, R^{1.5}, R⁵, and L⁴ are as described herein, including in embodiments. In embodiments, the compound has the formula:

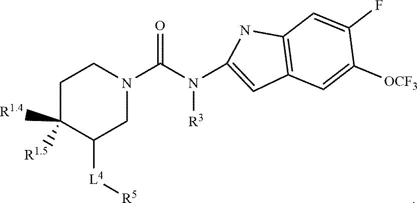

R^{1.4}, R^{1.5}, R³, R⁵, and L⁴ are as described herein, including in embodiments. In embodiments, the compound has the formula:

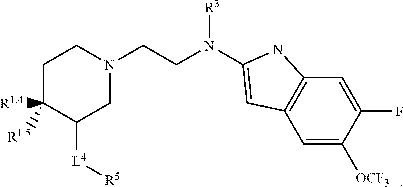

R^{1.4}, R^{1.5}, R³, R⁵ , and L⁴ are as described herein, including in embodiments. In embodiments, the compound has the formula:

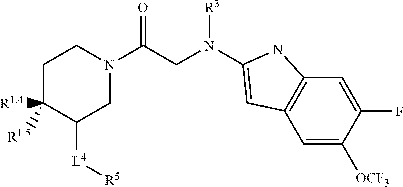

R^{1.4}, R^{1.5}, R³, R⁵, and L⁴ are as described herein, including in embodiments. In embodiments, the compound has the formula:

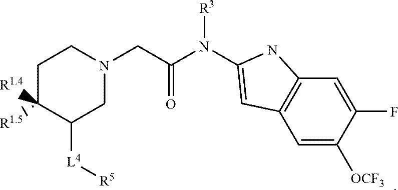

R^{1.4}, R^{1.5}, R³, R⁵, and L⁴ are as described herein, including in embodiments.

In embodiments, the compound has the formula:

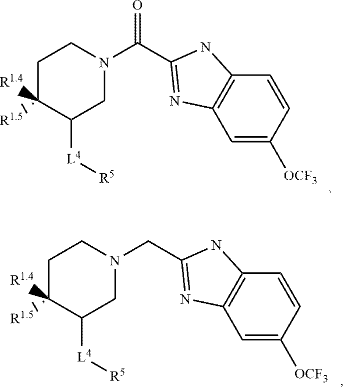

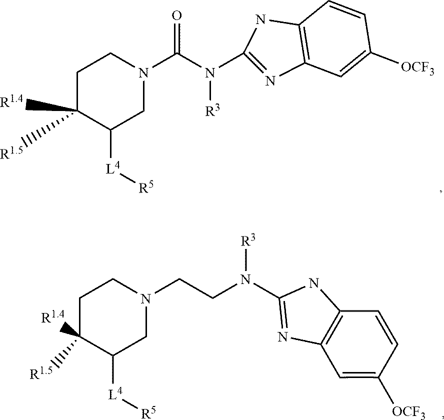

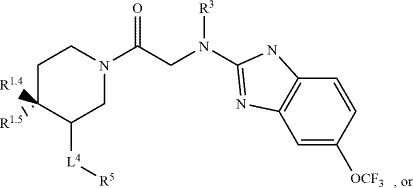

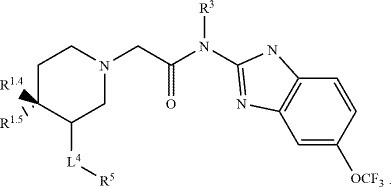

R^{1.4}, R^{1.5}, R³, R⁵, and L⁴ are as described herein, including in embodiments.

In embodiments, the compound has the formula:

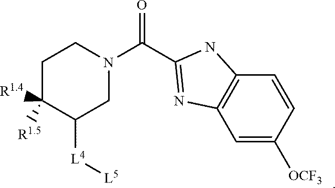

R^{1.4}, R^{1.5}, R⁵, and L⁴ are as described herein, including in embodiments. In embodiments, the compound has the formula:

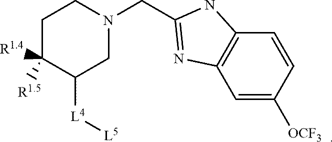

R^{1.4}, R^{1.5}, R⁵, and L⁴ are as described herein, including in embodiments. In embodiments, the compound has the formula:

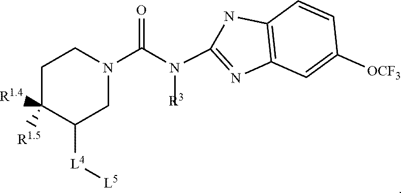

R^{1.4}, R^{1.5}, R³, R⁵, and L⁴ are as described herein, including in embodiments. In embodiments, the compound has the formula:

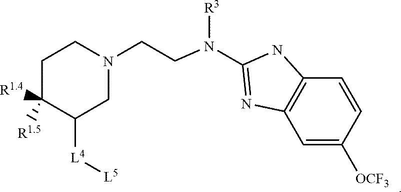

R^{1.4}, R^{1.5}, R³, R⁵, and L⁴ are as described herein, including in embodiments. In embodiments, the compound has the formula:

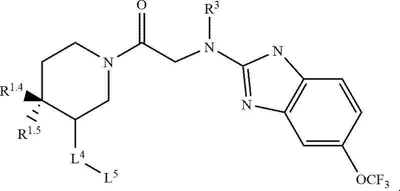

R^{1.4}, R^{1.5}, R³, R⁵, and L⁴ are as described herein, including in embodiments. In embodiments, the compound has the formula:

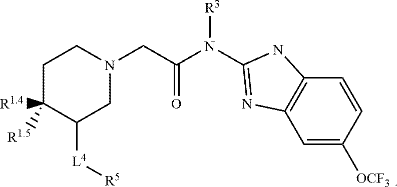

R^{1.4}, R^{1.5}, R³, R⁵, and L⁴ are as described herein, including in embodiments.

In embodiments, the compound has the formula:

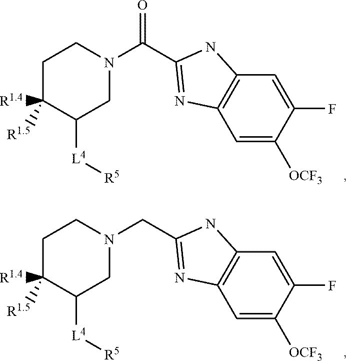

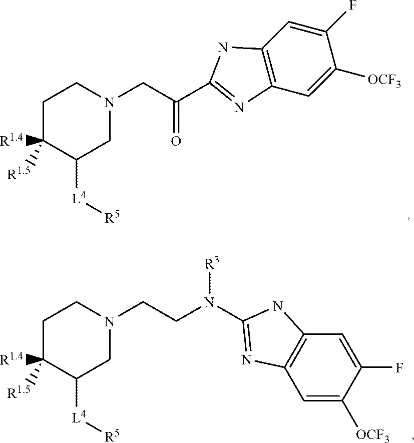

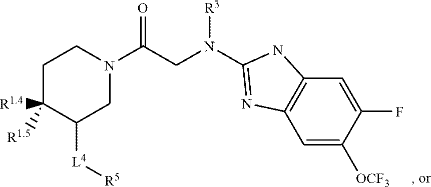

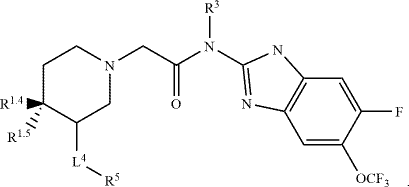

R^{1.4}, R^{1.5}, R³, R⁵, and L⁴ are as described herein, including in embodiments.

In embodiments, the compound has the formula:

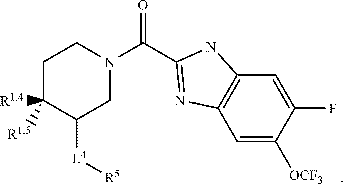

R^{1.4}, R^{1.5}, R⁵, and L⁴ are as described herein, including in embodiments. In embodiments, the compound has the formula:

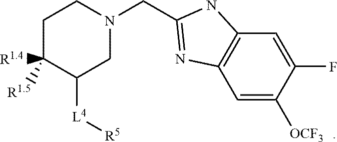

R^{1.4}, R^{1.5}, R⁵, and L⁴ are as described herein, including in embodiments. In embodiments, the compound has the formula:

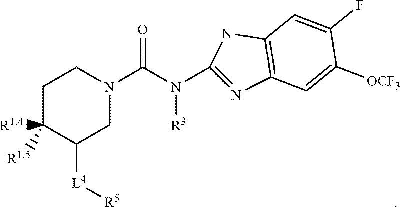

R^{1.4}, R^{1.5}, R³, R⁵, and L⁴ are as described herein, including in embodiments. In embodiments, the compound has the formula:

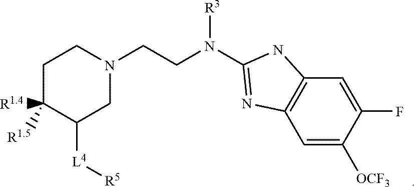

R^{1.4}, R^{1.5}, R³, R⁵, and L⁴ are as described herein, including in embodiments. In embodiments, the compound has the formula:

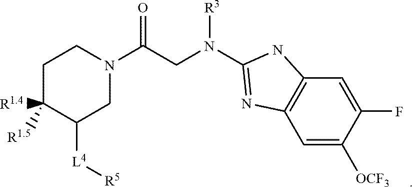

R^{1.4}, R^{1.5}, R³, R⁵, and L⁴ are as described herein, including in embodiments. In embodiments, the compound has the formula:

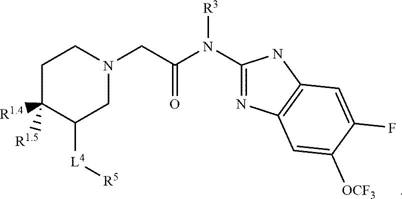

R^{1.4}, R^{1.5}, R³, R⁵, and L⁴ are as described herein, including in embodiments.

In embodiments, the compound has the formula:

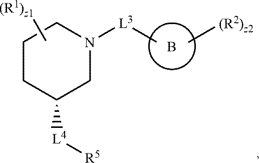

wherein R¹, R², R⁵, z1, z2, L³, L⁴, and Ring B are as described herein, including in embodiments.

In embodiments, the compound has the formula:

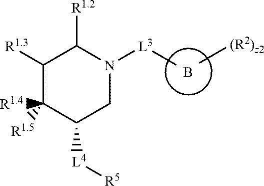

wherein R^{1.2}, R^{1.3}, R^{1.4}, R^{1.5}, R², R⁵, z2, L³, L⁴, and Ring B are as described herein, including in embodiments.

In embodiments, the compound has the formula:

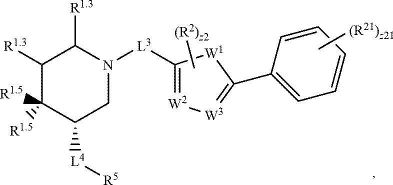

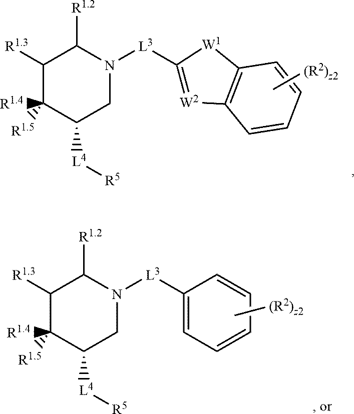

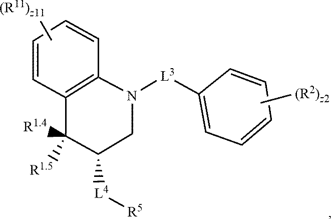

wherein R^{1.2}, R^{1.3}, R^{1.4}, R^{1.5}, R², z2, R^{11,} z11, R²¹, z21, R⁵, L³, L⁴, W¹, W², and W³ are as described herein, including in embodiments.

In embodiments, the compound has the formula:

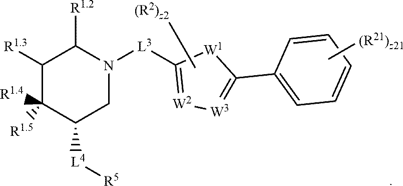

R^{1.2}, R^{1.3}, R^{1.4}, R^{1.5}, R,² z2, R²¹ , z21, R⁵, L³, L⁴, W¹, W², and W³ are as described herein, including in embodiments. In embodiments, the compound has the formula:

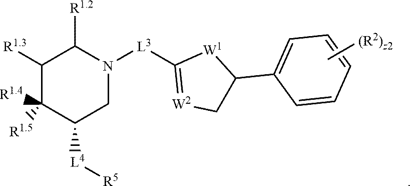

R^{1.2}, R^{1.3}, R^{1.4}, R^{1.5}, R², z2, R⁵, L³, L⁴, W¹, and W² are as described herein, including in embodiments. In embodiments, the compound has the formula:

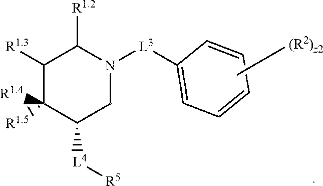

R^{1.2}, R^{1.3}, R^{1.4}, R^{1.5}, R², z2, R⁵, L³, and L⁴ are as described herein, including in embodiments. In embodiments, the compound has the formula:

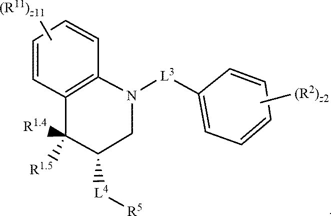

R^{1.4}, R^{1.5}, R², z2, R¹¹, z11, R⁵, L³, and L⁴ are as described herein, including in embodiments.

In embodiments, the compound has the formula:

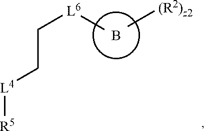

wherein Ring B is phenyl, 5 to 6 membered heteroaryl, or 9 membered heteroaryl; and R², z2, R⁵, L⁴, and L⁶ are as described herein, including in embodiments. In embodiments, the compound has the formula:

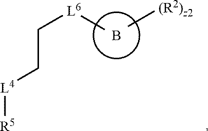

wherein Ring B is phenyl, 5 to 6 membered heteroaryl, or 9 membered heteroaryl; L⁴ is —NH— or —NR⁴—; R⁵ is

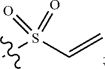

and R², z2, R⁴, and L⁶ are as described herein, including in embodiments. In embodiments, the compound has the formula:

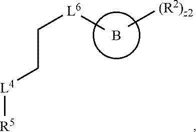

wherein Ring B is phenyl, 5 to 6 membered heteroaryl or 9 membered heteroaryl; L⁴ is —NH— or —NR⁴—; R⁵ is

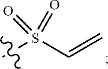

and R², z2, R⁴, and L⁶ are as described herein, including in embodiments.

In embodiments, the compound has the formula:

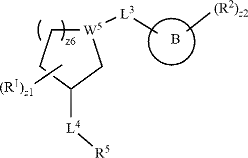

wherein W⁵ is CH, Ring B is phenyl; and R², z1, R², z2, z6, R⁵, L³, and L⁴ are as described herein, including in embodiments. In embodiments, the compound has the formula:

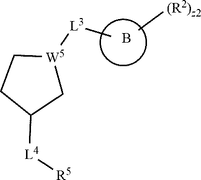

wherein W⁵ is CH, Ring B is phenyl; and R², z2, R⁵, L³, and L⁴ are as described herein, including in embodiments. In embodiments, the compound has the formula:

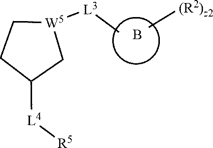

wherein W⁵ is CH, Ring B is phenyl, L⁴ is —NH—; R⁵ is

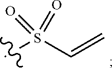

and R², z2, and L³ are as described herein, including in embodiments.

In embodiments, (Ring B)-(R²)_{z2} is

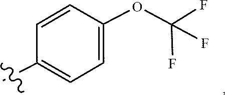

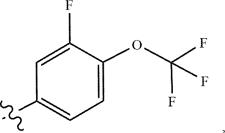

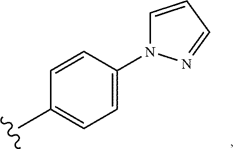

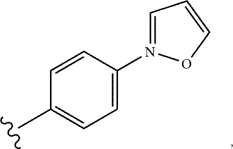

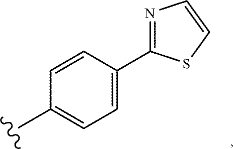

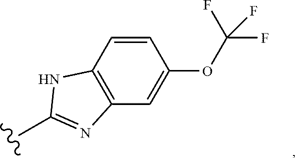

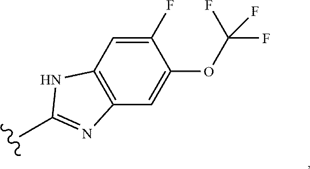

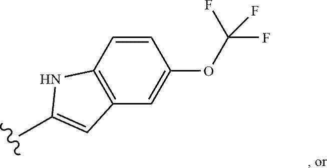

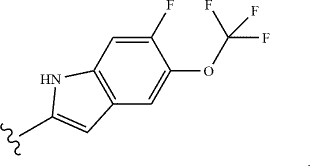

In embodiments, (Ring B)-(R²)_{z2} is

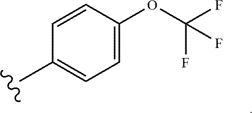

In embodiments, (Ring B)-(R²)_{z2} is

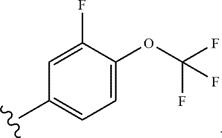

In embodiments, (Ring B)-(R²)_{z2}

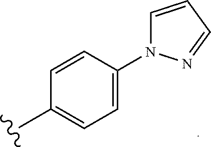

In embodiments, (Ring B)-(R²)_{z2}

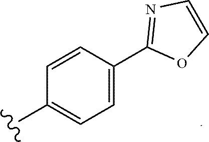

In embodiments, (Ring B)-(R²)_{z2}

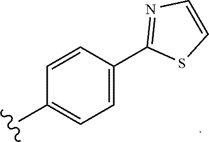

In embodiments, (Ring B)-(R²)_{z2}

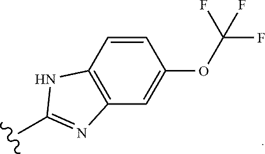

In embodiments, (Ring B)-(R²)_{z2}

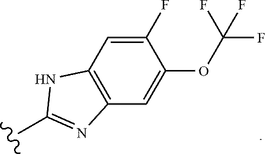

In embodiments, (Ring B)-(R²)_{z2}

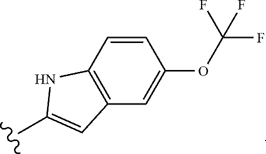

In embodiments, (Ring B)-(R²)_{z2}

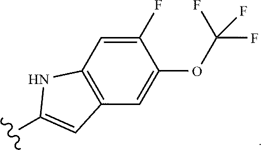

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O) NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents on adjacent carbons may optionally be joined to form a substituted or unsubstituted cycloalkyl, substituted or unsubstituted heterocycloalkyl, substituted or unsubstituted phenyl, or substituted or unsubstituted heteroaryl or two R¹ substituents bonded to the same carbon atom may optionally be joined to form a substituted or unsubstituted alkyl or substituted or unsubstituted heterocycloalkyl.

In embodiments, R¹ is independently halogen, —CX¹₃, —CHX¹₂, —CH₂X¹, —OCX¹₃, —OCH₂X¹, —OCHX¹₂, —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —SF₅, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl; two adjacent R¹ substituents on adjacent carbons may optionally be joined to form a substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl or two R¹ substituents bonded to the same carbon atom may optionally be joined to form a substituted or unsubstituted C₃-C₆ cycloalkyl or substituted or unsubstituted 3 to 6 membered heterocycloalkyl.

In embodiments, R¹ is independently halogen, —CCl₃, —CBr₃, —CF₃, —CI₃, —CN, —OH, —NH₂, —COOH, —CONH₂, —OCCl₃, —OCF₃, —OCBr₃, —OCI₃, —N₃, substituted or unsubstituted C₁-C₃ alkyl, substituted or unsubstituted 2 to 4 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R¹ is independently halogen, —CCl₃, —CBr₃, —CF₃, —CI₃, —CN, —OH, —NH₂, —COOH, —CONH₂, —OCCl₃, —OCF₃, —OCBr₃, —OCI₃, —N₃, substituted or unsubstituted C₁-C₃ alkyl, or substituted or unsubstituted 2 to 4 membered heteroalkyl. In embodiments, R¹ is independently halogen. In embodiments, R¹ is independently —F. In embodiments, R¹ is independently —Cl. In embodiments, R¹ is independently-Br. In embodiments, R¹ is independently —I. In embodiments, R¹ is independently —CCl₃. In embodiments, R¹ is independently —CBr₃. In embodiments, R¹ is independently —CF₃. In embodiments, R¹ is independently —CI₃. In embodiments, R¹ is independently —CN. In embodiments, R¹ is independently —OH. In embodiments, R¹ is independently —NH₂. In embodiments, R¹ is independently —COOH. In embodiments, R¹ is independently —CONH₂. In embodiments, R¹ is independently —OCCl₃. In embodiments, R¹ is independently —OCF₃. In embodiments, R¹ is independently —OCBr₃. In embodiments, R¹ is independently —OCI₃. In embodiments, R¹ is independently —N₃. In embodiments, R¹ is independently substituted or unsubstituted C₁-C₃ alkyl. In embodiments, R¹ is independently unsubstituted methyl. In embodiments, R¹ is independently unsubstituted ethyl. In embodiments, R¹ is independently unsubstituted propyl. In embodiments, R¹ is independently substituted or unsubstituted 2 to 4 membered heteroalkyl. In embodiments, R¹ is independently unsubstituted 2 to 4 membered heteroalkyl. In embodiments, R¹ is independently substituted or unsubstituted C₃-C₆ cycloalkyl. In embodiments, R¹ is independently substituted or unsubstituted 3 to 6 membered heterocycloalkyl. In embodiments, R¹ is independently substituted or unsubstituted phenyl. In embodiments, R¹ is independently substituted or unsubstituted 5 to 6 membered heteroaryl. In embodiments, R¹ is independently or substituted 5 to 6 membered heteroaryl. In embodiments, R¹ is independently unsubstituted 5 to 6 membered heteroaryl. In embodiments, R¹ is independently unsubstituted 5 membered heteroaryl. In embodiments, R¹ is independently unsubstituted 6 membered heteroaryl.

In embodiments, two R¹ substituents on adjacent carbons are joined to form a substituted or unsubstituted phenyl, or substituted or unsubstituted 5 to 6 membered heteroaryl. In embodiments, two R¹ substituents on adjacent carbons are joined to form a substituted or unsubstituted phenyl. In embodiments, two R¹ substituents on adjacent carbons are joined to form an R¹¹-substituted phenyl. In embodiments, two R¹ substituents on adjacent carbons are joined to form an unsubstituted phenyl. In embodiments, two R¹ substituents on adjacent carbons are joined to form a substituted or unsubstituted 5 to 6 membered heteroaryl.

In embodiments, R² is independently oxo, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, -NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O) NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl; two adjacent R² substituents on adjacent carbons may optionally be joined to form a substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl.

In embodiments, R² is independently halogen, —OCC1₃, —OCF₃, —OCBr₃, —OCI₃, or unsubstituted 5 to 6 membered heteroaryl. In embodiments, R² is independently halogen. In embodiments, R² is independently —F. In embodiments, R² is independently —Cl. In embodiments, R² is independently —Br. In embodiments, R² is independently —I. In embodiments, R² is independently —OCC1₃. In embodiments, R² is independently —OCF₃. In embodiments, R² is independently —OCBr₃. In embodiments, R² is independently —OCI₃. In embodiments, R² is unsubstituted 5 to 6 membered heteroaryl. In embodiments, R² is unsubstituted 5 membered heteroaryl. In embodiments, R² is unsubstituted 6 membered heteroaryl. In embodiments, R² is unsubstituted pyrrolyl, pyrazolyl, pyridazinyl, triazinyl, pyrimidinyl, imidazolyl, pyrazinyl, oxazolyl, isoxazolyl, thiazolyl, furyl, thienyl, pyridyl, pyrimidyl, benzothiazolyl, benzoxazoyl, benzimidazolyl, benzofuran, isobenzofuranyl, indolyl, or isoindolyl. In embodiments, R² is unsubstituted phenyl, benzimidazolyl, or indolyl. In embodiments, R² is unsubstituted pyrrolyl. In embodiments, R² is unsubstituted pyrazolyl. In embodiments, R² is unsubstituted pyridazinyl. In embodiments, R² is unsubstituted triazinyl. In embodiments, R² is unsubstituted pyrimidinyl. In embodiments, R² is unsubstituted imidazolyl. In embodiments, R² is unsubstituted pyrazinyl. In embodiments, R² is unsubstituted oxazolyl. In embodiments, R² is unsubstituted isoxazolyl. In embodiments, R² is unsubstituted thiazolyl. In embodiments, R² is unsubstituted furyl. In embodiments, R² is unsubstituted thienyl. In embodiments, R² is unsubstituted pyridyl. In embodiments, R² is unsubstituted pyrimidyl. In embodiments, R² is unsubstituted benzothiazolyl. In embodiments, R² is unsubstituted benzoxazoyl. In embodiments, R² is unsubstituted benzimidazolyl. In embodiments, R² is unsubstituted benzofuran. In embodiments, R² is unsubstituted isobenzofuranyl. In embodiments, R² is unsubstituted indolyl. In embodiments, R² is or unsubstituted isoindolyl. In embodiments, R² is independently —F or —OCF₃.

In embodiments, R² is independently halogen, —OCC1₃, —OCF₃, —OCBr₃, —OCI₃, or substituted 5 to 6 membered heteroaryl. In embodiments, R² is independently halogen. In embodiments, R² is independently —F. In embodiments, R² is independently —Cl. In embodiments, R² is independently —Br. In embodiments, R² is independently —I. In embodiments, R² is independently —OCC1₃. In embodiments, R² is independently —OCF₃. In embodiments, R² is independently —OCBr₃. In embodiments, R² is independently —OCI₃. In embodiments, R² is substituted 5 to 6 membered heteroaryl. In embodiments, R² is substituted 5 membered heteroaryl. In embodiments, R² is substituted 6 membered heteroaryl. In embodiments, R² is substituted pyrrolyl, pyrazolyl, pyridazinyl, triazinyl, pyrimidinyl, imidazolyl, pyrazinyl, oxazolyl, isoxazolyl, thiazolyl, furyl, thienyl, pyridyl, pyrimidyl, benzothiazolyl, benzoxazoyl, benzimidazolyl, benzofuran, isobenzofuranyl, indolyl, or isoindolyl. In embodiments, R² is substituted phenyl, benzimidazolyl, or indolyl. In embodiments, R² is substituted pyrrolyl. In embodiments, R² is substituted pyrazolyl. In embodiments, R² is substituted pyridazinyl. In embodiments, R² is substituted triazinyl. In embodiments, R² is substituted pyrimidinyl. In embodiments, R² is substituted imidazolyl. In embodiments, R² is substituted pyrazinyl. In embodiments, R² is substituted oxazolyl. In embodiments, R² is substituted isoxazolyl. In embodiments, R² is substituted thiazolyl. In embodiments, R² is substituted furyl. In embodiments, R² is substituted thienyl. In embodiments, R² is substituted pyridyl. In embodiments, R² is substituted pyrimidyl. In embodiments, R² is substituted benzothiazolyl. In embodiments, R² is substituted benzoxazoyl. In embodiments, R² is substituted benzimidazolyl. In embodiments, R² is substituted benzofuran. In embodiments, R² is substituted isobenzofuranyl. In embodiments, R² is substituted indolyl. In embodiments, R² is or substituted isoindolyl. In embodiments, R² is independently —F or —OCF₃.

In embodiments, L³ is a bond, —S(O)₂—, —NR³—, —NH—, —O—, —S—, —C(O)—, —C(O)NR³—, —NR³C(O)—, —N(R³)CH₂—, —NR³C(O)NH —, —NHC(O)NR³—, —C(O)O—, —OC(O)—, substituted or unsubstituted C₁-C₆ alkylene, or substituted or unsubstituted 2 to 6 membered heteroalkylene.

In embodiments, L³ is —C(O)—, —CH₂—, —C(O)NR³—, —CH₂CH₂NR³—, —C(O)CH₂NR³—, or —CH₂C(O)NR³. In embodiments, L³ is —C(O)—. In embodiments, L³ is —CH₂—. In embodiments, L³ is —C(O)NR³—. In embodiments, L³ is —CH₂CH₂NR³—. In embodiments, L³ is —C(O)CH₂NR³—. In embodiments, L³ is —CH₂C(O)NR³. In embodiments, L³ is —C(O)—, —CH₂—, —C(O)NH—, —CH₂CH₂NH—, —C(O)CH₂NH—, or —CH₂C(O)NH. In embodiments, wherein L³ is —CH₂— or —C(O)NH—. In embodiments, L³ is —C(O)NH—. In embodiments, L³ is —CH₂CH₂NH—. In embodiments, L³ is —C(O)CH₂NH—. In embodiments, L³ is or —CH₂C(O)NH. In embodiments, L³ is a bond, substituted or unsubstituted C₁-C₆ alkylene, or substituted or unsubstituted 2 to 6 membered heteroalkylene. In embodiments, L³ is a bond. In embodiments, L³ is substituted or unsubstituted C₁-C₆ alkylene. In embodiments, L³ is substituted or unsubstituted 2 to 6 membered heteroalkylene.

In embodiments, L⁴ is —NH—.

In embodiments, L⁴ is —CH₂—.

In embodiments, L⁴ is —N(CH₃)—.

In embodiments, L⁶ is —N(R⁶)—L³—. In embodiments, L⁶ is —N(R⁶)—L³—; L³ is —CH₂—; and R⁶ is as described herein, including embodiments.

In embodiments, L⁶ is —N(R⁶)—L³—; L³ is —CH₂—; and R⁶ is —CF₃, —COCH₃, or cyclopropyl.

In embodiments, L⁶ is —C(O)NH—.

In embodiments, W⁵ is CH. In embodiments, W⁵ is N.

In embodiments, R³, R⁴, and R⁶ are independently hydrogen, —CCl₃, —CBr₃, —CF₃, —CI₃, CHC1₂, —CHBr₂, —CHF₂, —CHI₂, —CH₂CI, —CH₂Br, —CH₂F, —CH₂I, —CN, —OH, —NH₂, —COOH, —CO(C₁-C₆ alkyl), —CONH₂, —OCCl₃, —OCF₃, —OCBr₃, —OCI₃, —OCHC1₂, —OCHBr₂, —OCHI₂, —OCHF₂, —OCH₂ C1, —OCH₂Br, —OCH₂I, —OCH₂F, unsubstituted C₁-C₆ alkyl, unsubstituted 2 to 6 membered heteroalkyl, unsubstituted C₃-C₆ cycloalkyl,or unsubstituted 3 to 6 membered heterocycloalkyl.

In embodiments, R³, R⁴, and R⁶ are independently hydrogen, —CCl₃, —CBr₃, —CF₃, —CI₃, CHC1₂, —CHBr₂, —CHF₂, —CHI₂, —CH₂C1, —CH₂Br, —CH₂F, —CH₂I, —CN, —OH, —NH₂, —COOH, —CONH₂, —OCCl₃, —OCF₃, —OCBr₃, —O CI₃, —OCHC1₂, —OCHBr₂, —OCHI₂, —OCHF₂, —OCH₂C1, —OCH₂Br, —OCH₂I, —OCH₂F, unsubstituted C₁-C₆ alkyl, or unsubstituted 2 to 6 membered heteroalkyl.

In embodiments, R³ is independently hydrogen, —CCl₃, —CBr₃, —CF₃, —CI₃, CHC1₂, —CHBr₂, —CHF₂, —CHI₂, —CH₂C1, —CH₂Br, —CH₂F, —CH₂I, —CN, —OH, —NH₂, —COOH, —COCH₃, —CONH₂, —OCCl₃, —OCF₃, —OCBr₃, —OCI₃, —OCHC1₂, —OCHBr₂, —OCHI₂, —OCHF₂, —OC H₂Cl, —OCH₂Br, —OCH₂I, —OCH₂F, unsubstituted C₁-C₆ alkyl, unsubstituted 2 to 6 membered heteroalkyl, unsubstituted C₃-C₆ cycloalkylene, or unsubstituted 3 to 6 membered heterocycloalkylene.

In embodiments, R³ is independently hydrogen, —CC1₃, —CBr₃, —CF₃, —CI₃, CHC1₂, —CHBr₂, —CHF₂, —CHI₂, —CH₂C1, —CH₂Br, —CH₂F, —CH₂I, —CN, —OH, —NH₂, —COOH, —CONH₂, —OCCl₃, —OCF₃, —OCBr₃, —O CI₃, —OCHC1₂, —OCHBr₂, —OCHI₂, —OCHF₂, —OCH₂C1, —OCH₂Br, —OCH₂I, —OCH₂F, unsubstituted C₁-C₆ alkyl, or unsubstituted 2 to 6 membered heteroalkyl.

In embodiments, R⁴ is independently hydrogen, —CC1₃, —CBr₃, —CF₃, —CI₃, CHC1₂, —CHBr₂, —CHF₂, —CHI₂, —CH₂C1, —CH₂Br, —CH₂F, —CH₂I, —CN, —OH, —NH₂, —COOH, —COCH₃, —CONH₂, —OCCl₃, —OCF₃, —OCBr₃, —OCI₃, —OCHC1₂, —OCHBr₂, —OCHI₂, —OCHF₂, —OC H₂Cl, —OCH₂Br, —OCH₂I, —OCH₂F, unsubstituted C₁-C₆ alkyl, unsubstituted 2 to 6 membered heteroalkyl, unsubstituted C₃-C₆ cycloalkylene, or unsubstituted 3 to 6 membered heterocycloalkylene.

In embodiments, R⁴ is independently hydrogen, —CC1₃, —CBr₃, —CF₃, —CI₃, CHC1₂, —CHBr₂, —CHF₂, —CHI₂, —CH₂C1, —CH₂Br, —CH₂F, —CH₂I, —CN, —OH, —NH₂, —COOH, —CONH₂, —OCCl₃, —OCF₃, —OCBr₃, —O CI₃, —OCHC1₂, —OCHBr₂, —OCHI₂, —OCHF₂, —OCH₂C1, —OCH₂Br, —OCH₂I, —OCH₂F, unsubstituted C₁-C₆ alkyl, or unsubstituted 2 to 6 membered heteroalkyl.

In embodiments, R⁶ is independently hydrogen, —CC1₃, —CBr₃, —CF₃, —CI₃, CHC1₂, —CHBr₂, —CHF₂, —CHI₂, —CH₂C1, —CH₂Br, —CH₂F, —CH₂I, —CN, —OH, —NH₂, —COOH, —COCH₃, —CONH₂, —OCCl₃, —OCF₃, —OCBr₃, —OCI₃, —OCHC1₂, —OCHBr₂, —OCHI₂, —OCHF₂, —OC H₂Cl, —OCH₂Br, —OCH₂I, —OCH₂F, unsubstituted C₁-C₆ alkyl, unsubstituted 2 to 6 membered heteroalkyl, unsubstituted C₃-C₆ cycloalkylene, or unsubstituted 3 to 6 membered heterocycloalkylene.

In embodiments, R⁶ is independently hydrogen, —CC1₃, —CBr₃, —CF₃, —CI₃, CHC1₂, —CHBr₂, —CHF₂, —CHI₂, —CH₂C1, —CH₂Br, —CH₂F, —CH₂I, —CN, —OH, —NH₂, —COOH, —CONH₂, —OCCl₃, —OCF₃, —OCBr₃, —O CI₃, —OCHC1₂, —OCHBr₂, —OCHI₂, —OCHF₂, —OCH₂C1, —OCH₂Br, —OCH₂I, —OCH₂F, unsubstituted C₁-C₆ alkyl, or unsubstituted 2 to 6 membered heteroalkyl.

In embodiments, R³ is independently hydrogen. In embodiments, R³ is independently —CC1₃. In embodiments, R³ is independently —CBr₃. In embodiments, R³ is independently —CF₃. In embodiments, R³ is independently —CI₃. In embodiments, R³ is independently CHCl₂. In embodiments, R³ is independently —CHBr₂. In embodiments, R³ is independently —CHF₂. In embodiments, R³ is independently —CHI₂. In embodiments, R³ is independently —C(O)CH₃. In embodiments, R³ is independently —CH₂Cl. In embodiments, R³ is independently —CH₂Br. In embodiments, R³ is independently —CH₂F. In embodiments, R³ is independently —CH₂I. In embodiments, R³ is independently unsubstituted C₁-C₆ alkyl. In embodiments, R³ is independently unsubstituted C₁-C₂ alkyl. In embodiments, R³ is independently unsubstituted methyl. In embodiments, R³ is independently unsubstituted 2 to 6 membered heteroalkyl. In embodiments, R³ is independently unsubstituted C₃-C₆ cycloalkyl. In embodiments, R³ is independently unsubstituted cyclopropyl. In embodiments, R³ is independently unsubstituted cyclobutyl. In embodiments, R³ is independently unsubstituted cyclopentyl. In embodiments, R³ is independently unsubstituted cyclohexyl. In embodiments, R³ is independently unsubstituted 3 to 6 membered heterocycloalkyl.

In embodiments, R⁴ is independently hydrogen. In embodiments, R⁴ is independently —CCl₃. In embodiments, R⁴ is independently —CBr₃. In embodiments, R⁴ is independently —CF₃. In embodiments, R⁴ is independently —CI₃. In embodiments, R⁴ is independently CHCl₂. In embodiments, R⁴ is independently —CHBr₂. In embodiments, R⁴ is independently —CHF₂. In embodiments, R⁴ is independently —CHI₂. In embodiments, R⁴ is independently —CH₂Cl. In embodiments, R⁴ is independently —CH₂Br. In embodiments, R⁴ is independently —CH₂F. In embodiments, R⁴ is independently —CH₂I. In embodiments, R⁴ is independently —C(O)CH₃. In embodiments, R⁴ is independently unsubstituted C₁-C₆ alkyl. In embodiments, R⁴ is independently unsubstituted C₁-C₂ alkyl. In embodiments, R⁴ is independently unsubstituted methyl. In embodiments, R⁴ is independently unsubstituted 2 to 6 membered heteroalkyl. In embodiments, R⁴ is independently unsubstituted C₃-C₆ cycloalkyl. In embodiments, R⁴ is independently unsubstituted cyclopropyl. In embodiments, R⁴ is independently unsubstituted cyclobutyl. In embodiments, R⁴ is independently unsubstituted cyclopentyl. In embodiments, R⁴ is independently unsubstituted cyclohexyl. In embodiments, R⁴ is independently unsubstituted 3 to 6 membered heterocycloalkyl.

In embodiments, R⁶ is independently hydrogen. In embodiments, R⁶ is independently —CCl₃. In embodiments, R⁶ is independently —CBr₃. In embodiments, R⁶ is independently —CF₃. In embodiments, R⁶ is independently —CI₃. In embodiments, R⁶ is independently CHCl₂. In embodiments, R⁶ is independently —CHBr₂. In embodiments, R⁶ is independently —CHF₂. In embodiments, R⁶ is independently —CHI₂. In embodiments, R⁶ is independently —CH₂Cl. In embodiments, R⁶ is independently —CH₂Br. In embodiments, R⁶ is independently —CH₂F. In embodiments, R⁶ is independently —CH₂I. In embodiments, R⁶ is independently —C(O)CH₃. In embodiments, R⁶ is independently unsubstituted C₁-C₆ alkyl. In embodiments, R⁶ is independently unsubstituted C₁-C₂ alkyl. In embodiments, R⁶ is independently unsubstituted methyl. In embodiments, R⁶ is independently unsubstituted 2 to 6 membered heteroalkyl. In embodiments, R⁶ unsubstituted C₃-C₆ cycloalkyl. In embodiments, R⁶ is independently unsubstituted cyclopropyl. In embodiments, R⁶ is independently unsubstituted cyclobutyl. In embodiments, R⁶ is independently unsubstituted cyclopentyl. In embodiments, R⁶ is independently unsubstituted cyclohexyl. In embodiments, R⁶ is independently unsubstituted 3 to 6 membered heterocycloalkyl.

In embodiments, R⁷ is independently hydrogen or unsubstituted C₁-C₁₀ alkyl. In embodiments, R⁷ is independently hydrogen or unsubstituted C₁-C₆ alkyl. In embodiments, R⁷ is independently hydrogen or unsubstituted C₁-C₄ alkyl. In embodiments, R⁷ is independently hydrogen or unsubstituted methyl. In embodiments, R⁷ is independently hydrogen or unsubstituted ethyl. In embodiments, R⁷ is independently hydrogen. In embodiments, R⁷ is independently unsubstituted methyl. In embodiments, R⁷ is independently unsubstituted ethyl.

In embodiments, R⁸ is independently hydrogen or unsubstituted C₁-C₁₀ alkyl. In embodiments, R⁸ is independently hydrogen or unsubstituted C₁-C₆ alkyl. In embodiments, R⁸ is independently hydrogen or unsubstituted C₁-C₄ alkyl. In embodiments, R⁸ is independently hydrogen or unsubstituted methyl. In embodiments, R⁸ is independently hydrogen or unsubstituted ethyl. In embodiments, R⁸ is independently hydrogen. In embodiments, R⁸ is independently unsubstituted methyl. In embodiments, R⁸ is independently unsubstituted ethyl.

In embodiments, R⁹ is independently hydrogen or unsubstituted C₁-C₁₀ alkyl. In embodiments, R⁹ is independently hydrogen or unsubstituted C₁-C₆ alkyl. In embodiments, R⁹ is independently hydrogen or unsubstituted C₁-C₄ alkyl. In embodiments, R⁹ is independently hydrogen or unsubstituted methyl. In embodiments, R⁹ is independently hydrogen or unsubstituted ethyl. In embodiments, R⁹ is independently hydrogen. In embodiments, R⁹ is independently unsubstituted methyl. In embodiments, R⁹ is independently unsubstituted ethyl.

In embodiments, R¹⁰ is independently hydrogen or unsubstituted C₁-C₁₀ alkyl. In embodiments, R¹⁰ is independently hydrogen or unsubstituted C₁-C₆ alkyl. In embodiments, R¹⁰ is independently hydrogen or unsubstituted C₁-C₄ alkyl. In embodiments, R¹⁰ is independently hydrogen or unsubstituted methyl. In embodiments, R¹⁰ is independently hydrogen or unsubstituted ethyl. In embodiments, R¹⁰ is independently hydrogen. In embodiments, R¹⁰ is independently unsubstituted methyl. In embodiments, R¹⁰ is independently unsubstituted ethyl.

In embodiments, R⁷ and R⁸ are hydrogen. In embodiments, R⁷ and R⁸ are unsubstituted C₁-C₆ alkyl. In embodiments, R⁷ and R⁸ are unsubstituted methyl. In embodiments, R⁹ and R¹⁰ are hydrogen. In embodiments, R⁹ and R¹⁰ are unsubstituted C₁-C₆ alkyl. In embodiments, R⁹ and R¹⁰ are unsubstituted methyl. In embodiments, R⁷ and R⁸ are hydrogen and R⁹ and R¹⁰ are unsubstituted C₁-C₆ alkyl. In embodiments, R⁷ and R⁸ are hydrogen and R⁹ and R¹⁰ are unsubstituted methyl. In embodiments, R⁹ and R¹⁰ are hydrogen and R⁷ and R⁸ are unsubstituted C₁-C₆ alkyl. In embodiments, R⁹ and R¹⁰ are hydrogen and R⁷ and R⁸ are unsubstituted methyl.

In embodiments, Ring B is aryl. In embodiments, Ring B is C₆-C₁₀ aryl. In embodiments, Ring B is phenyl. In embodiments, Ring B is C₉ aryl. In embodiments, Ring B is C₁₀ aryl. In embodiments, Ring B is heteroaryl. In embodiments, Ring B is 5 to 10 membered heteroaryl. In embodiments, Ring B is 5 to 6 membered heteroaryl. In embodiments, Ring B is 9 to 10 membered heteroaryl. In embodiments, Ring B is 5-membered heteroaryl. In embodiments, Ring B is 6-membered heteroaryl. In embodiments, Ring B is pyrrolyl, pyrazolyl, pyridazinyl, triazinyl, pyrimidinyl, imidazolyl, pyrazinyl, oxazolyl, isoxazolyl, thiazolyl, furyl, thienyl, pyridyl, pyrimidyl, benzothiazolyl, benzoxazoyl, benzimidazolyl, benzofuran, isobenzofuranyl, indolyl, or isoindolyl. In embodiments, Ring B is phenyl, benzimidazolyl, or indolyl. In embodiments, Ring B is pyrrolyl. In embodiments, Ring B is pyrazolyl. In embodiments, Ring B is pyridazinyl. In embodiments, Ring B is triazinyl. In embodiments, Ring B is pyrimidinyl. In embodiments, Ring B is imidazolyl. In embodiments, Ring B is pyrazinyl. In embodiments, Ring B is oxazolyl. In embodiments, Ring B is isoxazolyl. In embodiments, Ring B is thiazolyl. In embodiments, Ring B is furyl. In embodiments, Ring B is thienyl. In embodiments, Ring B is pyridyl. In embodiments, Ring B is pyrimidyl. In embodiments, Ring B is benzothiazolyl. In embodiments, Ring B is benzoxazoyl. In embodiments, Ring B is benzimidazolyl. In embodiments, Ring B is benzofuran. In embodiments, Ring B is isobenzofuranyl. In embodiments, Ring B is indolyl. In embodiments, Ring B is isoindolyl.

In embodiments, -(Ring B)-(R²)_{z2} is R²-substituted or unsubstituted C₆-C₁₀ aryl. In embodiments, Ring B is R²-substituted or unsubstituted phenyl. In embodiments, Ring B is R²-substituted or unsubstituted C₉ aryl. In embodiments, Ring B is R²-substituted or unsubstituted C₁₀ aryl. In embodiments, Ring B is R²-substituted or unsubstituted 5 to 10 membered heteroaryl. In embodiments, Ring B is R²-substituted or unsubstituted 5 to 6 membered heteroaryl. In embodiments, Ring B is R²-substituted or unsubstituted 9 to 10 membered heteroaryl. In embodiments, Ring B is R²-substituted or unsubstituted 5-membered heteroaryl. In embodiments, Ring B is R²-substituted or unsubstituted 6-membered heteroaryl. In embodiments, Ring B is R²-substituted or unsubstituted pyrrolyl, pyrazolyl, pyridazinyl, triazinyl, pyrimidinyl, imidazolyl, pyrazinyl, oxazolyl, isoxazolyl, thiazolyl, furyl, thienyl, pyridyl, pyrimidyl, benzothiazolyl, benzoxazoyl, benzimidazolyl, benzofuran, isobenzofuranyl, indolyl, or isoindolyl. In embodiments, Ring B is R²-substituted or unsubstituted phenyl, benzimidazolyl, or indolyl. In embodiments, Ring B is R²-substituted or unsubstituted pyrrolyl. In embodiments, Ring B is R²-substituted or unsubstituted pyrazolyl. In embodiments, Ring B is R²-substituted or unsubstituted pyridazinyl. In embodiments, Ring B is R²-substituted or unsubstituted triazinyl. In embodiments, Ring B is R²-substituted or unsubstituted pyrimidinyl. In embodiments, Ring B is R²-substituted or unsubstituted imidazolyl. In embodiments, Ring B is R²-substituted or unsubstituted pyrazinyl. In embodiments, Ring B is R²-substituted or unsubstituted oxazolyl. In embodiments, Ring B is R²-substituted or unsubstituted isoxazolyl. In embodiments, Ring B is R²-substituted or unsubstituted thiazolyl. In embodiments, Ring B is R²-substituted or unsubstituted furyl. In embodiments, Ring B is R²-substituted or unsubstituted thienyl. In embodiments, Ring B is R²-substituted or unsubstituted pyridyl. In embodiments, Ring B is R²-substituted or unsubstituted pyrimidyl. In embodiments, Ring B is R²-substituted or unsubstituted benzothiazolyl. In embodiments, Ring B is R²-substituted or unsubstituted benzoxazoyl. In embodiments, Ring B is R²-substituted or unsubstituted benzimidazolyl. In embodiments, Ring B is R²-substituted or unsubstituted benzofuran. In embodiments, Ring B is R²-substituted or unsubstituted isobenzofuranyl. In embodiments, Ring B is R²-substituted or unsubstituted indolyl. In embodiments, Ring B is R²-substituted or unsubstituted isoindolyl.

In embodiments, Ring B has the formula:

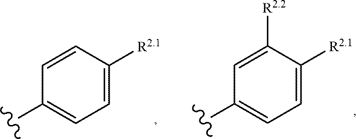

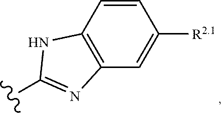

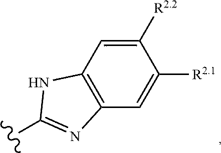

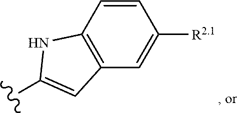

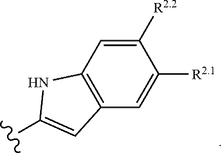

R^{2.1} and R^{2.2} are as described herein, including in embodiments.

In embodiments, Ring B has the formula:

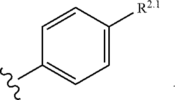

R^{2.1} is as described herein, including in embodiments. In embodiments, Ring B has the formula:

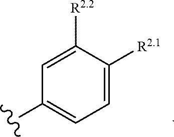

R^{2.1} and R^{2.2} are as described herein, including in embodiments. In embodiments, Ring B has the formula:

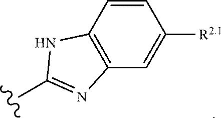

R^{2.1} is as described herein, including in embodiments. In embodiments, Ring B has the formula:

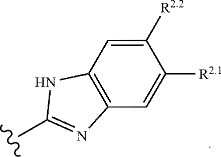

R^{2.1} and R^{2.2} are as described herein, including in embodiments. In embodiments, Ring B has the formula:

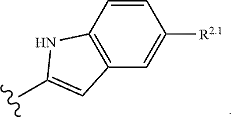

R^{2.1} is as described herein, including in embodiments. In embodiments, Ring B has the formula:

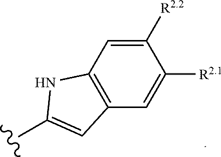

R^{2.1} and R^{2.2} are as described herein, including in embodiments.

In embodiments, Ring B has the formula:

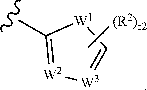

R², z2, W¹, W², and W³ are as described herein, including in embodiments.

In embodiments, R⁵ is independently

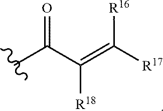

R¹⁶, R¹⁷, and R¹⁸ are as described herein, including in embodiments. In embodiments, R⁵ is independently

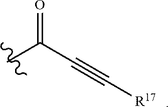

R¹⁷ is as described herein, including in embodiments. In embodiments, R⁵ is independently

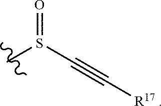

R¹⁷ is as described herein, including in embodiments. In embodiments, R⁵ is independently

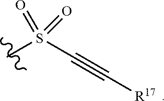

R¹⁷ is as described herein, including in embodiments. In embodiments, R⁵ is independently

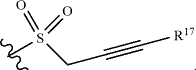

R¹⁷ is as described herein, including in embodiments. In embodiments, R⁵ is independently

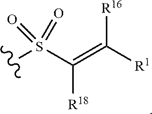

R¹⁶, R¹⁷, and R¹⁸ are as described herein, including in embodiments. In embodiments, R⁵ is independently

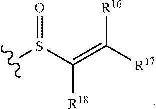

R¹⁶, R¹⁷, and R¹⁸ are as described herein, including in embodiments. In embodiments, R⁵ is independently

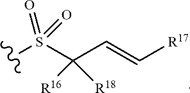

R¹⁶, R¹⁷, and R¹⁸ are as described herein, including in embodiments. In embodiments, R⁵ is independently

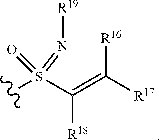

R¹⁶, R¹⁷, R¹⁸, and R¹⁹ are as described herein, including in embodiments. In embodiments, R⁵ is independently

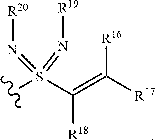

R¹⁶, R¹⁷, R¹⁸, R¹⁹, and R²⁰ are as described herein, including in embodiments. In embodiments, R⁵ is independently

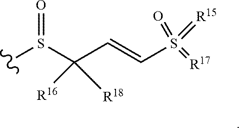

R¹⁵, R¹⁶, R¹⁷, and R¹⁸ are as described herein, including in embodiments. In embodiments, R⁵ is independently

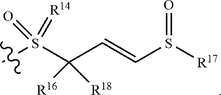

R¹⁴, R¹⁶, R¹⁷, and R¹⁸ are as described herein, including in embodiments. In embodiments, R⁵ is independently

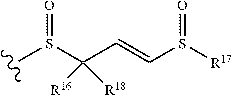

R¹⁶, R¹⁷, and R¹⁸ are as described herein, including in embodiments. In embodiments, R⁵ is independently

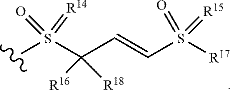

R¹⁴, R¹⁵, R¹⁶, R¹⁷, and R¹⁸ are as described herein, including in embodiments.

In embodiments, R⁵ is independently

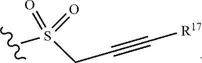

R¹⁷ is substituted or unsubstituted alkyl. In embodiments, R¹⁷ is unsubstituted alkyl. In embodiments, R¹⁷ is unsubstituted methyl. In embodiments, R¹⁷ is unsubstituted ethyl. In embodiments, R¹⁷ is unsubstituted propyl.

In embodiments, R⁵ is independently

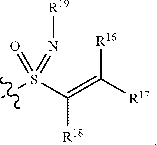

R¹⁶, R¹⁷, and R¹⁸ are as described herein, including in embodiments. R¹⁹ is hydrogen, —CF₃, —CN, or unsubstituted methyl. In embodiments, R¹⁹ is hydrogen. In embodiments, R¹⁹ is —CF₃. In embodiments, R¹⁹ is —CN. In embodiments, R¹⁹ is unsubstituted methyl. In embodiments, R¹⁶, R¹⁷, and R¹⁸ are hydrogen.

In embodiments, R⁵ is independently

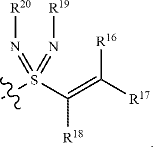

R¹⁶, R¹⁷, and R¹⁸ are as described herein, including in embodiments. R¹⁹ and R²⁰ are independently hydrogen, —CF₃, —CN, or unsubstituted methyl. In embodiments, R¹⁹ is hydrogen. In embodiments, R¹⁹ is —CF₃. In embodiments, R¹⁹ is —CN. In embodiments, R¹⁹ is unsubstituted methyl. In embodiments, R²⁰ is hydrogen. In embodiments, R²⁰ is —CF₃. In embodiments, R²⁰ is —CN. In embodiments, R²⁰ is unsubstituted methyl. In embodiments, R¹⁶, R¹⁷, and R¹⁸ are hydrogen.

In embodiments, R⁵ is independently

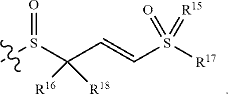

R¹⁵, R¹⁶, R¹⁷, and R¹⁸ are as described herein, including in embodiments. In embodiments, R¹⁷ is substituted or unsubstituted phenyl. In embodiments, R⁵ is independently

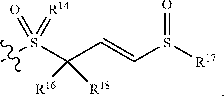

R¹⁴, R¹⁶, R¹⁷, and R¹⁸ are as described herein, including in embodiments. In embodiments, R¹⁷ is substituted or unsubstituted phenyl. In embodiments, R⁵ is independently

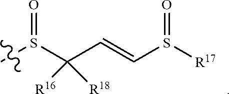

R¹⁶, R¹⁷, and R¹⁸ are as described herein, including in embodiments. In embodiments, R¹⁷ is substituted or unsubstituted phenyl. In embodiments, R⁵ is independently

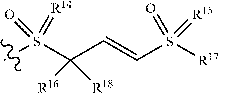

R¹⁴, R¹⁵, R¹⁶, R¹⁷, and R¹⁸ are as described herein, including in embodiments. In embodiments, R¹⁷ is substituted or unsubstituted phenyl.

In embodiments, R¹⁴ is independently ═O. In embodiments, R¹⁴ is independently ═NR¹⁹.

In embodiments, R¹⁵ is independently ═O. In embodiments, R¹⁵ is independently ═NR²⁰.

In embodiments, R⁵ is independently

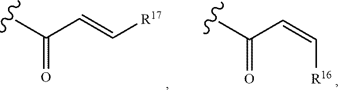

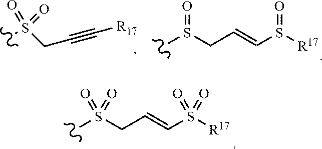

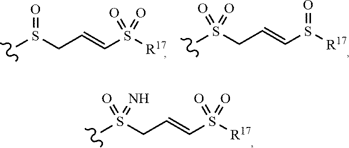

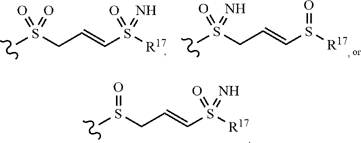

R¹⁶ and R¹⁷ are as described herein, including in embodiments.

In embodiments, R⁵ is independently

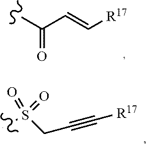

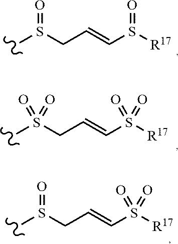

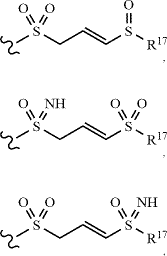

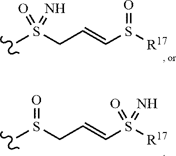

R¹⁷ is as described herein, including in embodiments.

In embodiments, R⁵ is independently

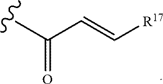

R¹⁷ is as described herein, including in embodiments. In embodiments, R¹⁷ is substituted or unsubstituted heteroaryl. In embodiments, R¹⁷ is substituted or unsubstituted 5 to 6 membered heteroaryl. In embodiments, R¹⁷ is substituted or unsubstituted pyrrolyl, pyrazolyl, pyridazinyl, triazinyl, pyrimidinyl, imidazolyl, pyrazinyl, oxazolyl, isoxazolyl, thiazolyl, furyl, thienyl, pyridyl, or pyrimidyl. In embodiments, R¹⁷ is substituted or unsubstituted triazinyl. In embodiments, R¹⁷ is unsubstituted triazinyl. In embodiments, R¹⁷ is substituted or unsubstituted benzothiazolyl, benzoxazoyl, benzimidazolyl, benzofuran, isobenzofuranyl, indolyl, isoindolyl, benzothiophenyl, isoquinolyl, quinoxalinyl, or quinolyl.

In embodiments, R⁵ is independently

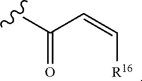

R¹⁶ is as described herein, including in embodiments. In embodiments, R¹⁶ is substituted or unsubstituted heteroaryl. In embodiments, R¹⁶ is substituted or unsubstituted 5 to 6 membered heteroaryl. In embodiments, R¹⁶ is substituted or unsubstituted pyrrolyl, pyrazolyl, pyridazinyl, triazinyl, pyrimidinyl, imidazolyl, pyrazinyl, oxazolyl, isoxazolyl, thiazolyl, furyl, thienyl, pyridyl, or pyrimidyl. In embodiments, R¹⁶ is substituted or unsubstituted triazinyl. In embodiments, R¹⁶ is unsubstituted triazinyl. In embodiments, R¹⁶ is substituted or unsubstituted benzothiazolyl, benzoxazoyl, benzimidazolyl, benzofuran, isobenzofuranyl, indolyl, isoindolyl, benzothiophenyl, isoquinolyl, quinoxalinyl, or quinolyl.

In embodiments, R⁵ is independently

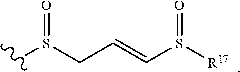

R¹⁷ is as described herein, including in embodiments. In embodiments, R⁵ is independently

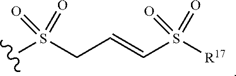

R¹⁷ is as described herein, including in embodiments. In embodiments, R⁵ is independently

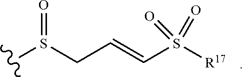

R¹⁷ is as described herein, including in embodiments. In embodiments, R⁵ is independently

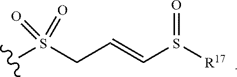

R¹⁷ is as described herein, including in embodiments. In embodiments, R⁵ is independently

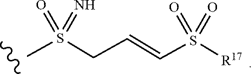

R¹⁷ is as described herein, including in embodiments. In embodiments, R⁵ is independently

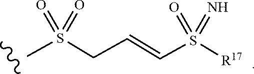

R¹⁷ is as described herein, including in embodiments. In embodiments, R⁵ is independently

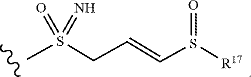

R¹⁷ is as described herein, including in embodiments. In embodiments, R⁵ is independently embodiments.

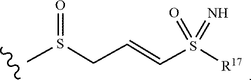

R¹⁷ is as described herein, including in

In embodiments, R⁵ is independently

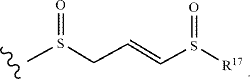

In embodiments, R¹⁷ is substituted or unsubstituted aryl. In embodiments, R¹⁷ is substituted or unsubstituted phenyl. In embodiments, R¹⁷ is unsubstituted phenyl. In embodiments, R⁵ is independently

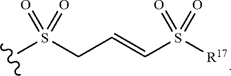

In embodiments, R¹⁷ is substituted or unsubstituted aryl. In embodiments, R¹⁷ is substituted or unsubstituted phenyl. In embodiments, R¹⁷ is unsubstituted phenyl. In embodiments, R⁵ is independently

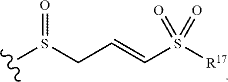

In embodiments, R¹⁷ is substituted or unsubstituted aryl. In embodiments, R¹⁷ is substituted or unsubstituted phenyl. In embodiments, R¹⁷ is unsubstituted phenyl. In embodiments, R⁵ is independently

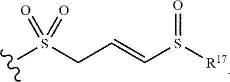

In embodiments, R¹⁷ is substituted or unsubstituted aryl. In embodiments, R¹⁷ is substituted or unsubstituted phenyl. In embodiments, R¹⁷ is unsubstituted phenyl. In embodiments, R⁵ is independently

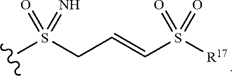

In embodiments, R¹⁷ is substituted or unsubstituted aryl. In embodiments, R¹⁷ is substituted or unsubstituted phenyl. In embodiments, R¹⁷ is unsubstituted phenyl. In embodiments, R⁵ is independently

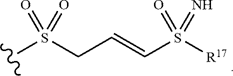

In embodiments, R¹⁷ is substituted or unsubstituted aryl. In embodiments, R¹⁷ is substituted or unsubstituted phenyl. In embodiments, R¹⁷ is unsubstituted phenyl. In embodiments, R⁵ is independently

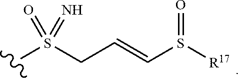

In embodiments, R¹⁷ is substituted or unsubstituted aryl. In embodiments, R¹⁷ is substituted or unsubstituted phenyl. In embodiments, R¹⁷ is unsubstituted phenyl. In embodiments, R⁵ is independently

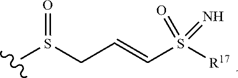

In embodiments, R¹⁷ is substituted or unsubstituted aryl. In embodiments, R¹⁷ is substituted or unsubstituted phenyl. In embodiments, R¹⁷ is unsubstituted phenyl.

In embodiments, R⁵ is independently:

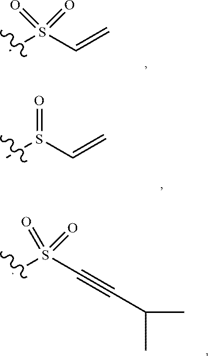

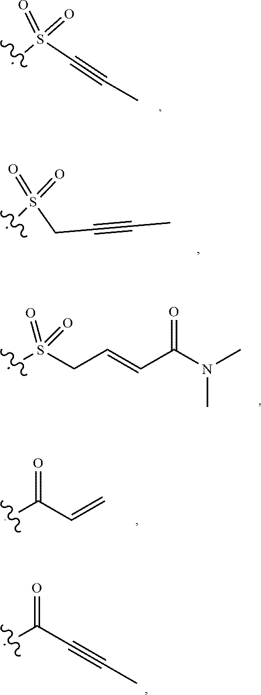

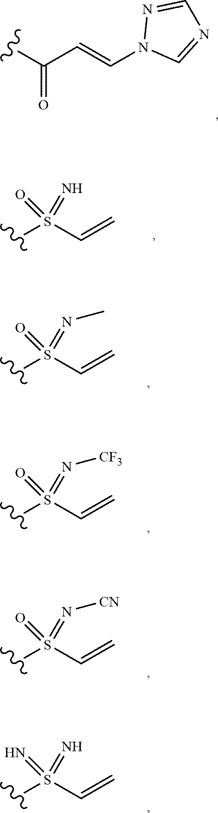

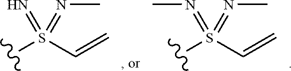

In embodiments, R⁵ is independently:

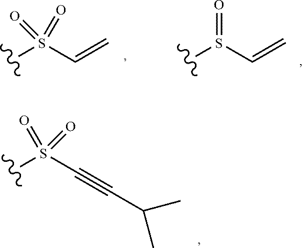

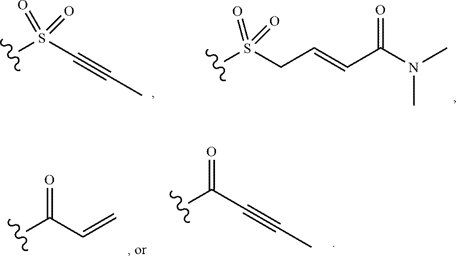

In embodiments, R⁵ is independently

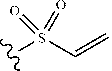

In embodiments, R⁵ is independently

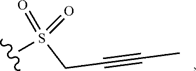

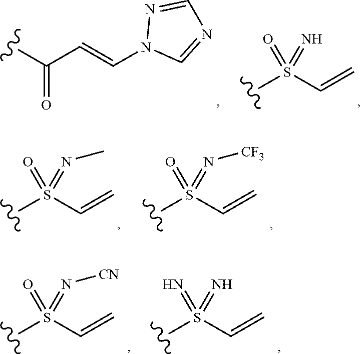

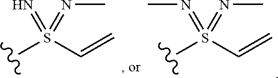

In embodiments, R⁵ is independently

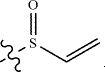

In embodiments, R⁵ is independently

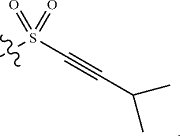

In embodiments, R⁵ is independently

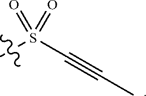

In embodiments, R⁵ is independently

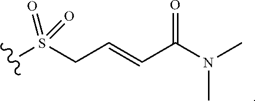

In embodiments, R⁵ is independently

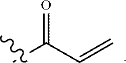

In embodiments, R⁵ is independently

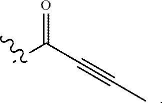

In embodiments, R⁵ is independently

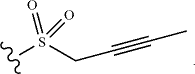

In embodiments, R⁵ is independently

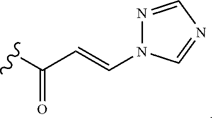

In embodiments, R⁵ is independently

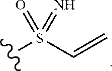

In embodiments, R⁵ is independently

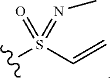

In embodiments, R⁵ is independently

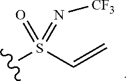

In embodiments, R⁵ is independently

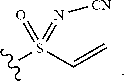

In embodiments, R⁵ is independently

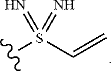

In embodiments, R⁵ is independently

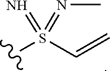

In embodiments, R⁵ is independently

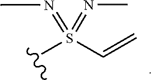

In embodiments, L⁴-R⁵ is independently

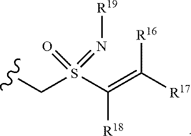

R¹⁶, R¹⁷, R¹⁸, and R¹⁹ are as described herein, including in embodiments. R¹⁶, R¹⁷, and R¹⁸ are as described herein, including in embodiments. R¹⁹ is hydrogen, —CF₃, —CN, or unsubstituted methyl. In embodiments, R¹⁹ is hydrogen. In embodiments, R¹⁹ is —CF₃. In embodiments, R¹⁹ is —CN. In embodiments, R¹⁹ is unsubstituted methyl. In embodiments, R¹⁶, R¹⁷, and R¹⁸ are hydrogen.

In embodiments, L⁴-R⁵ is independently

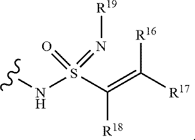

R¹⁶, R¹⁷, R¹⁸, and R¹⁹ are as described herein, including in embodiments. R¹⁶, R¹⁷, and R¹⁸ are as described herein, including in embodiments. R¹⁹ is hydrogen, —CF₃, —CN, or unsubstituted methyl. In embodiments, R¹⁹ is hydrogen. In embodiments, R¹⁹ is —CF₃. In embodiments, R¹⁹ is —CN. In embodiments, R¹⁹ is unsubstituted methyl. In embodiments, R¹⁶, R¹⁷, and R¹⁸ are hydrogen.

In embodiments, L⁴-R⁵ is independently

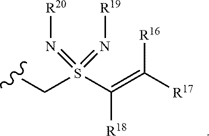

R¹⁶, R¹⁷, and R¹⁸ are as described herein, including in embodiments. R¹⁹ and R²⁰ are independently hydrogen, —CF₃, —CN, or unsubstituted methyl. In embodiments, R¹⁹ and R²⁰ are independently hydrogen or unsubstituted methyl. In embodiments, R¹⁹ is hydrogen. In embodiments, R¹⁹ is unsubstituted methyl. In embodiments, R²⁰ is hydrogen. In embodiments, R²⁰ is unsubstituted methyl. In embodiments, R¹⁶, R¹⁷, and R¹⁸ are hydrogen.

In embodiments, L⁴-R⁵ is independently

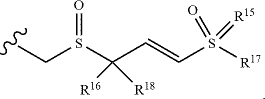

R¹⁵, R¹⁶, R¹⁷, and R¹⁸ are as described herein, including in embodiments. In embodiments, R¹⁷ is substituted or unsubstituted phenyl. In embodiments, L⁴-R⁵ is independently

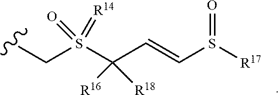

R¹⁴, R¹⁶, R¹⁷, and R¹⁸ are as described herein, including in embodiments. In embodiments, R¹⁷ is substituted or unsubstituted phenyl. In embodiments, L⁴-R⁵ is independently

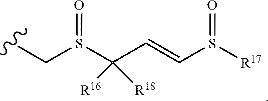

R¹⁶, R¹⁷, and R¹⁸ are as described herein, including in embodiments. In embodiments, R¹⁷ is substituted or unsubstituted phenyl. In embodiments, L⁴-R⁵ is independently

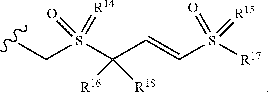

R¹⁴, R¹⁵, R¹⁶, R¹⁷, and R¹⁸ are as described herein, including in embodiments. In embodiments, R¹⁷ is substituted or unsubstituted phenyl.

In embodiments, L⁴-R⁵ is independently

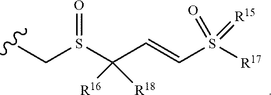

R¹⁵ is as described herein, including in embodiments; R¹⁷ is substituted or unsubstituted phenyl; and R¹⁶ and R¹⁸ are hydrogen. In embodiments, L⁴-R⁵ is independently

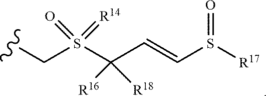

R¹⁵ is as described herein, including in embodiments; R¹⁷ is substituted or unsubstituted phenyl; and R¹⁶ and R¹⁸ are hydrogen. In embodiments, L⁴-R⁵ is independently

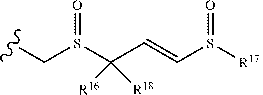

R¹⁵ is as described herein, including in embodiments; R¹⁷ is substituted or unsubstituted phenyl; and R¹⁶ and R¹⁸ are hydrogen. In embodiments, L⁴-R⁵ is independently

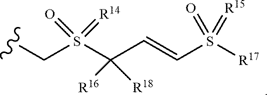

R¹⁵ is as described herein, including in embodiments; R¹⁷ is substituted or unsubstituted phenyl; and R¹⁶ and R¹⁸ are hydrogen.

In embodiments, L⁴-R⁵ is independently

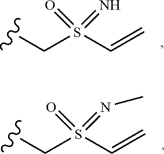

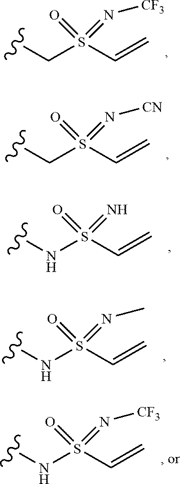

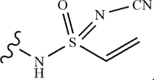

In embodiments, L⁴-R⁵ is independently

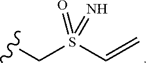

In embodiments, L⁴-R⁵ is independently

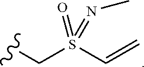

In embodiments, L⁴-R⁵ is independently

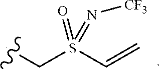

In embodiments, L⁴-R⁵ is independently

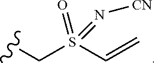

In embodiments, L⁴-R⁵ is independently

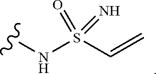

In embodiments, L⁴-R⁵ is independently

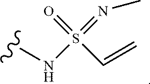

In embodiments, L⁴-R⁵ is independently

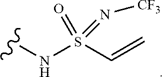

In embodiments, L⁴-R⁵ is independently

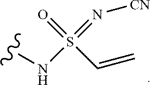

In embodiments, R¹⁶, R¹⁷, and R¹⁸ are independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl.

In embodiments, R¹⁶, R¹⁷, and R¹⁸ are independently hydrogen, substituted or unsubstituted C₁-C₆ alkyl, or substituted or unsubstituted 2 to 6 membered heteroalkyl. In embodiments, R¹⁶, R¹⁷, and R¹⁸ are independently hydrogen. In embodiments, R¹⁶, R¹⁷, and R¹⁸ are independently substituted or unsubstituted C₁-C₆ alkyl. In embodiments, R¹⁶, R¹⁷, and R¹⁸ are independently substituted or unsubstituted 2 to 6 membered heteroalkyl. In embodiments, R¹⁶, R¹⁷, and R¹⁸ are independently substituted or unsubstituted 2 to 3 membered heteroalkyl. In embodiments, R¹⁶, R¹⁷, and R¹⁸ are independently hydrogen or —C(O)N(CH₃)₂. In embodiments, R¹⁶, R¹⁷, and R¹⁸ are independently hydrogen or substituted or unsubstituted C₁-C₆ alkyl. In embodiments, R¹⁶, R¹⁷, and R¹⁸ are independently hydrogen or substituted or unsubstituted C₁-C₄ alkyl. In embodiments, R¹⁶, R¹⁷, and R¹⁸ are independently hydrogen or substituted or unsubstituted C₁-C₂ alkyl. In embodiments, R¹⁶, R¹⁷, and R¹⁸ are independently hydrogen or unsubstituted methyl. In embodiments, R¹⁶ and R¹⁷ are hydrogen and R¹⁸ is unsubstituted methyl. In embodiments, R¹⁶ and R¹⁸ are hydrogen and R¹⁷ is —C(O)N(CH₃)₂. In embodiments, R¹⁶, R¹⁷, and R¹⁸ are hydrogen. In embodiments, R¹⁷ is hydrogen or substituted or unsubstituted C₁-C₄ alkyl. In embodiments, R¹⁷ is hydrogen. In embodiments, R¹⁷ is unsubstituted methyl. In embodiments, R¹⁷ is ethyl. In embodiments, R¹⁷ is propyl. In embodiments, R¹⁷ is isopropyl. In embodiments, R¹⁷ is n-propyl.

In embodiments, R¹⁶ and R¹⁸ are hydrogen and R¹⁷ is substituted or unsubstituted aryl. In embodiments, R¹⁶ and R¹⁸ are hydrogen and R¹⁷ is unsubstituted C₆-C₁₂ aryl. In embodiments, R¹⁶ and R¹⁸ are hydrogen and R¹⁷ is substituted or unsubstituted phenyl. In embodiments, R¹⁶ and R¹⁸ are hydrogen and R¹⁷ is unsubstituted phenyl.

In embodiments, R¹⁶ and R¹⁸ are hydrogen and R¹⁷ is substituted or unsubstituted 5 to 6 membered heteroaryl. In embodiments, R¹⁶ and R¹⁸ are hydrogen and R¹⁷ is unsubstituted 5 to 6 membered heteroaryl. In embodiments, R¹⁶ and R¹⁸ are hydrogen and R¹⁷ is substituted or unsubstituted pyrrolyl. In embodiments, R¹⁶ and R¹⁸ are hydrogen and R¹⁷ is substituted or unsubstituted pyrazolyl. In embodiments, R¹⁶ and R¹⁸ are hydrogen and R¹⁷ is substituted or unsubstituted pyridazinyl. In embodiments, R¹⁶ and R¹⁸ are hydrogen and R¹⁷ is substituted or unsubstituted triazinyl. In embodiments, R¹⁶ and R¹⁸ are hydrogen and R¹⁷ is substituted or unsubstituted pyrimidinyl. In embodiments, R¹⁶ and R¹⁸ are hydrogen and R¹⁷ is substituted or unsubstituted imidazolyl. In embodiments, R¹⁶ and R¹⁸ are hydrogen and R¹⁷ is substituted or unsubstituted pyrazinyl. In embodiments, R¹⁶ and R¹⁸ are hydrogen and R¹⁷ is substituted or unsubstituted oxazolyl. In embodiments, R¹⁶ and R¹⁸ are hydrogen and R¹⁷ is substituted or unsubstituted isoxazolyl. In embodiments, R¹⁶ and R¹⁸ are hydrogen and R¹⁷ is substituted or unsubstituted thiazolyl. In embodiments, R¹⁶ and R¹⁸ are hydrogen and R¹⁷ is substituted or unsubstituted furyl. In embodiments, R¹⁶ and R¹⁸ are hydrogen and R¹⁷ is substituted or unsubstituted thienyl. In embodiments, R¹⁶ and R¹⁸ are hydrogen and R¹⁷ is substituted or unsubstituted pyridyl. In embodiments, R¹⁶ and R¹⁸ are hydrogen and R¹⁷ is substituted or unsubstituted pyrimidyl. In embodiments, R¹⁶ and R¹⁸ are hydrogen and R¹⁷ is unsubstituted pyrrolyl. In embodiments, R¹⁶ and R¹⁸ are hydrogen and R¹⁷ is unsubstituted pyrazolyl. In embodiments, R¹⁶ and R¹⁸ are hydrogen and R¹⁷ is unsubstituted pyridazinyl. In embodiments, R¹⁶ and R¹⁸ are hydrogen and R¹⁷ is unsubstituted triazinyl. In embodiments, R¹⁶ and R¹⁸ are hydrogen and R¹⁷ is unsubstituted pyrimidinyl. In embodiments, R¹⁶ and R¹⁸ are hydrogen and R¹⁷ is unsubstituted imidazolyl. In embodiments, R¹⁶ and R¹⁸ are hydrogen and R¹⁷ is unsubstituted pyrazinyl. In embodiments, R¹⁶ and R¹⁸ are hydrogen and R¹⁷ is unsubstituted oxazolyl. In embodiments, R¹⁶ and R¹⁸ are hydrogen and R¹⁷ is unsubstituted isoxazolyl. In embodiments, R¹⁶ and R¹⁸ are hydrogen and R¹⁷ is unsubstituted thiazolyl. In embodiments, R¹⁶ and R¹⁸ are hydrogen and R¹⁷ is unsubstituted furyl. In embodiments, R¹⁶ and R¹⁸ are hydrogen and R¹⁷ is unsubstituted thienyl. In embodiments, R¹⁶ and R¹⁸ are hydrogen and R¹⁷ is unsubstituted pyridyl. In embodiments, R¹⁶ and R¹⁸ are hydrogen and R¹⁷ is unsubstituted pyrimidyl.

In embodiments, R¹⁶ is independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl.

In embodiments, R¹⁷ is independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl.

In embodiments, R¹⁸ is independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl.

In embodiments, R¹⁶ is independently hydrogen, substituted or unsubstituted C₁-C₆ alkyl, or substituted or unsubstituted 2 to 6 membered heteroalkyl. In embodiments, R¹⁶ is independently hydrogen. In embodiments, R¹⁶ is independently substituted or unsubstituted C₁-C₆ alkyl. In embodiments, R¹⁶ is substituted methyl. In embodiments, R¹⁶ is substituted ethyl. In embodiments, R¹⁶ is substituted propyl. In embodiments, R¹⁶ is substituted isopropyl. In embodiments, R¹⁶ is substituted n-propyl. In embodiments, R¹⁶ is unsubstituted methyl. In embodiments, R¹⁶ is unsubstituted ethyl. In embodiments, R¹⁶ is unsubstituted propyl. In embodiments, R¹⁶ is unsubstituted isopropyl. In embodiments, R¹⁶ is unsubstituted n-propyl. In embodiments, R¹⁶ is independently substituted or unsubstituted 2 to 6 membered heteroalkyl. In embodiments, R¹⁶ is independently substituted or unsubstituted 2 to 3 membered heteroalkyl. In embodiments, R¹⁶ is independently hydrogen or —C(O)N(CH₃)₂. In embodiments, R¹⁶ is independently —C(O)N(CH₃)₂. In embodiments, R¹⁶ is independently hydrogen or substituted or unsubstituted C₁-C₆ alkyl. In embodiments, R¹⁶ is independently hydrogen or substituted or unsubstituted C₁-C₄ alkyl. In embodiments, R¹⁶ is independently hydrogen or substituted or unsubstituted C₁-C₂ alkyl. In embodiments, R¹⁶ is independently hydrogen or unsubstituted methyl.

In embodiments, R¹⁷ is independently hydrogen, substituted or unsubstituted C₁-C₆ alkyl, or substituted or unsubstituted 2 to 6 membered heteroalkyl. In embodiments, R¹⁷ is independently hydrogen. In embodiments, R¹⁷ is independently substituted or unsubstituted C₁-C₆ alkyl. In embodiments, R¹⁷ is substituted methyl. In embodiments, R¹⁷ is substituted ethyl. In embodiments, R¹⁷ is substituted propyl. In embodiments, R¹⁷ is substituted isopropyl. In embodiments, R¹⁷ is substituted n-propyl. In embodiments, R¹⁷ is unsubstituted methyl. In embodiments, R¹⁷ is unsubstituted ethyl. In embodiments, R¹⁷ is unsubstituted propyl. In embodiments, R¹⁷ is unsubstituted isopropyl. In embodiments, R¹⁷ is unsubstituted n-propyl. In embodiments, R¹⁷ is independently substituted or unsubstituted 2 to 6 membered heteroalkyl. In embodiments, R¹⁷ is independently substituted or unsubstituted 2 to 3 membered heteroalkyl. In embodiments, R¹⁷ is independently hydrogen or —C(O)N(CH₃)₂. In embodiments, R¹⁷ is independently —C(O)N(CH₃)₂. In embodiments, R¹⁷ is independently hydrogen or substituted or unsubstituted C₁-C₆ alkyl. In embodiments, R¹⁷ is independently hydrogen or substituted or unsubstituted C₁-C₄ alkyl. In embodiments, R¹⁷ is independently hydrogen or substituted or unsubstituted C₁-C₂ alkyl. In embodiments, R¹⁷ is independently hydrogen or unsubstituted methyl.

In embodiments, R¹⁷ is substituted or unsubstituted aryl. In embodiments, R¹⁷ is substituted or unsubstituted C₆-C₁₂ aryl. In embodiments, R¹⁷ is substituted or unsubstituted phenyl. In embodiments, R¹⁷ is unsubstituted phenyl.

In embodiments, R¹⁷ is substituted or unsubstituted heteroaryl. In embodiments, R¹⁷ is substituted or unsubstituted 5 to 6 membered heteroaryl. In embodiments, R¹⁷ is substituted or unsubstituted pyrrolyl. In embodiments, R¹⁷ is substituted or unsubstituted pyrazolyl. In embodiments, R¹⁷ is substituted or unsubstituted pyridazinyl. In embodiments, R¹⁷ is substituted or unsubstituted triazinyl. In embodiments, R¹⁷ is substituted or unsubstituted pyrimidinyl. In embodiments, R¹⁷ is substituted or unsubstituted imidazolyl. In embodiments, R¹⁷ is substituted or unsubstituted pyrazinyl. In embodiments, R¹⁷ is substituted or unsubstituted oxazolyl. In embodiments, R¹⁷ is substituted or unsubstituted isoxazolyl. In embodiments, R¹⁷ is substituted or unsubstituted thiazolyl. In embodiments, R¹⁷ is substituted or unsubstituted furyl. In embodiments, R¹⁷ is substituted or unsubstituted thienyl. In embodiments, R¹⁷ is substituted or unsubstituted pyridyl. In embodiments, R¹⁷ is substituted or unsubstituted pyrimidyl. In embodiments, R¹⁷ is substituted or unsubstituted benzothiazolyl. In embodiments, R¹⁷ is substituted or unsubstituted benzoxazoyl. In embodiments, R¹⁷ is substituted or unsubstituted benzimidazolyl. In embodiments, R¹⁷ is substituted or unsubstituted benzofuran. In embodiments, R¹⁷ is substituted or unsubstituted isobenzofuranyl. In embodiments, R¹⁷ is substituted or unsubstituted indolyl. In embodiments, R¹⁷ is substituted or unsubstituted isoindolyl. In embodiments, R¹⁷ is substituted or unsubstituted benzothiophenyl. In embodiments, R¹⁷ is substituted or unsubstituted isoquinolyl. In embodiments, R¹⁷ is substituted or unsubstituted quinoxalinyl. In embodiments, R¹⁷ is substituted or unsubstituted quinolyl.

In embodiments, R¹⁸ is independently hydrogen, substituted or unsubstituted C₁-C₆ alkyl, or substituted or unsubstituted 2 to 6 membered heteroalkyl. In embodiments, R¹⁸ is independently hydrogen. In embodiments, R¹⁸ is independently substituted or unsubstituted C₁-C₆ alkyl. In embodiments, R¹⁸ is substituted methyl. In embodiments, R¹⁸ is substituted ethyl. In embodiments, R¹⁸ is substituted propyl. In embodiments, R¹⁸ is substituted isopropyl. In embodiments, R¹⁸ is substituted n-propyl. In embodiments, R¹⁸ is unsubstituted methyl. In embodiments, R¹⁸ is unsubstituted ethyl. In embodiments, R¹⁸ is unsubstituted propyl. In embodiments, R¹⁸ is unsubstituted isopropyl. In embodiments, R¹⁸ is unsubstituted n-propyl. In embodiments, R¹⁸ is independently substituted or unsubstituted 2 to 6 membered heteroalkyl. In embodiments, R¹⁸ is independently substituted or unsubstituted 2 to 3 membered heteroalkyl. In embodiments, R¹⁸ is independently hydrogen or —C(O)N(CH₃)₂. In embodiments, R¹⁸ is independently —C(O)N(CH₃)₂. In embodiments, R¹⁸ is independently hydrogen or substituted or unsubstituted C₁-C₆ alkyl. In embodiments, R¹⁸ is independently hydrogen or substituted or unsubstituted C₁-C₄ alkyl. In embodiments, R¹⁸ is independently hydrogen or substituted or unsubstituted C₁-C₂ alkyl. In embodiments, R¹⁸ is independently hydrogen or unsubstituted methyl.

In embodiments, R¹⁹ and R²⁰ are independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —COOH, —CONH₂, —C(O)N(CH₃)₂, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl.

In embodiments, R¹⁹ is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —COOH, —CONH₂, —C(O)N(CH₃)₂, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl.

In embodiments, R²⁰ is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —COOH, —CONH₂, —C(O)N(CH₃)₂, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl.

In embodiments, R¹⁹ is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, substituted or unsubstituted C₁-C₆ alkyl, or substituted or unsubstituted 2 to 6 membered heteroalkyl. In embodiments, R¹⁹ is independently hydrogen. In embodiments, R¹⁹ is independently —CCl₃. In embodiments, R¹⁹ is independently —CBr₃. In embodiments, R¹⁹ is independently —CF₃. In embodiments, R¹⁹ is independently —CI₃. In embodiments, R¹⁹ is independently CHCl₂. In embodiments, R¹⁹ is independently —CHBr₂. In embodiments, R¹⁹ is independently —CHF₂. In embodiments, R¹⁹ is independently —CHI₂. In embodiments, R¹⁹ is independently —CH₂Cl. In embodiments, R¹⁹ is independently —CH₂Br. In embodiments, R¹⁹ is independently —CH₂F. In embodiments, R¹⁹ is independently —CH₂I. In embodiments, R¹⁹ is independently —CN. In embodiments, R¹⁹ is independently substituted or unsubstituted C₁-C₆ alkyl. In embodiments, R¹⁹ is substituted methyl. In embodiments, R¹⁹ is substituted ethyl. In embodiments, R¹⁹ is substituted propyl. In embodiments, R¹⁹ is substituted isopropyl. In embodiments, R¹⁹ is substituted n-propyl. In embodiments, R¹⁹ is unsubstituted methyl. In embodiments, R¹⁹ is unsubstituted ethyl. In embodiments, R¹⁹ is unsubstituted propyl. In embodiments, R¹⁹ is unsubstituted isopropyl. In embodiments, R¹⁹ is unsubstituted n-propyl. In embodiments, R¹⁹ is independently substituted or unsubstituted 2 to 6 membered heteroalkyl. In embodiments, R¹⁹ is independently substituted or unsubstituted 2 to 3 membered heteroalkyl. In embodiments, R¹⁹ is independently hydrogen or —C(O)N(CH₃)₂. In embodiments, R¹⁹ is independently —C(O)N(CH₃)₂. In embodiments, R¹⁹ is independently hydrogen or substituted or unsubstituted C₁-C₆ alkyl. In embodiments, R¹⁹ is independently hydrogen or substituted or unsubstituted C₁-C₄ alkyl. In embodiments, R¹⁹ is independently hydrogen or substituted or unsubstituted C₁-C₂ alkyl. In embodiments, R¹⁹ is independently hydrogen or unsubstituted methyl. In embodiments, R¹⁹ is independently hydrogen, —CF₃, —CN, or unsubstituted methyl.

In embodiments, R¹⁹ is independently hydrogen, —CF₃, —CN, or substituted or unsubstituted C₁-C₆ alkyl. In embodiments, R¹⁹ is independently hydrogen, —CF₃, —CN, or unsubstituted C₁-C₆ alkyl. In embodiments, R¹⁹ is independently hydrogen, —CF₃, —CN, or unsubstituted methyl. In embodiments, R¹⁹ is independently hydrogen or unsubstituted methyl. In embodiments, R¹⁹ is independently hydrogen or unsubstituted ethyl. In embodiments, R¹⁹ is independently hydrogen or unsubstituted propyl.

In embodiments, R²⁰ is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, substituted or unsubstituted C₁-C₆ alkyl, or substituted or unsubstituted 2 to 6 membered heteroalkyl. In embodiments, R²⁰ is independently hydrogen. In embodiments, R²⁰ is independently —CCl₃. In embodiments, R²⁰ is independently —CBr₃. In embodiments, R²⁰ is independently —CF₃. In embodiments, R²⁰ is independently —CI₃. In embodiments, R²⁰ is independently CHCl₂. In embodiments, R²⁰ is independently —CHBr₂. In embodiments, R²⁰ is independently —CHF₂. In embodiments, R²⁰ is independently —CHI₂. In embodiments, R²⁰ is independently —CH₂Cl. In embodiments, R²⁰ is independently —CH₂Br. In embodiments, R²⁰ is independently —CH₂F. In embodiments, R²⁰ is independently —CH₂I. In embodiments, R²⁰ is independently —CN. In embodiments, R²⁰ is independently substituted or unsubstituted C₁-C₆ alkyl. In embodiments, R²⁰ is substituted methyl. In embodiments, R²⁰ is substituted ethyl. In embodiments, R²⁰ is substituted propyl. In embodiments, R²⁰ is substituted isopropyl. In embodiments, R²⁰ is substituted n-propyl. In embodiments, R²⁰ is unsubstituted methyl. In embodiments, R²⁰ is unsubstituted ethyl. In embodiments, R²⁰ is unsubstituted propyl. In embodiments, R²⁰ is unsubstituted isopropyl. In embodiments, R²⁰ is unsubstituted n-propyl. In embodiments, R²⁰ is independently substituted or unsubstituted 2 to 6 membered heteroalkyl. In embodiments, R²⁰ is independently substituted or unsubstituted 2 to 3 membered heteroalkyl. In embodiments, R²⁰ is independently hydrogen or —C(O)N(CH₃)₂. In embodiments, R²⁰ is independently —C(O)N(CH₃)₂. In embodiments, R²⁰ is independently hydrogen or substituted or unsubstituted C₁-C₆ alkyl. In embodiments, R²⁰ is independently hydrogen or substituted or unsubstituted C₁-C₄ alkyl. In embodiments, R²⁰ is independently hydrogen or substituted or unsubstituted C₁-C₂ alkyl. In embodiments, R²⁰ is independently hydrogen or unsubstituted methyl. In embodiments, R²⁰ is independently hydrogen, —CF₃, —CN, or unsubstituted methyl.

In embodiments, R²⁰ is independently hydrogen, —CF₃, —CN, or substituted or unsubstituted C₁-C₆ alkyl. In embodiments, R²⁰ is independently hydrogen, —CF₃, —CN, or unsubstituted C₁-C₆ alkyl. In embodiments, R²⁰ is independently hydrogen, —CF₃, —CN, or unsubstituted methyl. In embodiments, R²⁰ is independently hydrogen or unsubstituted methyl. In embodiments, R²⁰ is independently hydrogen or unsubstituted ethyl. In embodiments, R²⁰ is independently hydrogen or unsubstituted propyl.

In embodiments, R^{1A} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{1A} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl.

In embodiments, R^{1A} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, substituted or unsubstituted C₁-C₄ alkyl, or substituted or unsubstituted 2 to 4 membered heteroalkyl. In embodiments, R^{1A} is independently hydrogen, substituted or unsubstituted C₁-C₄ alkyl, or substituted or unsubstituted 2 to 4 membered heteroalkyl. In embodiments, R^{1A} is independently hydrogen. In embodiments, R^{1A} is independently substituted or unsubstituted C₁-C₄ alkyl. In embodiments, R^{1A} is independently substituted or unsubstituted 2 to 4 membered heteroalkyl. In embodiments, R^{1A} is independently unsubstituted methyl.

In embodiments, R^{1B} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{1B} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl.

In embodiments, R^{1B} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, substituted or unsubstituted C₁-C₄ alkyl, or substituted or unsubstituted 2 to 4 membered heteroalkyl. In embodiments, R^{1B} is independently hydrogen, substituted or unsubstituted C₁-C₄ alkyl, or substituted or unsubstituted 2 to 4 membered heteroalkyl. In embodiments, R^{1B} is independently hydrogen. In embodiments, R^{1B} is independently substituted or unsubstituted C₁-C₄ alkyl. In embodiments, R^{1B} is independently substituted or unsubstituted 2 to 4 membered heteroalkyl. In embodiments, R^{1B} is independently unsubstituted methyl.

In embodiments, R^{1A} and R^{1B} substituents bonded to the same nitrogen atom may optionally be joined to form a substituted or unsubstituted heterocycloalkyl or substituted or unsubstituted heteroaryl.

In embodiments, R^{1A} and R^{1B} substituents bonded to the same nitrogen atom may optionally be joined to form a substituted or unsubstituted 3 to 6 membered heterocycloalkyl or substituted or unsubstituted 5 to 12 membered heteroaryl.

In embodiments, R^{1C} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{1C} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl.

In embodiments, R^{1C} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, substituted or unsubstituted C₁-C₄ alkyl, or substituted or unsubstituted 2 to 4 membered heteroalkyl. In embodiments, R^{1C} is independently hydrogen, substituted or unsubstituted C₁-C₄ alkyl, or substituted or unsubstituted 2 to 4 membered heteroalkyl. In embodiments, R^{1C} is independently hydrogen. In embodiments, R^{1C} is independently substituted or unsubstituted C₁-C₄ alkyl. In embodiments, R^{1C} is independently substituted or unsubstituted 2 to 4 membered heteroalkyl. In embodiments, R^{1C} is independently unsubstituted methyl.

In embodiments, R^{1D} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{1D} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl.

In embodiments, R^{1D} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, substituted or unsubstituted C₁-C₄ alkyl, or substituted or unsubstituted 2 to 4 membered heteroalkyl. In embodiments, R^{1D} is independently hydrogen, substituted or unsubstituted C₁-C₄ alkyl, or substituted or unsubstituted 2 to 4 membered heteroalkyl. In embodiments, R^{1D} is independently hydrogen. In embodiments, R^{1D} is independently substituted or unsubstituted C₁-C₄ alkyl. In embodiments, R^{1D} is independently substituted or unsubstituted 2 to 4 membered heteroalkyl. In embodiments, R^{1D} is independently unsubstituted methyl.

In embodiments, R^{2A} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{2A} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl.

In embodiments, R^{2A} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, substituted or unsubstituted C₁-C₄ alkyl, or substituted or unsubstituted 2 to 4 membered heteroalkyl. In embodiments, R^{2A} is independently hydrogen, substituted or unsubstituted C₁-C₄ alkyl, or substituted or unsubstituted 2 to 4 membered heteroalkyl. In embodiments, R^{2A} is independently hydrogen. In embodiments, R^{2A} is independently substituted or unsubstituted C₁-C₄ alkyl. In embodiments, R^{2A} is independently substituted or unsubstituted 2 to 4 membered heteroalkyl. In embodiments, R^{2A} is independently unsubstituted methyl.

In embodiments, R^{2B} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{2B} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl.

In embodiments, R^{2B} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, substituted or unsubstituted C₁-C₄ alkyl, or substituted or unsubstituted 2 to 4 membered heteroalkyl. In embodiments, R^{2B} is independently hydrogen, substituted or unsubstituted C₁-C₄ alkyl, or substituted or unsubstituted 2 to 4 membered heteroalkyl. In embodiments, R^{2B} is independently hydrogen. In embodiments, R^{2B} is independently substituted or unsubstituted C₁-C₄ alkyl. In embodiments, R^{2B} is independently substituted or unsubstituted 2 to 4 membered heteroalkyl. In embodiments, R^{2B} is independently unsubstituted methyl.

In embodiments, R^{2A} and R^{2B} substituents bonded to the same nitrogen atom may optionally be joined to form a substituted or unsubstituted heterocycloalkyl or substituted or unsubstituted heteroaryl.

In embodiments, R^{2A} and R^{2B} substituents bonded to the same nitrogen atom may optionally be joined to form a substituted or unsubstituted 3 to 6 membered heterocycloalkyl or substituted or unsubstituted 5 to 12 membered heteroaryl.

In embodiments, R^{2C} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{2C} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl.

In embodiments, R^{2C} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, substituted or unsubstituted C₁-C₄ alkyl, or substituted or unsubstituted 2 to 4 membered heteroalkyl. In embodiments, R^{2C} is independently hydrogen, substituted or unsubstituted C₁-C₄ alkyl, or substituted or unsubstituted 2 to 4 membered heteroalkyl. In embodiments, R^{2C} is independently hydrogen. In embodiments, R^{2C} is independently substituted or unsubstituted C₁-C₄ alkyl. In embodiments, R^{2C} is independently substituted or unsubstituted 2 to 4 membered heteroalkyl. In embodiments, R^{2C} is independently unsubstituted methyl.

In embodiments, R^{2D} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{2D} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl.

In embodiments, R^{2D} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, substituted or unsubstituted C₁-C₄ alkyl, or substituted or unsubstituted 2 to 4 membered heteroalkyl. In embodiments, R^{2D} is independently hydrogen, substituted or unsubstituted C₁-C₄ alkyl, or substituted or unsubstituted 2 to 4 membered heteroalkyl. In embodiments, R^{2D} is independently hydrogen. In embodiments, R^{2D} is independently substituted or unsubstituted C₁-C₄ alkyl. In embodiments, R^{2D} is independently substituted or unsubstituted 2 to 4 membered heteroalkyl. In embodiments, R^{2D} is independently unsubstituted methyl.

In embodiments, R^{1.2} is independently hydrogen, halogen, —CCl₃, —CBr₃, —CF₃, —CI₃, —CN, —OH, —NH₂, —COOH, —CONH₂, —OCCl₃, —OCF₃, —OCBr₃, —OCI₃, —N₃, substituted or unsubstituted C₁-C₃ alkyl, or substituted or unsubstituted 2 to 4 membered heteroalkyl. In embodiments, R^{1.2} is independently hydrogen. In embodiments, R^{1.2} is independently halogen. In embodiments, R^{1.2} is independently —F. In embodiments, R^{1.2} is independently —Cl. In embodiments, R^{1.2} is independently-Br. In embodiments, R^{1.2} is independently —I. In embodiments, R^{1.2} is independently —CCl₃. In embodiments, R^{1.2} is independently —CBr₃. In embodiments, R^{1.2} is independently —CF₃. In embodiments, R^{1.2} is independently —CI₃. In embodiments, R^{1.2} is independently —CN. In embodiments, R^{1.2} is independently —OH. In embodiments, R^{1.2} is independently —NH₂. In embodiments, R^{1.2} is independently —COOH. In embodiments, R^{1.2} is independently —CONH₂. In embodiments, R^{1.2} is independently —OCCl₃. In embodiments, R^{1.2} is independently —OCF₃. In embodiments, R^{1.2} is independently —OCBr₃. In embodiments, R^{1.2} is independently —OCI₃. In embodiments, R^{1.2} is independently —N₃. In embodiments, R^{1.2} is independently substituted or unsubstituted C₁-C₃ alkyl. In embodiments, R^{1.2} is independently unsubstituted methyl. In embodiments, R^{1.2} is independently unsubstituted ethyl. In embodiments, R^{1.2} is independently unsubstituted propyl. In embodiments, R^{1.2} is independently substituted or unsubstituted 2 to 4 membered heteroalkyl. In embodiments, R^{1.2} is independently unsubstituted 2 to 4 membered heteroalkyl.

In embodiments, R^{1.3} is independently hydrogen, halogen, —CCl₃, —CBr₃, —CF₃, —CI₃, —CN, —OH, —NH₂, -COOH, —CONH₂, —OCCl₃, —OCF₃, —OCBr₃, —OCI₃, —N₃, substituted or unsubstituted C₁-C₃ alkyl, or substituted or unsubstituted 2 to 4 membered heteroalkyl. In embodiments, R^{1.3} is independently halogen. In embodiments, R^{1.3} is independently —F. In embodiments, R^{1.3} is independently —Cl. In embodiments, R^{1.3} is independently—Br. In embodiments, R^{1.3} is independently —I. In embodiments, R^{1.3} is independently —CCl₃. In embodiments, R^{1.3} is independently —CBr₃. In embodiments, R^{1.3} is independently —CF₃. In embodiments, R^{1.3} is independently —CI₃. In embodiments, R^{1.3} is independently —CN. In embodiments, R^{1.3} is independently —OH. In embodiments, R^{1.3} is independently —NH₂. In embodiments, R^{1.3} is independently -COOH. In embodiments, R^{1.3} is independently —CONH₂. In embodiments, R^{1.3} is independently —OCCl₃. In embodiments, R^{1.3} is independently —OCF₃. In embodiments, R^{1.3} is independently —OCBr₃. In embodiments, R^{1.3} is independently —OCI₃. In embodiments, R^{1.3} is independently —N₃. In embodiments, R^{1.3} is independently substituted or unsubstituted C₁-C₃ alkyl. In embodiments, R^{1.3} is independently unsubstituted methyl. In embodiments, R^{1.3} is independently unsubstituted ethyl. In embodiments, R^{1.3} is independently unsubstituted propyl. In embodiments, R^{1.3} is independently substituted or unsubstituted 2 to 4 membered heteroalkyl. In embodiments, R^{1.3} is independently unsubstituted 2 to 4 membered heteroalkyl.

In embodiments, R^{1.2} and R^{1.3} substituents on adjacent carbons are joined to form a substituted or unsubstituted phenyl, or substituted or unsubstituted 5 to 6 membered heteroaryl. In embodiments, R^{1.2} and R^{1.3} substituents on adjacent carbons are joined to form a substituted or unsubstituted phenyl. In embodiments, R^{1.2} and R^{1.3} substituents on adjacent carbons are joined to form an R¹¹-substituted phenyl. In embodiments, R^{1.2} and R^{1.3} substituents on adjacent carbons are joined to form an unsubstituted phenyl. In embodiments, R^{1.2} and R^{1.3} substituents on adjacent carbons are joined to form a substituted or unsubstituted 5 to 6 membered heteroaryl. In embodiments, R^{1.2} and R^{1.3} substituents on adjacent carbons are joined to form a R¹¹-substituted or unsubstituted 5 to 6 membered heteroaryl. In embodiments, R^{1.2} and R^{1.3} substituents on adjacent carbons are joined to form an unsubstituted 5 to 6 membered heteroaryl.

In embodiments, R^{1.4} is independently hydrogen, halogen, —CCl₃, —CBr₃, —CF₃, —CI₃, —CN, —OH, —NH₂, —COOH, —CONH₂, —OCCl₃, —OCF₃, —OCBr₃, —OCI₃, —N₃, substituted or unsubstituted C₁-C₃ alkyl, or substituted or unsubstituted 2 to 4 membered heteroalkyl. In embodiments, R^{1.4} is independently halogen. In embodiments, R^{1.4} is independently —F. In embodiments, R^{1.4} is independently —Cl. In embodiments, R^{1.4} is independently —Br. In embodiments, R^{1.4} is independently —I. In embodiments, R^{1.4} is independently —CCl₃. In embodiments, R^{1.4} is independently —CBr₃. In embodiments, R^{1.4} is independently —CF₃. In embodiments, R^{1.4} is independently —CI₃. In embodiments, R^{1.4} is independently —CN. In embodiments, R^{1.4} is independently —OH. In embodiments, R^{1.4} is independently —NH₂. In embodiments, R^{1.4} is independently -COOH. In embodiments, R^{1.4} is independently —CONH₂. In embodiments, R^{1.4} is independently —OCCl₃. In embodiments, R^{1.4} is independently —OCF₃. In embodiments, R^{1.4} is independently —OCBr₃. In embodiments, R^{1.4} is independently —OCI₃. In embodiments, R^{1.4} is independently —N₃. In embodiments, R^{1.4} is independently substituted or unsubstituted C₁-C₃ alkyl. In embodiments, R^{1.4} is independently unsubstituted methyl. In embodiments, R^{1.4} is independently unsubstituted ethyl. In embodiments, R^{1.4} is independently unsubstituted propyl. In embodiments, R^{1.4} is independently substituted or unsubstituted 2 to 4 membered heteroalkyl. In embodiments, R^{1.4} is independently unsubstituted 2 to 4 membered heteroalkyl.

In embodiments, R^{1.5} is independently hydrogen, halogen, —CCl₃, —CBr₃, —CF₃, —CI₃, —CN, —OH, —NH₂, —COOH, —CONH₂, —OCCl₃, —OCF₃, —OCBr₃, —OCI₃, —N₃, substituted or unsubstituted C₁-C₃ alkyl, or substituted or unsubstituted 2 to 4 membered heteroalkyl. In embodiments, R^{1.5} is independently halogen. In embodiments, R^{1.5} is independently —F. In embodiments, R^{1.5} is independently —Cl. In embodiments, R^{1.5} is independently —Br. In embodiments, R^{1.5} is independently —I. In embodiments, R^{1.5} is independently —CCl₃. In embodiments, R^{1.5} is independently —CBr₃. In embodiments, R^{1.5} is independently —CF₃. In embodiments, R^{1.5} is independently —CI₃. In embodiments, R^{1.5} is independently —CN. In embodiments, R^{1.5} is independently —OH. In embodiments, R^{1.5} is independently —NH₂. In embodiments, R^{1.5} is independently —COOH. In embodiments, R^{1.5} is independently —CONH₂. In embodiments, R^{1.5} is independently —OCCl₃. In embodiments, R^{1.5} is independently —OCF₃. In embodiments, R^{1.5} is independently —OCBr₃. In embodiments, R^{1.5} is independently —OCI₃. In embodiments, R^{1.5} is independently —N₃. In embodiments, R^{1.5} is independently substituted or unsubstituted C₁-C₃ alkyl. In embodiments, R^{1.5} is independently unsubstituted methyl. In embodiments, R^{1.5} is independently unsubstituted ethyl. In embodiments, R^{1.5} is independently unsubstituted propyl. In embodiments, R^{1.5} is independently substituted or unsubstituted 2 to 4 membered heteroalkyl. In embodiments, R^{1.5} is independently unsubstituted 2 to 4 membered heteroalkyl.

In embodiments, R^{1.4} is hydrogen and R^{1.5} is —F. In embodiments, R^{1.4} is —F and R^{1.5} is hydrogen. In embodiments, R^{1.4} and R^{1.5} are —F. In embodiments, R^{1.4} and R^{1.5} are hydrogen.

In embodiments, W¹ is independently —O—. In embodiments, W¹ is independently —NH—. In embodiments, ¹ is independently —NR²—. In embodiments, W² is independently ═N—. In embodiments, ² is independently ═CH—. In embodiments, W² is independently ═CR²—. In embodiments, W³ is independently ═N—. In embodiments, ³ is independently ═CH—. In embodiments, W³ is independently ═CR²—.

In embodiments, R² is independently oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, , —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl.

In embodiments, R¹¹ is independently oxo, halogen, —CX¹¹₃, —CHX¹¹₂, —CH₂X¹¹, —OCX¹¹₃, —OCH₂X¹¹, —OCHX¹¹₂, —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl.

In embodiments, R¹¹ is independently oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, , —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl;

In embodiments, R²¹ is independently oxo, halogen, —CX²¹₃, —CHX²¹₂, —CH₂X²¹, —OCX²¹₃, —OCH₂X²¹, —OCHX²¹₂, —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl;

In embodiments, R²¹ is independently oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl.

In embodiments, R² is independently halogen, —OCX²₃, —OCH₂X², —OCHX²₂, unsubstituted C₁—C₃ alkyl, or unsubstituted 5 to 6 membered heteroaryl. In embodiments, R¹¹ is independently halogen, —OCX¹¹₃, —OCH₂X¹¹, -OCHX¹¹₂, unsubstituted C₁-C₃ alkyl, or unsubstituted 5 to 6 membered heteroaryl. In embodiments, R²¹ is independentlyhalogen, —OCX²¹₃, —OCH₂X²¹, —OCHX²¹₂, unsubstituted C₁—C₃ alkyl, or unsubstituted 5 to 6 membered heteroaryl.

In embodiments, R¹¹ is independently halogen, —OCCl₃, —OCF₃, —OCBr₃, —OCI₃, —OCHCl₂, —OCHBr₂, —OCHI₂, —OCHF₂, —OCH₂Cl, —OC H₂Br, —OCH₂I, —OCH₂F, unsubstituted C₁-C₃ alkyl, or unsubstituted 5 to 6 membered heteroaryl. In embodiments, R¹¹ is independently halogen. In embodiments, R¹¹ is independently —F. In embodiments, R¹¹ is independently —Cl. In embodiments, R¹¹ is independently —Br. In embodiments, R¹¹ is independently —I. In embodiments, R¹¹ is independently —OCCl₃. In embodiments, R¹¹ is independently —OCF₃. In embodiments, R¹¹ is independently —OCBr₃. In embodiments, R¹¹ is independently —OCI₃. In embodiments, R¹¹ is independently —OCHCl₂. In embodiments, R¹¹ is independently —OCHBr₂. In embodiments, R¹¹ is independently —OCHI₂. In embodiments, R¹¹ is independently —OCHF₂. In embodiments, R¹¹ is independently —OCH₂Cl. In embodiments, R¹¹ is independently —OCH₂Br. In embodiments, R¹¹ is independently —OCH₂I. In embodiments, R¹¹ is independently —OCH₂F. In embodiments, R¹¹ is independently unsubstituted methyl. In embodiments, R¹¹ is independently unsubstituted ethyl. In embodiments, R¹¹ is independently unsubstituted propyl. In embodiments, R¹¹ is independently unsubstituted 5 to 6 membered heteroaryl. In embodiments, R¹¹ is independently unsubstituted 5 membered heteroaryl. In embodiments, R¹¹ is independently unsubstituted 6 membered heteroaryl.

In embodiments, R^{11.1} is independently oxo, halogen, —CX¹¹₃, —CHX¹¹₂, —CH₂X¹¹, —OCX¹¹₃, —OCH₂X¹¹, —OCHX¹¹₂, —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl.

In embodiments, R^{11.1} is independently oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, , —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHC1₂, —OCHBr₂, —OCHI₂, —OCHF₂, —OCH₂Cl, —OCH₂Br, —OCH₂I, —OCH₂F, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl.

In embodiments, R^{11.1} is independently halogen, —OCCl₃, —OCF₃, —OCBr₃, —OCI₃, —OCHCl₂, —OCHBr₂, —OCHI₂, —OCHF₂, —OCH₂Cl, —OC H₂Br, —OCH₂I, —OCH₂F, unsubstituted C₁-C₃ alkyl, or unsubstituted 5 to 6 membered heteroaryl. In embodiments, R^{11.1} is independently halogen. In embodiments, R^{11.1} is independently —F. In embodiments, R^{11.1} is independently —Cl. In embodiments, R^{11.1} is independently —Br. In embodiments, R^{11.1} is independently —I. In embodiments, R^{11.1} is independently —OCCl₃. In embodiments, R^{11.1} is independently —OCF₃. In embodiments, R^{11.1} is independently —OCBr₃. In embodiments, R^{11.1} is independently —OCI₃. In embodiments, R^{11.1} is independently —OCHCl₂. In embodiments, R^{11.1} is independently —OCHBr₂. In embodiments, R^{11.1} is independently —OCHI₂. In embodiments, R^{11.1} is independently —OCHF₂. In embodiments, R^{11.1} is independently —OCH₂Cl. In embodiments, R^{11.1} is independently —OCH₂Br. In embodiments, R^{11.1} is independently —OCH₂I. In embodiments, R^{11.1} is independently —OCH₂F. In embodiments, R^{11.1} is independently unsubstituted methyl. In embodiments, R^{11.1} is independently unsubstituted ethyl. In embodiments, R^{11.1} is independently unsubstituted propyl. In embodiments, R^{11.1} is independently unsubstituted 5 to 6 membered heteroaryl. In embodiments, R^{11.1} is independently unsubstituted 5 membered heteroaryl. In embodiments, R^{11.1} is independently unsubstituted 6 membered heteroaryl.

In embodiments, R²¹ is independently halogen, —OCCl₃, —OCF₃, —OCBr₃, —OCI₃, —OCHCl₂, —OCHBr₂, —OCHI₂, —OCHF₂, —OCH₂Cl, —OC H₂Br, —OCH₂I, —OCH₂F, unsubstituted C₁-C₃ alkyl, or unsubstituted 5 to 6 membered heteroaryl. In embodiments, R²¹ is independently halogen. In embodiments, R²¹ is independently —F. In embodiments, R²¹ is independently —Cl. In embodiments, R²¹ is independently —Br. In embodiments, R²¹ is independently —I. In embodiments, R²¹ is independently —OCCl₃. In embodiments, R²¹ is independently —OCF₃. In embodiments, R²¹ is independently —OCBr₃. In embodiments, R²¹ is independently —OCI₃. In embodiments, R²¹ is independently —OCHCl₂. In embodiments, R²¹ is independently —OCHBr₂. In embodiments, R²¹ is independently —OCHI₂. In embodiments, R²¹ is independently —OCHF₂. In embodiments, R²¹ is independently —OCH₂Cl. In embodiments, R²¹ is independently —OCH₂Br. In embodiments, R²¹ is independently —OCH₂I. In embodiments, R²¹ is independently —OCH₂F. In embodiments, R²¹ is independently unsubstituted methyl. In embodiments, R²¹ is independently unsubstituted ethyl. In embodiments, R²¹ is independently unsubstituted propyl. In embodiments, R²¹ is independently unsubstituted 5 to 6 membered heteroaryl. In embodiments, R²¹ is independently unsubstituted 5 membered heteroaryl. In embodiments, R²¹ is independently unsubstituted 6 membered heteroaryl.

In embodiments, R^{2.1} is independently hydrogen, —OCCl₃, —OCF₃, —OCBr₃, —OCI₃, or unsubstituted 5 to 6 membered heteroaryl. In embodiments, R^{2.1} is independently hydrogen. In embodiments, R^{2.1} is independently —OCCl₃. In embodiments, R^{2.1} is independently —OCF₃. In embodiments, R^{2.1} is independently —OCBr₃. In embodiments, R^{2.1} is independently —OCI₃. In embodiments, R^{2.1} is independently unsubstituted 5 to 6 membered heteroaryl. In embodiments, R^{2.1} is independently unsubstituted 5 membered heteroaryl. In embodiments, R^{2.1} is independently unsubstituted 6 membered heteroaryl. In embodiments, R^{2.1} is independently pyrrolyl, pyrazolyl, pyridazinyl, triazinyl, pyrimidinyl, imidazolyl, pyrazinyl, oxazolyl, isoxazolyl, thiazolyl, furyl, thienyl, pyridyl, pyrimidyl, benzothiazolyl, benzoxazoyl, benzimidazolyl, benzofuran, isobenzofuranyl, indolyl, or isoindolyl. In embodiments, R^{2.1} is independently phenyl, benzimidazolyl, or indolyl. In embodiments, R^{2.1} is independently pyrrolyl. In embodiments, R^{2.1} is independently pyrazolyl. In embodiments, R^{2.1} is independently pyridazinyl. In embodiments, R^{2.1} is independently triazinyl. In embodiments, R^{2.1} is independently pyrimidinyl. In embodiments, R^{2.1} is independently imidazolyl. In embodiments, R^{2.1} is independently pyrazinyl. In embodiments, R^{2.1} is independently oxazolyl. In embodiments, R^{2.1} is independently isoxazolyl. In embodiments, R^{2.1} is independently thiazolyl. In embodiments, R^{2.1} is independently furyl. In embodiments, R^{2.1} is independently thienyl. In embodiments, R^{2.1} is independently pyridyl. In embodiments, R^{2.1} is independently pyrimidyl. In embodiments, R^{2.1} is independently benzothiazolyl. In embodiments, R^{2.1} is independently benzoxazoyl. In embodiments, R^{2.1} is independently benzimidazolyl. In embodiments, R^{2.1} is independently benzofuran. In embodiments, R^{2.1} is independently isobenzofuranyl. In embodiments, R^{2.1} is independently indolyl. In embodiments, R^{2.1} is independently isoindolyl. In embodiments, R^{2.1} is independently unsubstituted pyrrolyl, pyrazolyl, pyridazinyl, triazinyl, pyrimidinyl, imidazolyl, pyrazinyl, oxazolyl, isoxazolyl, thiazolyl, furyl, thienyl, pyridyl, pyrimidyl, benzothiazolyl, benzoxazoyl, benzimidazolyl, benzofuran, isobenzofuranyl, indolyl, or isoindolyl. In embodiments, R^{2.1} is independently unsubstituted phenyl, benzimidazolyl, or indolyl. In embodiments, R^{2.1} is independently unsubstituted pyrrolyl. In embodiments, R^{2.1} is independently unsubstituted pyrazolyl. In embodiments, R^{2.1} is independently unsubstituted pyridazinyl. In embodiments, R^{2.1} is independently unsubstituted triazinyl. In embodiments, R^{2.1} is independently unsubstituted pyrimidinyl. In embodiments, R^{2.1} is independently unsubstituted imidazolyl. In embodiments, R^{2.1} is independently unsubstituted pyrazinyl. In embodiments, R^{2.1} is independently unsubstituted oxazolyl. In embodiments, R^{2.1} is independently unsubstituted isoxazolyl. In embodiments, R^{2.1} is independently unsubstituted thiazolyl. In embodiments, R^{2.1} is independently unsubstituted furyl. In embodiments, R^{2.1} is independently unsubstituted thienyl. In embodiments, R^{2.1} is independently unsubstituted pyridyl. In embodiments, R^{2.1} is independently unsubstituted pyrimidyl. In embodiments, R^{2.1} is independently unsubstituted benzothiazolyl. In embodiments, R^{2.1} is independently unsubstituted benzoxazoyl. In embodiments, R^{2.1} is independently unsubstituted benzimidazolyl. In embodiments, R^{2.1} is independently unsubstituted benzofuran. In embodiments, R^{2.1} is independently unsubstituted isobenzofuranyl. In embodiments, R^{2.1} is independently unsubstituted indolyl. In embodiments, R^{2.1} is independently unsubstituted isoindolyl.

In embodiments, R^{2.2} is independently hydrogen, —F, —Cl, —Br, or —I. In embodiments, R^{2.2} is independently hydrogen. In embodiments, R^{2.2} is independently —F. In embodiments, R^{2.2} is independently —Cl. In embodiments, R^{2.2} is independently —Br. In embodiments, R^{2.2} is independently —I.

In embodiments, z1 is an integer from 0 to 9. In embodiments, z1 is 0. In embodiments, z1 is 1. In embodiments, z1 is 2. In embodiments, z1 is 3. In embodiments, z1 is 4. In embodiments, z1 is 5. In embodiments, z1 is 6. In embodiments, z1 is 7. In embodiments, z1 is 8. In embodiments, z1 is 9. In embodiments, z2 is an integer from 0 to 6. In embodiments, z2 is 0. In embodiments, z2 is 1. In embodiments, z2 is 2. In embodiments, z2 is 3. In embodiments, z2 is 4. In embodiments, z2 is 5. In embodiments, z2 is 6. In embodiments, z6 is 1 or 2. In embodiments, z6 is 1. In embodiments, z6 is 2. In embodiments, z11 is an integer from 0 to 4. In embodiments, z11 is 0. In embodiments, z11 is 1. In embodiments, z11 is 2. In embodiments, z11 is 3. In embodiments, z11 is 4. In embodiments, z21 is an integer from 0 to 5. In embodiments, z21 is 0. In embodiments, z21 is 1. In embodiments, z21 is 2. In embodiments, z21 is 3. In embodiments, z21 is 4. In embodiments, z21 is 5.

In embodiments, n1 and n2 are independently an integer from 0 to 4. In embodiments, n1 is independently 0. In embodiments, n1 is independently 1. In embodiments, n1 is independently 2. In embodiments, n1 is independently 3. In embodiments, n1 is independently 4. In embodiments, m1, m2, v1, and v2 are independently 1 or 2. In embodiments, m1 is independently 1. In embodiments, m1 is independently 2. In embodiments, m2 is independently 1. In embodiments, m2 is independently 2. In embodiments, v1 is independently 1. In embodiments, v1 is independently 2. In embodiments, v2 is independently 1. In embodiments, v2 is independently 2.

In embodiments, X¹ and X² are independently —F, —Cl, —Br, or —I. In embodiments, X¹ is independently —F, —Cl, —Br, or —I. In embodiments, X¹ is independently —F. In embodiments, X¹ is independently —Cl. In embodiments, X¹ is independently —Br. In embodiments, X¹ is independently —I. In embodiments, X² is independently —F, —Cl, —Br, or —I. In embodiments, X² is independently —F. In embodiments, X² is independently —Cl. In embodiments, X² is independently —Br. In embodiments, X² is independently —I. In embodiments, X¹¹ is independently —F, —Cl, —Br, or —I. In embodiments, X²¹ is independently —F, —Cl, —Br, or —I. In embodiments, X¹¹ is independently —F. In embodiments, X¹¹ is independently —Cl. In embodiments, X¹¹ is independently —Br. In embodiments, X¹¹ is independently —I. In embodiments, X²¹ is independently —F. In embodiments, X²¹ is independently —Cl. In embodiments, X²¹ is independently —Br. In embodiments, X²¹ is independently —I.

In embodiments, R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O) NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered); two adjacent R¹ substituents on adjacent carbons may optionally be joined to form a substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O) NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted (e.g., substituted with at least one substituent group, size-limited substituent group, or lower substituent group) or unsubstituted C₁-C₆ alkyl, substituted (e.g., substituted with at least one substituent group, size-limited substituent group, or lower substituent group) or unsubstituted 2 to 6 membered heteroalkyl, substituted (e.g., substituted with at least one substituent group, size-limited substituent group, or lower substituent group) or unsubstituted C₃-C₆ cycloalkyl, substituted (e.g., substituted with at least one substituent group, size-limited substituent group, or lower substituent group) or unsubstituted 3 to 6 membered heterocycloalkyl, substituted (e.g., substituted with at least one substituent group, size-limited substituent group, or lower substituent group) or unsubstituted C₆-C₁₀ aryl, or substituted (e.g., substituted with at least one substituent group, size-limited substituent group, or lower substituent group) or unsubstituted 5 to 10 membered heteroaryl; two adjacent R¹ substituents on adjacent carbons may optionally be joined to form a substituted (e.g., substituted with at least one substituent group, size-limited substituent group, or lower substituent group) or unsubstituted C₃-C₆ cycloalkyl, substituted (e.g., substituted with at least one substituent group, size-limited substituent group, or lower substituent group) or unsubstituted 3 to 6 membered heterocycloalkyl, substituted (e.g., substituted with at least one substituent group, size-limited substituent group, or lower substituent group) or unsubstituted phenyl, or substituted (e.g., substituted with at least one substituent group, size-limited substituent group, or lower substituent group) or unsubstituted 5 to 6 membered heteroaryl.

In embodiments, a substituted R¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹ is substituted with a plurality of groups selected from substituent groups, size-limited substituent groups, and lower substituent groups; each substituent group, size-limited substituent group, and/or lower substituent group may optionally be different. In embodiments, when R¹ is substituted, it is substituted with at least one substituent group. In embodiments, when R¹ is substituted, it is substituted with at least one size-limited substituent group. In embodiments, when R¹ is substituted, it is substituted with at least one lower substituent group.

In embodiments, R² is independently oxo, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}N^{R2}AR^{2B},—NHC(O)NR^{2A}R^{2B}, —N(O)ₘ₂, —NR^{2A}R^{2B}, —C(O)R^{2C}, —C(O)—OR^{2C}, —C(O) NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, R² is independently oxo, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B},—NHC(O)NR^{2A}R^{2B}, —N(O)ₘ₂, —NR^{2A}R^{2B}, —C(O)R^{2C}, —C(O)—OR^{2C}, —C(O) NR2AR2B, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted C₁-C₆ alkyl, substituted (e.g., substituted with at least one substituent group, size-limited substituent group, or lower substituent group) or unsubstituted 2 to 6 membered heteroalkyl, substituted (e.g., substituted with at least one substituent group, size-limited substituent group, or lower substituent group) or unsubstituted C₃-C₆ cycloalkyl, substituted (e.g., substituted with at least one substituent group, size-limited substituent group, or lower substituent group) or unsubstituted 3 to 6 membered heterocycloalkyl, substituted (e.g., substituted with at least one substituent group, size-limited substituent group, or lower substituent group) or unsubstituted C₆-C₁₀ aryl, or substituted (e.g., substituted with at least one substituent group, size-limited substituent group, or lower substituent group) or unsubstituted 5 to 10 membered heteroaryl.

In embodiments, a substituted R² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R² is substituted with a plurality of groups selected from substituent groups, size-limited substituent groups, and lower substituent groups; each substituent group, size-limited substituent group, and/or lower substituent group may optionally be different. In embodiments, when R² is substituted, it is substituted with at least one substituent group. In embodiments, when R² is substituted, it is substituted with at least one size-limited substituent group. In embodiments, when R² is substituted, it is substituted with at least one lower substituent group.

In embodiments, L³ is a bond, —S(O)₂—, —NR³—, —NH—, —O—, —S—, —C(O)—, —C(O)NR³—, —NR³C(O)—, —N(R³)CH₂—, —NR³C(O)NH —, —NHC(O)NR³—, —C(O)O—, —OC(O)—, substituted (e.g., substituted with at least one substituent group, size-limited substituent group, or lower substituent group) or unsubstituted C₁-C₆ alkylene, or substituted (e.g., substituted with at least one substituent group, size-limited substituent group, or lower substituent group) or unsubstituted 2 to 6 membered heteroalkylene.

In embodiments, L³ is a bond, —S(O)₂—, —NR³—, —NH—, —O—, —S—, —C(O)—, —C(O)NR³—, —NR³C(O)—, —N(R³)CH₂—, —NR³C(O)NH —, —NHC(O)NR³—, —C(O)O—, —OC(O)—, substituted (e.g., substituted with at least one substituent group, size-limited substituent group, or lower substituent group) or unsubstituted alkylene (e.g., C₁-C₁₀, C₁-C₈, C₁-C₆, C₁-C₄, C₁-C₂, C₂-C₁₀, C₂-C₈, C₂-C₆, or C₂-C₄), or substituted (e.g., substituted with at least one substituent group, size-limited substituent group, or lower substituent group) or unsubstituted heteroalkylene (e.g., 2 to 8 membered, 2 to 6 membered, 4 to 6 membered, 2 to 3 membered, or 4 to 5 membered).

In embodiments, a substituted L³ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L³ is substituted with a plurality of groups selected from substituent groups, size-limited substituent groups, and lower substituent groups; each substituent group, size-limited substituent group, and/or lower substituent group may optionally be different. In embodiments, when L³ is substituted, it is substituted with at least one substituent group. In embodiments, when L³ is substituted, it is substituted with at least one size-limited substituent group. In embodiments, when L³ is substituted, it is substituted with at least one lower substituent group.

In embodiments, L⁴ is a bond, —NH—, —NR⁴—, or substituted (e.g., substituted with at least one substituent group, size-limited substituent group, or lower substituent group) or unsubstituted alkylene (e.g., C₁-C₁₀, C₁-C₈, C₁-C₆, C₁-C₄, C₁-C₂, C₂-C₁₀, C₂-C₈, C₂-C₆, or C₂-C₄).

In embodiments, a substituted L⁴ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L⁴ is substituted with a plurality of groups selected from substituent groups, size-limited substituent groups, and lower substituent groups; each substituent group, size-limited substituent group, and/or lower substituent group may optionally be different. In embodiments, when L⁴ is substituted, it is substituted with at least one substituent group. In embodiments, when L⁴ is substituted, it is substituted with at least one size-limited substituent group. In embodiments, when L⁴ is substituted, it is substituted with at least one lower substituent group.

In embodiments, R¹⁶ is independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, R¹⁷ is independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, R¹⁸ is independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered , 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, R¹⁶, R¹⁷, and R¹⁸ are independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted (e.g., substituted with at least one substituent group, size-limited substituent group, or lower substituent group) or unsubstituted C₁-C₆ alkyl, substituted (e.g., substituted with at least one substituent group, size-limited substituent group, or lower substituent group) or unsubstituted 2 to 6 membered heteroalkyl, substituted (e.g., substituted with at least one substituent group, size-limited substituent group, or lower substituent group) or unsubstituted C₃-C₆ cycloalkyl, substituted (e.g., substituted with at least one substituent group, size-limited substituent group, or lower substituent group) or unsubstituted 3 to 6 membered heterocycloalkyl, substituted (e.g., substituted with at least one substituent group, size-limited substituent group, or lower substituent group) or unsubstituted C₆-C₁₂ aryl, or substituted (e.g., substituted with at least one substituent group, size-limited substituent group, or lower substituent group) or unsubstituted 5 to 12 membered heteroaryl.

In embodiments, a substituted R¹⁶ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁶ is substituted with a plurality of groups selected from substituent groups, size-limited substituent groups, and lower substituent groups; each substituent group, size-limited substituent group, and/or lower substituent group may optionally be different. In embodiments, when R¹⁶ is substituted, it is substituted with at least one substituent group. In embodiments, when R¹⁶ is substituted, it is substituted with at least one size-limited substituent group. In embodiments, when R¹⁶ is substituted, it is substituted with at least one lower substituent group.

In embodiments, a substituted R¹⁷ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁷ is substituted with a plurality of groups selected from substituent groups, size-limited substituent groups, and lower substituent groups; each substituent group, size-limited substituent group, and/or lower substituent group may optionally be different. In embodiments, when R¹⁷ is substituted, it is substituted with at least one substituent group. In embodiments, when R¹⁷ is substituted, it is substituted with at least one size-limited substituent group. In embodiments, when R¹⁷ is substituted, it is substituted with at least one lower substituent group.

In embodiments, a substituted R¹⁸ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁸ is substituted with a plurality of groups selected from substituent groups, size-limited substituent groups, and lower substituent groups; each substituent group, size-limited substituent group, and/or lower substituent group may optionally be different. In embodiments, when R¹⁸ is substituted, it is substituted with at least one substituent group. In embodiments, when R¹⁸ is substituted, it is substituted with at least one size-limited substituent group. In embodiments, when R¹⁸ is substituted, it is substituted with at least one lower substituent group.

In embodiments, R¹⁹ is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —COOH, —CONH₂, —C(O)N(CH₃)₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, R²⁰ is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —COOH, —CONH₂, —C(O)N(CH₃)₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R¹⁹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁹ is substituted with a plurality of groups selected from substituent groups, size-limited substituent groups, and lower substituent groups; each substituent group, size-limited substituent group, and/or lower substituent group may optionally be different. In embodiments, when R¹⁹ is substituted, it is substituted with at least one substituent group. In embodiments, when R¹⁹ is substituted, it is substituted with at least one size-limited substituent group. In embodiments, when R¹⁹ is substituted, it is substituted with at least one lower substituent group.

In embodiments, a substituted R²⁰ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R²⁰ is substituted with a plurality of groups selected from substituent groups, size-limited substituent groups, and lower substituent groups; each substituent group, size-limited substituent group, and/or lower substituent group may optionally be different. In embodiments, when R²⁰ is substituted, it is substituted with at least one substituent group. In embodiments, when R²⁰ is substituted, it is substituted with at least one size-limited substituent group. In embodiments, when R²⁰ is substituted, it is substituted with at least one lower substituent group.

In embodiments, R^{1A}, R^{1B}, R^{1C}, R^{1D}, R^{2A}, R^{2B}, R^{2C}, and R^{2D} are independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, substituted (e.g., substituted with at least one substituent group, size-limited substituent group, or lower substituent group) or unsubstituted alkyl, substituted (e.g., substituted with at least one substituent group, size-limited substituent group, or lower substituent group) or unsubstituted heteroalkyl, substituted (e.g., substituted with at least one substituent group, size-limited substituent group, or lower substituent group) or unsubstituted cycloalkyl, substituted (e.g., substituted with at least one substituent group, size-limited substituent group, or lower substituent group) or unsubstituted heterocycloalkyl, substituted (e.g., substituted with at least one substituent group, size-limited substituent group, or lower substituent group) or unsubstituted aryl, or substituted (e.g., substituted with at least one substituent group, size-limited substituent group, or lower substituent group) or unsubstituted heteroaryl; R^{1A} and R^{1B} substituents bonded to the same nitrogen atom may optionally be joined to form a substituted (e.g., substituted with at least one substituent group, size-limited substituent group, or lower substituent group) or unsubstituted heterocycloalkyl or substituted (e.g., substituted with at least one substituent group, size-limited substituent group, or lower substituent group) or unsubstituted heteroaryl; R^{2A} and R^{2B} substituents bonded to the same nitrogen atom may optionally be joined to form a substituted (e.g., substituted with at least one substituent group, size-limited substituent group, or lower substituent group) or unsubstituted heterocycloalkyl or substituted (e.g., substituted with at least one substituent group, size-limited substituent group, or lower substituent group) or unsubstituted heteroaryl.

In embodiments, R^{1A} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R^{1A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1A} is substituted with a plurality of groups selected from substituent groups, size-limited substituent groups, and lower substituent groups; each substituent group, size-limited substituent group, and/or lower substituent group may optionally be different. In embodiments, when R^{1A} is substituted, it is substituted with at least one substituent group. In embodiments, when R^{1A} is substituted, it is substituted with at least one size-limited substituent group. In embodiments, when R^{1A} is substituted, it is substituted with at least one lower substituent group.

In embodiments, R^{1B} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R^{1B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1B} is substituted with a plurality of groups selected from substituent groups, size-limited substituent groups, and lower substituent groups; each substituent group, size-limited substituent group, and/or lower substituent group may optionally be different. In embodiments, when R^{1B} is substituted, it is substituted with at least one substituent group. In embodiments, when R^{1B} is substituted, it is substituted with at least one size-limited substituent group. In embodiments, when R^{1B} is substituted, it is substituted with at least one lower substituent group.

In embodiments, R^{1C} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R^{1C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1C} is substituted with a plurality of groups selected from substituent groups, size-limited substituent groups, and lower substituent groups; each substituent group, size-limited substituent group, and/or lower substituent group may optionally be different. In embodiments, when R^{1C} is substituted, it is substituted with at least one substituent group. In embodiments, when R^{1C} is substituted, it is substituted with at least one size-limited substituent group. In embodiments, when R^{1C} is substituted, it is substituted with at least one lower substituent group.

In embodiments, R^{1D} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R^{1D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1D} is substituted with a plurality of groups selected from substituent groups, size-limited substituent groups, and lower substituent groups; each substituent group, size-limited substituent group, and/or lower substituent group may optionally be different. In embodiments, when R^{1D} is substituted, it is substituted with at least one substituent group. In embodiments, when R^{1D} is substituted, it is substituted with at least one size-limited substituent group. In embodiments, when R^{1D} is substituted, it is substituted with at least one lower substituent group.

In embodiments, R^{1A} and R^{1B} substituents bonded to the same nitrogen atom may optionally be joined to form a substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered) or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, R^{1A} and R^{1B} substituents bonded to the same nitrogen atom that are optionally joined to form a substituted heterocycloalkyl can be substituted with at least one substituent group, size-limited substituent group, or lower substituent group; wherein if the substituted heterocycloalkyl is substituted with a plurality of groups selected from substituent groups, size-limited substituent groups, and lower substituent groups; each substituent group, size-limited substituent group, and/or lower substituent group may optionally be different. In embodiments, when the substituted heterocycloalkyl is substituted, it is substituted with at least one substituent group. In embodiments, when the substituted heterocycloalkyl is substituted, it is substituted with at least one size-limited substituent group. In embodiments, when the substituted heterocycloalkyl is substituted, it is substituted with at least one lower substituent group.

In embodiments, R^{2A} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered , 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R^{2A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2A} is substituted with a plurality of groups selected from substituent groups, size-limited substituent groups, and lower substituent groups; each substituent group, size-limited substituent group, and/or lower substituent group may optionally be different. In embodiments, when R^{2A} is substituted, it is substituted with at least one substituent group. In embodiments, when R^{2A} is substituted, it is substituted with at least one size-limited substituent group. In embodiments, when R^{2A} is substituted, it is substituted with at least one lower substituent group.

In embodiments, R^{2B} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R^{2B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2B} is substituted with a plurality of groups selected from substituent groups, size-limited substituent groups, and lower substituent groups; each substituent group, size-limited substituent group, and/or lower substituent group may optionally be different. In embodiments, when R^{2B} is substituted, it is substituted with at least one substituent group. In embodiments, when R^{2B} is substituted, it is substituted with at least one size-limited substituent group. In embodiments, when R^{2B} is substituted, it is substituted with at least one lower substituent group.

In embodiments, R^{2C} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R^{2C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2C} is substituted with a plurality of groups selected from substituent groups, size-limited substituent groups, and lower substituent groups; each substituent group, size-limited substituent group, and/or lower substituent group may optionally be different. In embodiments, when R^{2C} is substituted, it is substituted with at least one substituent group. In embodiments, when R^{2C} is substituted, it is substituted with at least one size-limited substituent group. In embodiments, when R^{2C} is substituted, it is substituted with at least one lower substituent group.

In embodiments, R^{2D} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R^{2D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2D} is substituted with a plurality of groups selected from substituent groups, size-limited substituent groups, and lower substituent groups; each substituent group, size-limited substituent group, and/or lower substituent group may optionally be different. In embodiments, when R^{2D} is substituted, it is substituted with at least one substituent group. In embodiments, when R^{2D} is substituted, it is substituted with at least one size-limited substituent group. In embodiments, when R^{2D} is substituted, it is substituted with at least one lower substituent group.

In embodiments, R^{2A} and R^{2B} substituents bonded to the same nitrogen atom may optionally be joined to form a substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered) or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, R^{2A} and R^{2B} substituents bonded to the same nitrogen atom that are optionally joined to form a substituted heterocycloalkyl can be substituted with at least one substituent group, size-limited substituent group, or lower substituent group; wherein if the substituted heterocycloalkyl is substituted with a plurality of groups selected from substituent groups, size-limited substituent groups, and lower substituent groups; each substituent group, size-limited substituent group, and/or lower substituent group may optionally be different. In embodiments, when the substituted heterocycloalkyl is substituted, it is substituted with at least one substituent group. In embodiments, when the substituted heterocycloalkyl is substituted, it is substituted with at least one size-limited substituent group. In embodiments, when the substituted heterocycloalkyl is substituted, it is substituted with at least one lower substituent group.

In embodiments, R^{1.2} is independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted (e.g., substituted with at least one substituent group, size-limited substituent group, or lower substituent group) or unsubstituted alkyl (e.g., C₁-C₈, C₁-C₆, C₁-C₄, or C₁-C₂), or substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered).

In embodiments, R^{1.3} is independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted (e.g., substituted with at least one substituent group, size-limited substituent group, or lower substituent group) or unsubstituted alkyl (e.g., C₁-C₈, C₁-C₆, C₁-C₄, or C₁-C₂), or substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered).

In embodiments, R^{1.2} and R^{1.3} substituents on adjacent carbons may optionally be joined to form a substituted (e.g., substituted with at least one substituent group, size-limited substituent group, or lower substituent group) or unsubstituted phenyl, or substituted (e.g., substituted with at least one substituent group, size-limited substituent group, or lower substituent group) or unsubstituted 5 to 6 membered heteroaryl.

In embodiments, R^{1.2} and R^{1.3} are independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted (e.g., substituted with at least one substituent group, size-limited substituent group, or lower substituent group) or unsubstituted C₁-C₆ alkyl, or substituted (e.g., substituted with at least one substituent group, size-limited substituent group, or lower substituent group) or unsubstituted 2 to 6 membered heteroalkyl; or R^{1.2} and R^{1.3} substituents on adjacent carbons may optionally be joined to form a substituted (e.g., substituted with at least one substituent group, size-limited substituent group, or lower substituent group) or unsubstituted phenyl, or substituted (e.g., substituted with at least one substituent group, size-limited substituent group, or lower substituent group) or unsubstituted 5 to 6 membered heteroaryl.

In embodiments, a substituted R^{1.2} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1.2} is substituted with a plurality of groups selected from substituent groups, size-limited substituent groups, and lower substituent groups; each substituent group, size-limited substituent group, and/or lower substituent group may optionally be different. In embodiments, when R^{1.2} is substituted, it is substituted with at least one substituent group. In embodiments, when R^{1.2} is substituted, it is substituted with at least one size-limited substituent group. In embodiments, when R^{1.2} is substituted, it is substituted with at least one lower substituent group.

In embodiments, a substituted R^{1.3} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1.3} is substituted with a plurality of groups selected from substituent groups, size-limited substituent groups, and lower substituent groups; each substituent group, size-limited substituent group, and/or lower substituent group may optionally be different. In embodiments, when R^{1.3} is substituted, it is substituted with at least one substituent group. In embodiments, when R^{1.3} is substituted, it is substituted with at least one size-limited substituent group. In embodiments, when R^{1.3} is substituted, it is substituted with at least one lower substituent group.

In embodiments, R^{1.2} and R^{1.3} substituents on adjacent carbons may optionally be joined to form a substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, R^{1.4} is independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted (e.g., substituted with at least one substituent group, size-limited substituent group, or lower substituent group) or unsubstituted alkyl (e.g., C₁-C₈, C₁-C₆, C₁-C₄, or C₁-C₂), or substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered).

In embodiments, R^{1.5} is independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted (e.g., substituted with at least one substituent group, size-limited substituent group, or lower substituent group) or unsubstituted alkyl (e.g., C₁-C₈, C₁-C₆, C₁-C₄, or C₁-C₂), or substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered).

In embodiments, R^{1.4} and R^{1.5} are independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted (e.g., substituted with at least one substituent group, size-limited substituent group, or lower substituent group) or unsubstituted C₁-C₆ alkyl, or substituted (e.g., substituted with at least one substituent group, size-limited substituent group, or lower substituent group) or unsubstituted 2 to 6 membered heteroalkyl.

In embodiments, a substituted R^{1.4} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1.4} is substituted with a plurality of groups selected from substituent groups, size-limited substituent groups, and lower substituent groups; each substituent group, size-limited substituent group, and/or lower substituent group may optionally be different. In embodiments, when R^{1.4} is substituted, it is substituted with at least one substituent group. In embodiments, when R^{1.4} is substituted, it is substituted with at least one size-limited substituent group. In embodiments, when R^{1.4} is substituted, it is substituted with at least one lower substituent group.

In embodiments, a substituted R^{1.5} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1.5} is substituted with a plurality of groups selected from substituent groups, size-limited substituent groups, and lower substituent groups; each substituent group, size-limited substituent group, and/or lower substituent group may optionally be different. In embodiments, when R^{1.5} is substituted, it is substituted with at least one substituent group. In embodiments, when R^{1.5} is substituted, it is substituted with at least one size-limited substituent group. In embodiments, when R^{1.5} is substituted, it is substituted with at least one lower substituent group.

In embodiments, R^{2.1} is independently oxo, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B},—NHC(O)NR^{2A}R^{2B}, —N(O)ₘ₂, —NR^{2A}R^{2B}, —C(O)R^{2C}, —C(O)—OR^{2C}, —C(O) NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R^{2.1} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2.1} is substituted with a plurality of groups selected from substituent groups, size-limited substituent groups, and lower substituent groups; each substituent group, size-limited substituent group, and/or lower substituent group may optionally be different. In embodiments, when R^{2.1} is substituted, it is substituted with at least one substituent group. In embodiments, when R^{2.1} is substituted, it is substituted with at least one size-limited substituent group. In embodiments, when R^{2.1} is substituted, it is substituted with at least one lower substituent group.

In embodiments, R^{2.2} is independently oxo, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B},—NHC(O)NR^{2A}R^{2B}, —N(O)ₘ₂, —NR^{2A}R^{2B}, —C(O)R^{2C}, —C(O)—OR^{2C}, —C(O) NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R^{2.2} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2.2} is substituted with a plurality of groups selected from substituent groups, size-limited substituent groups, and lower substituent groups; each substituent group, size-limited substituent group, and/or lower substituent group may optionally be different. In embodiments, when R^{2.2} is substituted, it is substituted with at least one substituent group. In embodiments, when R^{2.2} is substituted, it is substituted with at least one size-limited substituent group. In embodiments, when R^{2.2} is substituted, it is substituted with at least one lower substituent group.

In embodiments, R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O) NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, R¹¹-substituted or unsubstituted alkyl, R¹¹-substituted or unsubstituted heteroalkyl, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl; two adjacent R¹ substituents on adjacent carbons may optionally be joined to form a R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl.

In embodiments, R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O) NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, R¹¹-substituted or unsubstituted C₁-C₆ alkyl, R¹¹-substituted or unsubstituted 2 to 6 membered heteroalkyl, R¹¹-substituted or unsubstituted C₃-C₆ cycloalkyl, R¹¹-substituted or unsubstituted 3 to 6 membered heterocycloalkyl, R¹¹-substituted or unsubstituted C₆-C₁₀ aryl, or R¹¹-substituted or unsubstituted 5 to 10 membered heteroaryl; two adjacent R¹ substituents on adjacent carbons may optionally be joined to form a R¹¹-substituted or unsubstituted C₃-C₆ cycloalkyl, R¹¹-substituted or unsubstituted 3 to 6 membered heterocycloalkyl, R¹¹-substituted or unsubstituted phenyl, or R¹¹-substituted or unsubstituted 5 to 6 membered heteroaryl.

In embodiments, R^{1.2} and R^{1.3} are independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, R¹¹-substituted or unsubstituted C₁-C₆ alkyl, or R¹¹-substituted or unsubstituted 2 to 6 membered heteroalkyl; or R^{1.2} and R^{1.3} substituents on adjacent carbons may optionally be joined to form a R¹¹-substituted or unsubstituted phenyl, or R¹¹-substituted or unsubstituted 5 to 6 membered heteroaryl.

In embodiments, R^{1.2} is independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, R¹¹-substituted or unsubstituted C₁-C₆ alkyl, or R¹¹-substituted or unsubstituted 2 to 6 membered heteroalkyl.

In embodiments, R^{1.3} is independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, R¹¹-substituted or unsubstituted C₁-C₆ alkyl, or R¹¹-substituted or unsubstituted 2 to 6 membered heteroalkyl.

In embodiments, R^{1.2} and R^{1.3} substituents on adjacent carbons may optionally be joined to form a R¹¹-substituted or unsubstituted phenyl, or R¹¹-substituted or unsubstituted 5 to 6 membered heteroaryl.

In embodiments, R^{1.4} and R^{1.5} are independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, R¹¹-substituted or unsubstituted C₁-C₆ alkyl, or R¹¹-substituted or unsubstituted 2 to 6 membered heteroalkyl.

In embodiments, R^{1.4} is independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, R¹¹-substituted or unsubstituted C₁-C₆ alkyl, or R¹¹-substituted or unsubstituted 2 to 6 membered heteroalkyl.

In embodiments, R^{1.5} is independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, R¹¹-substituted or unsubstituted C₁-C₆ alkyl, or R¹¹-substituted or unsubstituted 2 to 6 membered heteroalkyl.

In embodiments, R^{1A}, R^{1B}, R^{1C}, and R^{1D} independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, R¹¹-substituted or unsubstituted alkyl, R¹¹-substituted or unsubstituted heteroalkyl, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl; R^{1A} and R^{1B} substituents bonded to the same nitrogen atom may optionally be joined to form a R¹¹-substituted or unsubstituted heterocycloalkyl or R¹¹-substituted or unsubstituted heteroaryl.

In embodiments, R^{1A} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, R¹¹-substituted or unsubstituted alkyl, R¹¹-substituted or unsubstituted heteroalkyl, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl.

In embodiments, R^{1B} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, R¹¹-substituted or unsubstituted alkyl, R¹¹-substituted or unsubstituted heteroalkyl, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl.

In embodiments, R^{1A} and R^{1B} substituents bonded to the same nitrogen atom may optionally be joined to form a R¹¹-substituted or unsubstituted heterocycloalkyl or R¹¹-substituted or unsubstituted heteroaryl.

In embodiments, R^{1C} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, R¹¹-substituted or unsubstituted alkyl, R¹¹-substituted or unsubstituted heteroalkyl, R¹¹ -substituted or unsubstituted cycloalkyl, R¹¹-substituted or unsubstituted heterocycloalkyl, R¹¹-substituted or unsubstituted aryl, or R¹¹-substituted or unsubstituted heteroaryl.

In embodiments, R^{1D} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, R¹¹-substituted or unsubstituted alkyl, R¹¹-substituted or unsubstituted heteroalkyl, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl.[0487] In embodiments, R² is independently oxo, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B},—NHC(O)NR^{2A}R^{2B}, —N(O)ₘ₂, —NR^{2A}R^{2B}, —C(O)R^{2C}, —C(O)—OR^{2C}, —C(O) NR2AR2B, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, R²¹-substituted or unsubstituted C₁-C₆ alkyl, R²¹-substituted or unsubstituted 2 to 6 membered heteroalkyl, R²¹-substituted or unsubstituted C₃-C₆ cycloalkyl, R²¹-substituted or unsubstituted 3 to 6 membered heterocycloalkyl, R²¹-substituted or unsubstituted C₆-C₁₀ aryl, or R²¹-substituted or unsubstituted 5 to 10 membered heteroaryl.

In embodiments, R^{2A}, R^{2B}, R^{2C}, and R^{2D} are independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, R²¹-substituted or unsubstituted alkyl, R²¹-substituted or unsubstituted heteroalkyl, R²¹-substituted or unsubstituted cycloalkyl, R²¹-substituted or unsubstituted heterocycloalkyl, R²¹-substituted or unsubstituted aryl, or R²¹-substituted or unsubstituted heteroaryl; R^{2A} and R^{2B} substituents bonded to the same nitrogen atom may optionally be joined to form a R²¹-substituted or unsubstituted heterocycloalkyl or R²¹-substituted or unsubstituted heteroaryl.

In embodiments, R^{2A} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, R²¹-substituted or unsubstituted alkyl, R²¹-substituted or unsubstituted heteroalkyl, R²¹-substituted or unsubstituted cycloalkyl, R²¹-substituted or unsubstituted heterocycloalkyl, R²¹-substituted or unsubstituted aryl, or R²¹-substituted or unsubstituted heteroaryl.

In embodiments, R^{2B} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, R²¹-substituted or unsubstituted alkyl, R²¹-substituted or unsubstituted heteroalkyl, R²¹-substituted or unsubstituted cycloalkyl, R²¹-substituted or unsubstituted heterocycloalkyl, R²¹-substituted or unsubstituted aryl, or R²¹-substituted or unsubstituted heteroaryl.

In embodiments, R^{2A} and R^{2B} substituents bonded to the same nitrogen atom may optionally be joined to form a R²¹-substituted or unsubstituted heterocycloalkyl or R²¹-substituted or unsubstituted heteroaryl.

In embodiments, R^{2C} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, R²¹-substituted or unsubstituted alkyl, R²¹-substituted or unsubstituted heteroalkyl, R²¹-substituted or unsubstituted cycloalkyl, R²¹-substituted or unsubstituted heterocycloalkyl, R²¹-substituted or unsubstituted aryl, or R²¹-substituted or unsubstituted heteroaryl.

In embodiments, R^{2D} is independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, R²¹-substituted or unsubstituted alkyl, R²¹-substituted or unsubstituted heteroalkyl, R²¹-substituted or unsubstituted cycloalkyl, R²¹-substituted or unsubstituted heterocycloalkyl, R²¹-substituted or unsubstituted aryl, or R²¹-substituted or unsubstituted heteroaryl.

In embodiments, R¹¹ is independently oxo, halogen, -CX¹¹₃, -CHX¹¹₂, —CH₂X¹¹, —OCX¹¹₃, —OCH₂X¹¹, —OCHX¹¹₂, —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R¹¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹¹ is substituted with a plurality of groups selected from substituent groups, size-limited substituent groups, and lower substituent groups; each substituent group, size-limited substituent group, and/or lower substituent group may optionally be different. In embodiments, when R¹¹ is substituted, it is substituted with at least one substituent group. In embodiments, when R¹¹ is substituted, it is substituted with at least one size-limited substituent group. In embodiments, when R¹¹ is substituted, it is substituted with at least one lower substituent group.

In embodiments, R²¹ is independently oxo, halogen, -CX²¹3, -CHX²¹₂, —CH₂X²¹, —OCX²¹₃, —OCH₂X²¹, —OCHX²¹₂, —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R²¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R²¹ is substituted with a plurality of groups selected from substituent groups, size-limited substituent groups, and lower substituent groups; each substituent group, size-limited substituent group, and/or lower substituent group may optionally be different. In embodiments, when R²¹ is substituted, it is substituted with at least one substituent group. In embodiments, when R²¹ is substituted, it is substituted with at least one size-limited substituent group. In embodiments, when R²¹ is substituted, it is substituted with at least one lower substituent group.

In embodiments, R^{11.1} is independently oxo, halogen, -CX¹¹₃, -CHX¹¹₂, —CH₂X¹¹, —OCX¹¹₃, —OCH₂X¹¹, —OCHX¹¹₂, —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R^{11.1} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{11.1} is substituted with a plurality of groups selected from substituent groups, size-limited substituent groups, and lower substituent groups; each substituent group, size-limited substituent group, and/or lower substituent group may optionally be different. In embodiments, when R ^{11.1} is substituted, it is substituted with at least one substituent group. In embodiments, when R^{11.1} is substituted, it is substituted with at least one size-limited substituent group. In embodiments, when R^{11.1} is substituted, it is substituted with at least one lower substituent group.

In embodiments, the compound has the formula:

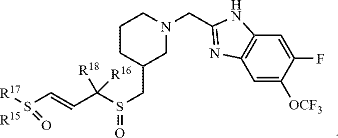

R¹⁷ is substituted or unsubstituted aryl; and R¹⁵, R¹⁶, and R¹⁸ are as described herein, including embodiments. In embodiments, R¹⁷ is substituted or unsubstituted phenyl. In embodiments, the compound has the formula:

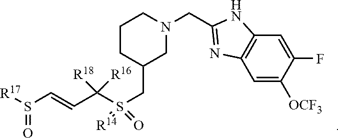

R¹⁷ is substituted or unsubstituted aryl; and R¹⁴, R¹⁶, and R¹⁸ are as described herein, including embodiments. In embodiments, R¹⁷ is substituted or unsubstituted phenyl. In embodiments, the compound has the formula:

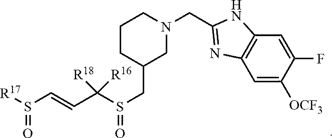

R¹⁷ is substituted or unsubstituted aryl; and R¹⁶, and R¹⁸ are as described herein, including embodiments. In embodiments, R¹⁷ is substituted or unsubstituted phenyl. In embodiments, the compound has the formula:

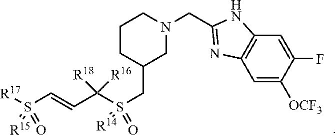

R¹⁷ is substituted or unsubstituted aryl; and R¹⁴, R¹⁵, R¹⁶, and R¹⁸ are as described herein, including embodiments. In embodiments, R¹⁷ is substituted or unsubstituted phenyl. In embodiments, the compound has the formula:

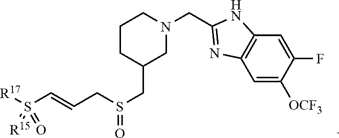

R¹⁷ is substituted or unsubstituted aryl; and R¹⁵ is as described herein, including embodiments. In embodiments, R¹⁷ is substituted or unsubstituted phenyl. In embodiments, the compound has the formula:

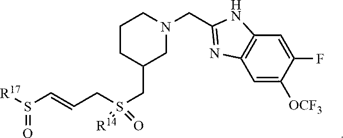

R¹⁷ is substituted or unsubstituted aryl; and R¹⁴ is as described herein, including embodiments. In embodiments, R¹⁷ is substituted or unsubstituted phenyl. In embodiments, the compound has the formula:

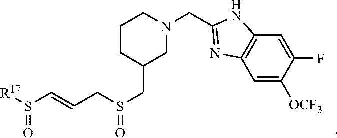

R¹⁷ is substituted or unsubstituted aryl. In embodiments, R¹⁷ is substituted or unsubstituted phenyl. In embodiments, the compound has the formula:

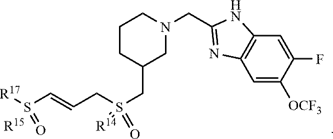

R¹⁷ is substituted or unsubstituted aryl; and R¹⁴ and R¹⁵ are as described herein, including embodiments. In embodiments, R¹⁷ is substituted or unsubstituted phenyl. In embodiments, the compound has the formula:

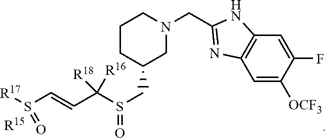

R¹⁷ is substituted or unsubstituted aryl; and R¹⁵, R¹⁶, and R¹⁸ are as described herein, including embodiments. In embodiments, R¹⁷ is substituted or unsubstituted phenyl. In embodiments, the compound has the formula:

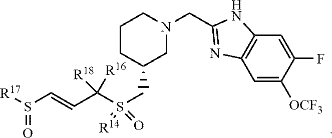

R¹⁷ is substituted or unsubstituted aryl; and R¹⁴, R¹⁶, and R¹⁸ are as described herein, including embodiments. In embodiments, R¹⁷ is substituted or unsubstituted phenyl. In embodiments, the compound has the formula:

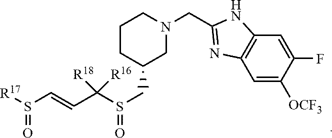

R¹⁷ is substituted or unsubstituted aryl; and R¹⁶, and R¹⁸ are as described herein, including embodiments. In embodiments, R¹⁷ is substituted or unsubstituted phenyl. In embodiments, the compound has the formula:

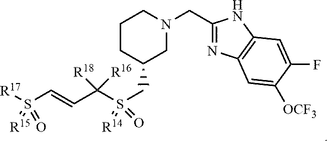

R¹⁷ is substituted or unsubstituted aryl; and R¹⁴, R¹⁵, R¹⁶, and R¹⁸ are as described herein, including embodiments. In embodiments, R¹⁷ is substituted or unsubstituted phenyl. In embodiments, the compound has the formula:

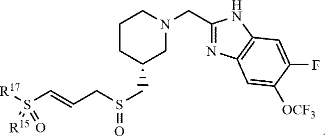

R¹⁷ is substituted or unsubstituted aryl; and R¹⁵ is as described herein, including embodiments. In embodiments, R¹⁷ is substituted or unsubstituted phenyl. In embodiments, the compound has the formula:

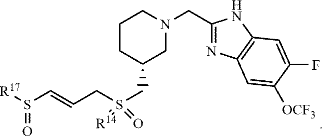

R¹⁷ is substituted or unsubstituted aryl; and R¹⁴ is as described herein, including embodiments. In embodiments, R¹⁷ is substituted or unsubstituted phenyl. In embodiments, the compound has the formula:

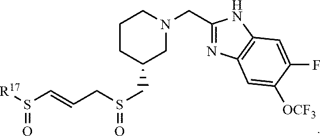

R¹⁷ is substituted or unsubstituted aryl. In embodiments, R¹⁷ is substituted or unsubstituted phenyl. In embodiments, the compound has the formula:

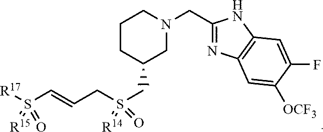

R¹⁷ is substituted or unsubstituted aryl; and R¹⁴ and R¹⁵ are as described herein, including embodiments. In embodiments, R¹⁷ is substituted or unsubstituted phenyl.

In embodiments, the compound has the formula:

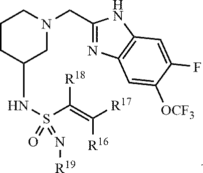

R¹⁹ is hydrogen, —CH₃, —CF₃, or —CN; and R¹⁶, R¹⁷, and R¹⁸ are as described herein, including embodiments. In embodiments, the compound has the formula:

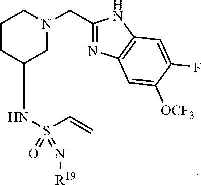

R¹⁹ is hydrogen, —CH₃, —CF₃, or —CN. In embodiments, the compound has the formula:

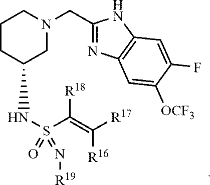

R¹⁹ is hydrogen, —CH₃, —CF₃, or —CN; and R¹⁶, R¹⁷, and R¹⁸ are as described herein, including embodiments. In embodiments, the compound has the formula:

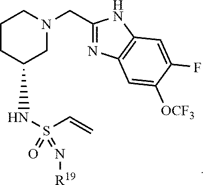

R¹⁹ is hydrogen, —CH₃, —CF₃, or —CN.

In embodiments, the compound has the formula:

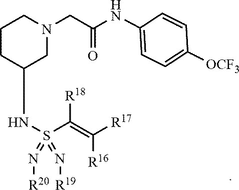

R¹⁹ and R²⁰ are independently hydrogen, —CH₃, —CF₃, or —CN; and R¹⁶, R¹⁷, and R¹⁸ are as described herein, including embodiments. In embodiments, R¹⁹ is hydrogen or —CH₃. In embodiments, R²⁰ is hydrogen or —CH₃. In embodiments, the compound has the formula:

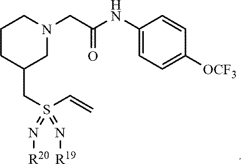

R¹⁹ and R²⁰ are independently hydrogen, —CH₃, —CF₃, or —CN. In embodiments, R¹⁹ is hydrogen or —CH₃. In embodiments, R²⁰ is hydrogen or —CH₃. In embodiments, the compound has the formula:

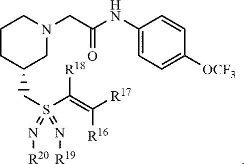

R¹⁹ and R²⁰ are independently hydrogen, —CH₃, —CF₃, or —CN; and R¹⁶, R¹⁷, and R¹⁸ are as described herein, including embodiments. In embodiments, R¹⁹ is hydrogen or —CH₃. In embodiments, R²⁰ is hydrogen or —CH₃. In embodiments, the compound has the formula:

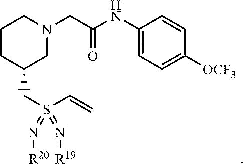

R¹⁹ and R²⁰ are independently hydrogen, —CH₃, —CF₃, or —CN. In embodiments, R¹⁹ is hydrogen or —CH₃. In embodiments, R²⁰ is hydrogen or —CH₃.

In embodiments, the compound has the formula:

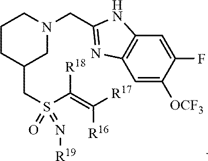

R¹⁹ is hydrogen, —CH₃, —CF₃, or —CN; and R¹⁶, R¹⁷, and R¹⁸ are as described herein, including embodiments. In embodiments, the compound has the formula:

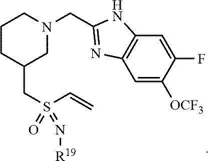

R¹⁹ is hydrogen, —CH₃, —CF₃, or —CN. In embodiments, the compound has the formula:

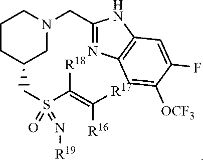

R¹⁹ is hydrogen, —CH₃, —CF₃, or —CN; and R¹⁶, R¹⁷, and R¹⁸ are as described herein, including embodiments. In embodiments, the compound has the formula:

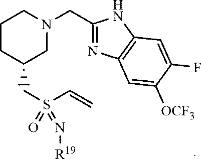

R¹⁹ is hydrogen, —CH₃, —CF₃, or —CN.

In embodiments, the compound has the formula:

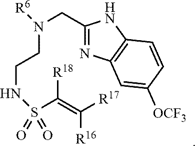

R⁶ is —CF₃, —COCH₃, or unsubstituted cyclopropyl; and R¹⁶, R¹⁷, and R¹⁸ are as described herein, including embodiments. In embodiments, the compound has the formula:

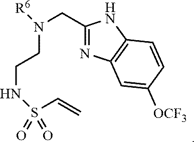

R⁶ is —CF₃, —COCH₃, or unsubstituted cyclopropyl. In embodiments, the compound has the formula:

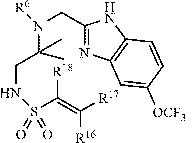

R⁶ is —CF₃, —COCH₃, or unsubstituted cyclopropyl; and R¹⁶, R¹⁷, and R¹⁸ are as described herein, including embodiments. In embodiments, the compound has the formula:

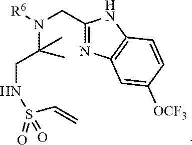

R⁶ is —CF₃, —COCH₃, or unsubstituted cyclopropyl. In embodiments, the compound has the formula:

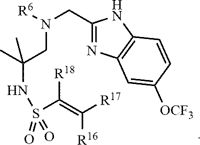

. R⁶ is —CF₃, —COCH₃, or unsubstituted cyclopropyl; and R¹⁶, R¹⁷, and R¹⁸ are as described herein, including embodiments. In embodiments, the compound has the formula: or unsubstituted cyclopropyl.

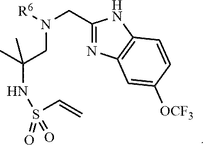

R⁶ is —CF₃, —COCH₃,

In embodiments, the compound is:

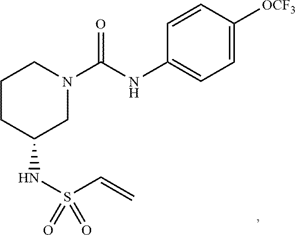

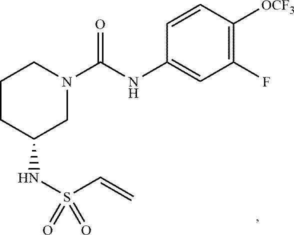

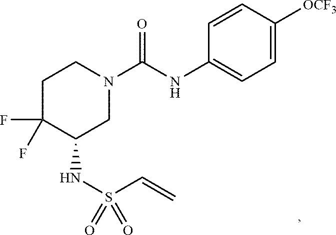

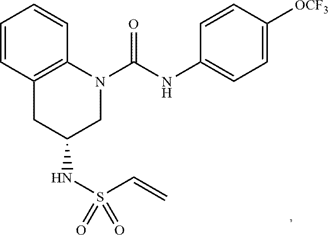

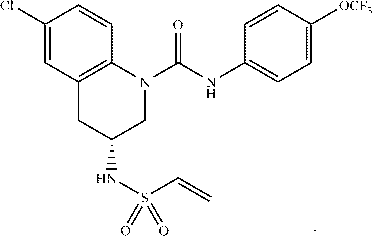

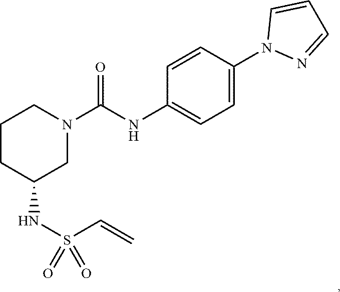

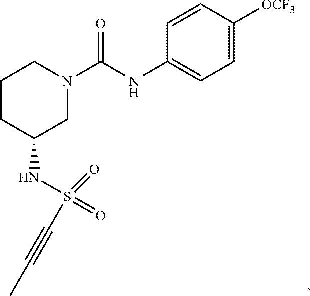

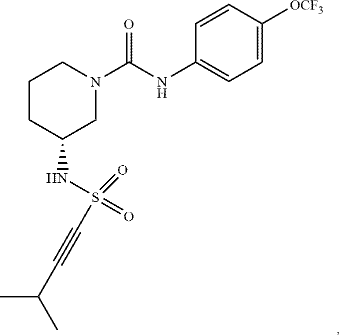

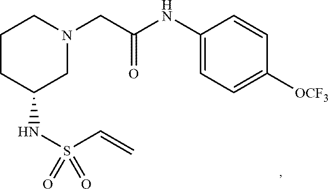

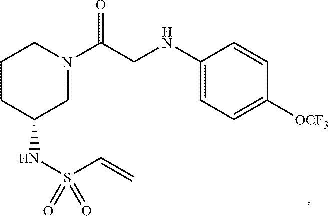

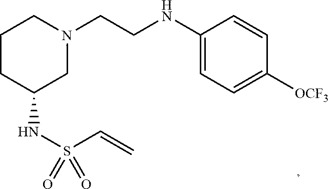

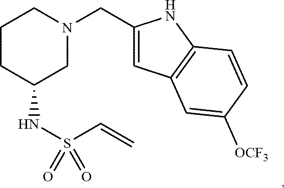

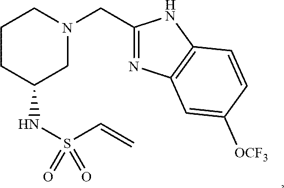

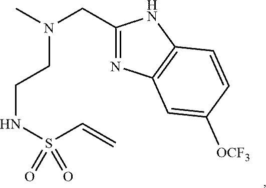

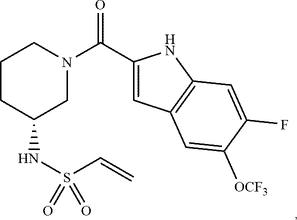

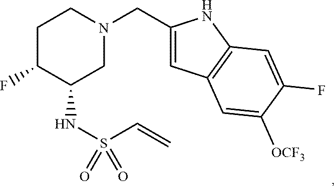

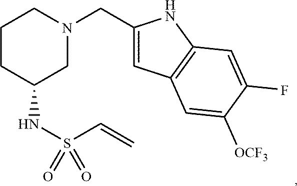

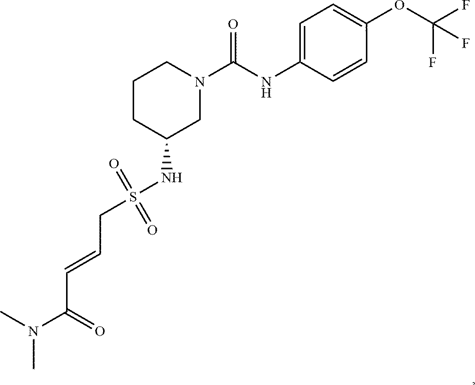

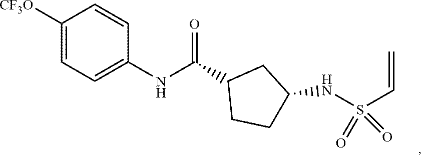

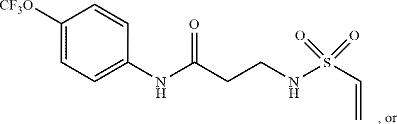

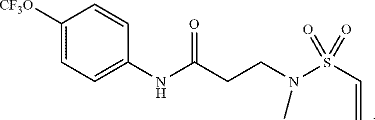

In embodiments, the compound is:

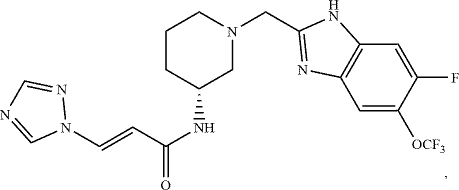

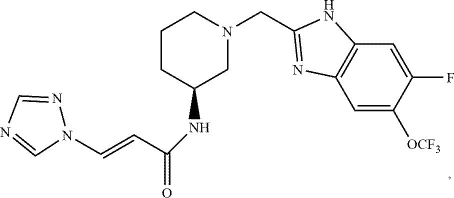

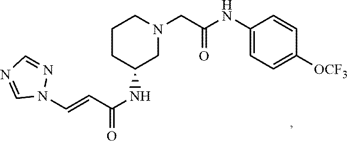

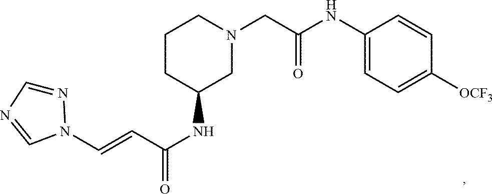

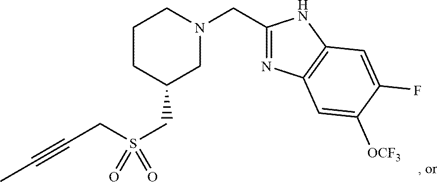

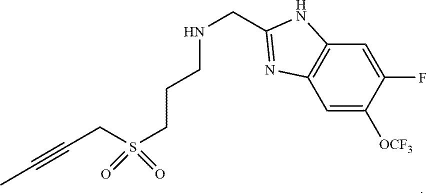

In embodiments, the compound is:

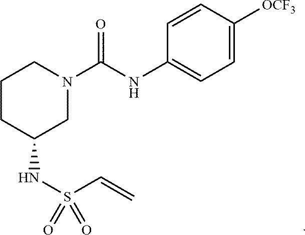

In embodiments, the compound is:

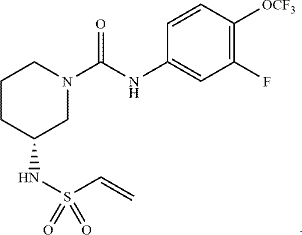

In embodiments, the compound is:

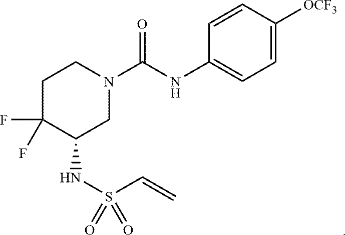

In embodiments, the compound is:

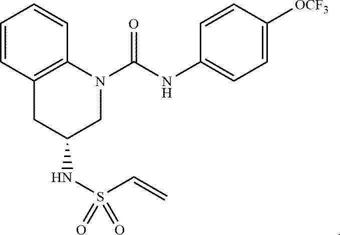

In embodiments, the compound is:

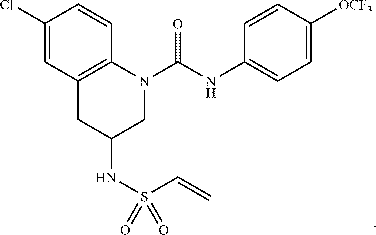

In embodiments, the compound is:

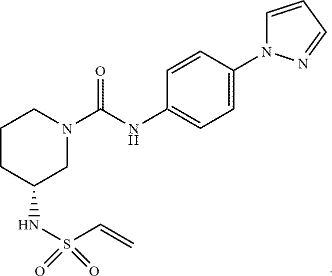

In embodiments, the compound is:

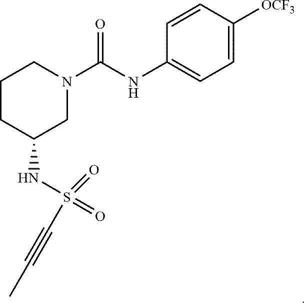

In embodiments, the compound is:

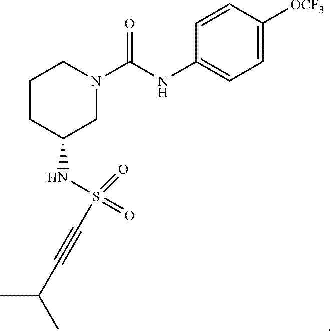

In embodiments, the compound is:

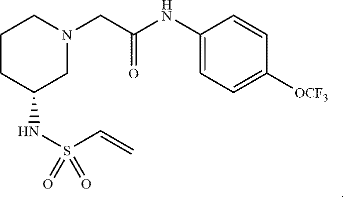

In embodiments, the compound

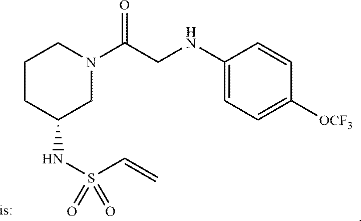

In embodiments, the compound is:

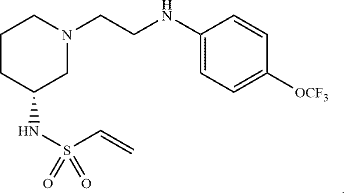

In embodiments, the compound is:

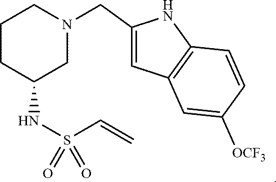

In embodiments, the compound is:

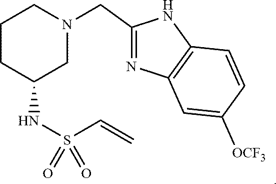

In embodiments, the compound is:

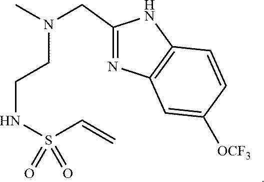

In embodiments, the compound is:

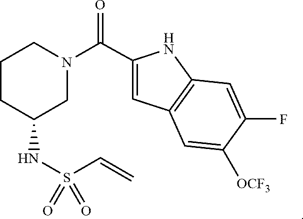

In embodiments, the compound is:

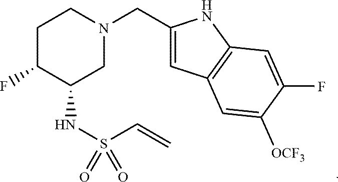

In embodiments, the compound is:

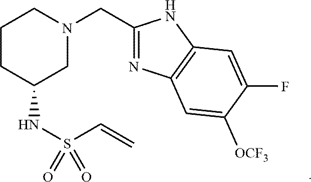

In embodiments, the compound is:

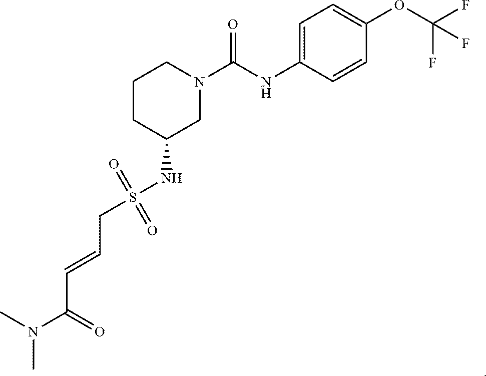

In embodiments, the compound is:

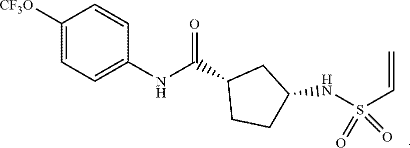

In embodiments, the compound is:

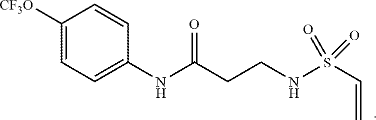

In embodiments, the compound is:

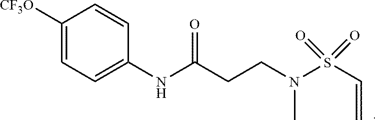

In embodiments, the compound is:

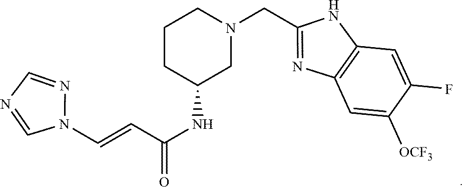

In embodiments, the compound is:

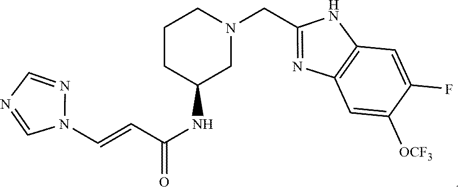

In embodiments, the compound is:

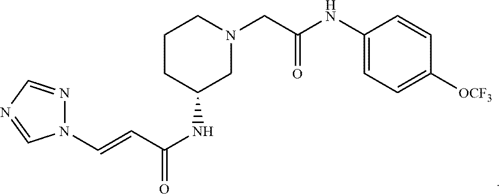

In embodiments, the compound is:

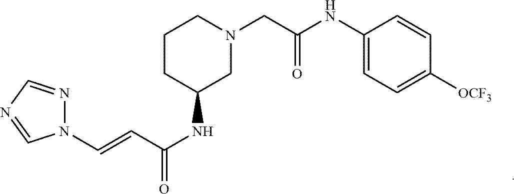

In embodiments, the compound is:

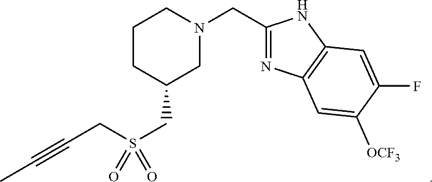

In embodiments, the compound is:

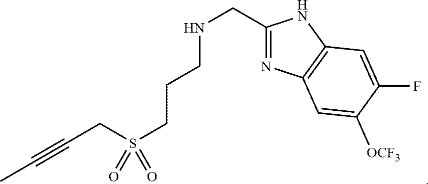

In embodiments, the compound is useful as a comparator compound. In embodiments, the comparator compound can be used to assess the activity of a test compound in an assay (e.g., an assay as described herein, for example in the examples section, figures, or tables).

In embodiments, the compound is a compound described herein (e.g., in an aspect, embodiment, example, table, figure, or claim).

### III. Pharmaceutical Compositions

In an aspect is provided a pharmaceutical composition including a compound as described herein, including in embodiments, and a pharmaceutically acceptable excipient. In embodiments, the compound as described herein is included in a therapeutically effective amount.

In embodiments of the pharmaceutical compositions, the compound, or pharmaceutically acceptable salt thereof, is included in a therapeutically effective amount.

In embodiments of the pharmaceutical compositions, the pharmaceutical composition includes a second agent (e.g. therapeutic agent). In embodiments of the pharmaceutical compositions, the pharmaceutical composition includes a second agent (e.g. therapeutic agent) in a therapeutically effective amount. In embodiments of the pharmaceutical compositions, the second agent is an agent for treating a neurodegenerative disease. In embodiments of the pharmaceutical compositions, the second agent is an agent for treating Alzheimer’s disease, Huntington’s disease, Amyotrophic lateral sclerosis, Lewy body disease, Progressive Supranuclear Palsy, or Parkinson’s disease. In embodiments of the pharmaceutical compositions, the second agent is an agent for treating a liver disease. In embodiments of the pharmaceutical compositions, the second agent is an agent for treating nonalcoholic steatohepatitis or nonalcoholic fatty liver disease. In embodiments, the administering does not include administration of any active agent other than the recited active agent (e.g., a compound described herein).

### IV. Methods of Use

In an aspect is provided a method of inhibiting human Caspase 6 protein activity, the method including: contacting the human Caspase 6 protein with a compound as described herein.

In embodiments, the compound covalently binds C264 of the human Caspase 6 protein. In embodiments, the compound covalently binds an amino acid corresponding to cysteine 264 of the human Caspase 6 protein. In embodiments, the compound forms a covalent bond with the protein. In embodiments, the compounds binds via an irreversible covalent bond.

In embodiments, the compound inhibits the activity of human Caspase 6 protein more than other human Caspase proteins.

In embodiments, the compound inhibits the activity of human Caspase 6 protein more than human Caspase 2 and human Caspase 3. In embodiments, the compound inhibits human Caspase 6 protein at least 1.1, 1.2, 1.3, 1.4, 1.5, 1.6, 1.7, 1.8, 1.9, 2, 3, 4, 5, 6, 7, 8, 9, 10, 20, 30, 40, 50, 60, 70, 80, 90, 100, 200, 300, 400, 500 600, 700, 800, 900, 1000, 2000, 3000, 4000, 5000, 6000, 7000, 8000, 9000, 10000, 20000, 30000, 40000, 50000, 60000, 70000, 80000, 90000, or 100000 fold more than human Caspase 2. In embodiments, the compound inhibits human Caspase 6 protein at least 1.1, 1.2, 1.3, 1.4, 1.5, 1.6, 1.7, 1.8, 1.9, 2, 3, 4, 5, 6, 7, 8, 9, 10, 20, 30, 40, 50, 60, 70, 80, 90, 100, 200, 300, 400, 500 600, 700, 800, 900, 1000, 2000, 3000, 4000, 5000, 6000, 7000, 8000, 9000, 10000, 20000, 30000, 40000, 50000, 60000, 70000, 80000, 90000, or 100000 fold more than human Caspase 3.

In an aspect is provided a method of treating a neurodegenerative disease, the method including administering to a subject in need thereof an effective amount of a compound as disclosed herein.

In embodiments, the neurodegenerative disease is a tauopathy.

In embodiments, the neurodegenerative disease is Alzheimer’s disease, Huntington’s disease, Amyotrophic lateral sclerosis, Lewy body disease, Progressive Supranuclear Palsy, Parkinson’s disease, frontotemporal degeneration (FTD), frontotemporal lobar degeneration (FTLD), or Pick’s disease.

In embodiments, the neurodegenerative disease is Alzheimer’s disease, Huntington’s disease, Amyotrophic lateral sclerosis, Lewy body disease, Progressive Supranuclear Palsy, or Parkinson’s disease.

In embodiments, the neurodegenerative disease is Alzheimer’s disease. In embodiments, the neurodegenerative disease is Huntington’s disease. In embodiments, the neurodegenerative disease is Amyotrophic lateral sclerosis. In embodiments, the neurodegenerative disease is Lewy body disease. In embodiments, the neurodegenerative disease is Progressive Supranuclear Palsy. In embodiments, the neurodegenerative disease is Parkinson’s disease. In embodiments, the neurodegenerative disease is frontotemporal degeneration (FTD). In embodiments, the neurodegenerative disease is frontotemporal lobar degeneration (FTLD). In embodiments, the neurodegenerative disease is Pick’s disease.

In an aspect is provided a method of treating a memory loss, the method including administering to a subject in need thereof an effective amount of a compound as disclosed herein.

In an aspect is provided a method of treating axonal degradation, the method including administering to a subject in need thereof an effective amount of a compound as disclosed herein. In embodiments, the method includes treating neuronal loss. In embodiments, the method includes treating brain volume loss.

In an aspect is provided a method of treating an inflammatory disease, the method including administering to a subject in need thereof an effective amount of a compound as disclosed herein. In embodiments, the inflammatory disease is an autoimmune diseases. In embodiments, the inflammatory disease is arthritis. In embodiments, the inflammatory disease is rheumatoid arthritis. In embodiments, the inflammatory disease is psoriatic arthritis. In embodiments. In embodiments, the inflammatory disease is the inflammatory disease is juvenile idiopathic arthritis. In embodiments, the inflammatory disease is multiple sclerosis. In embodiments, the inflammatory disease is systemic lupus erythematosus (SLE). In embodiments, the inflammatory disease is myasthenia gravis. In embodiments, the inflammatory disease is juvenile onset diabetes. In embodiments, the inflammatory disease is diabetes mellitus type 1. In embodiments, the inflammatory disease is Guillain-Barre syndrome. In embodiments, the inflammatory disease is Hashimoto’s encephalitis. In embodiments, the inflammatory disease is Hashimoto’s thyroiditis. In embodiments, the inflammatory disease is ankylosing spondylitis. In embodiments, the inflammatory disease is psoriasis. In embodiments, the inflammatory disease is Sjogren’s syndrome. In embodiments, the inflammatory disease is vasculitis. In embodiments, the inflammatory disease is glomerulonephritis. In embodiments, the inflammatory disease is auto-immune thyroiditis. In embodiments, the inflammatory disease is Behcet’s disease. In embodiments, the inflammatory disease is Crohn’s disease. In embodiments, the inflammatory disease is ulcerative colitis. In embodiments, the inflammatory disease is bullous pemphigoid. In embodiments, the inflammatory disease is sarcoidosis. In embodiments, the inflammatory disease is ichthyosis. In embodiments, the inflammatory disease is Graves ophthalmopathy. In embodiments, the inflammatory disease is inflammatory bowel disease. In embodiments, the inflammatory disease is Addison’s disease. In embodiments, the inflammatory disease is Vitiligo. In embodiments, the inflammatory disease is asthma. In embodiments, the inflammatory disease is allergic asthma. In embodiments, the inflammatory disease is acne vulgaris. In embodiments, the inflammatory disease is celiac disease. In embodiments, the inflammatory disease is chronic prostatitis. In embodiments, the inflammatory disease is inflammatory bowel disease. In embodiments, the inflammatory disease is pelvic inflammatory disease. In embodiments, the inflammatory disease is reperfusion injury. In embodiments, the inflammatory disease is ischemia reperfusion injury. In embodiments, the inflammatory disease is stroke. In embodiments, the inflammatory disease is sarcoidosis. In embodiments, the inflammatory disease is transplant rejection. In embodiments, the inflammatory disease is interstitial cystitis. In embodiments, the inflammatory disease is atherosclerosis. In embodiments, the inflammatory disease is scleroderma. In embodiments, the inflammatory disease is atopic dermatitis.

In an aspect is provided a method of treating neuroinflammation, the method including administering to a subject in need thereof an effective amount of a compound as disclosed herein.

In an aspect is provided a method of treating liver disease, the method including administering to a subject in need thereof an effective amount of a compound as disclosed herein. In embodiments, the liver disease is nonalcoholic steatohepatitis or nonalcoholic fatty liver disease. In embodiments, the liver disease is nonalcoholic steatohepatitis. In embodiments, the liver disease is nonalcoholic fatty liver disease.

In an aspect is provided a method of treating nonalcoholic steatohepatitis or nonalcoholic fatty liver disease, the method including administering to a subject in need thereof an effective amount of a compound as disclosed herein. In embodiments is provided a method of treating nonalcoholic steatohepatitis, the method including administering to a subject in need thereof an effective amount of a compound as disclosed herein. In an aspect is provided a method of treating nonalcoholic fatty liver disease, the method including administering to a subject in need thereof an effective amount of a compound as disclosed herein.

In an aspect is provided a method of treating a fibrotic disease, the method including administering to a subject in need thereof an effective amount of a compound as disclosed herein. In embodiments, the fibrotic disease occurs in the lung, liver, or brain. In embodiments, the fibrotic disease is pulmonary fibrosis. In embodiments, the fibrotic disease is pulmonary fibrosis. In embodiments, the fibrotic disease is cystic fibrosis. In embodiments, the fibrotic disease is idiopathic pulmonary fibrosis. In embodiments, the fibrotic disease is radiation-induced lung injury. In embodiments, the fibrotic disease is bridging fibrosis. In embodiments, the fibrotic disease is cirrhosis. In embodiments, the fibrotic disease is myocardial fibrosis. In embodiments, the fibrotic disease is interstitial fibrosis. In embodiments, the fibrotic disease is replacement fibrosis. In embodiments, the fibrotic disease is glial scar. In embodiments, the fibrotic disease is arterial stiffness. In embodiments, the fibrotic disease is arthrofibrosis. In embodiments, the fibrotic disease is Crohn’s disease. In embodiments, the fibrotic disease is Dupuytren’s contracture. In embodiments, the fibrotic disease is Keloid. In embodiments, the fibrotic disease is Mediastinal fibrosis. In embodiments, the fibrotic disease is Myelofibrosis. In embodiments, the fibrotic disease is Peyronie’s disease. In embodiments, the fibrotic disease is Nephrogenic systemic fibrosis. In embodiments, the fibrotic disease is Progressive massive fibrosis. In embodiments, the fibrotic disease is Retroperitoneal fibrosis. In embodiments, the fibrotic disease is Scleroderma (systemic sclerosis). In embodiments, the fibrotic disease is adhesive capsulitis.

### V. Caspase 6 Protein

In an aspect is provided a Caspase 6 protein covalently bonded to a compound as described herein. In embodiments, the compound is bonded (e.g., covalently bonded) to a cysteine residue of the protein.

In an aspect is provided a Caspase protein covalently bonded to a portion of a compound as described herein.

Where the compound covalently binds to Caspase 6, a Caspase 6 protein (e.g., human Caspase 6) covalently bonded to a Caspase 6 inhibitor is formed (also referred to herein as a “Caspase 6-compound adduct”), as described below. In embodiments, the resulting covalent bond is reversible. Where the resulting covalent bond is reversible, the bonding reverses upon denaturation of the protein. Thus, in embodiments, the reversibility of a covalent bond between the compound and the Caspase 6 upon denaturation of the Caspase 6 avoids or decreases autoimmune response in a subject subsequent to administration of the compound (relative to irreversibility).

In embodiments, the Caspase 6 protein (e.g., human Caspase 6) is covalently bonded to a Caspase 6 inhibitor (e.g., compound described herein or a portion of a compound described herein). In embodiments, the Caspase 6 protein (e.g., human Caspase 6) is irreversibly covalently bonded to a Caspase 6 inhibitor (e.g., compound described herein or a portion of a compound described herein). In embodiments, the Caspase 6 protein (e.g., human Caspase 6) is reversibly covalently bonded to a Caspase 6 inhibitor (e.g., compound described herein or a portion of a compound described herein). In embodiments, the Caspase 6 protein (e.g., human Caspase 6) is covalently bonded to a portion of a Caspase 6 inhibitor (e.g., compound described herein). In embodiments, the Caspase 6 protein (e.g., human Caspase 6) is irreversibly covalently bonded to a portion of a Caspase 6 inhibitor (e.g., compound described herein). In embodiments, the Caspase 6 protein (e.g., human Caspase 6) is reversibly covalently bonded to a portion of a Caspase 6 inhibitor (e.g., compound described herein). In embodiments, the Caspase 6 inhibitor (e.g., compound described herein) is bonded to a cysteine residue (e.g., Cys264 of human Caspase 6 or cysteine corresponding to Cys264 of human Caspase 6) of the Caspase 6 protein (e.g., human Caspase 6).

In embodiments, the Caspase 6 protein covalently bonded to a Caspase 6 inhibitor or compound described herein is the product of a reaction between the Caspase 6 protein and a Caspase 6 inhibitor or compound described herein. It will be understood that the covalently bonded Caspase 6 protein and Caspase 6 inhibitor (e.g., compound described herein) are the remnants of the reactant Caspase 6 protein and Caspase 6 inhibitor or compound, wherein each reactant now participates in the covalent bond between the Caspase 6 protein and Caspase 6 inhibitor or compound. In embodiments of the covalently bonded Caspase 6 protein and compound described herein, the remnant of the E substituent is a linker including a covalent bond between the Caspase 6 protein and the remainder of the compound described herein. It will be understood by a person of ordinary skill in the art that when a Caspase 6 protein is covalently bonded to a Caspase 6 inhibitor (e.g., compound described herein), the Caspase 6 inhibitor (e.g., compound described herein) forms a remnant of the pre-reacted Caspase 6 inhibitor (e.g., compound described herein) wherein a bond connects the remnant of the Caspase 6 inhibitor (e.g., compound described herein) to the remnant of the Caspase 6 protein (e.g., cysteine sulfur, sulfur of amino acid corresponding to C264 of human Caspase 6, sulfur of C264 of human Caspase 6). The remnant of the Caspase 6 inhibitor (compound described herein) may also be called a portion of the Caspase 6 inhibitor.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### VI. Embodiments

Embodiment P1. A compound having the formula:

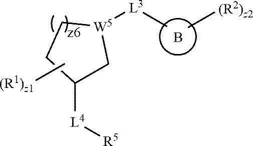

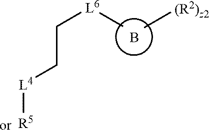

wherein,
   R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O) NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl; two adjacent R¹ substituents on adjacent carbons may optionally be joined to form a substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl; or two R¹ substituents bonded to the same carbon atom may optionally be joined to form a substituted or unsubstituted C₃-C₆ alkyl or substituted or unsubstituted 3 to 6 membered heterocycloalkyl;
   z1 is an integer from 0 to 9;
   R² is independently oxo, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B},—NHC(O)NR^{2A}R^{2B}, —N(O)ₘ₂, —NR^{2A}R^{2B}, —C(O)R^{2C}, —C(O)—OR^{2C}, —C(O) NR2AR2B, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl;
   z2 is an integer from 0 to 6;
   L³ is a bond, —S(O)₂—, —NR³—, —NH—, —O—, —S—, —C(O)—, —C(O)NR³—, —NR³C(O)—, —N(R³)CH₂—, —NR³C(O)NH —, —NHC(O)NR³—, —C(O)O—, —OC(O)—, substituted or unsubstituted C₁-C₆ alkylene, or substituted or unsubstituted 2 to 6 membered heteroalkylene;
   L⁴ is a bond, —NH—, —NR4—, or substituted or unsubstituted C₁-C₂ alkylene;
   L⁶ is —N(R⁶)—L³— or —C(O)NH—;
   W⁵ is CH orN;
   z6 is 1 or 2;
   R³, R⁴, and R⁶ are independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, unsubstituted C₁-C₆ alkyl, unsubstituted 2 to 6 membered heteroalkyl;
   Ring B is C₆-C₁₀ aryl, or 5 to 10 membered heteroaryl;
   R⁵ is independently 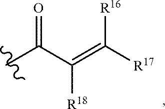 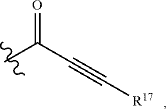 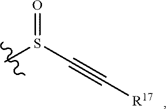 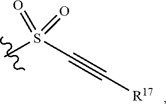 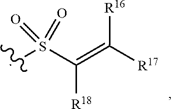 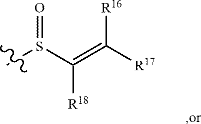 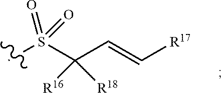
   wherein R¹⁶, R¹⁷, and R¹⁸ are independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl
   R^{1A}, R^{1B}, R^{1C,} R^{1D}, R^{2A} R^{2B}, R^{2C}, and R^{2D} are independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl;
   X¹ and X² are independently —F, —Cl, —Br, or —I;
   n1 and n2 are independently an integer from 0 to 4; and
   m1, m2, v1, and v2 are independently 1 or 2.
Embodiment P2. The compound of embodiment P1, having the formula:

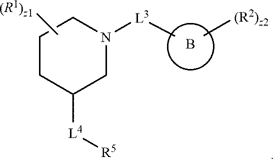

Embodiment P3. The compound of embodiment P2, having the formula:

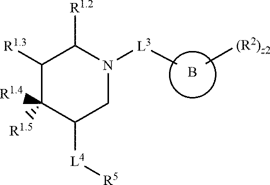

wherein,
   R^{1.2} and R^{1.3} are independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted C₁-C₆ alkyl, or substituted or unsubstituted 2 to 6 membered heteroalkyl; or R^{1.2} and R^{1.3} substituents on adjacent carbons may optionally be joined to form a substituted or unsubstituted phenyl, or substituted or unsubstituted 5 to 6 membered heteroaryl; and
   R^{1.4} and R^{1.5} are independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted C₁-C₆ alkyl, or substituted or unsubstituted 2 to 6 membered heteroalkyl.
Embodiment P4. The compound of embodiment P3, having the formula:

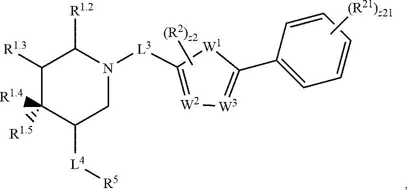

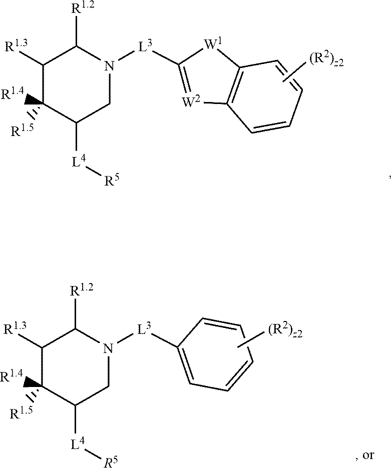

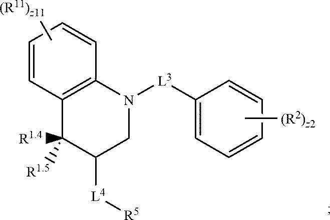

wherein,
   W¹ is independently —O—, —NH—, or —NR²—;
   W² and W³ are independently ═N—, ═CH—, or ═CR²—;
   R^{1.2}, R^{1.3}, R^{1.4} and R^{1.5} are independently hydrogen or halogen;
   R² is independently oxo, halogen, —CX²₃, —CHX²₂, —CH₂X², —OCX²₃, —OCH₂X², —OCHX²₂, —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted C₁-C₆ alkyl, unsubstituted 2 to 6 membered heteroalkyl, unsubstituted C₃—C₆ cycloalkyl, unsubstituted 3 to 6 membered heterocycloalkyl, unsubstituted C₆—C₁₂ aryl, or unsubstituted 5 to 12 membered heteroaryl;
   R¹¹ is independently oxo, halogen, —CX¹¹₃, —CHX¹¹₂, —CH₂X¹¹, —OCX¹¹₃, —OCH₂X¹¹, —OCHX¹¹₂, —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted C₁—C₆ alkyl, unsubstituted 2 to 6 membered heteroalkyl, unsubstituted C₃—C₆ cycloalkyl, unsubstituted 3 to 6 membered heterocycloalkyl, unsubstituted C₆—C₁₂ aryl, or unsubstituted 5 to 12 membered heteroaryl;
   R²¹ is independently oxo, halogen, —CX²¹₃, —CHX²¹₂, —CH₂X²¹, —OCX²¹₃, —OCH₂X²¹, —OCHX²¹₂, —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted C₁—C₆ alkyl, unsubstituted 2 to 6 membered heteroalkyl, unsubstituted C₃—C₆ cycloalkyl, unsubstituted 3 to 6 membered heterocycloalkyl, unsubstituted C₆—C₁₂ aryl, or unsubstituted 5 to 12 membered heteroaryl;
   X¹¹ and X²¹ are independently —F, —Cl, —Br, or —I;
   z2 is an integer from 0 to 6;
   z11 is an integer from 0 to 4;
   z21 is an integer from 0 to 5;
   L³ is —C(O)—, —CH₂—, —C(O)NH—, —NHC(O)—, —NHCH₂—, —CH₂CH₂NH—, —C(O)CH₂NH—, or —CH₂C(O)NH—;
   L⁴ is a bond, —NH—, or —CH₂—;
   R⁵ is independently 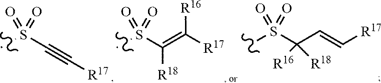
   wherein R¹⁶, R¹⁷, and R¹⁸ are independently hydrogen, —C(O)N(CH₃)₂, or unsubstituted C₁-C₃ alkyl.
Embodiment P5. The compound of embodiment P4, wherein: R² is independently halogen, —OCX²₃, —OCH₂X², —OCHX²₂, unsubstituted C₁—C₃ alkyl, or unsubstituted 5 to 6 membered heteroaryl; R¹¹ is independently halogen, —OCX¹¹₃, —OCH₂X¹¹, —OCHX¹¹₂, unsubstituted C₁—C₃ alkyl, or unsubstituted 5 to 6 membered heteroaryl; and R²¹ is independently halogen, —OCX²¹₃, —OCH₂X²¹, —OCHX²¹₂, unsubstituted C₁—C₃ alkyl, or unsubstituted 5 to 6 membered heteroaryl.
Embodiment P6. The compound of embodiments P4 or P5, having the formula:

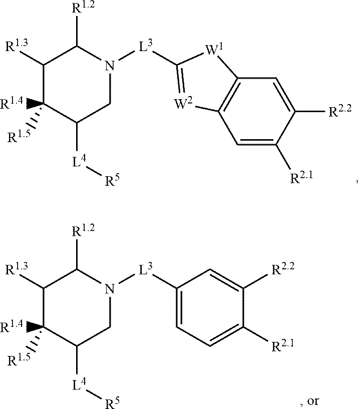

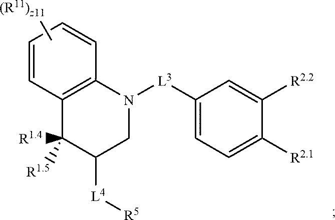

wherein,
   R^{2.1} is independently hydrogen, —OCX²₃, or unsubstituted 5 to 6 membered heteroaryl; and
   R^{2.2} is independently hydrogen or halogen.
Embodiment P7. The compound of one of embodiments P4 to P6, wherein R¹¹ is independently halogen.
Embodiment P8. The compound of embodiment P1, having the formula:

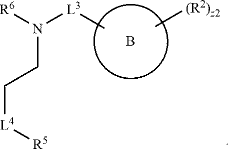

Embodiment P9. The compound of embodiment P8, having the formula:

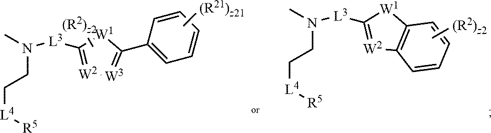

wherein,
   W¹ is independently —O—, —NH—, or —NR²—;
   W² and W³ are independently ═N—, ═CH—, or ═CR²—
   R² and R²¹ are independently halogen, —OCX²₃, —OCH₂X², —OCHX²₂, unsubstituted C₁—C₃ alkyl, or unsubstituted 5 to 6 membered heteroaryl;
   z2 is an integer from 0 to 6;
   z21 is an integer from 0 to 5;
   L³ is —C(O)— or —CH₂—;
   L⁴ is a bond, —NH—, —NR⁴—, or —CH₂—;
   R⁵ is independently 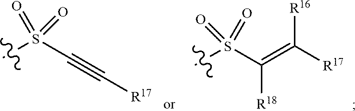
   wherein R¹⁶, R¹⁷, and R¹⁸ are independently hydrogen, —C(O)N(CH₃)₂, or unsubstituted C₁-C₃ alkyl.
Embodiment P10. The compound of embodiment P9, having the formula:

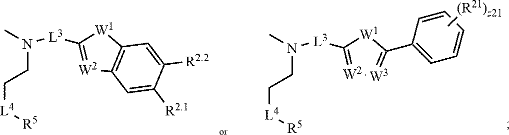

wherein,
   R^{2.1} is independently hydrogen, —OCX²₃, or unsubstituted 5 to 6 membered heteroaryl; and
   R^{2.2} is independently hydrogen or halogen.
Embodiment P11. The compound of one of embodiments P1 to P3 or P8, wherein
   (Ring B)-(R²)_{z2} is 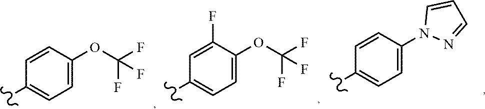 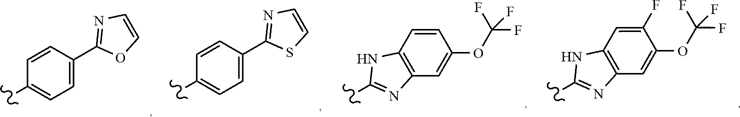 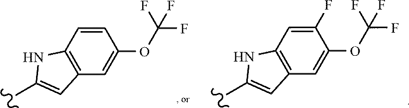
Embodiment P12. The compound of one of embodiments P1 to P11, wherein L⁴ is —NH—.
Embodiment P13. The compound of one of embodiments P1 to P11, wherein L⁴ is —CH₂—.
Embodiment P14. The compound of one of embodiments P1 to P11, wherein L⁴ is —N(CH₃)—.
Embodiment P15. The compound of one of embodiments P1 to P14, wherein
   R⁵ is independently 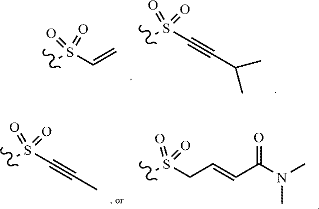
Embodiment P16. The compound of one of embodiments P1 to P6 and P10 to P15, wherein L³ is —C(O)—, —CH₂—, —C(O)NH—, —CH₂CH₂NH—, —C(O)CH₂NH—, or —CH₂C(O)NH.
Embodiment P17. The compound of one of embodiments P1 to P6 and P10 to P15, wherein L³ is —CH2— or —C(O)NH—.
Embodiment P18. The compound of one of embodiments P1 to P17, wherein R² is independently —F or —OCF₃.
Embodiment P19. The compound of embodiment P1 having the formula:

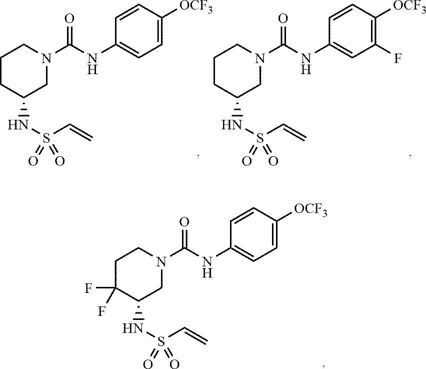

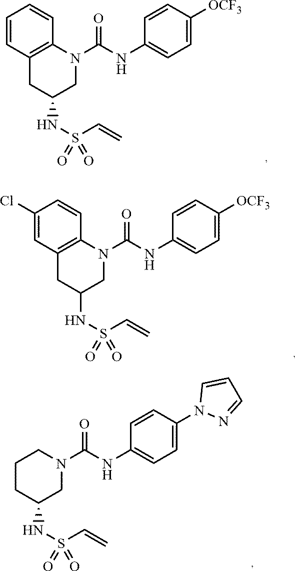

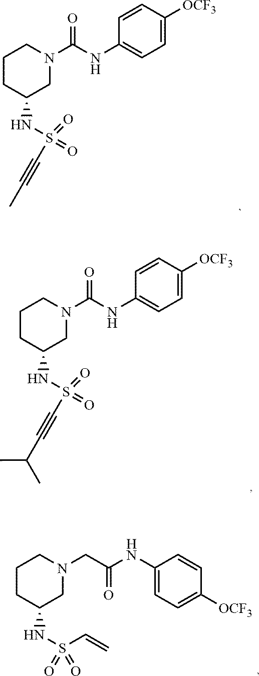

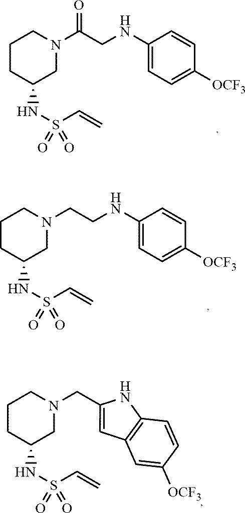

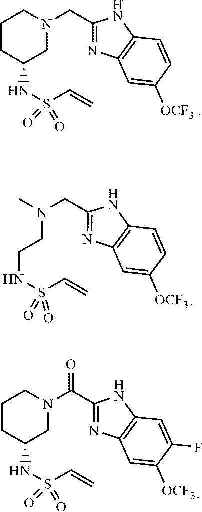

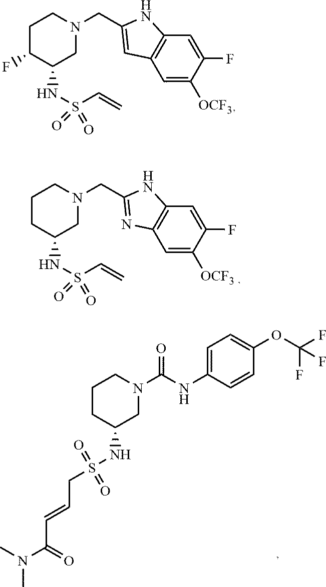

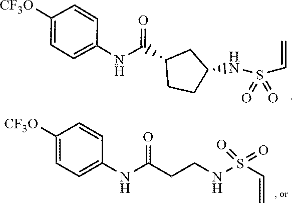

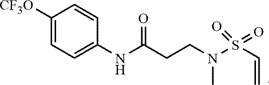

Embodiment P20. A pharmaceutical composition comprising the compound of any one of embodiments P1 to P19 and a pharmaceutically acceptable excipient.
Embodiment P21. A method of inhibiting human Caspase 6 protein activity, said method comprising: contacting the human Caspase 6 protein with a compound of one of embodiments P1 to P19.
Embodiment P22. The method of embodiment P21, wherein the compound covalently binds C264 of the human Caspase 6 protein.
Embodiment P23. The method of embodiment P21, wherein the compound inhibits the activity of human Caspase 6 protein more than other human Caspase proteins.
Embodiment P24. The method of embodiment P21, wherein the compound inhibits the activity of human Caspase 6 protein more than human Caspase 2 and human Caspase 3.
Embodiment P25. A method of treating a neurodegenerative disease, said method comprising administering to a subject in need thereof an effective amount of a compound of one of embodiments P1 to P19.
Embodiment P26. The method of embodiment P25 wherein the neurodegenerative disease is a tauopathy.
Embodiment P27. The method of embodiment P25, wherein the neurodegenerative disease is Alzheimer’s disease, Huntington’s disease, Amyotrophic lateral sclerosis, Lewy body disease, Progressive Supranuclear Palsy, or Parkinson’s disease.
Embodiment P28. The method of embodiment P25, wherein the neurodegenerative disease is Alzheimer’s disease.
Embodiment P29. A method of treating a memory loss, said method comprising administering to a subject in need thereof an effective amount of a compound of one of embodiments P1 to P19.
Embodiment P30. A method of treating axonal degradation, said method comprising administering to a subject in need thereof an effective amount of a compound of one of embodiments P1 to P19.
Embodiment P31. A method of treating neuroinflammation, said method comprising administering to a subject in need thereof an effective amount of a compound of one of embodiments P1 to P19.
Embodiment P32. A method of treating liver disease, said method comprising administering to a subject in need thereof an effective amount of a compound of one of embodiments P1 to P19.
Embodiment P33. A method of treating nonalcoholic steatohepatitis or nonalcoholic fatty liver disease, said method comprising administering to a subject in need thereof an effective amount of a compound of one of embodiments P1 to P19.

### VII. Additional Embodiments

Embodiment 1. A compound having the formula:

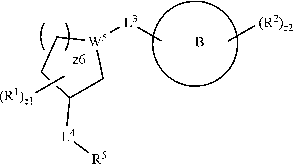

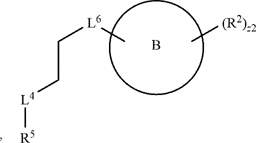

or

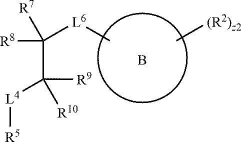

wherein,
   R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O) NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents on adjacent carbons may optionally be joined to form a substituted or unsubstituted cycloalkyl, substituted or unsubstituted heterocycloalkyl, substituted or unsubstituted aryl, or substituted or unsubstituted heteroaryl; or two R¹ substituents bonded to the same carbon atom may optionally be joined to form a substituted or unsubstituted cycloalkyl or substituted or unsubstituted heterocycloalkyl;
   z1 is an integer from 0 to 9;
   R² is independently oxo, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B},—NHC(O)NR^{2A}R^{2B}, —N(O)ₘ₂, —NR^{2A}R^{2B}, —C(O)R^{2C}, —C(O)—OR^{2C}, —C(O) NR2AR2B, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   z2 is an integer from 0 to 6;
   L³ is a
   bond, —S(O)₂—, —NR³—, —NH—, —O—, —S—, —C(O)—, —C(O)NR³—, —NR³C(O)—, —N(R³)CH₂—, —NR³C(O)NH —, —NHC(O)NR³—, —C(O)O—, —OC(O)—, substituted or unsubstituted alkylene, or substituted or unsubstituted heteroalkylene;
   L⁴ is a bond, —NH—, —NR⁴—, or substituted or unsubstituted alkylene;
   L⁶ is —N(R⁶)—L³— or —C(O)NH—;
   W⁵ is CH orN;
   z6 is 1 or 2;
   R³, R⁴, and R⁶ are independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CO(C₁—C₆ alkyl), —CONH₂, —OCCl₃, —OCF₃, —OCBr₃, —OCI₃, —OCHCl₂, —OCHBr₂, —OCHI₂, —OCHF₂, —OCH₂ Cl, —OCH₂Br, —OCH₂I, —OCH₂F, unsubstituted alkyl, unsubstituted heteroalkyl, unsubstituted cycloalkyl, or unsubstituted heterocycloalkyl;
   R⁷, R⁸, R⁹, and R¹⁰ are independently hydrogen or unsubstituted C₁-C₁₀ alkyl;
   Ring B is aryl, or membered heteroaryl;
   R⁵ is independently 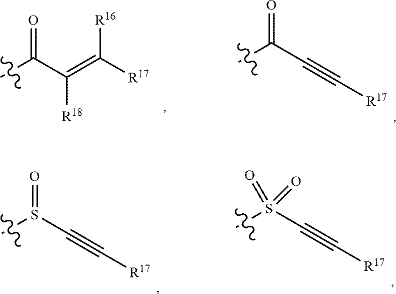 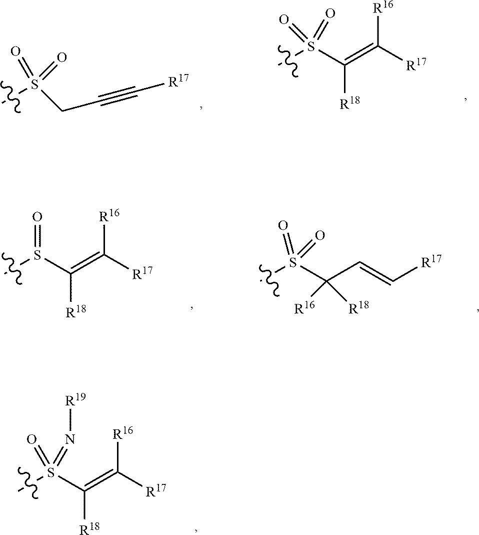 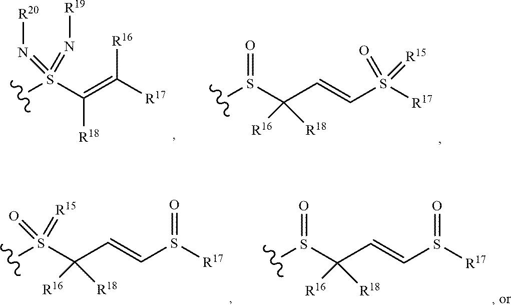 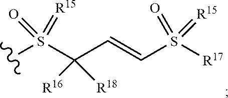
   wherein
   R¹⁴ is independently ═O or ═NR¹⁹;
   R¹⁵ is independently ═O or ═NR²⁰;
   R¹⁶, R¹⁷, and R¹⁸ are independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   R¹⁹ and R²⁰ are independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —COOH, —CONH₂, —C(O)N(CH₃)₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   R^{1A}, R^{1B}, R^{1C}, R^{1D}, R^{2A}, R^{2B}, R^{2C}, and R^{2D} are independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl;
   X¹ and X² are independently —F, —Cl, —Br, or —I;
   n1 and n2 are independently an integer from 0 to 4; and
   m1, m2, v1, and v2 are independently 1 or 2.
Embodiment 2. The compound of embodiment 1, having the formula:

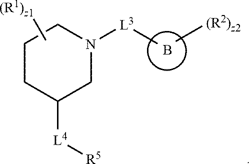

Embodiment 3. The compound of embodiment 2, having the formula:

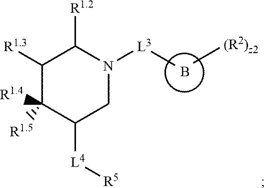

wherein,
   R^{1.2} and R^{1.3} are independently hydrogen, halogen, —CCl₃, —CBr₃, —CF₃, —CI₃, CHCl₂, —CHBr₂, —CHF₂, —CHI₂, -
   CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂,
   —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted C₁-C₆ alkyl, or substituted or unsubstituted 2 to 6 membered heteroalkyl; or R^{1.2} and R^{1.3} substituents on adjacent carbons may optionally be joined to form a substituted or unsubstituted phenyl, or substituted or unsubstituted 5 to 6 membered heteroaryl; and
   R^{1.4} and R^{1.5} are independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted C₁—C₆ alkyl, or substituted or unsubstituted 2 to 6 membered heteroalkyl.
Embodiment 4. The compound of embodiment 3, having the formula:

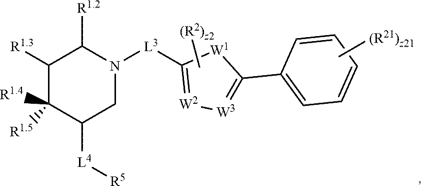

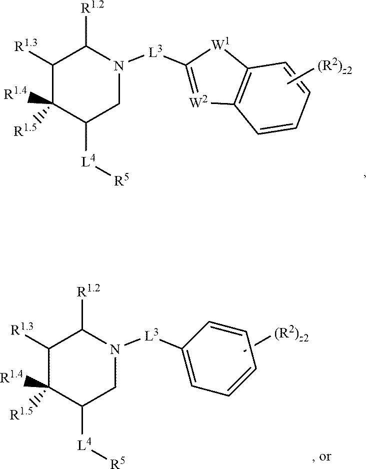

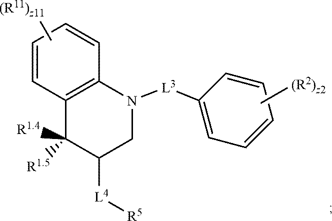

wherein,
   W¹ is independently —O—, —NH—, or —NR²—;
   W² and W³ are independently ═N—, ═CH—, or ═CR²—; R^{1.2}, R^{1.3}, R^{1.4} and R^{1.5} are independently hydrogen or halogen;
   R² is independently oxo, halogen, —CX²₃, —CHX²₂, —CH₂X², —OCX²₃, —OCH₂X², —OCHX²₂, —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted C₁—C₆ alkyl, unsubstituted 2 to 6 membered heteroalkyl, unsubstituted C₃—C₆ cycloalkyl, unsubstituted 3 to 6 membered heterocycloalkyl, unsubstituted C₆—C₁₂ aryl, or unsubstituted 5 to 12 membered heteroaryl;
   R¹¹ is independently oxo, halogen, —CX¹¹₃, —CHX¹¹₂, —CH₂X¹¹, —OCX¹¹₃, —OCH₂X¹¹, —OCHX¹¹₂, —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted C₁—C₆ alkyl, unsubstituted 2 to 6 membered heteroalkyl, unsubstituted C₃—C₆ cycloalkyl, unsubstituted 3 to 6 membered heterocycloalkyl, unsubstituted C₆—C₁₂ aryl, or unsubstituted 5 to 12 membered heteroaryl;
   R²¹ is independently oxo, halogen, —CX²¹₃, —CHX²¹₂, —CH₂X²¹, —OCX²¹₃, —OCH₂X²¹, —OCHX²¹₂, —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted C₁—C₆ alkyl, unsubstituted 2 to 6 membered heteroalkyl, unsubstituted C₃—C₆ cycloalkyl, unsubstituted 3 to 6 membered heterocycloalkyl, unsubstituted C₆—C₁₂ aryl, or unsubstituted 5 to 12 membered heteroaryl;
   X¹¹ and X²¹ are independently —F, —Cl, —Br, or —I;
   z2 is an integer from 0 to 6;
   z11 is an integer from 0 to 4;
   z21 is an integer from 0 to 5;
   L³ is —C(O)—, —CH₂—, —C(O)NH—, —NHC(O)—, —NHCH₂—, —CH₂CH₂NH—, —C(O)CH₂NH—, or —CH₂C(O)NH—;
   L⁴ is a bond, —NH—, or —CH₂—;
   R⁵ is independently 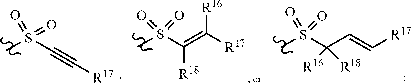
   wherein R¹⁶, R¹⁷, and R¹⁸ are independently hydrogen, —C(O)N(CH₃)₂, or unsubstituted C₁-C₃ alkyl.
Embodiment 5. The compound of embodiment 4, wherein: R² is independently halogen, —OCX²₃, —OCH₂X², —OCHX²₂, unsubstituted C₁—C₃ alkyl, or unsubstituted 5 to 6 membered heteroaryl; R¹¹ is independently halogen, —OCX¹¹₃, —OCH₂X¹¹, —OCHX¹¹₂, unsubstituted C₁—C₃ alkyl, or unsubstituted 5 to 6 membered heteroaryl; and R²¹ is independently halogen, —OCX²¹₃, —OCH₂X²¹, —OCHX²¹₂, unsubstituted C₁—C₃ alkyl, or unsubstituted 5 to 6 membered heteroaryl.
Embodiment 6. The compound of embodiment 4 or 5, having the formula:

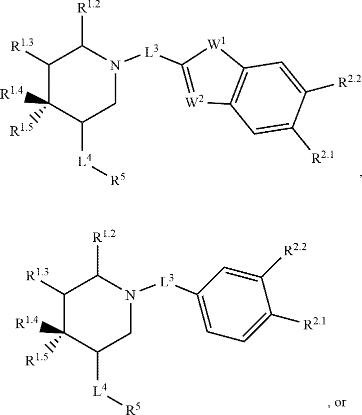

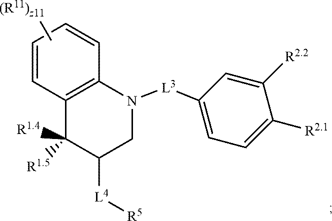

wherein,
   R^{2.1} is independently hydrogen, —OCX²₃, or unsubstituted 5 to 6 membered heteroaryl; and
   R^{2.2} is independently hydrogen or halogen.
Embodiment 7. The compound of one of embodiment 4 to 6, wherein R¹¹ is independently halogen.
Embodiment 8. The compound of embodiment 1, having the formula:

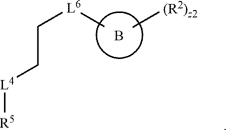

Embodiment 9. The compound of embodiment 1, having the formula:

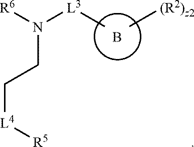

Embodiment 10. The compound of embodiment 9, having the formula:

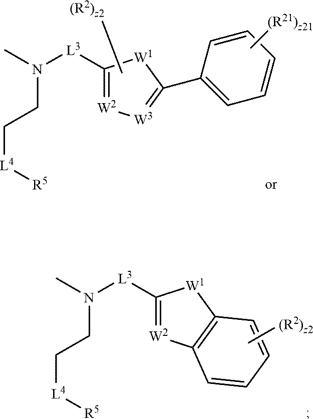

wherein,
   W¹ is independently —O—, —NH—, or —NR²—;
   W² and W³ are independently ═N—, ═CH—, or ═CR²—
   R² and R²¹ are independently halogen, —OCX²₃, —OCH₂X², —OCHX²₂, unsubstituted C₁—C₃ alkyl, or unsubstituted 5 to 6 membered heteroaryl;
   z2 is an integer from 0 to 6;
   z21 is an integer from 0 to 5;
   L³ is —C(O)— or —CH₂—;
   L⁴ is a bond, —NH—, —NR4—, or —CH₂—;
   R⁵ is independently 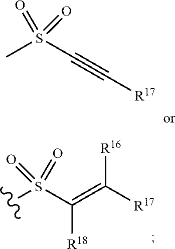
   wherein R¹⁶, R¹⁷, and R¹⁸ are independently hydrogen, —C(O)N(CH₃)₂, or unsubstituted C₁-C₃ alkyl.
Embodiment 11. The compound of embodiment 10, having the formula:

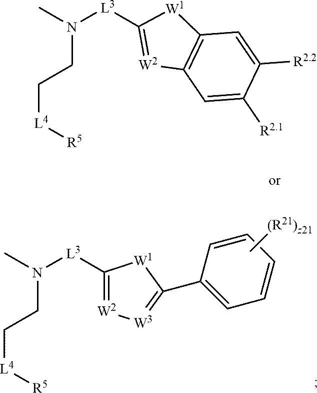

wherein,
   R^{2.1} is independently hydrogen, —OCX²₃, or unsubstituted 5 to 6 membered heteroaryl; and
   R^{2.2} is independently hydrogen or halogen.
Embodiment 12. The compound of one of embodiments 1 to 3 or 8 to 9, wherein
   (Ring B)-(R²)_{z2} is 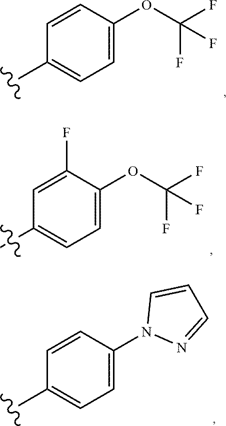 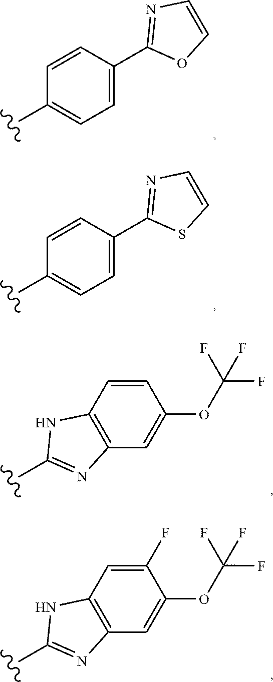 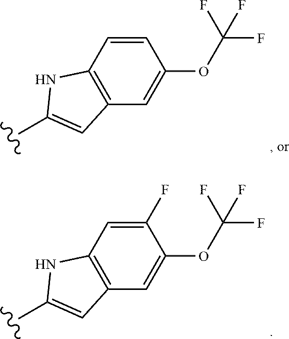
Embodiment 13. The compound of one of embodiments 1 to 12, wherein L⁴ is —NH—.
Embodiment 14. The compound of one of embodiments 1 to 12, wherein L⁴ is —CH₂—.
Embodiment 15. The compound of one of embodiments 1 to 12, wherein L⁴ is —N(CH₃)—.
Embodiment 16. The compound of one of embodiments 1 to 15, wherein
   R⁵ is independently 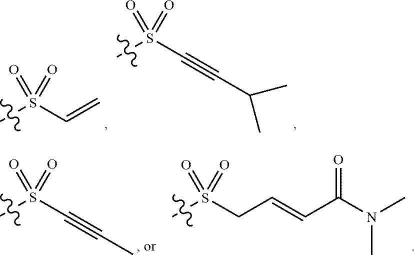
Embodiment 17. The compound of one of embodiments 1 to 6 and 11 to 16, wherein L³ is —C(O)—, —CH₂—, —C(O)NH—, —CH₂CH₂NH—, —C(O)CH₂NH—, or —CH₂C(O)NH.
Embodiment 18. The compound of one of embodiments 1 to 6 and 11 to 16, wherein L³ is —CH₂— or —C(O)NH—.
Embodiment 19. The compound of one of embodiments 1 to 18, wherein R² is independently —F or —OCF₃.
Embodiment 20. The compound of embodiment 1 having the formula:

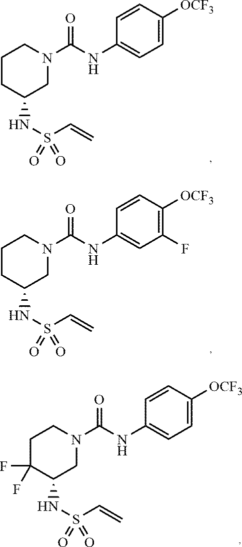

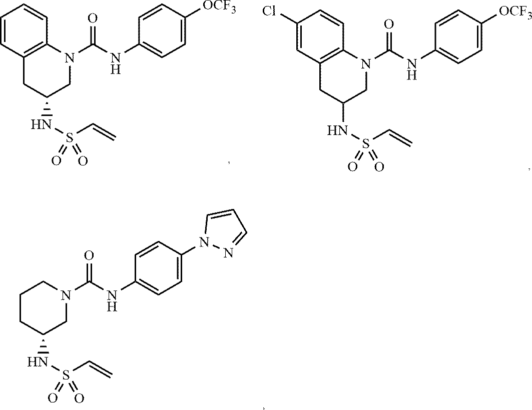

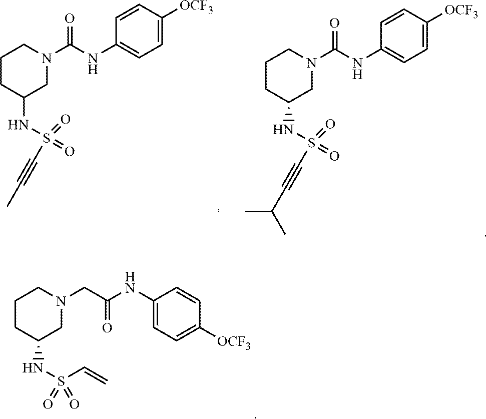

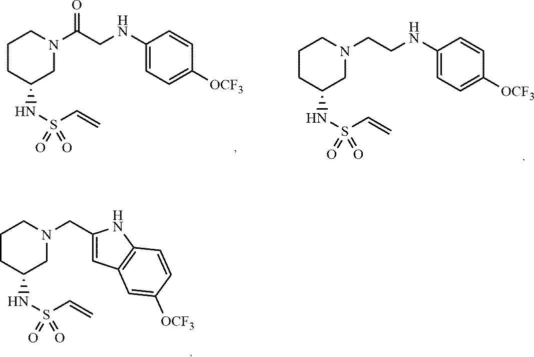

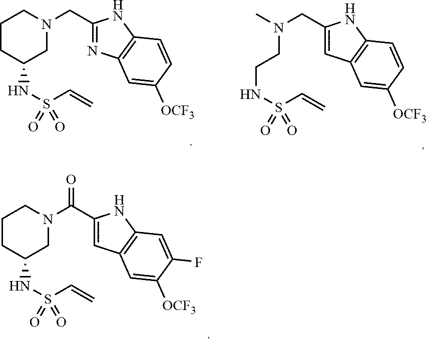

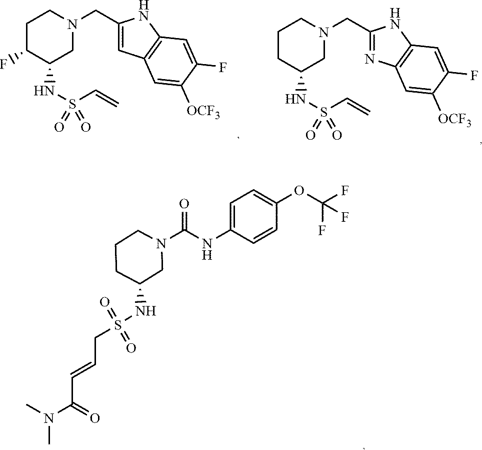

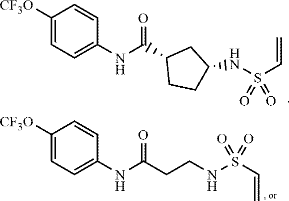

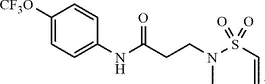

Embodiment 21. A pharmaceutical composition comprising the compound of any one of embodiments 1 to 20 and a pharmaceutically acceptable excipient.
Embodiment 22. A method of inhibiting human Caspase 6 protein activity, said method comprising: contacting the human Caspase 6 protein with a compound of one of embodiments 1 to 20.
Embodiment 23. The method of embodiment 22, wherein the compound covalently binds C264 of the human Caspase 6 protein.
Embodiment 24. The method of embodiment 22, wherein the compound inhibits the activity of human Caspase 6 protein more than other human Caspase proteins.
Embodiment 25. The method of embodiment 22, wherein the compound inhibits the activity of human Caspase 6 protein more than human Caspase 2 and human Caspase 3.
Embodiment 26. A method of treating a neurodegenerative disease, said method comprising administering to a subject in need thereof an effective amount of a compound of one of embodiments 1 to 20.
Embodiment 27. The method of embodiment 26, wherein the neurodegenerative disease is a tauopathy.
Embodiment 28. The method of embodiment 26, wherein the neurodegenerative disease is Alzheimer’s disease, Huntington’s disease, Amyotrophic lateral sclerosis, Lewy body disease, Progressive Supranuclear Palsy, or Parkinson’s disease.
Embodiment 29. The method of embodiment 26, wherein the neurodegenerative disease is Alzheimer’s disease.
Embodiment 30. A method of treating a memory loss, said method comprising administering to a subject in need thereof an effective amount of a compound of one of embodiments 1 to 20.
Embodiment 31. A method of treating axonal degradation, said method comprising administering to a subject in need thereof an effective amount of a compound of one of embodiments 1 to 20.
Embodiment 32. A method of treating neuroinflammation, said method comprising administering to a subject in need thereof an effective amount of a compound of one of embodiments 1 to 20.
Embodiment 33. A method of treating liver disease, said method comprising administering to a subject in need thereof an effective amount of a compound of one of embodiments 1 to 20.
Embodiment 34. A method of treating nonalcoholic steatohepatitis or nonalcoholic fatty liver disease, said method comprising administering to a subject in need thereof an effective amount of a compound of one of embodiments 1 to 20.
Embodiment 35. A compound of any one of embodiments 1 to 20, or pharmaceutically acceptable salt thereof, for use in a method of treating a neurodegenerative disease, comprising administering to a subject in need thereof an effective amount of the compound.
Embodiment 36. A compound for the use of embodiment 35, wherein the neurodegenerative disease is a tauopathy.
Embodiment 37. A compound for the use of embodiment 35, wherein the neurodegenerative disease is Alzheimer’s disease, Huntington’s disease, Amyotrophic lateral sclerosis, Lewy body disease, Progressive Supranuclear Palsy, or Parkinson’s disease.
Embodiment 38. A compound for the use of embodiment 35, wherein the neurodegenerative disease is Alzheimer’s disease.
Embodiment 39. A compound of any one of embodiments 1 to 20, or pharmaceutically acceptable salt thereof, for use in a method of treating a memory loss, comprising administering to a subject in need thereof an effective amount of the compound.
Embodiment 40. A compound of any one of embodiments 1 to 20, or pharmaceutically acceptable salt thereof, for use in a method of treating axonal degradation, comprising administering to a subject in need thereof an effective amount of the compound.
Embodiment 41. A compound of any one of embodiments 1 to 20, or pharmaceutically acceptable salt thereof, for use in a method of treating neuroinflammation, comprising administering to a subject in need thereof an effective amount of the compound.
Embodiment 42. A compound of any one of embodiments 1 to 20, or pharmaceutically acceptable salt thereof, for use in a method of treating liver disease, comprising administering to a subject in need thereof an effective amount of the compound.
Embodiment 43. A compound of any one of embodiments 1 to 20, or pharmaceutically acceptable salt thereof, for use in a method of treating nonalcoholic steatohepatitis or nonalcoholic fatty liver disease, comprising administering to a subject in need thereof an effective amount of the compound.

### EXAMPLES

### Example 1: Synthetic Methods

General information: All evaporations were carried out in vacuo with a rotary evaporator. Analytical samples were dried in vacuo (1-5 mmHg) at rt. Thin layer chromatography (TLC) was performed on silica gel plates, spots were visualized by UV light (214 and 254 nm). Purification by column and flash chromatography was carried out using silica gel (200-300 mesh). Solvent systems are reported as mixtures by volume. All NMR spectra were recorded on a Bruker 400 (400 MHz) spectrometer. ¹H chemical shifts are reported in δ values in ppm with the deuterated solvent as the internal standard. Data are reported as follows: chemical shift, multiplicity (s = singlet, d = doublet, t = triplet, q = quartet, br = broad, m = multiplet), coupling constant (Hz), integration. LCMS spectra were obtained on an Agilent 1200 series 6110 or 6120 mass spectrometer with electrospray ionization and excepted as otherwise indicated, the general LCMS condition was as follows: Waters X Bridge C18 column (50 mm × 4.6 mm x 3.5 um), Flow Rate: 2.0 ml/min, the column temperature: 40° C.

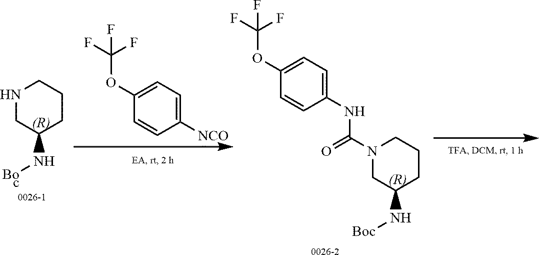

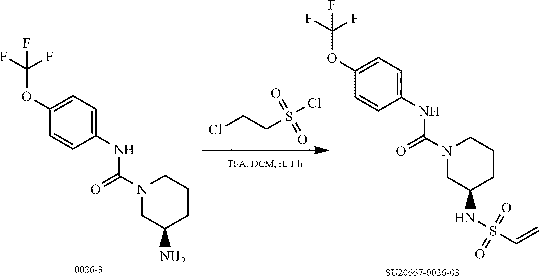

The synthesis of (R)-tert-butyl 1-(4-(trifluoromethoxy)phenylcarbamoyl)piperidin-3-ylcarbamate (0026-2).

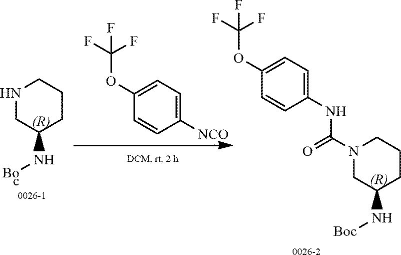

To a solution of 0026-1 (5.00 g, 25.0 mmol) in DCM (20 mL) was added 1-isocyanato-4-(trifluoromethoxy)benzene (5.08 g, 25.0 mmol) dropwise at room temperature. The mixture was stirred for 2 h. The solid was filtered to give 0026-2 (8.00 g, yield: 79%) as a white solid.

The synthesis of (R)-3-amino-N-(4-(trifluoromethoxy)phenyl)piperidine-1-carboxamide (0026-3).

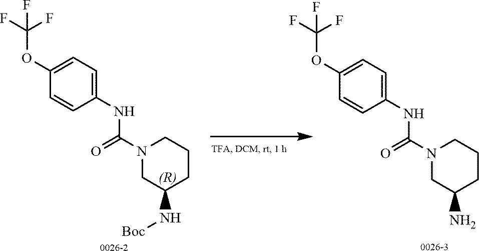

To a suspension of solution of 0026-2 (4.00 g, 9.93 mmol) in DCM (20.0 mL), was added TFA (6.00 mL) at room temperature. The reaction mixture was stirred for 1 h. The solvent was removed. The residue was partitioned between DCM (50.0 mL) and saturated aq. NaHCO₃ (50.0 mL). The organic layer was separated. The aqueous layer was extracted with DCM (2 × 50.0 mL). The combined organic layers were washed with brine (2 × 50.0 mL), dried over anhydrous Na₂SO₄, filtered and concentrated in vacuo to give 0026-3 (3.00 g, yield: 100.0%) as a yellow solid.

The synthesis of (R)-N-(4-(trifluoromethoxy)phenyl)-3-(vinylsulfonamido)piperidine-1-carboxamide (SU20667-0026-03)

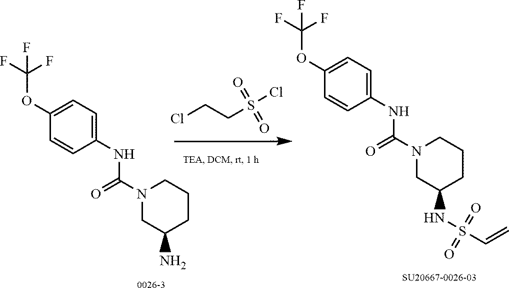

To a solution of 0026-3 (3.66 g, 12.1 mmol) and TEA (3.05 g, 30.2 mmol) in DCM (50.0 mL) was added 2-chloroethanesulfonyl chloride (1.97 g, 12.1 mmol) under ice-water bath. The mixture was allowed to warm to room temperature and stirred for 1 h. Water (50.0 mL) and DCM (100 mL) was added. The organic layer was separated. The aqueous layer was extracted with DCM (2 × 50.0 mL). The combined organic layers were washed with brine (2 × 50.0 mL), dried over anhydrous Na₂SO₄, filtered and concentrated in vacuo to give crude product which was purified by c.c. (PE/EA=2:1) to give SU20667-0026-03 (2.40 g, yield: 51%) as a white solid. LC-MS (Agilent LCMS 1200-6120, Column: Waters X-Bridge C18 (50 mm*4.6 mm*3.5 µm); Column Temperature: 40° C.; Flow Rate: 2.0 mL/min; Mobile Phase: from 95% [water + 10 mM NH₄HCO₃] and 5% [CH3CN] to 0% [water + 10 mM NH₄HCO3] and 100% [CH3CN] in 1.6 min, then under this condition for 1.4 min, finally changed to 95% [water + 10 mM NH₄HCO₃] and 5% [CH₃CN] in 0.1 min and under this condition for 0.7 min), Purity: 98.43%, Rt =1.933 min; MS Calcd: 393.10; MS Found: 394.2 [M+H]⁺. HPLC (Agilent HPLC 1200, Column: L-column2 ODS (150 mm*4.6 mm*5.0 µm); Column Temperature: 40° C.; Flow Rate: 1.5 mL/min; Mobile Phase: from 90% [(total 10 mM AcONH4) water/CH₃CN=900/100 (v/v)] and 10% [total 10 mM AcONH₄) water/CH₃CN=100/900 (v/v)] to 15% [total 10 mM AcONH₄) water/CH₃CN=900/100 (v/v)] and 85% [total 10 mM AcONH4) water/CH₃CN=100/900 (v/v)] in 5 min, then under this condition for 10 min, finally changed to 90% [(total 10 mM AcONH4) water/CH₃CN=900/100 (v/v)] and 10% [total 10 mM AcONH4) water/CH₃CN=100/900 (v/v)] in 0.1 min and under this condition for 5 min), Purity: 100.0%, Rt =9.072 min. ¹H NMR (400 MHz, DMSO-d₆) δ 8.70 (s, 1 H), 7.50-7.56 (m, 3 H), 7.23 (d, J=8.4 Hz, 2 H), 6.77 (dd, J=16.4 Hz, 10.0 Hz, 1 H), 6.05 (d, J=16.4 Hz, 1 H), 5.95 (d, J=10.0 Hz, 1 H), 4.03-4.07 (m, 1 H), 3.83 (d, J=13.6 Hz, 1 H), 3.04-3.06 (m, 1 H), 2.85-2.90 (m, 1 H), 2.70-2.75 (m, 1 H), 1.87-1.89 (m, 1 H), 1.69-1.71 (m, 1 H), 1.38-1.43 (m, 2 H). Chemical Formula: C₁₅H₁₈F₃N₃O₄S. Molecular Weight: 393.38. Melting point: 47.2-57.8° C. Optical rotation: [a]²⁵D = 17.00 (c = 0.10, CH₃OH).

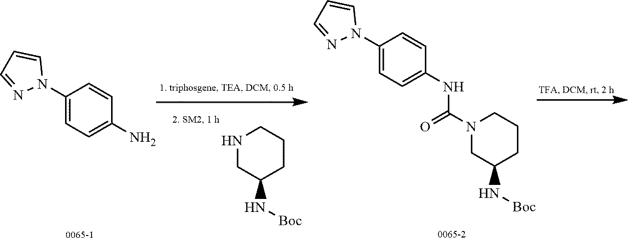

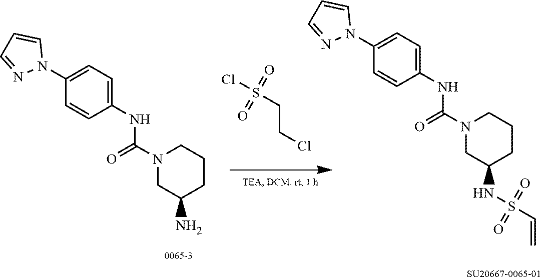

The synthesis of (R)-tert-butyl 1-(4-(1H-pyrazol-1-yl)phenylcarbamoyl)piperidin-3-ylcarbamate (0065-2).

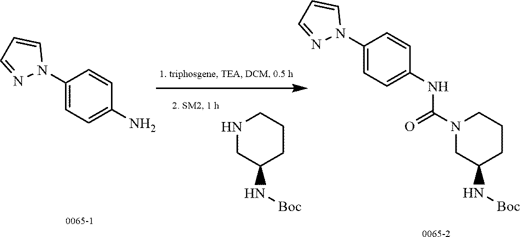

To a solution of 0065-1 (250 mg, 1.57 mmol) and bis(trichloromethyl) carbonate (163 mg, 0.55 mmol) in DCM (5 ml) was added TEA (380 mg, 3.77 mmol) was stirred at rt for 30 min. Added (R)-tert-butyl piperidin-3-ylcarbamate (314 mg, 1.57 mmol). The mixture was stirred at rt for 1 h and purified by C.C. (PE/EA= 4:1) to get 0065-2 (300 mg, 50%) as a yellow solid.

The synthesis of (R)-N-(4-(1H-pyrazol-1-yl)phenyl)-3-aminopiperidine-1-carboxamide (0065-3).

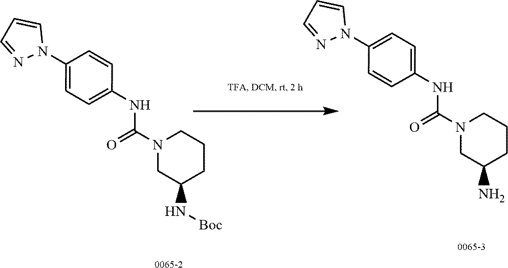

To a solution of 0065-2 (300 mg, 0.78 mmol) in TFA/DCM (5 ml/5 ml) was stirred at room temperature for 2 h, The solvent was removed in vacuo to give 0065-3 (155 mg, 70%) as yellow oil.

The synthesis of (R)-N-(4-(1H-pyrazol-1-yl)phenyl)-3-(vinylsulfonamido)piperidine-1-carboxamide (SU20667-0065-01)

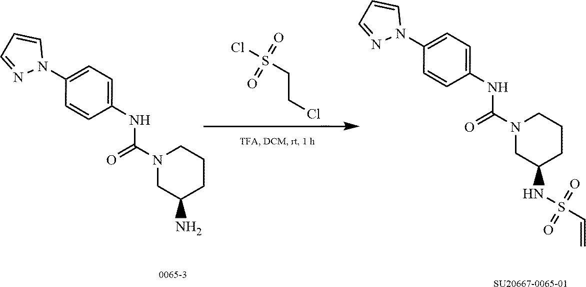

To a solution of 0065-3 (155 mg, 0.54) and TEA (137 mg, 1.35 mmol) in DCM (3 ml) was added dropwise 2-chloroethanesulfonyl chloride (80 mg, 0.49 mmol), the mixture was stirred at room temperature for 1 h and the solvent was removed in vacuo, the resulting residue was purified by prep-HPLC to give SU20667-0065-01 (78.93 mg, 39%) as a white solid. LC-MS (Agilent LCMS 1200-6120, Column: Waters X-Bridge C18 (50 mm*4.6 mm*3.5 µm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min), Purity: 99.52%, Rₜ =1.674 min; MS Calcd: 375.14; MS Found: 376.3 [M+H]⁺. HPLC (Agilent HPLC 1200, Column: L-column2 ODS (150 mm*4.6 mm*5.0 µm); Column Temperature: 40° C.; Flow Rate: 1.5 mL/min; Mobile Phase: from 90% [(total 10 mM AcONH4) water/CH₃CN=900/100 (v/v)] and 10% [total 10 mM AcONH₄) water/CH₃CN=100/900 (v/v)] to 15% [total 10 mM AcONH₄) water/CH₃CN=900/100 (v/v)] and 85% [total 10 mM AcONH4) water/CH₃CN=100/900 (v/v)] in 5 min, then under this condition for 10 min, finally changed to 90% [(total 10 mM AcONH4) water/CH₃CN=900/100 (v/v)] and 10% [total 10 mM AcONH4) water/CH₃CN=100/900 (v/v)] in 0.1 min and under this condition for 5 min), Purity: 99.72%, Rt =7.224 min. ¹H NMR (400 MHz, DMSO-d₆) δ 8.64 (s, 1 H), 8.38 (d, J=2.4 Hz, 1 H), 7.66-7.69 (m, 3 H), 7.50-7.58 (m, 3 H), 6.78 (dd, J=16.8 Hz, 10.0 Hz, 1 H), 6.49-6.50 (m, 1 H), 6.05 (d, J=16.8 Hz, 1 H), 5.96 (d, J=10.0 Hz, 1 H), 4.05-4.09 (m, 1 H), 3.85 (d, J=12.8 Hz, 1 H), 3.05 (br, 1 H), 2.86-2.88 (m, 1 H), 2.70-2.87 (m, 1 H), 1.89-1.91 (m, 1 H), 1.69-1.70 (m, 1 H), 1.39-1.44 (m, 2 H). Chemical Formula: C₁₇H₂₁N₅O₃S. Molecular Weight: 375.45. Melting point: 52.7-59.0° C. Optical rotation: [a]²⁵D = 13.00 (c = 0.10, CH₃OH).

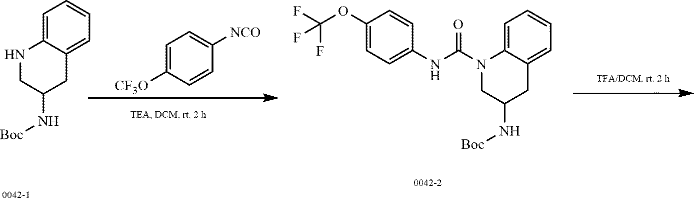

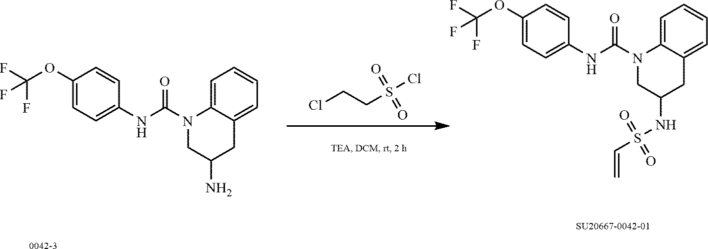

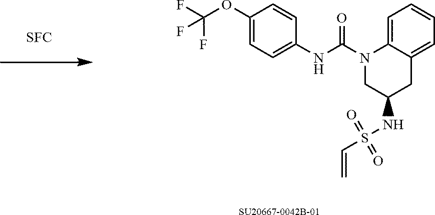

The synthesis of tert-butyl 1-(4-(trifluoromethoxy)phenylcarbamoyl)-1,2,3,4-tetrahydroquinolin-3-ylcarbamate (0042-2)

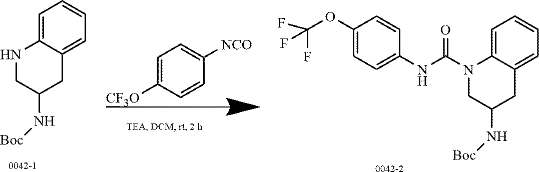

To a solution of 0042-1 (620 mg, 2.5 mmol) in DCM (30 mL) was added TEA (757 mg, 7.5 mmol) and 4-(Trifluoromethoxy)phenyl isocyanate (659 mg, 3.25 mmol). The mixture was stirred at rt for 2 h. The mixture was pour into ice water and extracted with DCM (30 mL × 3). The organic layer was dried over Na₂SO₄, filtered and concentrated. The residue was purified by column chromatography (PE/EA = 4:1) to give 0042-2 (800 mg, 70.9%) as yellow oil.

The synthesis of 3-amino-N-(4-(trifluoromethoxy)phenyl)-3,4-dihydroquinoline-1(2H)-carboxamide (0042-3)

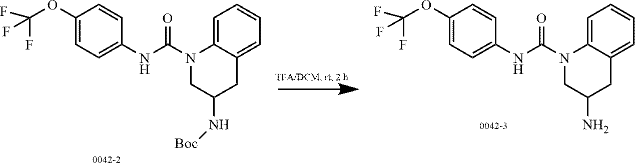

To a solution of 0042-2 (800 mg, 1.77 mmol) in DCM (20 mL) was added TFA (2 mL). It was stirred at rt for 2 h. The mixture was adjusted to pH 9 with Na₂CO₃ (aq.) and extracted with DCM (20 mL × 3). The organic layer was dried over Na₂SO₄, filtered and concentrated to give 0042-3 (500 mg, 80.5%) as yellow oil.

The synthesis of N-(4-(trifluoromethoxy)phenyl)-3-(vinylsulfonamido)-3,4-dihydroquinoline-1(2H)-carboxamide (SU20667-0042-01)

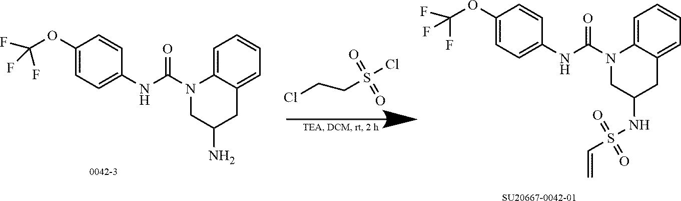

To a solution of 0042-3 (150 mg, 0.43 mmol) in DCM (10 mL) was added TEA (123 mg, 1.29 mmol) and 2-chloroethanesulfonyl chloride (84 mg, 0.51 mmol). The mixture was stirred at rt for 2 h. The mixture was pour into ice water and extracted with DCM (20 mL × 3). The organic layer was dried over Na₂SO₄, filtered and concentrated. The residue was purified by prep-HPLC to give SU20667-0042-01 (67.96 mg, 35.8%) as a white solid. Agilent LCMS 1200-6120, Column: Waters X-Bridge C18 (50 mm*4.6 mm*3.5 µm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min, Purity 100.00%, Rt = 1.882 min; MS Calcd.: 441.1; MS Found: 442.2[M+H]⁺. Agilent LCMS 1200-6120, Column: Waters X-Bridge C18 (50 mm*4.6 mm*3.5 µm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min, Purity 97.08%, Rt = 9.552 min; MS Calcd.: 441.1; MS Found: 442.2 [M+H]⁺. 1HNMR (400 MHz, DMSO-d₆) δ 9.17 (s, 1 H), 7.64 (br, 1 H), 7.52-7.56 (m, 2 H), 7.27-7.31 (m, 3 H), 7.12-7.18 (m, 2 H), 6.99-7.03 (m, 1 H), 6.81 (dd, J = 16.8 Hz, 10.0 Hz, 1 H), 6.06 (d, J = 16.8 Hz, 1 H), 5.97 (d, J = 9.6 Hz, 1 H), 4.00-4.04 (m, 1 H), 3.50-3.57 (m, 1 H), 3.35-3.41 (m, 1 H), 3.05-3.11 (m, 1 H), 2.66-2.77 (m, 1 H). Melting point: 50.4-52.7° C.

The SU20667-0042-01 (300 mg) was further purified by chiral-HPLC to give SU20667-0042A-01 (75.37 mg, 25.1%) as a white solid and SU20667-0042B-01 (76.18 mg, 25.4%) as a white solid.

SU20667-0042A-01: Agilent LCMS 1200-6120, Column: Waters X-Bridge C18 (50 mm*4.6 mm*3.5 µm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min, Purity 98.47 %, Rt = 2.408 min; MS Calcd.: 441.1; MS Found: 442.2[M+H]⁺. Agilent LCMS 1200-6120, Column: Waters X-Bridge C18 (50 mm*4.6 mm*3.5 µm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min, Purity 95.24%, Rt = 10.013 min; MS Calcd.: 441.1; MS Found: 442.2 [M+H]⁺. 1 H NMR (400 MHz, DMSO-d6) δ 9.16 (s, 1 H), 7.67 (s, 1 H), 7.52-7.56 (m, 2 H), 7.27-7.31 (m, 3 H), 7.13-7.18 (m, 2 H), 6.99-7.03 (m, 1 H), 6.81 (dd, J= 16.8 Hz, 10.0 Hz, 1 H), 6.06 (d, J = 16.8 Hz, 1 H), 5.97 (d, J = 10.0 Hz, 1 H), 3.99-4.04 (m, 1 H), 3.49-3.50 (m, 1 H), 3.36-3.41 (m, 1 H), 3.05-3.11 (m, 1 H), 2.66-2.77 (m, 1 H). Melting point: 147.7-148.5° C. Optical rotation: [a]²⁵D = -22.00 (c = 0.10, CH₃CN). ee: 100%.

SU20667-0042B-01: Agilent LCMS 1200-6120, Column: Waters X-Bridge C18 (50 mm*4.6 mm*3.5 µm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min, Purity 97.81 %, Rt = 2.430 min; MS Calcd.: 441.1; MS Found: 442.0[M+H]⁺. Agilent LCMS 1200-6120, Column: Waters X-Bridge C18 (50 mm*4.6 mm*3.5 µm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min, Purity 97.55%, Rt = 10.016 min; MS Calcd.: 441.1; MS Found: 442.0 [M+H]⁺. 1 H NMR (400 MHz, DMSO-d6) δ 9.16 (s, 1 H), 7.67 (s, 1 H), 7.52-7.56 (m, 2 H), 7.27-7.31 (m, 3 H), 7.12-7.18 (m, 2 H), 6.99-7.03 (m, 1 H), 6.81 (dd, J= 16.4 Hz, 9.6 Hz, 1 H), 6.06 (d, J= 16.4 Hz, 1 H), 5.97 (d, J= 10.0 Hz, 1 H), 3.99-4.04 (m, 1 H), 3.52-3.53 (m, 1 H), 3.36-3.41 (m, 1 H), 3.05-3.11 (m, 1 H), 2.66-2.77 (m, 1 H). Chemical Formula: C₁₉H₁₈F₃N₃O₄S. Molecular Weight: 441.42 Melting point: 146.2-147.9° C. Optical rotation: [a]²⁵D = 14.0 (c = 0.10, CH₃CN). ee: 100%.

Scheme 4: Route for SU20667-0067-01

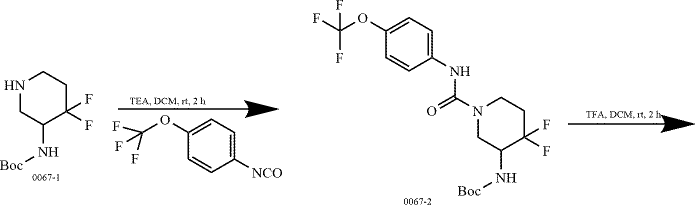

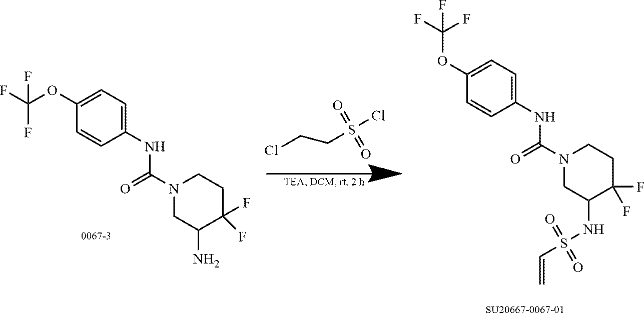

The synthesis of (S)-tert-butyl 4,4-difluoro-1-(4-(trifluoromethoxy)phenylcarbamoyl)piperidin-3-ylcarbamate (0067-2).

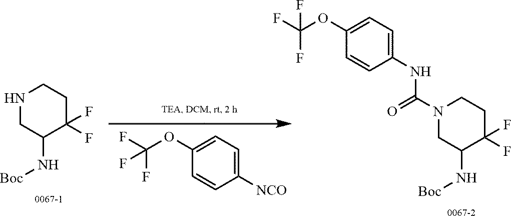

To a solution of 0067-1 (118 mg, 0.5 mmol) in DCM (10 mL) was added TEA (151 mg, 1.5 mmol) and 4-(Trifluoromethoxy)phenyl isocyanate (114 mg, 0.56 mmol). The mixture was stirred at rt for 2 h. The mixture was pour into ice water and extracted with DCM (20 mL × 3). The organic layer was dried over Na₂SO₄, filtered and concentrated. The residue was purified by column chromatography (PE/EA= 4: 1) to give 0067-2 (163 mg, 74.4%) as yellow oil.

The synthesis of (S)-3-amino-4,4-difluoro-N-(4-(trifluoromethoxy)phenyl)piperidine-1-carboxamide (0067-3)

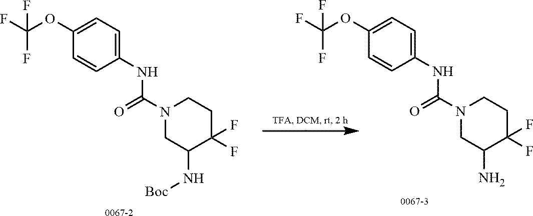

To a solution of 0067-2 (163 mg, 0.37 mmol) in DCM (10 mL) was added TFA (1 mL). It was stirred at rt for 2 h. The mixture was adjusted to pH 9 with Na₂CO₃ (aq.) and extracted with DCM (20 mL × 3). The organic layer was dried over Na₂SO₄, filtered and concentrated to give 0067-3 (123 mg, 98.1%) as yellow oil.

The synthesis of (S)-4,4-difluoro-N-(4-(trifluoromethoxy)phenyl)-3-(vinyl-sulfonamido)piperidine-1-carboxamide (SU20667-0067-01)

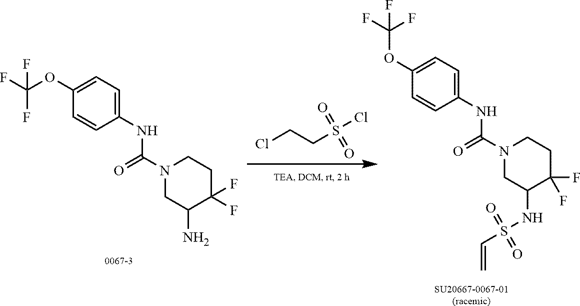

To a solution of 0067-3 (123 mg, 0.36 mmol) in DCM (10 mL) was added TEA (109 mg, 1.08 mmol) and 2-chloroethanesulfonyl chloride (60 mg, 0.37 mmol). The mixture was stirred at rt. for 2 h. The mixture was pour into ice water and extracted with DCM (10 mL × 3). The organic layer was dried over Na₂SO₄, filtered and concentrated. The residue was purified by prep-HPLC to give SU20667-0067-01 (106.67 mg, 69.1%) as a white solid. Agilent LCMS 1200-6120, Column: Waters X-Bridge C18 (50 mm*4.6 mm*3 \. 5 µm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min, Purity 97.63 %, Rt = 1.966 min; MS Calcd.: 429.0; MS Found: 430.0[M+H]⁺. Agilent LCMS 1200-6120, Column: Waters X-Bridge C18 (50 mm*4.6 mm*3.5 µm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min, Purity 98.44%, Rt = 9.282 min; MS Calcd.: 429.0; MS Found: 430.0 [M+H]⁺. 1 H NMR (400 MHz, DMSO-d₆) δ 8.92 (s, 1 H), 7.98 (br, 1 H), 7.54-7.58 (m, 2 H), 7.25 (d, J= 8.4 Hz, 2 H), 6.75 (dd, J = 16.8 Hz, 10.0 Hz, 1 H), 6.06 (d, J = 16.4 Hz, 1 H), 5.96 (d, J = 10.0 Hz, 1 H), 3.92-4.04 (m, 2 H), 3.52-3.60 (m, 1 H), 3.03-3.18 (m, 2 H), 2.01-2.19 (m, 2 H). Chemical Formula: C₁₅H₁₆F₅N₃O₄S. Molecular Weight: 429.36. Melting point: 56.9-69.1° C.

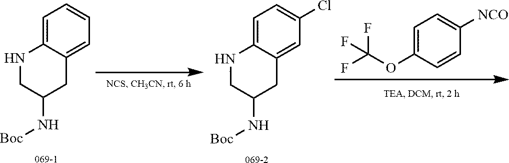

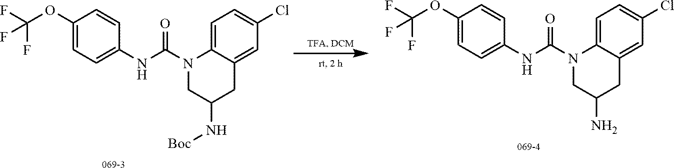

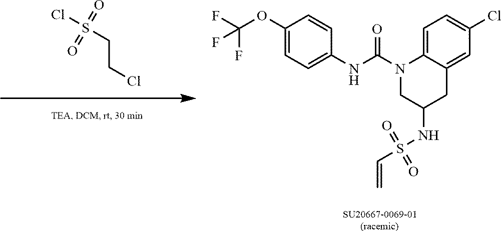

The synthesis of tert-butyl 6-chloro-1,2,3,4-tetrahydroquinolin-3-ylcarbamate (0069-2)

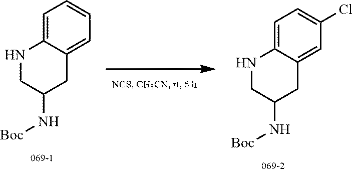

To a solution of 0069-1 (500 mg, 2.02 mmol) in CH₃CN (10 ml) was added NBS (360 mg, 2.02 mmol) was stirred at rt for 6 h. Removing the solvent and the resulting residue was purified by CC (PE/EA= 4:1) to get 0069-2 (390 mg, 69%) as a yellow solid.

The synthesis of tert-butyl 6-chloro-1-(4-(trifluoromethoxy)phenylcarbamoyl)-1,2,3,4-tetrahydroquinolin-3-ylcarbamate (0069-3)

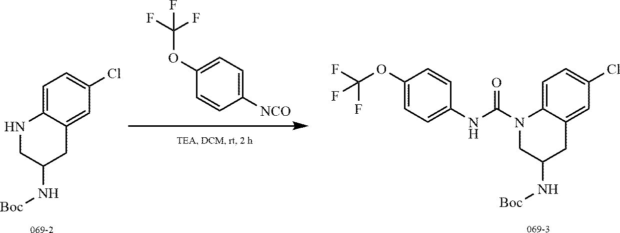

To a solution of 0069-2 (390 mg, 1.38) and TEA (348 mg, 3.45 mmol) in DCM (6 ml) was added 4-(Trifluoromethoxy)phenyl isocyanate (280 mg, 1.38 mmol) was stirred at rt for 2 h. Removing the solvent and the resulting residue was purified by CC (PE/EA= 5:1) to get 0069-3 (400 mg, 60%) as yellow oil.

The synthesis of 3-amino-6-chloro-N-(4-(trifluoromethoxy)phenyl)-3,4-dihydroquinoline-1(2H)-carboxamide (0069-4)

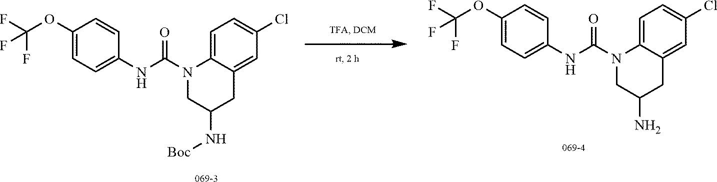

To a solution of 0069-3 (400 mg, 0.82 mmol) in TFA/DCM (5 ml/5 ml) was stirred at room temperature for 2 h, The solvent was removed in vacuo to give 0069-4 (390 mg, crude) as colorless oil.

The synthesis of 6-chloro-N-(4-(trifluoromethoxy)phenyl)-3-(vinylsulfonamido)-3,4-dihydroquinoline-1(2H)-carboxamide (SU20667-0069-01).

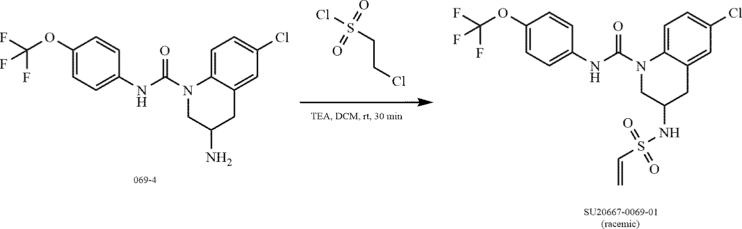

To a solution of 0069-4 (390 mg, crude) and TEA (323 mg, 3.2 mmol) in DCM (5 ml) was added dropwise 2-chloroethanesulfonyl chloride (209 mg, 1.28 mmol), the mixture was stirred at room temperature for 30 min and the solvent was removed in vacuo and the resulting residue was purified by prep-HPLC to give SU20667-0069-01 (152.87 mg, 25%) as a white solid. LC-MS (Agilent LCMS 1200-6120, Column: Waters X-Bridge C18 (50 mm*4.6 mm*3.5 µm); Column Temperature: 40° C.; Flow Rate: 2.0 mL/min; Mobile Phase: from 90% [(total 10 mM AcONH₄) water/CH₃CN=900/100 (v/v)] and 10% [(total 10 mM AcONH₄) water/CH₃CN=100/900 (v/v)] to 10% [(total 10 mM AcONH₄) water/CH₃CN=900/100 (v/v)] and 90% [(total 10 mM AcONH₄) water/CH₃CN=100/900 (v/v)] in 1.6 min, then under this condition for 2.4 min, finally changed to 90% [(total 10 mM AcONH₄) water/CH₃CN=900/100 (v/v)] and 10% [(total 10 mM AcONH₄) water/CH₃CN=100/900 (v/v)] in 0.1 min and under this condition for 0.7 min), Purity: 97.93%, Rₜ=2.199 min; MS Calcd: 475.06; MS Found: 476.0 [M+H]⁺. HPLC (Agilent HPLC 1200, Column: L-column2 ODS (150 mm*4.6 mm*5.0 µm); Column Temperature: 40° C.; Flow Rate: 1.5 mL/min; Mobile Phase: from 90% [(total 10 mM AcONH₄) water/CH₃CN=900/100 (v/v)] and 10% [total 10 mM AcONH₄) water/CH₃CN=100/900 (v/v)] to 15% [total 10 mM AcONH₄) water/CH₃CN=900/100 (v/v)] and 85% [total 10 mM AcONH₄) water/CH₃CN=100/900 (v/v)] in 5 min, then under this condition for 10 min, finally changed to 90% [(total 10 mM AcONH₄) water/CH₃CN=900/100 (v/v)] and 10% [total 10 mM AcONH₄) water/CH₃CN=100/900 (v/v)] in 0.1 min and under this condition for 5 min), Purity:98.44%, Rt =10.532 min. ¹H NMR (400 MHz, DMSO-d₆) δ 9.20 (s, 1 H), 7.67 (s, 1 H), 7.52-7.55 (m, 2 H), 7.26-7.35 (m, 4 H), 7.17-7.20 (m, 1 H), 6.80 (dd, J=16.8 Hz, 10.0 Hz, 1 H), 6.06 (d, J=16.8 Hz, 1 H), 5.97 (d, J=10.0 Hz, 1 H), 3.96-4.00 (m, 1 H), 3.54-3.55 (m, 1 H), 3.40-3.45 (m, 1 H), 3.05-3.11 (m, 1 H), 2.70-2.75 (m, 1 H). Chemical Formula: C₁₉H₁₇ClF₃N₃O₄S. Molecular Weight: 475.87. Melting point: 156.5-157.4° C.

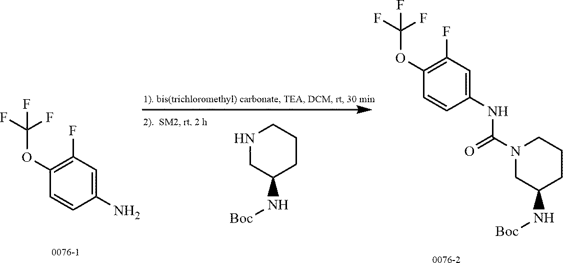

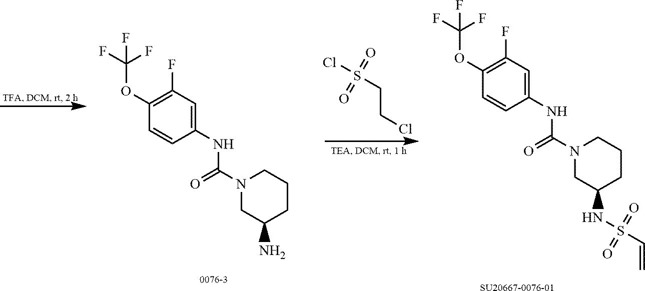

The synthesis of (R)-tert-butyl 1-(3-fluoro-4-(trifluoromethoxy)phenylcarbamoyl)piperidin-3-ylcarbamate (0076-2).

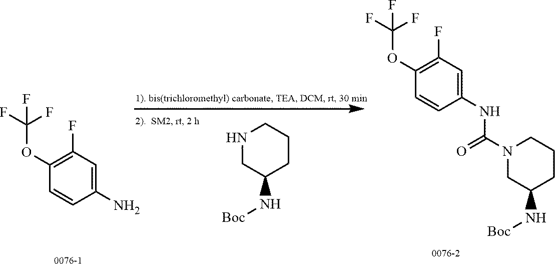

To a solution of 0076-1 (100 mg, 0.51 mmol) and bis(trichloromethyl) carbonate (53 mg, 0.35 mmol) in DCM (3 ml) was added TEA (122 mg, 1.12 mmol) was stirred at rt for 30 min. Added (R)-tert-butyl piperidin-3-ylcarbamate (103 mg, 0.51 mmol). The mixture was stirred at rt for 2 h and purified by CC to get 0076-2 (350 mg, crude) as a white solid.

The synthesis of (R)-3-amino-N-(3-fluoro-4-(trifluoromethoxy)phenyl)piperidine-1-carboxamide (0076-3)

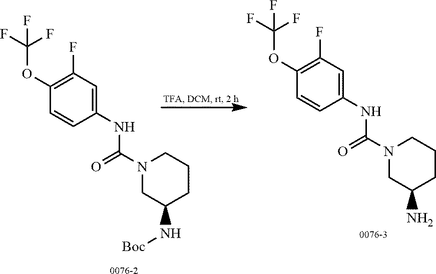

To a solution of 0076-2 (350 mg, crude) in TFA/DCM (5 ml/5 ml) was stirred at room temperature for 2 h, The solvent was removed in vacuo to give 0076-3 (160 mg, 60%) as yellow oil.

The synthesis of (R)-N-(3-fluoro-4-(trifluoromethoxy)phenyl)-3-(vinylsulfonamido)piperidine-1-carboxamide (SU20667-0076-01)

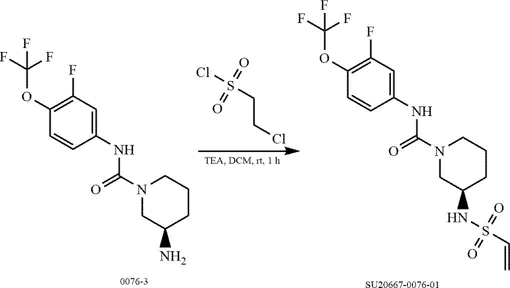

To a solution of 0076-3 (160 mg, 0.5 mmol) and TEA (126 mg, 1.25 mmol) in DCM (3 ml) was added dropwise 2-chloroethanesulfonyl chloride (82 mg, 0.5 mmol), the mixture was stirred at room temperature for 1 h and the solvent was removed in vacuo and the resulting residue was purified by prep-HPLC to get SU20667-0076-01 (34.39 mg, 17%) as a white solid. LC-MS (Agilent LCMS 1200-6120, Column: Waters X-Bridge C18 (50 mm*4.6 mm*3.5 µm); Column Temperature: 40° C.; Flow Rate: 2.0 mL/min; Mobile Phase: from 90% [(total 10 mM AcONH₄) water/CH₃CN=900/100 (v/v)] and 10% [(total 10 mM AcONH₄) water/CH₃CN=100/900 (v/v)] to 10% [(total 10 mM AcONH₄) water/CH₃CN=900/100 (v/v)] and 90% [(total 10 mM AcONH₄) water/CH₃CN=100/900 (v/v)] in 1.6 min, then under this condition for 2.4 min, finally changed to 90% [(total 10 mM AcONH₄) water/CH₃CN=900/100 (v/v)] and 10% [(total 10 mM AcONH₄) water/CH₃CN=100/900 (v/v)] in 0.1 min and under this condition for 0.7 min), Purity: 99.14%, Rₜ=1.977 min; MS Calcd: 411.09; MS Found: 412.2 [M+H]⁺. HPLC (Agilent HPLC 1200, Column: L-column2 ODS (150 mm*4.6 mm*5.0 µm); Column Temperature: 40° C.; Flow Rate: 1.5 mL/min; Mobile Phase: from 90% [(total 10 mM AcONH₄) water/CH₃CN=900/100 (v/v)] and 10% [total 10 mM AcONH₄) water/CH₃CN=100/900 (v/v)] to 15% [total 10 mM AcONH₄) water/CH₃CN=900/100 (v/v)] and 85% [total 10 mM AcONH₄) water/CH₃CN=100/900 (v/v)] in 5 min, then under this condition for 10 min, finally changed to 90% [(total 10 mM AcONH₄) water/CH₃CN=900/100 (v/v)] and 10% [total 10 mM AcONH₄) water/CH₃CN=100/900 (v/v)] in 0.1 min and under this condition for 5 min), Purity:99.18%, Rt =9.313 min. ¹H NMR (400 MHz, DMSO-d₆) δ 8.89 (s, 1 H), 7.66 (dd, J=13.6 Hz, 2.8 Hz, 1 H), 7.50-7.52 (m, 1 H), 7.39-7.43 (m, 1 H), 7.30-7.32 (m, 1 H), 6.77 (dd, J=16.8 Hz, 10.0 Hz, 1 H), 6.04 (d, J=16.4 Hz, 1 H), 5.95 (d, J=10.0 Hz, 1 H), 4.03 (dd, J=12,4 Hz, 4.0 Hz, 1 H), 3.81 (d, J=12.8 Hz, 1 H), 3.04-3.05 (m, 1 H), 2.70-2.90 (m, 1 H), 2.73-2.90 (m, 1 H), 1.88-1.89 (m, 1 H), 1.60-1.70 (m, 1 H), 1.42 (t, J=9.2 Hz, 2 H). Chemical Formula: C₁₅H₁₇F₄N₃O₄S. Molecular Weight: 411.37. Melting point: 41.1-49.2° C. Optical rotation: [a]²⁵D = 5.00 (c = 0.10, CH₃CN)

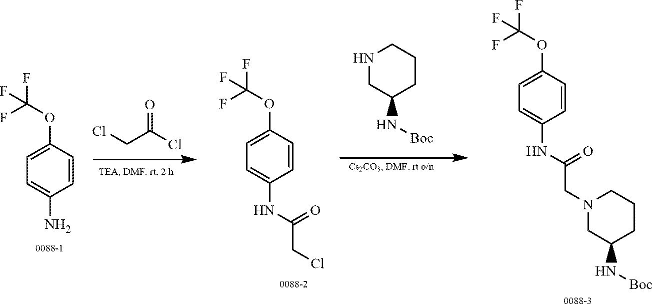

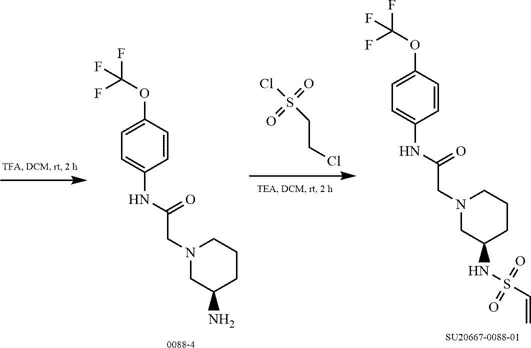

The synthesis of 2-chloro-N-(4-(trifluoromethoxy)phenyl)acetamide (0088-2)

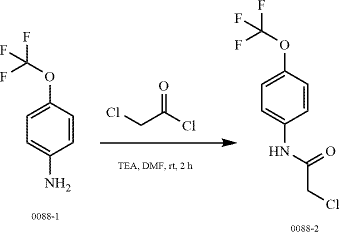

To a mixture of 0088-1 (200 mg, 1.13 mmol) in DCM (10 mL) was added 2-chloroacetyl chloride (152 mg, 1.36 mmol). It was stirred at rt for 2 h. The reaction mixture was concentrated to give 0088-2 (220 mg crude) as yellow oil.

The synthesis of (R)-tert-butyl 1-(2-oxo-2-(4-(trifluoromethoxy)phenylamino)ethyl)piperidin-3-ylcarbamate (0088-3)

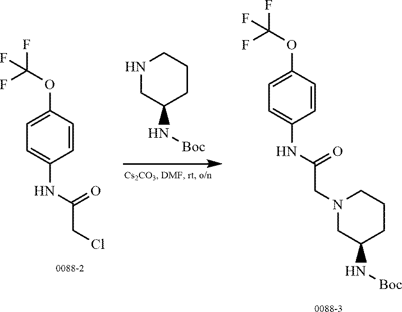

To a solution of 0088-2 (220 mg, 0.87 mmol) in DMF (5 mL) was added (R)-tert-butyl piperidin-3-ylcarbamate (226 mg, 1.13 mmol) and Cs₂CO₃ (850 mg, 2.61 mmol). It was stirred at rt overnight. Water (40 mL) was added and extracted with EA (20 mL × 3). The organic layer was dried over Na₂SO₄, filtered and concentrated. The residue was purified by column chromatography (PE/EA= 4:1) to give 0088-3 (333 mg, 91.9%) as a white solid.

The synthesis of (R)-2-(3-aminopiperidin-1-yl)-N-(4-(trifluoromethoxy)phenyl)acetamide (0088-4)

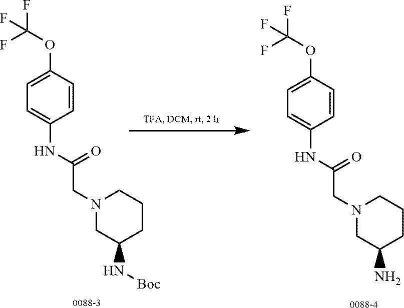

To a solution of 0088-3 (333 mg, 0.8 mmol) in DCM (10 mL) was added TFA (1 mL). It was stirred at rt for 2 h. The mixture was adjusted to pH 9 with Na₂CO₃ (aq.) and extracted with DCM (20 mL × 3). The organic layer was dried over Na₂SO₄, filtered and concentrated to give 0088-4 (150 mg, 59.1%) as yellow oil.

The synthesis of (R)-N-(4-(trifluoromethoxy)phenyl)-2-(3-(vinylsulfonamido)piperidin-1-yl)acetamide (SU20667-0088-01)

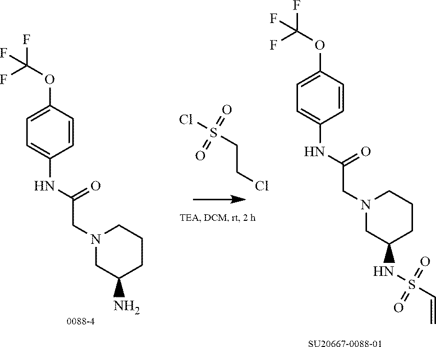

To a solution of 0088-4 (150 mg, 0.47 mmol) in DCM (10 mL) was added TEA (142 mg, 1.41 mmol) and 2-chloroethanesulfonyl chloride (99 mg, 0.61 mmol). The mixture was stirred at rt for 2 h. The mixture was pour into ice water and extracted with DCM (20 mL × 3). The organic layer was dried over Na₂SO₄, filtered and concentrated. The residue was purified by prep-HPLC to give SU20667-0088-01 (48.22 mg, 25.2%) as a white solid. Agilent LCMS 1200-6120, Column: Waters X-Bridge C18 (50 mm*4.6 mm*3.5 µm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min, Purity 98.51 %, Rt = 2.034 min; MS Calcd.: 407.1; MS Found: 407.8 [M+H]⁺. Agilent LCMS 1200-6120, Column: Waters X-Bridge C18 (50 mm*4.6 mm*3.5 µm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min, Purity 97.62%, Rt = 9.509 min; MS Calcd.: 407.1; MS Found: 408.2 [M+H]⁺. 1 H NMR (400 MHz, DMSO-d6) δ 9.86 (s, 1 H), 7.72-7.76 (m, 2 H), 7.50 (d, J = 4.4 Hz, 1 H), 7.33 (d, J = 8.4 Hz, 2 H), 6.03 (dd, J = 16.4 Hz, 9.6 Hz, 1 H), 6.02 (d, J= 16.4 Hz, 1 H), 5.87 (d, J= 10.0 Hz, 1 H), 3.06-3.33 (m, 4 H), 2.72-2.75 (m, 1 H), 2.19-2.26 (m, 2 H), 1.68-1.71 (m, 2 H), 1.50-1.53 (m, 1 H), 1.28-1.30 (m, 1 H). Chemical Formula: C₁₆H₂₀F₃N₃O₄S. Molecular Weight: 407.41. Melting point: 112.8-113.5° C. Optical rotation: [a]²⁵D = 19.50 (c = 0.20, CH₃OH).

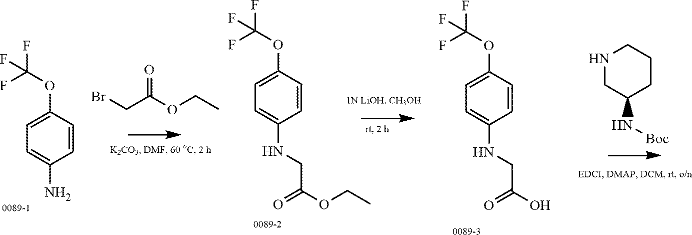

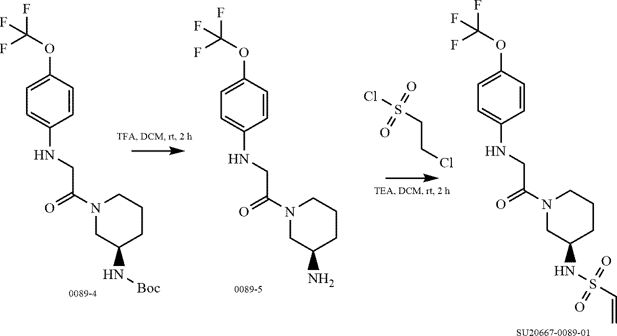

The synthesis of ethyl 2-(4-(trifluoromethoxy)phenylamino)acetate (0089-2)

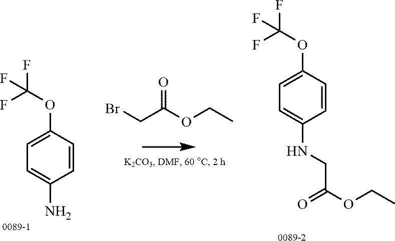

To a solution of 0089-1 (500 mg, 2.82 mmol) in DMF (30 mL) was added K₂CO₃ (1.17 g, 8.46 mmol) and ethyl 2-bromoacetate (562 mg, 3.38 mmol) and stirred at 60° C. for 2 h. The mixture was pour into ice water and extracted with DCM (30 mL × 3). The organic layer was dried over Na₂SO₄, filtered and concentrated. The residue was purified by column chromatography (PE/EA= 3: 1) to give 0089-2 (550 mg, 74.1%) as a white solid.

The synthesis of ethyl 2-(4-(trifluoromethoxy)phenylamino)acetic acid (0089-3)

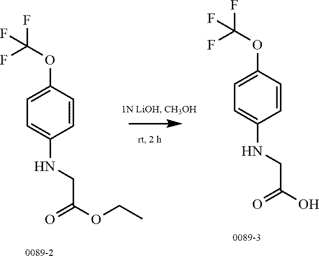

To a solution of 0089-2 (550 mg, 1.6 mmol) in CH₃OH (20 mL) was added 1 N LiOH (20 mL). It was stirred at rt for 2 h. The mixture was adjusted to pH 5 with 2 N HCl (in water) and extracted with DCM (20 mL × 3). The organic layer was dried over Na₂SO₄, filtered and concentrated to give 0089-3 (250 mg, 66.2%) as yellow oil.

The synthesis of ethyl (R)-tert-butyl 1-(2-(4-(trifluoromethoxy)phenylamino)acetyl)piperidin-3-ylcarbamate (0089-4)

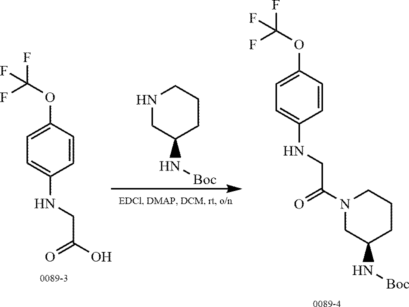

To a solution of 0089-3 (249 mg, 1.06 mmol) in DMF (10 mL) was EDCI (265 mg, 1.38 mmol), DMAP (194 mg, 1.59 mmol) and (R)-tert-butyl piperidin-3-ylcarbamate (276 mg, 1.38 mmol) and stirred at rt overnight. The mixture was pour into ice water and extracted with DCM (30 mL × 3). The organic layer was dried over Na₂SO₄, filtered and concentrated. The residue was purified by column chromatography (PE:EA= 2:1) to give 0089-4 (300 mg, 67.8%) as a white solid.

The synthesis of (R)-1-(3-aminopiperidin-1-yl)-2-(4-(trifluoromethoxy)phenyl-amino)ethanone (0089-5)

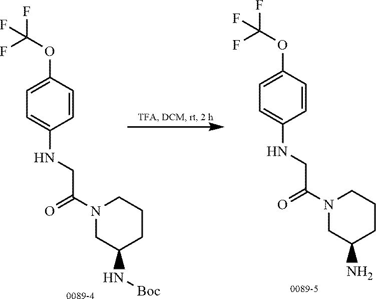

To a solution of 0089-4 (300 mg, 0.72 mmol) in DCM (10 mL) was added TFA (1 mL). It was stirred at rt for 2 h. The mixture was adjusted to pH 9 with Na₂CO₃ (aq.) and extracted with DCM (20 mL × 3). The organic layer was dried over Na₂SO₄, filtered and concentrated to give 0089-5 (200 mg, 87.6%) as yellow oil.

The synthesis of (R)-N-(1-(2-(4-(trifluoromethoxy)phenylamino)acetyl)piperidin-3-yl)ethenesulfonamide (SU20667-0089-01)

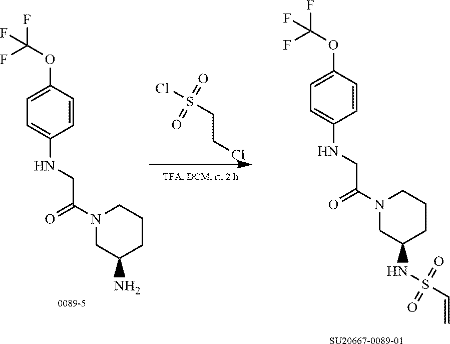

To a solution of 0089-5 (200 mg, 0.63 mmol) in DCM (10 mL) was added TEA (191 mg, 1.89 mmol) and 2-chloroethanesulfonyl chloride (133 mg, 0.82 mmol). The mixture was stirred at rt for 2 h. The mixture was pour into ice water and extracted with DCM (20 mL × 3). The organic layer was dried over Na₂SO₄, filtered and concentrated. The residue was purified by prep-HPLC to give SU20667-0089-01 (32.19 mg, 12.6%) as a white solid. Agilent LCMS 1200-6120, Column: Waters X-Bridge C18 (50 mm*4.6 mm*3.5 µm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min, Purity 97.95 %, Rt = 2.001 min; MS Calcd.: 407.1; MS Found: 408.1 [M+H]⁺. Agilent LCMS 1200-6120, Column: Waters X-Bridge C18 (50 mm*4.6 mm*3.5 µm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min, Purity 95.41%, Rt = 9.329 min; MS Calcd.: 407.1; MS Found: 408.2 [M+H]⁺. 1HNMR (400 MHz, DMSO-d₆) δ 7.52-7.58 (m, 1 H), 7.04-7.06 (m, 2 H), 6.64-6.68 (m, 3 H), 5.84-6.09 (m, 3 H), 3.94-4.17 (m, 1 H), 3.62-3.88 (m, 3 H), 2.73-3.20 (m, 3 H), 1.32-1.87 (m, 4 H). Chemical Formula: C₁₆H₂₀F₃N₃O₄S. Molecular Weight: 407.41. Melting point: 151.6-153.9° C. Optical rotation: [a]²⁵D = 15.00 (c = 0.20, CH₃OH)

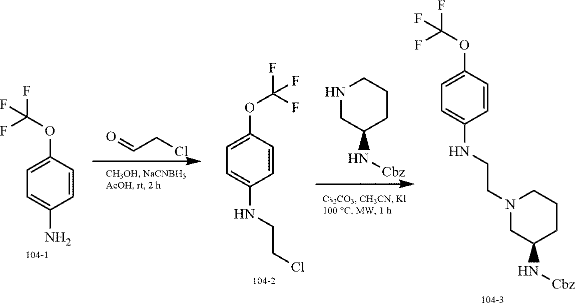

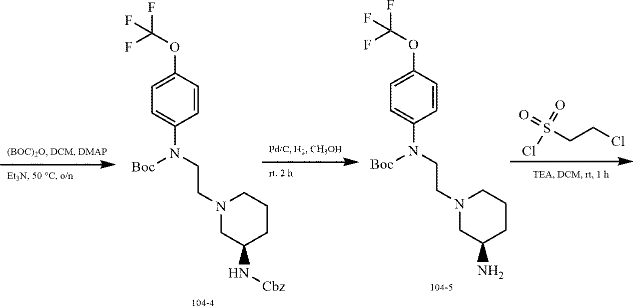

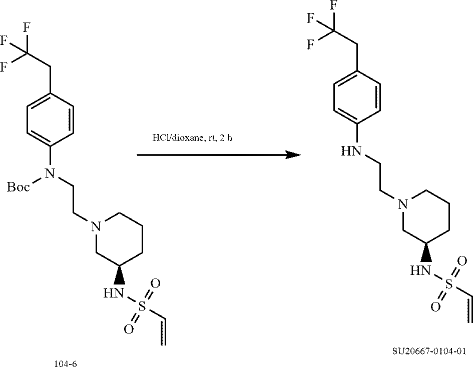

The synthesis of N-(2-chloroethyl)-4-(trifluoromethoxy)aniline (104-2)

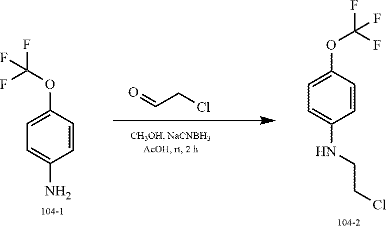

To a mixture of 104-1 (1 g, 5.65 mmol) in CH₃OH (20 mL) was added 2-chloroacetaldehyde (529 mg, 6.78 mmol) and AcOH (407 mg, 6.78 mmol). It was stirred at rt for 1 h. Then NaCNBH3 (712 mg, 11.3 mmol) was added and stirred at rt for 1 h. Water (20 mL) was added and extracted with EA (20 mL × 3). The organic layer was dried over Na₂SO₄, filtered and concentrated. The residue was purified by column chromatography (PE/EA= 2:1) to give 104-2 (1.1 g, 81.4%) as yellow oil.

The synthesis of (R)-benzyl 1-(2-(4-(trifluoromethoxy)phenylamino)ethyl)piperidin-3-ylcarbamate (104-3)

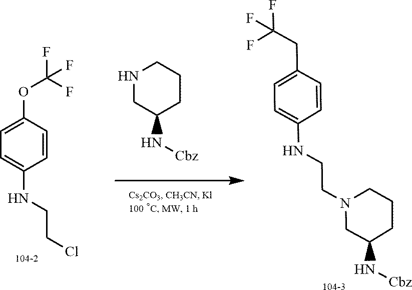

To a solution of 104-2 (1.1 g, 4.6 mmol) in CH₃CN (50 mL) was added (R)-tert-butyl piperidin-3-ylcarbamate (1.4 g, 5.98 mmol), KI (992 mg, 5.98 mmol) and Cs₂CO₃ (4.5 g, 13.8 mmol). It was subjected to MW condition at 100° C. for 1 h. Water (40 mL) was added and extracted with EA (20 mL × 3). The organic layer was dried over Na₂SO₄, filtered and concentrated. The residue was purified by column chromatography (PE/EA= 1: 1) to give 104-3 (1 g, 49.7%) as yellow oil.

The synthesis of 104-4

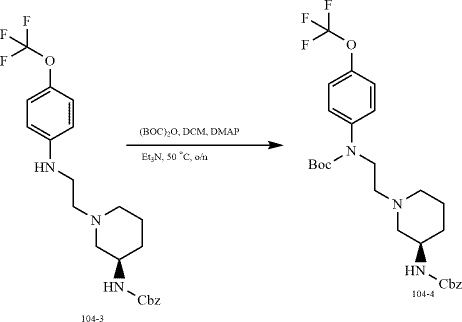

To a solution of 104-3 (1 g, 2.3 mmol) in DCM (30 mL) was added (Boc)₂O (602 mg, 2.76 mmol), DMAP (28 mg, 0.23 mmol) and Et₃ N (697 mg, 6.9 mmol). It was stirred at 50° C. overnight. Water (40 mL) was added and extracted with DCM (20 mL × 3). The organic layer was dried over Na₂SO₄, filtered and concentrated. The residue was purified by column chromatography (PE/EA=4:1) to give 104-4 (500 mg, 40.4%) as yellow oil.

The synthesis of (R)-tert-butyl 2-(3-aminopiperidin-1-yl)ethyl(4-(trifluoromethoxy)phenyl)carbamate (104-5)

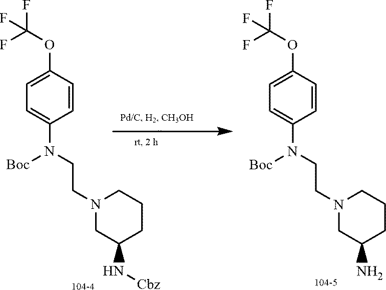

To a solution of 104-4 (500 mg, 0.93 mmol) in CH₃OH (10 mL) was added Pd/C (50 mg). The mixture was stirred at rt overnight under H₂ (1.0 atm) overnight. The mixture was filtered through a Celite pad, and the filtrate was concentrated to give 104-5 (300 mg crude) as yellow oil.

The synthesis of (R)-tert-butyl 4-(trifluoromethoxy)phenyl(2-(3-(vinylsulfonamido)piperidin-1-yl)ethyl)carbamate (104-6)

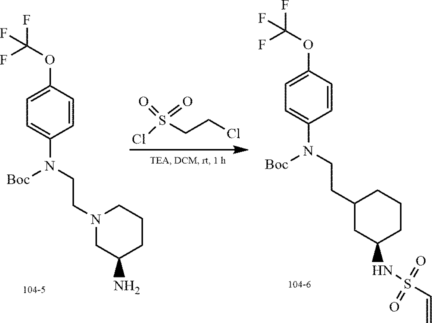

To a solution of 104-5 (300 mg, 0.74 mmol) in DCM (20 mL) was added TEA (97 mg, 0.96 mmol) and 2-chloroethanesulfonyl chloride (133 mg, 0.81 mmol). The mixture was stirred at rt for 1 h. The mixture was pour into ice water and extracted with DCM (20 mL × 3). The organic layer was dried over Na₂SO₄, filtered and concentrated give 104-6 (250 mg, 68.5%) as yellow oil.

The synthesis of (R)-N-(1-(2-(4-(trifluoromethoxy)phenylamino)ethyl)piperidin-3-yl)ethenesulfonamide (SU20667-0104-01)

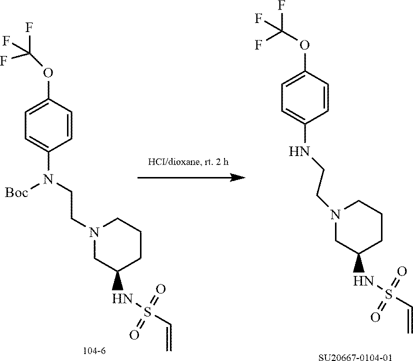

To a solution of 104-6 (250 mg, 0.5 mmol) in dixoxane (10 mL) was added 4 N HCl (10 mL, in dixoxane). It was stirred at rt for 2 h. The mixture concentrated and purified by prep-HPLC to give SU20667-0104-01 (11.01 mg, 5.6%) as a white solid. Agilent LCMS 1200-6120, Column: Waters X-Bridge C18 (50 mm*4.6 mm*3.5 µm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min, Purity 97.52 %, Rt = 2.151 min; MS Calcd.: 393.1; MS Found: 394.1[M+H]⁺. Agilent HPLC 1200, Column: Waters X-Bridge C18 (150 mm*4.6 mm*3.5 µm); Column Temperature: 40° C.; Flow Rate: 1.0 mL/min; Mobile Phase: from 95% [water + 10 mM NH₄HCO₃] and 5% [CH₃CN] to 0% [water + 10 mM NH₄HCO₃] and 100% [CH₃CN] in 10 min, then under this condition for 5 min, finally changed to 95% [water + 10 mM NH₄HCO₃] and 5% [CH₃CN] in 0.1 min and under this condition for 5 min, Purity 99.19%, Rt = 10.185 min; MS Calcd.: 393.1; MS Found: 394.3 [M+H]⁺. 1HNMR (400 MHz, CD₃OD) δ 7.02 (d, J= 8.4 Hz, 2 H), 6.62-6.68 (m, 3 H), 6.13 (t, J= 16.8 Hz, 1 H), 5.87 (d, J= 10.0 Hz, 1 H), 3.15-3.33 (m, 3 H), 2.86-2.91 (m, 1 H), 2.58-2.70 (m, 3 H), 2.11-2.20 (m, 2 H), 1.74-1.88 (m, 2 H), 1.55-1.64 (m, 1 H), 1.33-1.40 (m, 1 H). Chemical Formula: C₁₆H₂₂F₃N₃O₃S. Molecular Weight: 393.42

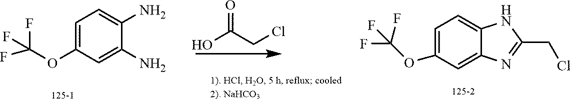

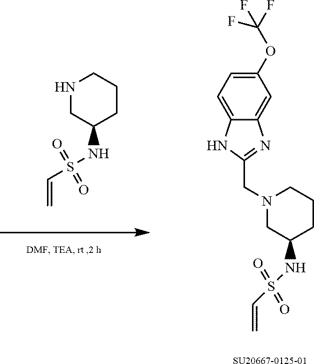

The synthesis of 2-(chloromethyl)-5-(trifluoromethoxy)-1H-benzo[d]imidazole (125-2)

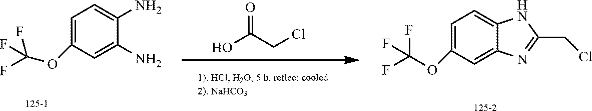

A mixture of 125-1 (1 g, 5.2 mmol) and 2-chloroacetic acid (1 g, 10.4 mmol) in 37% aq.HCl solution (20 ml), the resulting mixture was stirred for 5 h at 100° C. The mixture solution was cooled to room temperature and acidified to pH = 6~8 with 2 N aq.NaHCO₃ and extracted with EA (15 ml × 3). The organic layer dried over Na₂SO₄, and evaporated to get 125-2 (700 mg, 54 %) as a yellow solid.

The synthesis of (R)-N-(1-((5-(trifluoromethoxy)-1H-benzo[d]imidazol-2-yl)methyl)piperidin-3-yl)ethenesulfonamide (SU20667-125-01)

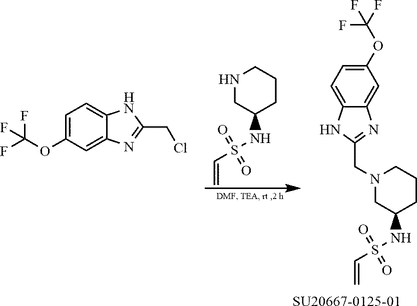

The solution of 125-2 (180 mg, 0.72 mmol), (R)-N-(piperidin-3-yl)ethenesulfonamide (137 mg, 0.72 mmol), in DMF (2 ml) was added TEA (218 mg, 2.16 mmol), the mixture was stirred at room temperature for 2 h, then filtered and the solution was purified prep-HPLC to give SU20667-0125-01 (33.08 mg, 11%) as a white solid. LC-MS (Agilent LCMS 1200-6120, Column: Waters X-Bridge C18 (50 mm*4.6 mm*3.5 µm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min), Purity: 99.09%, Rₜ =1.945 min; MS Calcd.: 404.41; MS Found: 405.2 [M+H]⁺. HPLC (Agilent HPLC 1200, Column: L-column2 ODS (150 mm*4.6 mm*5.0 µm); Column Temperature: 40° C.; Flow Rate: 1.5 mL/min; Mobile Phase: from 90% [(total 10 mM AcONH₄) water/CH₃CN=900/100 (v/v)] and 10% [total 10 mM AcONH₄) water/CH3CN=100/900 (v/v)] to 15% [total 10 mM AcONH4) water/CH₃CN=900/100 (v/v)] and 85% [total 10 mM AcONH4) water/CH₃CN=100/900 (v/v)] in 5 min, then under this condition for 10 min, finally changed to 90% [(total 10mM AcONH4) water/CH₃CN=900/100 (v/v)] and 10% [total 10 mM AcONH4) water/CH₃CN=100/900 (v/v)] in 0.1 min and under this condition for 5 min), Purity:98.01%, Rt =8.973 min. ¹H NMR (400 MHz, DMSO-d₆) δ 12.54 (s, 1 H), 7.50-7.57 (m, 2 H), 7.36 (s, 1 H), 7.14 (d, J=8.4 Hz, 1 H), 6.70 (dd, J=16.8 Hz, 10.0 Hz, 1 H), 5.99 (d, J=16.4 Hz, 1 H), 5.83 (d, J=10.0 Hz, 1 H), 3.69-3.78 (m, 2 H), 3.19 (s, 1 H), 2.82 (d, J=9.2 Hz, 1 H), 2.54-2.58 (m, 1 H), 2.06 (t, J=8.8 Hz, 2 H), 1.71-1.74 (m, 1 H), 1.62-1.66 (m, 1 H), 1.41-1.49 (m, 1 H), 1.21-1.26 (m, 1 H). Chemical Formula: C₁₆H₁₉F₃N₄O₃S. Molecular Weight: 404.41. Melting point: 65.5-78.4° C. Optical rotation: [a]²⁵D = 30.00 (c = 0.20, CH₃OH)

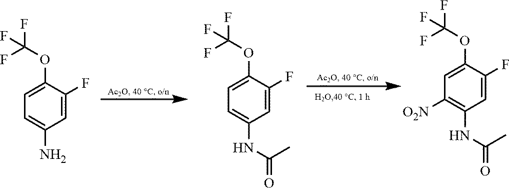

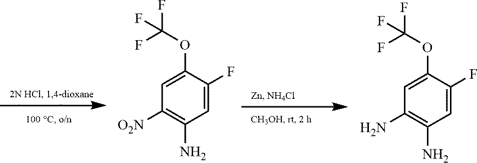

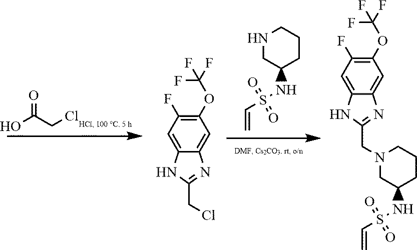

The synthesis of 2-fluoro-4-nitrobenzoic acid (130-2)

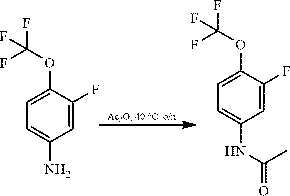

A mixture of 134-1 (5 g, 25.6 mmol) in Ac₂O (30 mL) was stirred at 40° C. overnight. The reaction mixture was concentrated to give 134-2 (4 g, 66%) as yellow oil.

The synthesis of N-(5-fluoro-2-nitro-4-(trifluoromethoxy)phenyl)acetamide (134-3)

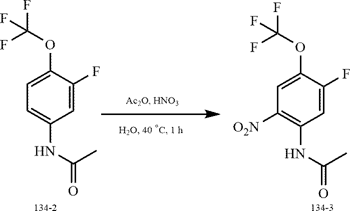

To a solution of 134-2 (4 g, 16.8 mmol) in Ac₂O (10 mL) was added HNO₃ (1.6 mL, 16.8 mmol) and H₂O (10 mL). It was stirred at 40° C. for 1 hour. Water (20 mL) was added and extracted with EA (10 mL × 3). The organic layer was dried over Na₂SO₄, filtered and concentrated. The residue was purified by column chromatography (PE/EA=1:1) to give 134-3 (2.3 g, 48.5%) as yellow oil.

The synthesis of 5-fluoro-2-nitro-4-(trifluoromethoxy)aniline (134-4)

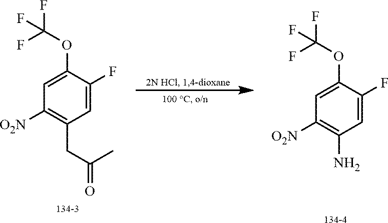

To a solution of 134-3 (1 g, 3.5 mmol) in 1, 4-dioxane (10 mL) was added 2 N HCl (in water) (10 mL). It was stirred at 100° C. overnight. The mixture was adjusted to pH 9 with Na₂CO₃ (aq.) and extracted with EA (10 mL × 3). The organic layer was dried over Na₂SO₄, filtered and concentrated to give 134-4 (700 mg, 83.3%) as yellow oil.

The synthesis of 4-fluoro-5-(trifluoromethoxy)benzene-1,2-diamine (134-5)

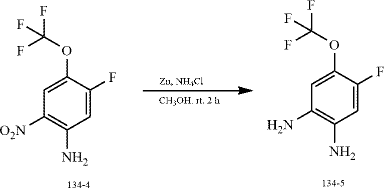

To a solution of 134-4 (700 mg, 2.9 mmol) in CH₃OH (10 mL) was added Zn (565 mg, 8.7 mmol) and NH₄Cl (922 mg, 17.4 mmol). The mixture was stirred at rt for 2 h. The mixture was filtered through a Celite pad, and the filtrate was concentrated to give 134-5 (430 mg, 70.6%) as brown oil.

The synthesis of 2-(chloromethyl)-6-fluoro-5-(trifluoromethoxy)-1H-benzo[d]imidazole (134-6)

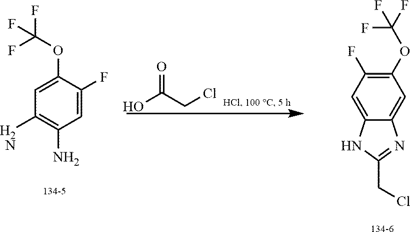

To a solution of 134-5 (430 mg, 2 mmol) in HCl (10.0 N, 10 mL) was added 2-chloroacetic acid (207 mg, 2.2 mmol). It was stirred at 100° C. for 5 h. The mixture was adjusted to pH 9 with Na₂CO₃ (aq.) and extracted with EA (10 mL × 3). The organic layer was dried over Na₂SO₄, filtered and concentrated to give 134-6 (300 mg, 56%) as yellow oil.

The synthesis of (R)-N-(1-((6-fluoro-5-(trifluoromethoxy)-1H-benzo[d]imidazol-2-yl) methyl) piperidin-3-yl) ethenesulfonamide (SU20667-0134-01)

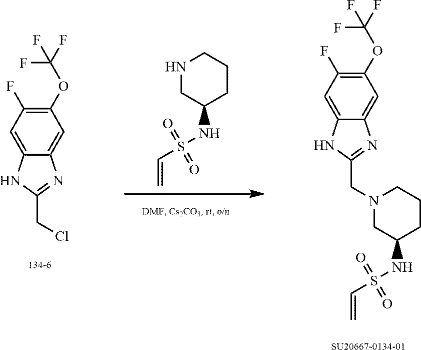

To a solution of 134-6 (300 mg, 1.1 mmol) in DMF (5 mL) was added (R)-N-(piperidin-3-yl)ethenesulfonamide (373 mg, 1.65 mmol) and Cs₂CO₃ (1.1 g, 3.3 mmol). It was stirred at rt overnight. Water (40 mL) was added and extracted with EA (20 mL × 3). The organic layer was dried over Na₂SO₄, filtered and concentrated. The residue was purified by prep-HPLC to give SU20667-0134-01 (67.99 mg, 14.6%) as a white solid. Agilent LCMS 1200-6120, Column: Waters X-Bridge C18 (50 mm*4.6 mm*3.5 µm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min, Purity 100.00 %, Rt = 1.956 min; MS Calcd.: 422.1; MS Found: 423.2 [M+H]⁺. Agilent HPLC 1200, Column: Waters X-Bridge C18 (150 mm*4.6 mm*3.5 µm); Column Temperature: 40° C.; Flow Rate: 1.0 mL/min; Mobile Phase: from 95% [water + 10 mM NH₄HCO₃] and 5% [CH₃CN] to 0% [water + 10 mM NH₄HCO₃] and 100% [CH₃CN] in 10 min, then under this condition for 5 min, finally changed to 95% [water + 10 mM NH₄HCO₃] and 5% [CH₃CN] in 0.1 min and under this condition for 5 min, Purity 99.46 %, Rt = 8.701 min; MS Calcd.: 422.1; MS Found: 423.2 [M+H]⁺. 1 H NMR (400 MHz, DMSO-d₆) δ 12.67 (s, 1 H), 7.74-7.53 (m, 2 H), 7.38 (d, J = 7.6 Hz, 1 H), 6.71 (dd, J = 16.4 Hz, 10.0 Hz, 1 H), 5.99 (d, J = 16.4 Hz, 1 H), 5.84 (d, J = 9.6 Hz, 1 H), 3.69-3.77 (m, 2 H), 3.17-3.19 (m, 1 H), 2.82-2.84 (m, 1 H), 2.49-2.82 (m, 1 H), 2.03-2.08 (m, 2 H), 1.62-1.75 (m, 2 H), 1.40-1.49 (m, 1 H), 1.18-1.26 (m, 1 H). Chemical Formula: C₁₆H₁₈F₄N₄O₃S. Molecular Weight: 422.40. Melting point: 51.8-57.5° C. Optical rotation: [a]²⁰D = 29.50 (c = 0.20, CH₃OH).

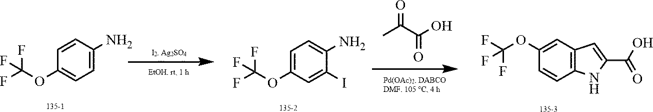

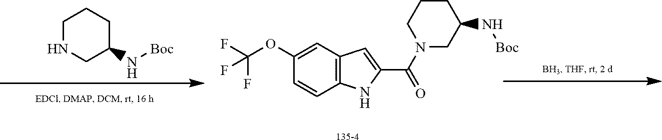

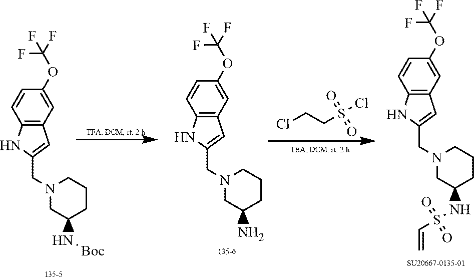

The synthesis of 2-iodo-4-(trifluoromethoxy)aniline (135-2)

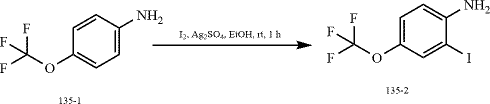

To a solution of 135-1 (3 g, 16.9 mmol) in EtOH (40 mL) was added Ag₂SO₄ (312 mg, 16.9 mmol) and I₂ (4.29 g, 16.9 mmol). The mixture was stirred at rt for 1 h. The mixture was filtered through a Celite pad, and the filtrate was concentrated. The residue was purified by column chromatography (PE/EA= 2:1) to give 135-2 (4 g, 73.3%) as yellow oil.

The synthesis of 5-(trifluoromethoxy)-1H-indole-2-carboxylic acid (135-3)

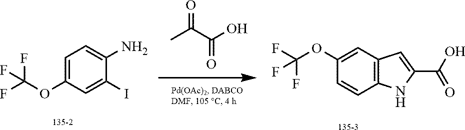

To a solution of 135-2 (4 g, 13.2 mmol) in DMF (20 mL) was added 2-oxopropanoic acid (3.48 g, 39.6 mmol), DABCO (4.43 g, 39.6 mmol) and Pd(OAc)₂ (296 mg, 1.32 mmol). It was stirred under N₂ at 105° C. for 4 h. Water (60 mL) was added and extracted with EA (40 mL × 3). The organic layer was dried over Na₂SO₄, filtered and concentrated. The residue was purified by column chromatography (DCM/MeOH= 15:1) to give 135-3 (2 g, 61.8%) as yellow oil.

The synthesis of (R)-tert-butyl 1-(5-(trifluoromethoxy)-1H-indole-2-carbonyl)piperidin-3-ylcarbamate (135-4)

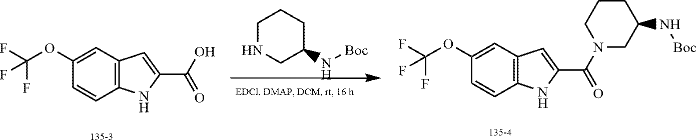

To a solution of 135-3 (0.6 g, 1.4 mmol) in DCM (20 mL) was added EDCI (349 mg, 1.82 mmol), DMAP (342 mg, 2.8 mmol) and (R)-tert-butyl piperidin-3-ylcarbamate (364 mg, 1.82 mmol). It was stirred at rt for 16 h. The mixture was washed with 1 N Citric acid (in water) (20 mL × 3). The organic layer was dried over Na₂SO₄, filtered and concentrated to give 135-4 (700 mg, crude) as yellow oil.

The synthesis of (R)-tert-butyl 1-((5-(trifluoromethoxy)-1H-indol-2-yl)methyl)piperidin-3-ylcarbamate (135-5)

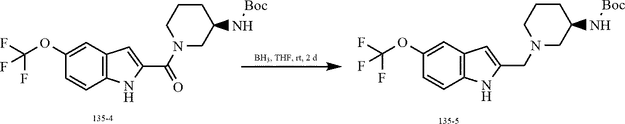

To a solution of 135-4 (700 mg, 1.6 mmol) in THF (20 mL) was added BH₃/THF (1.0 N, 16 mL, 16 mmol). The mixture was stirred at rt for 2 d. The mixture was pour into ice water and extracted with DCM (20 mL × 3). The organic layer was dried over Na₂SO₄, filtered and concentrated. The residue was purified by column chromatography (DCM/MeOH= 20:1) to give 135-5 (450 mg, 68.1%) as yellow oil.

The synthesis of (R)-1-((5-(trifluoromethoxy)-1H-indol-2-yl)methyl)piperidin-3-amine (135-6)

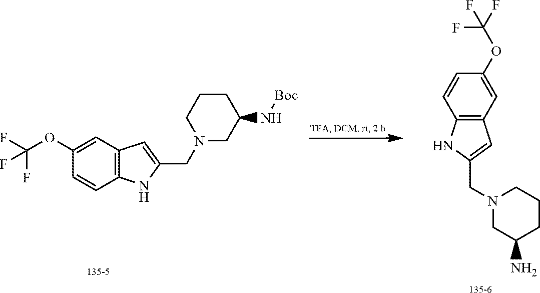

To a solution of 135-5 (450 mg, 1.09 mmol) in DCM (20 mL) was added TFA (2 mL). It was stirred at rt for 2 h. The mixture was adjusted to pH 9 with Na₂CO₃ (aq.) and extracted with DCM (20 mL × 3). The organic layer was dried over Na₂SO₄, filtered and concentrated to give 135-6 (300 mg, 88.7%) as yellow oil.

The synthesis of (R)-N-(1-((5-(trifluoromethoxy)-1H-indol-2-yl)methyl)piperidin-3-yl)ethenesulfonamide (SU20667-0135-01)

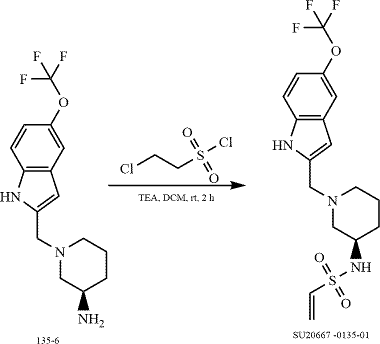

To a solution of 135-6 (300 mg, 0.96 mmol) in DCM (20 mL) was added TEA (194 mg, 1.92 mmol) and 2-chloroethanesulfonyl chloride (171 mg, 1.05 mmol). The mixture was stirred at rt for 2 h. The mixture was pour into ice water and extracted with DCM (20 mL × 3). The organic layer was dried over Na₂SO₄, filtered and concentrated. The residue was purified by prep-HPLC to give SU20667-0135-01 (11.19 mg, 2.9%) as a white solid. Agilent LCMS 1200-6120, Column: Waters X-Bridge C18 (50 mm*4.6 mm*3.5 µm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min, Purity 98.43%, Rt = 2.174 min; MS Calcd.: 403.1; MS Found: 404.3[M+H]⁺. Agilent HPLC 1200, Column: Waters X-Bridge C18 (150 mm*4.6 mm*3.5 µm); Column Temperature: 40° C.; Flow Rate: 1.0 mL/min; Mobile Phase: from 95% [water + 10 mM NH₄HCO₃] and 5% [CH₃CN] to 0% [water + 10 mM NH₄HCO₃] and 100% [CH₃CN] in 10 min, then under this condition for 5 min, finally changed to 95% [water + 10 mM NH₄HCO₃] and 5% [CH₃CN] in 0.1 min and under this condition for 5 min, Purity 96.96 %, Rt = 9.885 min; MS Calcd.: 403.1; MS Found: 404.3 [M+H]⁺. 1H NMR (400 MHz, DMSO-d₆) δ 11.28 (s, 1 H), 7.42 (s, 1 H), 7.38 (d, J = 8.8 Hz, 1 H), 6.99 (dd, J = 8.8 Hz, 1.2 Hz, 1 H), 6.69 (dd, J= 16.4 Hz, 10.0 Hz, 1 H), 6.33 (s, 1 H), 5.98 (d, J= 16.8 Hz, 1 H), 5.82 (d, J= 10.0 Hz, 1 H), 3.57-3.66 (m, 2 H), 3.12-3.17 (m, 1 H), 2.81-2.83 (m, 1 H), 2.50-2.58 (m, 1 H), 1.60-1.94 (m, 4 H), 1.40-1.43 (m, 1 H), 1.18-1.21 (m, 1 H). Chemical Formula: C₁₇H₂₀F₃N₃O₃S. Molecular Weight: 403.42. Melting point: 36.8-40.2° C. Optical rotation: [a]²⁰D = 34.00 (c = 0.10, CH₃OH)

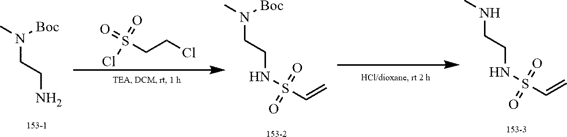

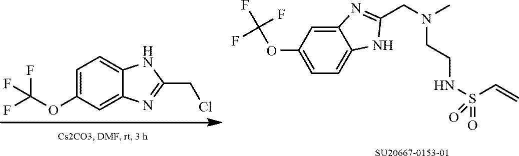

The synthesis of tert-butyl methyl(2-(vinylsulfonamido)ethyl)carbamate (153-2)

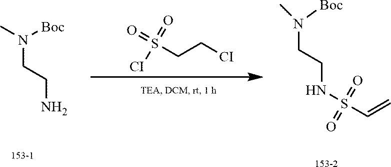

To a solution of tert-butyl N-(2-aminoethyl)-N-methyl-carbamate (153-1, 500 mg, 2.87 mmol) and TEA (725.93 mg, 7.17 mmol, 999.91 uL) in DCM (10 ml) was added dropwise 2-chloroethanesulfonyl chloride (467.81 mg, 2.87 mmol), the mixture was stirred at room temperature for 1 h and the solvent was removed in vacuo to give 153-2 (750 mg, 98.9% yield) as yellow oil.

The synthesis of N-(2-(methylamino)ethyl)ethenesulfonamide (0153-3)

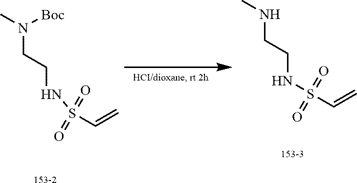

To a solution of 0153-2 (750 mg, 2.84 mmol) in HCl/dioxane (12 mL) was stirred at room temperature for 2 h, The solvent was removed in vacuo to give 0153-3 (450 mg, 96.6% yield) as yellow oil.

The synthesis of N-(2-(methyl((5-(trifluoromethoxy)-1H-benzo[d]imidazol-2-yl)methyl)amino)ethyl)ethenesulfonamide (SU20667-0153-01)

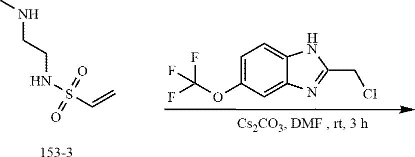

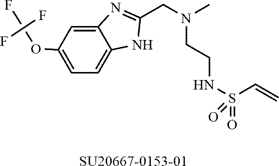

The solution of 153-3 (125 mg, 761.15 umol), 2-(chloromethyl)-5-(trifluoromethoxy)-1H-benzimidazole (190.75 mg, 761.15 umol), in DMF (3 ml) was added cesium carbonate (248.00 mg, 761.15 umol), the mixture was stirred at room temperature for 3 h, The mixture was filtered and the solution was purified prep-HPLC to give SU20667-0153-01 (73.49 mg, 25.5% yield) as yellow oil. LC-MS (Agilent LCMS 1200-6110, Column: Waters X-Bridge C18 (50 mm*4.6 mm*3.5 µm); Column Temperature: 40° C.; Flow Rate: 2.0 mL/min; Mobile Phase: from 95% [water + 0.05% TFA] and 5% [CH₃CN + 0.05% TFA] to 0% [water + 0.05% TFA] and 100% [CH₃CN + 0.05 % TFA] in 1.6 min, then under this condition for 1.4 min, finally changed to 95% [water + 0.05% TFA] and 5% [CH₃CN + 0.05% TFA] in 0.05 min and under this condition for 0.7 min), Purity: 99.54%, Rₜ =1.521 min; MS Calcd: 378.37; MS Found: 379.1 [M+H]⁺. HPLC (Agilent HPLC 1200; Column: L-column2 ODS (150 mm*4.6 mm*5.0 µm); Column Temperature: 40° C.; Flow Rate: 1.0 mL/min; Mobile Phase: from 95% [water + 0.1% TFA] and 5% [CH₃CN + 0.1% TFA] to 0% [water + 0.1% TFA] and 100% [CH₃CN + 0.1% TFA] in 10 min, then under this condition for 5 min, finally changed to 95% [water + 0.1% TFA] and 5% [CH₃CN + 0.1% TFA] in 0.1 min and under this condition for 5 min), Purity: 100.00%, Rt =7.502 min. ¹H NMR (400 MHz, DMSO-d₆) δ 12.52-12.55 (m, 1 H), 7.43-7.64 (m, 2 H), 7.11-7.22 (m, 2 H), 6.70 (dd, J = 16.8 Hz, 10.0 Hz, 1 H), 6.01 (d, J = 16.8 Hz, 1 H), 5.91 (d, J= 10.0 Hz, 1 H), 3.78 (s, 2 H), 3.00-3.01 (m, 2 H), 2.56-2.57 (m, 2 H), 2.22 (s, 3 H). Chemical Formula: C₁₄H₁₇F₃N₄O₃S. Molecular Weight: 378.37.

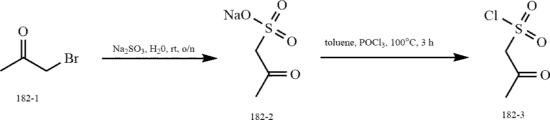

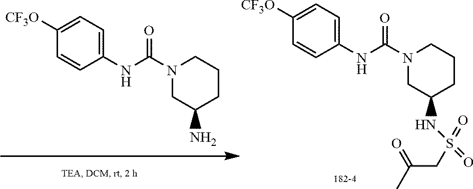

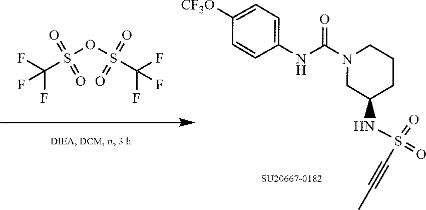

The synthesis of sodium 2-oxopropane-1-sulfonate (182-2)

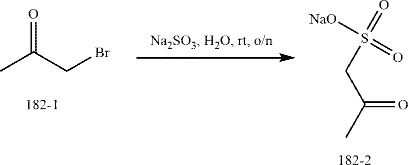

To a solution of 182-1 (500 mg, 3.7 mmol) in H₂O (10 mL) was added Na₂SO₃ (699 mg, 5.55 mmol). The mixture was stirred at rt for 48 h then concentrated in vacuo to give 182-2 (1 g, crude) as a white solid.

The synthesis of 2-oxopropane-1-sulfonyl chloride (182-3)

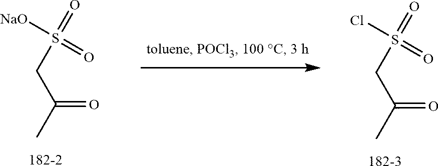

To a solution of 182-2 (1 g, crude) in toluene (10 mL) was added POCl₃ (2 mL). The reaction mixture was stirred at 100° C. for 3 h and concentrated in vacuo, DCM (20 mL) was added and the reaction mixture was filtered, the filtrate was concentrated to give 182-3 (500 mg, crude) as yellow oil.

The synthesis of (R)-3-(2-oxopropylsulfonamido)-N-(4-(trifluoromethoxy)phenyl)piperidine-1-carboxamide (182-4)

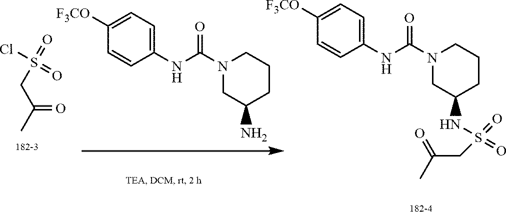

To a solution of (R)-3-amino-N-(4-(trifluoromethoxy)phenyl)piperidine-1-carboxamide (496 mg, 1.9 mmol) in DCM (20 mL) was added Et₃N (1.15 g, 11.4 mmol) and 182-3 (500 mg, crude). The reaction mixture was stirred at rt for 2 h and quenched with water (20 mL). The organic layer was concentrated and purified by column chromatography (PE/EA= 1:2) to give 182-4 (100 mg, 6.3% three steps) as a white solid.

The synthesis of (R)-3-(prop-1-yn-1-ylsulfonamido)-N-(4-(trifluoromethoxy)phenyl)piperidine-1-carboxamide (SU20667-0182-01)

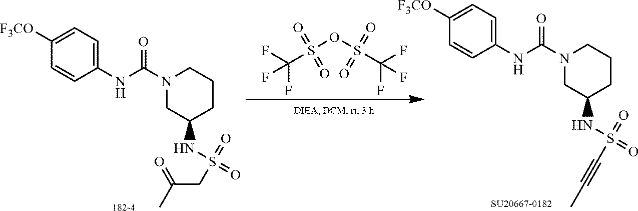

To a solution of 182-4 (100 mg, 0.23 mmol) in DCM (2 mL) was added DIEA (90 mg, 0.7 mmol) and Tf₂O (77 mg, 0.27 mmol). The mixture was stirred at rt for 3 h. The mixture was purified by prep-TLC to give the crude product. The residue was further purified by prep-HPLC to give SU20667-0182-01 (7.29 mg, 7.8%) as a white solid. Agilent LCMS 1200-6120, Column: Waters X-Bridge C18 (50 mm*4.6 mm*3.5 µm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min, Purity 100.00 %, Rt = 1.803 min; MS Calcd.: 405.1; MS Found: 406.1 [M+H]⁺. Agilent HPLC 1200, Column: Waters X-Bridge C18 (150 mm*4.6 mm*3.5 µm); Column Temperature: 40° C.; Flow Rate: 1.0 mL/min; Mobile Phase: from 95% [water + 10 mM NH₄HCO₃] and 5% [CH₃CN] to 0% [water + 10 mM NH₄HCO₃] and 100% [CH₃CN] in 10 min, then under this condition for 5 min, finally changed to 95% [water + 10 mM NH₄HCO₃] and 5% [CH₃CN] in 0.1 min and under this condition for 5 min, Purity 100.00%, Rt = 8.791 min; MS Calcd.: 405.1; MS Found: 406.1 [M+H]⁺. 1 H NMR (400 MHz, CD₃OD) δ 7.40-7.43 (m, 2 H), 7.15 (d, J = 8.0 Hz, 2 H), 4.09-4.14 (m, 1 H), 3.72-3.76 (m, 1 H), 3.40-3.43 (m, 1 H), 3.14-3.20 (m, 1 H), 3.04-3.09 (m, 1 H), 2.06-2.09 (m, 1 H), 2.04 (s, 3 H), 1.79-1.81 (m, 1 H), 1.58-1.63 (m, 2 H).

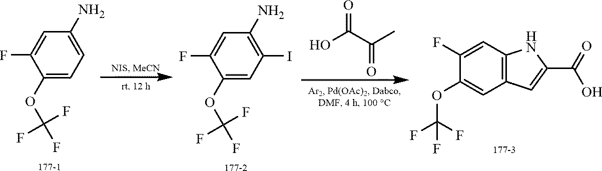

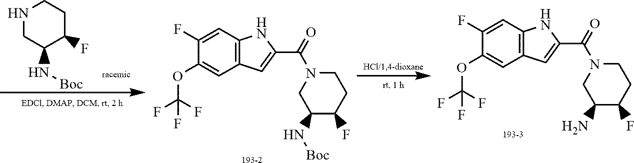

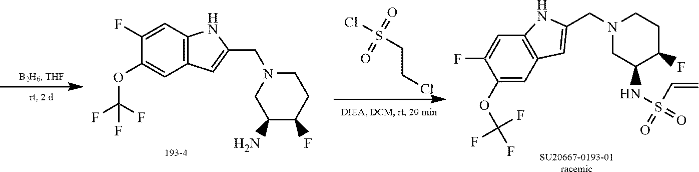

The synthesis of 5-fluoro-2-iodo-4-(trifluoromethoxy)aniline (177-2)

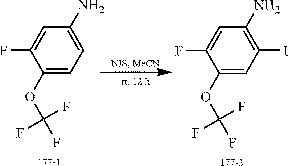

To a solution of 3-fluoro-4-(trifluoromethoxy)aniline (3 g, 15.38 mmol) in acetonitrile (50 mL) was added NIS (3.39 g, 15.07 mmol) and stirred at rt for 12 h. Removing the solution and the residue was purified by C.C. (PE/EA= 2:1) to give 177-2 (3.2 g, 9.97 mmol, 64.81% yield) as black oil.

The synthesis of 6-fluoro-5-(trifluoromethoxy)-1H-indole-2-carboxylic acid (177-3)

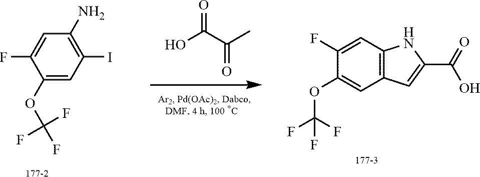

To a solution of 177-2 (4 g, 12.46 mmol) and 2-oxopropanoic acid (1.21 g, 13.71 mmol) in DMF (60 mL) was added palladium (II) acetate (279.75 mg, 1.25 mmol) and Dabco (2.80 g, 24.92 mmol, 2.45 mL) was stirred at 100° C. for 4 h. Removing the solvent and MeOH was added and filtered, the filtrate was concentrated and used to next step without further purification.

The synthesis of tert-butyl (3S,4R)-4-fluoro-1-(6-fluoro-5-(trifluoromethoxy)-1H-indole-2-carbonyl)piperidin-3-ylcarbamate (0193-2)

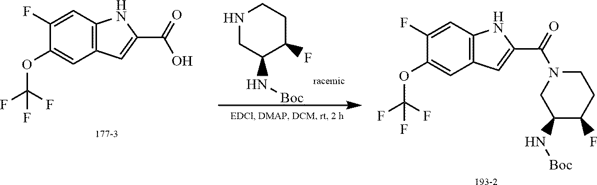

To a solution of 177-3 (400 mg, 1.52 mmol) and tert-butyl N-[(3S,4R)-4-fluoro-3-piperidyl]carbamate (331.78 mg, 1.52 mmol) in DCM (6 mL) was added DMAP (278.56 mg, 2.28 mmol) and EDCI (377.43 mg, 1.98 mmol), the mixture was stirred at rt for 2 h, Removing the solvent and the residue was purified by C.C (DCM/MeOH = 15:1) to give 193-2 (450 mg, 971.09 umol, 63.88% yield) as a white solid.

The synthesis of ((3S,4R)-3-amino-4-fluoropiperidin-1-yl)(6-fluoro-5-(trifluoromethoxy)-1H-indol-2-yl)methanone (0193-3)

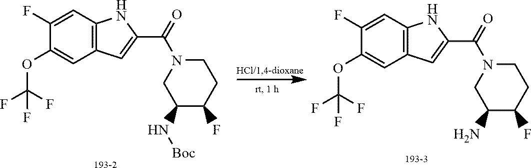

To a solution of 193-2 (200 mg, 431.59 umol) in HCl/1,4-dioxane (3 mL) was stirred at rt for 1 h, the solvent was removed to give the desired product 193-3 (156 mg, 99.5% yield) as a white solid.

The synthesis of (3S,4R)-4-fluoro-1-((6-fluoro-5-(trifluoromethoxy)-1H-indol-2-yl)methyl)piperidin-3-amine (0193-4)

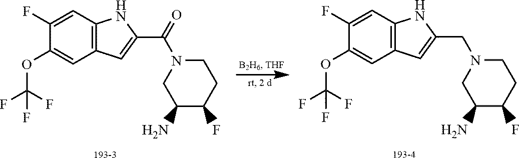

To a solution of 193-3 (200 mg, 550.54 umol) in THF (2 mL) was added BH₃/THF (1.0 N, 2 mL), the mixture was stirred at rt for 2 d. The mixture was pour into ice water and extracted with DCM (20 mL × 3). The organic layer was dried over Na₂SO₄, filtered and concentrated. The residue was purified by column chromatography (DCM/MeOH= 20:1) to give 193-4 (70 mg, 200.40 umol, 36.40% yield) as colorless oil.

The synthesis of N-((3S,4R)-4-fluoro-1-((6-fluoro-5-(trifluoromethoxy)-1H-indol-2-yl)methyl)piperidin-3-yl)ethenesulfonamide (SU20667-0193-01)

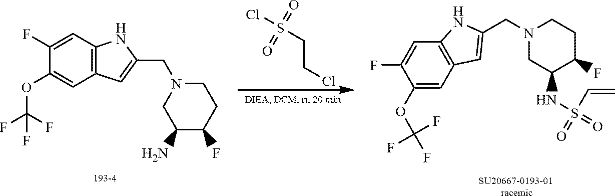

To a solution of 193-4 (70 mg, 200.40 umol) in DCM (2 mL) was added TEA (60.84 mg, 601.21 umol) and 2-chloroethanesulfonyl chloride (32.67 mg, 200.40 umol). The mixture was stirred at rt for 20 min. The mixture was pour into ice water and extracted with DCM (10 mL × 3). The organic layer was dried over Na₂SO₄, filtered and concentrated. The residue was purified by prep-HPLC to give SU20667-0193-01 (23.16 mg, 26.3% yield) as a white solid. LC-MS (Agilent LCMS 1200-6120, Column: Waters X-Bridge C18 (50 mm*4.6 mm*3.5 µm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min), Purity: 97.25%, Rₜ =2.147 min; MS Calcd: 439.40; MS Found: 440.2 [M+H]⁺. HPLC (Agilent HPLC 1200, Column: Waters X-Bridge C18 (150 mm*4.6 mm*3.5 µm); Column Temperature: 40° C.; Flow Rate: 1.0 mL/min; Mobile Phase: from 95% [water + 10 mM NH₄HCO₃] and 5% [CH₃CN] to 0% [water + 10 mM NH₄HCO₃] and 100% [CH₃CN] in 10 min, then under this condition for 5 min, finally changed to 95% [water + 10 mM NH₄HCO₃] and 5% [CH₃CN] in 0.1 min and under this condition for 5 min), Purity:98.90%, Rt =7.331 min. ¹H NMR (400 MHz, DMSO-d₆) δ 11.38 (s, 1 H), 7.62 (d, J=6.4 Hz, 2 H), 7.34 (d, J=10.8 Hz, 1 H), 6.71 (dd, J=16.4 Hz, J=10.0 Hz, 1 H), 6.37 (s, 1 H), 6.00 (d, J=16.4 Hz, 1 H), 5.82 (d, J=10.0 Hz, 1 H), 4.74 (d, J=47.6 Hz, 1 H), 3.67 (s, 2 H), 3.37-3.38 (m, 1 H), 2.64-2.66 (m, 1 H), 2.49-2.50 (m, 1 H), 2.25-2.32 (m, 2 H), 1.88-1.91 (m, 2 H). Chemical Formula: C₁₇H₁₈F₅N₃O₃S. Molecular Weight: 439.40. Melting point: 165.2-169.9° C.

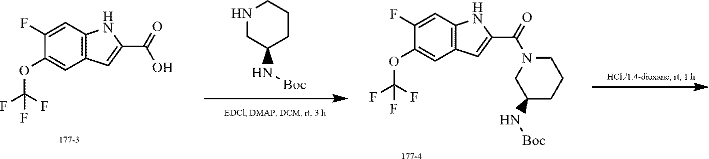

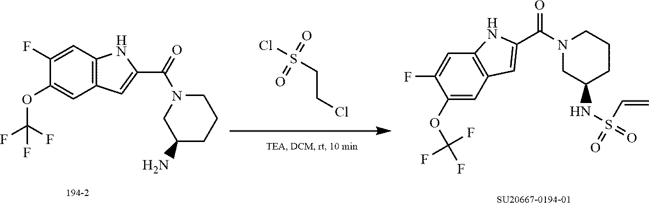

The synthesis of (R)-tert-butyl 1-(6-fluoro-5-(trifluoromethoxy)-1H-indole-2-carbonyl)piperidin-3-ylcarbamate (177-4)

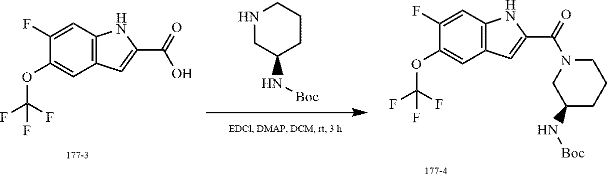

To a solution of 177-3 (740 mg, 2.81 mmol) and tert-butyl N-[(3R)-3-piperidyl]carbamate (563.21 mg, 2.81 mmol) in DCM (15 mL), was added DMAP (687.12 mg, 5.62 mmol) and EDCI (700.81 mg, 3.66 mmol), the mixture was stirred at rt for 3 h. Removing the solvent and the residue was purified by CC (PE/EA= 2:1) to give 177-4 (600 mg, 47.9% yield) as a yellow solid.

The synthesis of (R)-(3-aminopiperidin-1-yl)(6-fluoro-5-(trifluoromethoxy)-1H-indol-2-yl)methanone (0194-2)

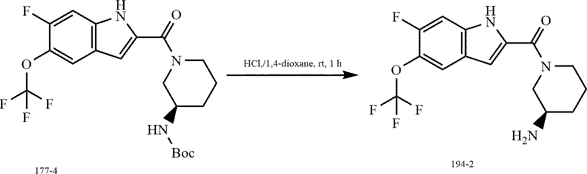

To a solution of 177-4 (150 mg, 336.77 umol) in HCl/1,4-dioxane (3 mL) was stirred at rt for 1 h, the solvent was removed to give the desired product 194-2 (116 mg, 99.76% yield) as a yellow solid.

The synthesis of (R)-N-(1-(6-fluoro-5-(trifluoromethoxy)-1H-indole-2-carbonyl)piperidin-3-yl)ethenesulfonamide (SU20667-0194-01)

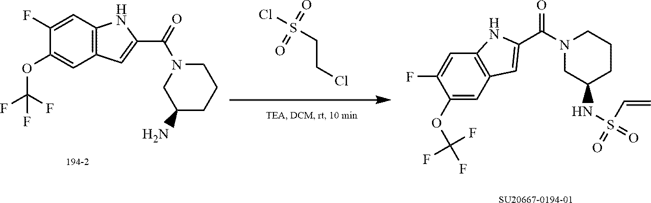

To a solution of 194-2(116 mg, 335.95 umol) in DCM (3 mL) was added TEA (140 mg, 1.38 mmol) and 2-chloroethanesulfonyl chloride (54 mg, 331.24 umol). The mixture was stirred at rt for 10 min. The mixture was pour into ice water and extracted with DCM (10 mL× 3). The organic layer was dried over Na₂SO₄, filtered and concentrated. The residue was purified by prep-HPLC to give SU20667-0194-01 (15.67 mg, 10.7% yield) as a white solid. LC-MS (Agilent LCMS 1200-6120, Column: Waters X-Bridge C18 (50 mm*4.6 mm*3.5 µm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min), Purity: 98.41%, Rₜ =2.033 min; MS Calcd: 435.39; MS Found: 436.2 [M+H]⁺. HPLC (Agilent HPLC 1200, Column: Waters X-Bridge C18 (150 mm*4.6 mm*3.5 µm); Column Temperature: 40° C.; Flow Rate: 1.0 mL/min; Mobile Phase: from 95% [water + 10 mM NH₄HCO₃] and 5% [CH₃CN] to 0% [water + 10 mM NH₄HCO₃] and 100% [CH₃CN] in 10 min, then under this condition for 5 min, finally changed to 95% [water + 10 mM NH₄HCO₃] and 5% [CH₃CN] in 0.1 min and under this condition for 5 min), Purity:99.21%, Rt =9.584 min. ¹H NMR (400 MHz, DMSO-d₆) δ 11.92 (s, 1 H), 7.80 (d, J=6.8 Hz, 1 H), 7.62 (d, J=2.0 Hz, 1 H), 7.39 (d, J=10.0 Hz, 1 H), 6.86 (s, 1 H), 6.76 (dd, J=16.8 Hz, J=9.6 Hz, 1 H), 6.04 (d, J=16.4 Hz, 1 H), 5.90-5.92 (m, 1 H), 4.29-4.32 (m, 1 H), 4.07-4.11 (m, 1 H), 3.16-3.32 (m, 3 H), 1.93-1.95 (m, 1 H), 1.76-1.79 (m, 1 H), 1.46-1.54 (m, 2 H). Chemical Formula: C₁₇H₁₇F₄N₃O₄S. Molecular Weight: 435.39 Melting point: 91.7-98.6° C. Optical rotation: [a]²⁵D = 8.0 (c = 0.20, CH₃OH).

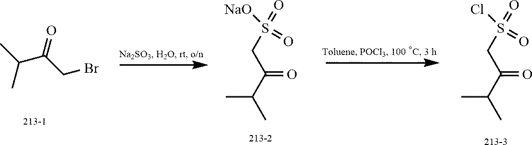

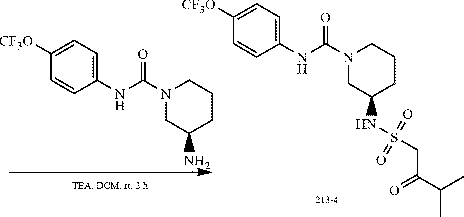

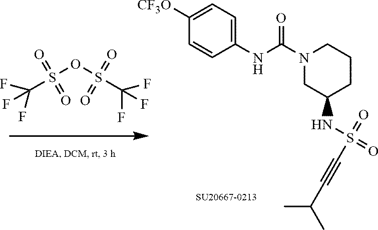

The synthesis of sodium 3-methyl-2-oxobutane-1-sulfonate (213-2)

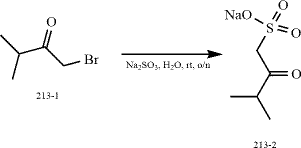

To a solution of 213-1 (1.7 g, 10.3 mmol) in H₂O (10 mL) was added Na₂SO₃ (1.9 g, 15.45 mmol). The mixture was stirred at rt for 48 h and concentrated in vacuo to give 213-2 (2 g, crude) as a white solid.

The synthesis of 3-methyl-2-oxobutane-1-sulfonyl chloride (213-3)

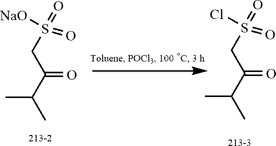

To a solution of 213-2 (2 g, crude) in toluene (20 mL) was added POCl₃ (4 mL). The reaction mixture was stirred at 100° C. for 3 h and concentrated in vacuo, DCM (20 mL) was added and the reaction mixture was filtered, the filtrate was concentrated to give 213-3 (1 g, crude) as yellow oil.

The synthesis of (R)-3-(3-methyl-2-oxobutylsulfonamido)-N-(4-(trifluoromethoxy)phenyl)piperidine-1-carboxamide (213-4)

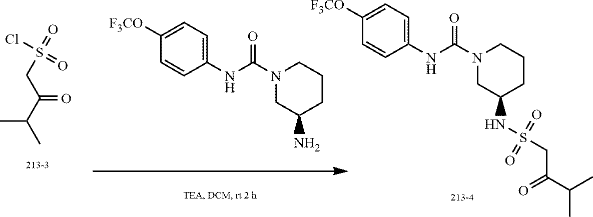

To a solution of (R)-3-amino-N-(4-(trifluoromethoxy)phenyl)piperidine-1-carboxamide (1 g, 4 mmol) in DCM (20 mL) was added Et₃ N (2.4 g, 24 mmol) and 213-3 (1 g, crude). The reaction mixture was stirred at rt for 2 h and quenched with water (20 mL). The organic layer was concentrated and purified by column chromatography (PE/EA= 1:2) to give 213-4 (120 mg, 2.6% for three steps) as a white solid.

The synthesis of (R)-3-(3-methylbut-1-yn-1-ylsulfonamido)-N-(4-(trifluoromethoxy)phenyl)piperidine-1-carboxamide (SU20667-0213-01)

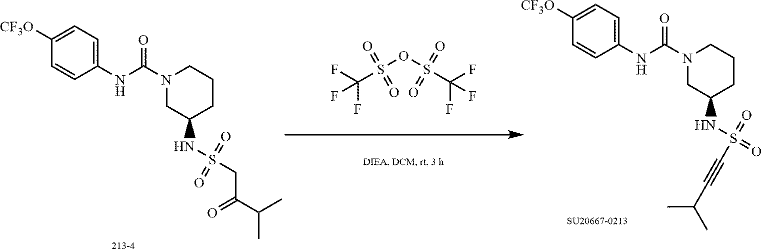

To a solution of 213-4 (120 mg, 0.26 mmol) in DCM (2 mL) was added DIEA (103 mg, 0.8 mmol) and Tf₂O (88 mg, 0.31 mmol). The mixture was stirred at rt for 3 h. The mixture was purified by prep-TLC to give the crude product. The residue was further purified by prep-HPLC to give SU20667-0213-01 (6.29 mg, 5.5%) as a white solid. Agilent LCMS 1200-6120, Column: Waters X-Bridge C18 (50 mm*4.6 mm*3.5 µm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min, Purity 100.0 %, Rt = 1.621 min; MS Calcd.: 433.1; MS Found: 433.8 [M+H]⁺. Agilent HPLC 1200, Column: Waters X-Bridge C18 (150 mm*4.6 mm*3.5 µm); Column Temperature: 40° C.; Flow Rate: 1.0 mL/min; Mobile Phase: from 95% [water + 10 mM NH₄HCO₃] and 5% [CH₃CN] to 0% [water + 10 mM NH₄HCO₃] and 100% [CH₃CN] in 10 min, then under this condition for 5 min, finally changed to 95% [water + 10 mM NH₄HCO₃] and 5% [CH₃CN] in 0.1 min and under this condition for 5 min, Purity 100.0%, Rt = 10.380 min; MS Calcd.: 433.1; MS Found: 434.2 [M+H]⁺. 1 H NMR (400 MHz, DMSO-d₆) δ 8.72 (s, 1 H), 8.38 (d, J = 7.2 Hz, 1 H), 7.53-7.55 (m, 2 H), 7.22 (d, J = 8.4 Hz, 2 H), 4.14-4.20 (m, 1 H), 3.82-3.85 (m, 1 H), 3.23-3.34 (m, 1 H), 2.75-2.92 (m, 3 H), 1.98-1.99 (m, 1 H), 1.72-1.74 (m, 1 H), 1.45-1.50 (m, 2 H), 1.16 (d, J = 5.6 Hz, 6 H).

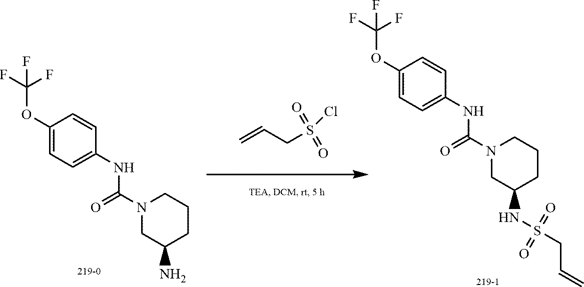

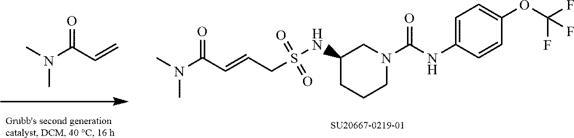

The synthesis of (R)-3-(allylsulfonamido)-N-(4-(trifluoromethoxy)phenyl) piperidine-1-carboxamide (219-1)

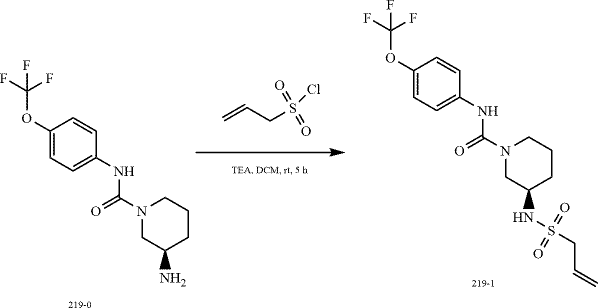

To a solution of 219-0 (450 mg, 1.49 mmol) in DCM (10 mL) was added TEA (451 mg, 4.46 mmol), then prop-2-ene-1-sulfonyl chloride (417 mg, 2.98 mmol) was added dropwise at room temperature. Then the mixture was stirred at room temperature for 5 h. Water (30 mL) was added and extracted with DCM (30 mL × 3). The organic layer was dried over Na₂SO₄, filtered and concentrated to give the oil residue, which was purified by prep-HPLC to give 219-1 (80 mg, 13%) as yellow oil.

The synthesis of (R,E)-3-(4-(dimethylamino)-4-oxobut-2-enylsulfonamido)-N- (4-(trifluoromethoxy)phenyl)piperidine-1-carboxamide (SU20667-0219-01)

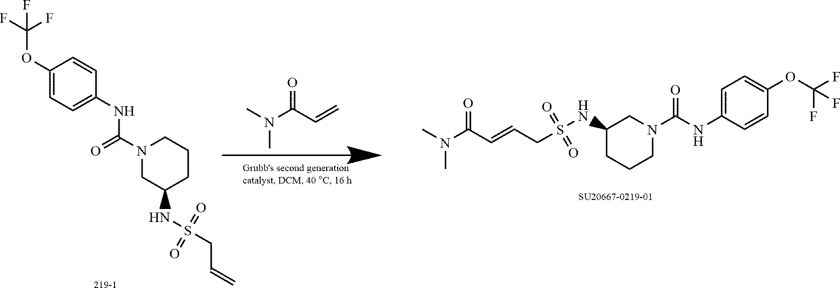

To a solution of 219-1 (60 mg, 0.15 mmol) in DCM (5 mL) was added N,N-dimethylacrylamide (18 mg, 0.18 mmol) and Grubb’s second generation catalyst (127 mg, 0.15 mmol). It was stirred at 40° C. for 16 hr. Diluted with water (10 mL) and extracted with DCM (10 mL × 2). The organic layer was dried over Na₂SO₄, filtered and concentrated. The residue was purified by Prep-HPLC to give SU20667-0219-01 (5 mg, 7%) as a white solid. Agilent LCMS 1200-6120, Column: Waters X-Bridge C18 (50 mm*4.6 mm*3.5 µm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min. Purity is 100%. Rt = 1.877 min; MS Calcd.: 478.2; MS Found: 479.4 [M+H]⁺. Agilent HPLC 1200, Column: Waters X-Bridge C18 (150 mm*4.6 mm*3.5 µm); Column Temperature: 40° C.; Flow Rate: 1.0 mL/min; Mobile Phase: from 95% [water + 10 mM NH₄HCO₃] and 5% [CH₃CN] to 0% [water + 10 mM NH₄HCO₃] and 100% [CH₃CN] in 10 min, then under this condition for 5 min, finally changed to 95% [water + 10 mM NH₄HCO₃] and 5% [CH₃CN] in 0.1 min and under this condition for 5 min. Purity is 93.7%. Rt = 8.660 min. MS Calcd.: 478.2; MS Found: 479.2 [M+H]⁺. ¹H NMR (400 MHz, DMSO-d₆) δ 8.74 (s, 1 H), 7.52 (d, J= 9.2 Hz, 2 H), 7.20 (d, J= 8.8 Hz, 2 H), 6.73 (d, J= 15.2 Hz, 1 H), 6.47-6.57 (m, 1 H), 3.95-4.08 (m, 3 H), 3.77-3.87 (m, 1 H), 3.13-3.22 (m, 1 H), 3.02 (s, 3 H), 2.78-2.88 (m, 5 H), 2.65-2.75 (m, 1 H), 1.86-1.96 (m, 1 H), 1.62-1.75 (m, 1 H), 1.33-1.46 (m, 2 H). Chemical Formula: C₁₉H₂₅F₃N₄O₅S. Molecular Weight: 478.49

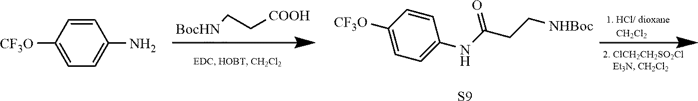

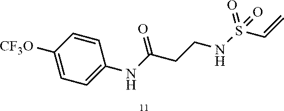

tert-Butyl N-(2-{[4-(trifluoromethoxy)phenyl]carbamoyl}ethyl)carbamate (S9): 0.20 mL (1.4 mmol) 4-(trifluoromethoxy)aniline, 0.20 g (1.0 mmol) Boc-β-alanine, 0.23 g (1.2 mmol) 1-(3-dimethylaminopropyl)-3-ethylcarbodiimide hydrochloride and 0.016 g (0.10 mmol) 1-hydroxybenzotriazole hydrate were mixed in 4 mL methylene chloride and stirred overnight. The solution was diluted with 30 mL water and extracted thrice with 25 mL methylene chloride. Flash chromatography using a hexanes/ethyl acetate gradient resulted in 0.33 g of the final product as a white solid in 91% yield. ¹H NMR (300 MHz, CDCl₃) δ 8.86 (s, 1H), 7.55 (d, J = 8.7 Hz, 2 H), 7.09 (d, J= 8.6 Hz, 2 H), 5.43 (t, J= 6.2 Hz, 1H), 3.45 (q, J= 6.2 Hz, 2 H), 2.57 (t, J = 6.1 Hz, 2 H), 1.39 (s, 9 H). ¹³C NMR (75 MHz, CDCl₃) δ 170.22, 156.68, 145.53, 136.93, 125.76, 122.36, 121.48, 118.96, 115.56, 79.87, 37.68, 36.95, 28.44. Mass (ESI): [M+H-Boc]⁺ 249.1; found 249.1, [M+acetonitrile+Na]⁺ 412.1; found 412.1. R_{f}= 0.40 H:EA 1:1.

3-Ethenesulfonamido-N-[4-(trifluoromethoxy)phenyl]propanamide (11): To 0.33 g (0.95 mmol) tert-butyl N-(2-{[4-(trifluoromethoxy)phenyl]carbamoyl}ethyl)carbamate (S9) in 7 mL methylene chloride was added 2.4 mL (9.5 mmol) 4 M hydrochloric acid in 1,4-dioxane. After 4 hours the solvent was removed using a rotary evaporator. Mass (ESI): [M+H]⁺ 249.1; found 249.0. R_{f} = 0.07 DCM:MeOH 9:1. To the crude 3-amino-N-[4-(trifluoromethoxy)phenyl]propanamide in 15 mL of methylene chloride was added 0.66 mL (4.7 mmol) triethylamine followed by 0.50 mL (4.7 mmol) 2-chloroethanesulfonyl chloride. After stirring overnight the solution was diluted with 20 mL water and the aqueous extracted thrice with 10 mL of methylene chloride. Flash chromatography using a hexanes/ethyl acetate gradient resulted in 0.085 g of the final product as a white solid in 27% yield over two steps. ¹H NMR (300 MHz, CDCl₃) δ 8.30 (s, 1 H), 7.51 (d, J = 8.7 Hz, 2H), 7.09 (d, J = 8.6 Hz, 2 H), 6.49 (dd, J = 16.5, 9.9 Hz, 1 H), 6.19 (d, J = 16.7 Hz, 1H), 5.91 (d, J = 9.9 Hz, 1H), 5.64 (t, J = 6.4 Hz, 1 H), 3.30 (q, J= 6.1 Hz, 2 H), 2.64 (t, J= 6.1 Hz, 2 H). ¹⁹F NMR (282 MHz, CDCl₃) δ -58.17. ¹³C NMR (75 MHz, CDCl₃) δ 169.77, 145.82, 136.46, 135.92, 126.96, 121.66, 120.69 (q, J=256.8 Hz), 39.33, 37.20. Mass (ESI): [M+H]⁺ 339.1; found 339.0. Rf = 0.18 H:EA 1:1.

The synthesis of N-[(3 S)-1-[5-(4-Chlorophenyl)furan-2-carbonyl]piperidin-3-yl]ethene-1-sulfonamide.

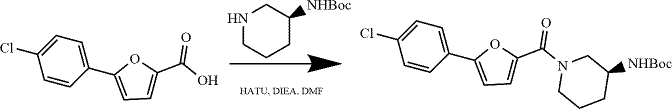

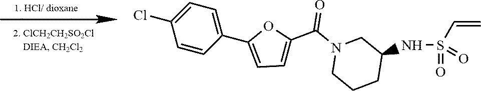

tert-Butyl N-[(3S)-1-[5-(4-chlorophenyl)furan-2-carbonyl]piperidin-3-yl]carbamate. To a solution of 5-(4-chlorophenyl)furan-2-carboxylic acid (0.16 g, 0.72 mmol) in N,N′-dimethylformamide (1 mL) were added (S)-3-(Boc-amino)piperidine (0.144 g, 0.72 mmol), HATU (0.3 g, 0.79 mmol) and N,N-diisopropylethylamine (0.25 mL, 1.44 mmol). The reaction was stirred at ambient temperature for 2 h then partitioned between ethyl acetate and saturated aqueous ammonium chloride. The layers were separated, and the organic phase was washed with water and brine, dried over MgSO₄, filtered, and concentrated. The crude concentrate was purified by flash silica gel chromatography (12 g Silicycle column), eluting with ethyl acetate in hexanes (50%) to provide 0.26 g (89%) of the title compound as an off-white solid. ¹H NMR (400 MHz, CHLOROFORM-d, 25° C.): δ = 7.66 (d, J= 7.8 Hz, 2 H), 7.36 (d, J= 8.6 Hz, 2 H), 7.12 (br s, 1 H), 6.69 (d, J= 3.9 Hz, 1 H), 3.80 (br s, 1 H), 3.58 (br s, 1 H), 1.96 (br dd, J= 7.8, 3.5 Hz, 1 H), 1.80 (br s, 1 H), 1.60-1.74 (m, 2 H), 1.51-1.58 (m, 2 H), 1.42-1.48 (m, 1H), 1.39 ppm (s, 9 H). LC-MS: m/z = 427 [M+Na]⁺.

N-[(3S)-1-[5-(4-Chlorophenyl)furan-2-carbonyl]piperidin-3-yl]ethene-1-sulfonamide. To a solution of tert-butyl N-[(3S)-1-[5-(4-chlorophenyl)furan-2-carbonyl]piperidin-3-yl]carbamate (0.23 g, 0.57 mmol) in dioxane (2.5 mL), was added 4 M solution of hydrochloric acid in dioxane (2.5 mL). The reaction was stirred at ambient temperature for 3 h and then concentrated to dryness to obtain crude (3S)-1-[5-(4-chlorophenyl)furan-2-carbonyl]piperidin-3-amine (~ 0.2 g) as a hydrochloride salt.

The crude (3S)-1-[5-(4-chlorophenyl)furan-2-carbonyl]piperidin-3-amine hydrochloride (0.02 g, 0.059 mmol) was taken in dichloromethane (1 mL) and cooled to 0° C. To this solution, was added 2-chloroethane sulfonyl chloride (6.25 µL, 0.059 mmol) and N,N-diisopropylethylamine (0.02 mL, 0.12 mmol). The mixture was stirred at 0° C. for an hour, followed by addition of N,N-diisopropylethylamine (0.01 mL, 0.06 mmol). The reaction mixture was stirred at 0 ·C and then partitioned between dichloromethane and saturated aqueous ammonium chloride. The layers were separated, and the organic phase was washed with water and brine, dried over MgSO₄, filtered, and concentrated. The crude concentrate was purified by flash silica gel chromatography (4 g Silicycle column), eluting with ethyl acetate in hexanes (50%) to provide 0.014 g (61%) of the title compound as a pale yellow solid. ¹H NMR (400 MHz, CHLOROFORM-d, 25° C.): δ = 7.64 (br d, J= 8.2 Hz, 2 H), 7.38 (br d, J= 8.6 Hz, 2H), 7.09-7.11 (m, 1 H), 6.71 (br d, J= 3.5 Hz, 1 H), 6.49-6.58 (m, 1 H), 6.25 (br d, J= 16.4 Hz, 1 H), 5.89 (br d, J= 9.8 Hz, 1 H), 3.81 (br s, 1 H), 3.62-3.75 (m, 2 H), 3.53 (br s, 1 H), 1.95-2.06 (m, 1 H), 1.75-1.92 (m, 1 H), 1.61-1.74 (m, 1 H), 1.21-1.39 ppm (m, 2 H). LC-MS: m/z = 395 [M+H]⁺.

Synthesis of 5-(4-Chlorophenyl)-N-methyl-N-[2-(N-methylethenesulfonamido)ethyl]furan-2-carboxamide.

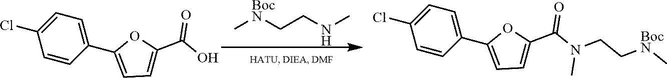

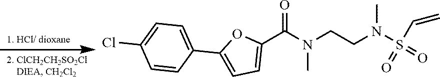

tert-Butyl N-(2-{1-[5-(4-chlorophenyl)furan-2-yl]-N-methylformamido}ethyl)-N-methylcarbamate. To a solution of 5-(4-chlorophenyl)furan-2-carboxylic acid (0.1 g, 0.45 mmol) in N,N′-dimethylformamide (0.5 mL) were added tert-butyl N-methyl-N-[2-(methylamino)ethyl]carbamate (0.085 g, 0.45 mmol), HATU (0.19 g, 0.5 mmol) and N,N-diisopropylethylamine (0.16 mL, 0.9 mmol). The reaction was stirred at ambient temperature for 18 h then partitioned between ethyl acetate and saturated aqueous ammonium chloride. The layers were separated, and the organic phase was washed with water and brine, dried over MgSO₄, filtered, and concentrated. The crude concentrate was purified by flash silica gel chromatography (12 g Silicycle column), eluting with ethyl acetate in hexanes (50%) to provide 0.17 g (96%) of the title compound as pale yellow oil. ¹H NMR (400 MHz, CHLOROFORM-d, 27° C.): δ = 7.65 (br d, J= 7.3 Hz, 2H), 7.40 (br d, J= 8.3 Hz, 2 H), 7.15 (br s, 1 H), 6.73 (br s, 1 H), 3.74 (br s, 1 H), 3.53 (br t, J = 5.7 Hz, 2 H), 3.44 (br s, 1 H), 2.94 (br s, 2 H), 1.43 (br s, 9H). LC-MS: m/z = 393 [M+H]⁺.

5-(4-Chlorophenyl)-N-methyl-N-[2-(N-methylethenesulfonamido)ethyl]furan-2-carboxamide. To a solution of tert-butyl N-(2-{ 1-[5-(4-chlorophenyl)furan-2-yl]-N-methylformamido}ethyl)-N-methylcarbamate (0.018 g, 0.046 mmol) in dioxane (0.2 mL), was added 4 M solution of hydrochloric acid in dioxane (0.2 mL). The reaction was stirred at 0° C. for 2 h and then concentrated to dryness to obtain crude 5-(4-chlorophenyl)-N-methyl-N-[2-(methylamino)ethyl]furan-2-carboxamide as a hydrochloride salt.

The crude 5-(4-chlorophenyl)-N-methyl-N-[2-(methylamino)ethyl]furan-2-carboxamide hydrochloride was taken in dichloromethane (0.5 mL) and cooled to 0° C. To this solution, was added 2-chloroethane sulfonyl chloride (5 µL, 0.046 mmol) and N,N-diisopropylethylamine (16 µL, 0.092 mmol). The mixture was stirred at 0° C. for an hour, followed by addition of N,N-diisopropylethylamine (8 µL, 0.046 mmol). The reaction mixture was stirred at 0 ·C and concentrated. The crude concentrate was purified by flash silica gel chromatography (4 g Silicycle column), eluting with ethyl acetate in hexanes (50-100%) to provide 5 mg (29%) of the title compound as a pale yellow solid. ¹H NMR (400 MHz, CHLOROFORM-d, 27° C.): δ = 7.61-7.71 (m, 2 H), 7.37-7.47 (m, 2 H), 7.16 (br d, J = 3.2 Hz, 1 H), 6.75 (d, J= 3.7 Hz, 1H), 6.45 (dd, J= 16.6, 10.0 Hz, 1 H), 6.25 (d, J= 16.6 Hz, 1 H), 5.99 (d, J= 10.0 Hz, 1 H), 3.80 (br dd, J= 5.1, 1.2 Hz, 2 H), 3.35-3.55 (m, 5 H), 2.80-2.99 ppm (m, 3 H). LC-MS: m/z = 405 [M+Na]⁺.

Synthesis of 5-(4-Chlorophenyl)-N-(2-ethenesulfonamidoethyl)furan-2-carboxamide

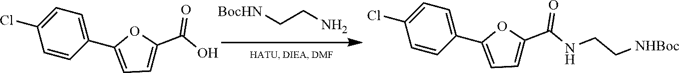

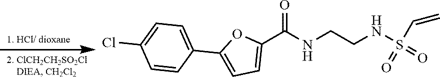

tert-Butyl N-(2-{[5-(4-chlorophenyl)furan-2-yl]formamido}ethyl)carbamate. To a solution of 5-(4-chlorophenyl)furan-2-carboxylic acid (0.25 g, 1.12 mmol) in N,N′-dimethylformamide (1.0 mL) were added tert-butyl N-(2-aminoethyl)carbamate (0.18 mL, 1.12 mmol), HATU (0.47 g, 1.23 mmol) and N,N-diisopropylethylamine (0.39 mL, 2.24 mmol). The reaction was stirred at ambient temperature for 18 h then partitioned between ethyl acetate and saturated aqueous ammonium chloride. The layers were separated, and the organic phase was washed with water and brine, dried over MgSO₄, filtered, and concentrated. The crude concentrate was purified by flash silica gel chromatography (25 g Silicycle column), eluting with ethyl acetate in hexanes (50%) to provide 0.33 g (81%) of the title compound as cream colored solid. ¹H NMR (400 MHz, CHLOROFORM-d, 27° C.): δ = 7.74 (br d, J= 8.3 Hz, 2 H), 7.52 (br s, 1 H), 7.40 (d, J = 8.5 Hz, 2 H), 7.18 (d, J = 3.4 Hz, 1 H), 6.74 (d, J = 3.4 Hz, 1 H), 5.01 (br s, 1 H), 3.58 (q, J= 5.2 Hz, 2 H), 3.44 (br d, J= 4.9 Hz, 2 H), 1.46 ppm (s, 9 H). LC-MS: m/z = 365 [M+H]⁺.

5-(4-Chlorophenyl)-N-(2-ethenesulfonamidoethyl)furan-2-carboxamide. To a solution of tert-butyl N-(2-{[5-(4-chlorophenyl)furan-2-yl]formamido}ethyl)carbamate (0.027 g, 0.074 mmol) in dioxane (0.3 mL), was added 4 M solution of hydrochloric acid in dioxane (0.3 mL). The reaction was stirred at 0° C. for 2 h and then concentrated to dryness to obtain crude N-(2-aminoethyl)-5-(4-chlorophenyl)furan-2-carboxamide as a hydrochloride salt.

The crude N-(2-aminoethyl)-5-(4-chlorophenyl)furan-2-carboxamide hydrochloride was taken in dichloromethane (0.5 mL) and cooled to 0° C. To this solution, was added 2-chloroethane sulfonyl chloride (8 µL, 0.046 mmol) and N,N-diisopropylethylamine (26 µL, 0.14 mmol). The mixture was stirred at 0° C. for an hour, followed by addition of N,N-diisopropylethylamine (13 µL, 0.07 mmol). The reaction mixture was stirred at 0° C. and concentrated. The crude concentrate was purified by flash silica gel chromatography (4 g Silicycle column), eluting with ethyl acetate in hexanes (50-100%) to provide 10 mg (38%) of the title compound as a clear oil. ¹H NMR (400 MHz, CHLOROFORM-d, 27° C.): δ = 7.69 (d, J = 8.5 Hz, 2 H), 7.43 (d, J= 8.8 Hz, 2 H), 7.17-7.25 (m, 1 H), 6.94 (br s, 1 H), 6.76 (d, J= 3.4 Hz, 1 H), 6.50-6.62 (m, 1 H), 6.23-6.37 (m, 1 H), 5.98 (d, J= 10.0 Hz, 1 H), 3.64-3.68 (m, 2 H), 3.32-3.36 (m, 2 H). LC-MS: m/z = 377 [M+Na]⁺.

### Example 2: Biological Data

tion at which 50% of the subunit is bound to compound, based on mass spectrometry. ^{b} for biochemical assay, see Heise, et al. PLoS One, 2012, 7, e50864.

TABLE 1

Biological Data for select compounds

| | | 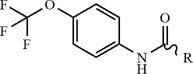 | |
|---|---|---|---|
| 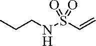 | 1.5 | 120 | 1.2 |
| 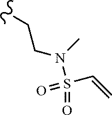 | 0.37 | | 0.99 |
| 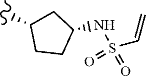 | 14.4 | | 38.9 |

^{a} SS and LS refer to the short subunit and large subunit of caspase-6; DR₅₀ refers to the concentration at which 50% of the subunit is bound to compound, based on mass spectrometry. ^{b} for biochemical assay, see Heise, et al. PLoS One, 2012, 7, e50864.

Scheme 19: Caspase 6 IC₅₀ data (nM) for select compounds. For assay method, see Heise, et al. PLoS One, 2012, 7, e50864.

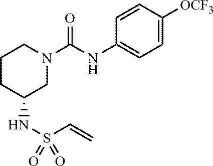 [SMILES: C=CS(=O)(=O)N[C@@H]1CCCN(C(=O)Nc2ccc(OC(F)(F)F)cc2)C1]

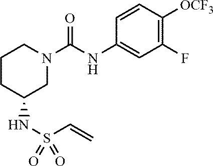 [SMILES: C=CS(=O)(=O)N[C@@H]1CCCN(C(=O)Nc2ccc(OC(F)(F)F)c(F)c2)C1]

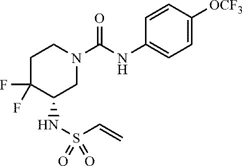 [SMILES: C=CS(=O)(=O)N[C@H]1CN(C(=O)Nc2ccc(OC(F)(F)F)cc2)CCC1(F)F]

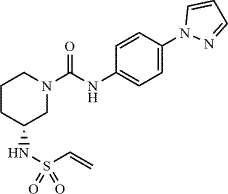 [SMILES: C=CS(=O)(=O)N[C@@H]1CCCN(C(=O)Nc2ccc(-n3cccn3)cc2)C1]

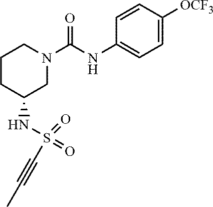 [SMILES: CC#CS(=O)(=O)N[C@@H]1CCCN(C(=O)Nc2ccc(OC(F)(F)F)cc2)C1]

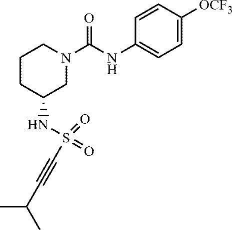 [SMILES: CC(C)C#CS(=O)(=O)N[C@@H]1CCCN(C(=O)Nc2ccc(OC(F)(F)F)cc2)C1]

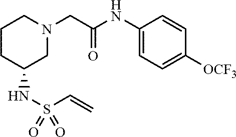 [SMILES: C=CS(=O)(=O)N[C@@H]1CCCN(CC(=O)Nc2ccc(OC(F)(F)F)cc2)C1]

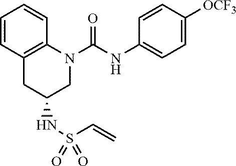 [SMILES: C=CS(=O)(=O)N[C@@H]1Cc2ccccc2N(C(=O)Nc2ccc(OC(F)(F)F)cc2)C1]

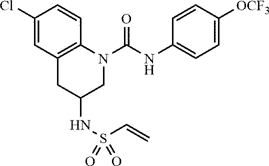 [SMILES: C=CS(=O)(=O)NC1Cc2cc(Cl)ccc2N(C(=O)Nc2ccc(OC(F)(F)F)cc2)C1]

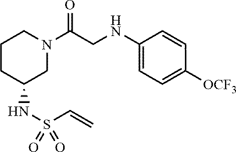 [SMILES: C=CS(=O)(=O)N[C@@H]1CCCN(C(=O)CNc2ccc(OC(F)(F)F)cc2)C1]

Scheme 19 (cont).

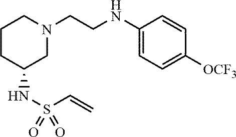 [SMILES: C=CS(=O)(=O)N[C@@H]1CCCN(CCNc2ccc(OC(F)(F)F)cc2)C1]

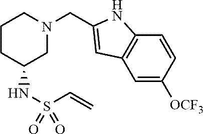 [SMILES: C=CS(=O)(=O)N[C@@H]1CCCN(Cc2cc3cc(OC(F)(F)F)ccc3[nH]2)C1]

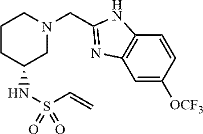 [SMILES: C=CS(=O)(=O)N[C@@H]1CCCN(Cc2nc3cc(OC(F)(F)F)ccc3[nH]2)C1]

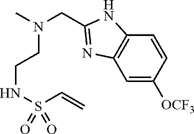 [SMILES: C=CS(=O)(=O)NCCN(C)Cc1nc2cc(OC(F)(F)F)ccc2[nH]1]

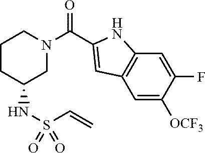 [SMILES: C=CS(=O)(=O)N[C@@H]1CCCN(C(=O)c2cc3cc(OC(F)(F)F)c(F)cc3[nH]2)C1]

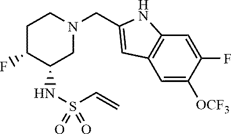 [SMILES: C=CS(=O)(=O)N[C@H]1CN(Cc2cc3cc(OC(F)(F)F)c(F)cc3[nH]2)CC[C@H]1F]

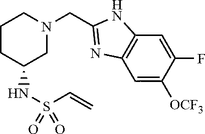 [SMILES: C=CS(=O)(=O)N[C@@H]1CCCN(Cc2nc3cc(OC(F)(F)F)c(F)cc3[nH]2)C1]

### Example 3: Additional Warheads

The synthesis of sulfoximines (e.g.

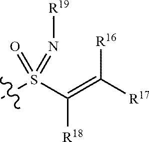

) and sulfondiimines (

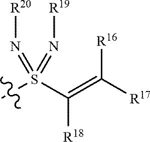

) can be carried out according to methods known to one of skill in the art, including but not limited to those methods reported Org. Chem. Front., 2019, 6, 1319-1324.

### REFERENCES

Lucking, U. Org. Chem. Front., 2019, 6, 1319-1324.
Nguyen, C., etal. Nature, 2014, 505, 427-431.
Kiemele, E., etal. Org. Lett., 2016, 18, 492-495.
Heise, C. E., Murray J, Augustyn, K. E., Bravo, B., et al. PLoS One, 2012, 7, e50864.
Mintzer, R., Ramaswamy, S., Shah, K., Hannoush, R. N., et al. PLoS One, 2012, 7, e30376.
Sohn P.D., Huang, C. T-L., Yan, R., Fan. Li, et al. Neuron 2019, 104, 458-470.
Wang C., Ward M.E., Chen R., Liu, K., et al. Stem Cell Reports 2017, 9, 1221-1233.
Karch C. M., Kao A.W., Karydas A., Onanuga K., et al. Stem cell reports. 2019, 13, 939-55.
van de Leemput J., Boles N.C., Kiehl T.R., Corneo B., et al. Neuron. 2014, 83, 51-68.
Chambers S.M., Fasano C.A., Papapetrou E.P., Tomishima M., et al. Nat Biotechnol. 2009, 27,
275-80.

1. A compound having the formula: 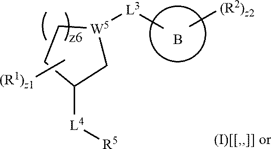 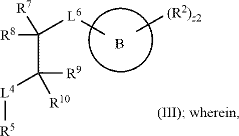
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents on adjacent carbons may optionally be joined to form a substituted or unsubstituted cycloalkyl, substituted or unsubstituted heterocycloalkyl, substituted or unsubstituted aryl, or substituted or unsubstituted heteroaryl; or two R¹ substituents bonded to the same carbon atom may optionally be joined to form a substituted or unsubstituted cycloalkyl or substituted or unsubstituted heterocycloalkyl;
z1 is an integer from 0 to 9;
R² is independently oxo, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B},—NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z2 is an integer from 0 to 6;
L³ is a bond, —S(O)₂—, —NR³—, —NH—, —O—, —S—, —C(O)—, —C(O)NR³—, —NR³C(O)—, —N(R³)CH₂—, —NR³C(O)NH—, —NHC(O)NR³—, —C(O)O—, —OC(O)—, substituted or unsubstituted alkylene, substituted or unsubstituted heteroalkylene;
L⁴ is a bond, —NH—, —NR⁴—, or substituted or unsubstituted alkylene;
L⁶ is —N(R⁶)—L³— or —C(O)NH—;
W⁵ is CH orN;
z6 is 1 or 2;
R³, R⁴, and R⁶ are independently hydrogen, —CCl₃, —CBr₃, —CF₃, —CI₃, —CHC₂, —CHBr₂, —CHF₂, —CHI₂, —CH₂C1, —CH₂Br, —CH₂F, —CH₂I, —CN, —OH, —NH₂, —COOH, —COCH₃, —CONH₂, —OCC1₃, —OCF₃, —OCBr₃, —OCI₃, —OCHCl₂, —OCHBr₂, —OCHI₂, —OCHF₂, —OCH₂C1, —OCH₂Br, —OCH₂I, —OCH₂F, unsubstituted alkyl, unsubstituted heteroalkyl, unsubstituted cycloalkyl, or unsubstituted heterocycloalkyl;
R⁷, R⁸, R⁹, and R¹⁰ are independently hydrogen or unsubstituted C₁-C₁₀ alkyl;
Ring B is aryl or heteroaryl;
R⁵ is independently 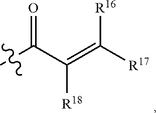 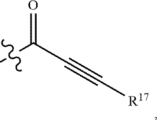 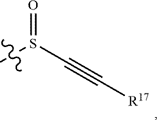 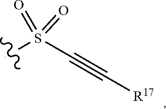 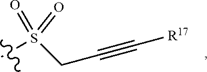 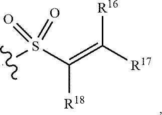 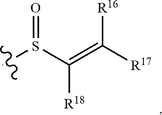 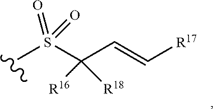 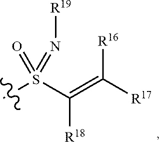 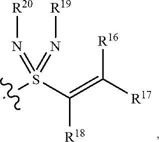 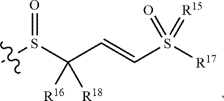 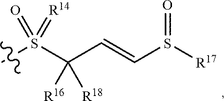 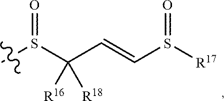 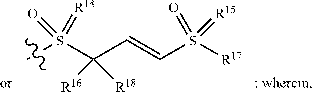
R¹⁴ is ═O or ═NR¹⁹;
R¹⁵ is ═O or ═NR²⁰;
R¹⁶, R¹⁷, and R¹⁸ are independently hydrogen, oxo, halogen, —CCl₃, —CBr₃, —CF₃, —CI₃, —CHCl₂, —CHBr₂, —CHF₂, —CHI₂, —CH₂C1, —CH₂Br, —CH₂F, —CH₂I, —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCC1₃, —OCF₃, —OCBr₃, —OCI₃, —OCHCl₂, —OCHBr₂, —OCHI₂, —OCHF₂, —OCH₂C1, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁹ and R²⁰ are independently hydrogen, —CCl₃, —CBr₃, —CF₃, —CI₃, —CHCl₂, —CHBr₂, —CHF₂, —CHI₂, —CH₂C1, —CH₂Br, —CH₂F, —CH₂I, —CN, —COOH, —CONH₂, —C(O)N(CH₃)₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C} R^{1D} R^{2A} R^{2B}, R^{2C}, and R^{2D} are independently hydrogen, —CCl₃, —CBr₃, —CF₃, —CI₃, CHC1₂, —CHBr₂, —CHF₂, —CHI₂, —CH₂C1, —CH₂Br, —CH₂F, —CH₂I, —CN, —OH, —NH₂, , —CONH₂, —OCC1₃, —OCF₃, —OCBr₃, —OCI₃, —OCHCl₂, —OCHBr₂, —OCHI₂, —OCHF₂, —OCH₂C1, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl;
X¹ and X² are independently —F, —Cl, —Br, or —I;
n1 and n2 are independently an integer from 0 to 4; and
m1, m2, v1, and v2 are independently 1 or 2.

2. The compound of claim 1, having the formula: 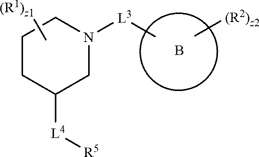
.

3. The compound of claim 2, having the formula: 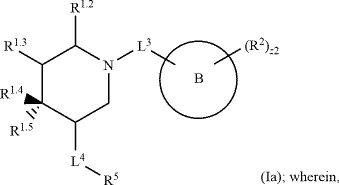
R^{1.2} and R^{1.3} are independently hydrogen, halogen, —CCl₃, —CBr₃, —CF₃, —CI₃, —CHCl₂, —CHBr₂, —CHF₂, —CHI₂, —CH₂C1, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCC1₃, —OCF₃, —OCBr₃, —OCI₃, —OCHCl₂, —OCHBr₂, —OCHI₂, —OCHF₂, —OCH₂C1, —OCH₂Br, —OCH₂I, —OCH₂F, —N₃, substituted or unsubstituted C₁-C₆ alkyl, or substituted or unsubstituted 2 to 6 membered heteroalkyl; or R^{1.2} and R^{1.3} substituents on adjacent carbons may optionally be joined to form a substituted or unsubstituted phenyl, or substituted or unsubstituted 5 to 6 membered heteroaryl; and
R^{1.4} and R^{1.5} are independently hydrogen, halogen, —CCl₃, —CBr₃, —CF₃, —CI₃, —CHCl₂, —CHBr₂, —CHF₂, —CHI₂, —CH₂C1, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂C1, —OCH₂Br, —OCH₂I, —OCH₂F, —N₃, substituted or unsubstituted C₁-C₆ alkyl, or substituted or unsubstituted 2 to 6 membered heteroalkyl.

4. The compound of claim 3, having the formula: 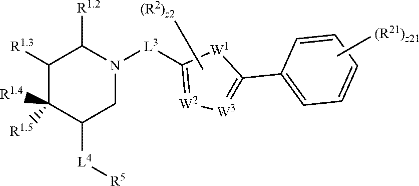 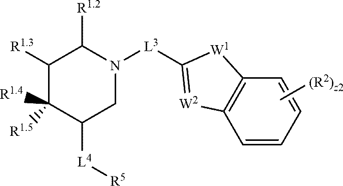 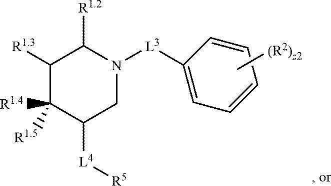 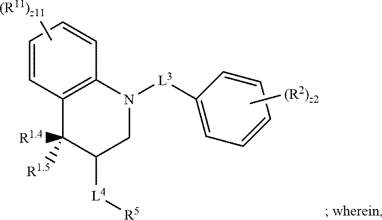
W¹ is O—, —NH—, or —NR²—;
W² and W³ are independently ═N—, ═CH—, or ═CR²—;
R^{1.2}, R^{1.3 ,} R^{1.4} and R^{1.5} are independently hydrogen or halogen;
R² is independently oxo, halogen, -CX²₃, —CHX²₂, —CH₂X², —OCX²₃, —OCH₂X², —OCHX²₂, —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted C₆-C₁₂ aryl, or unsubstituted 5 to 12 membered heteroaryl;
R¹¹ is independently oxo, halogen, —CX¹¹₃, —CHX¹¹₂, —CH₂X¹¹, —OCX¹¹₃, —OCH₂X¹¹, —OCHX¹¹₂, —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted C₆-C₁₂ aryl, or unsubstituted 5 to 12 membered heteroaryl;
R²¹ is independently halogen, —CX²¹₃, —CHX²¹₂, —CH₂X²¹, —OCX²¹₃, —OCH₂X²¹, —OCHX²¹₂, —CN, —OH, —NH₂, —COOH, —CONH₂, —C(O)N(CH₃)₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted C₆-C₁₂ aryl, or unsubstituted 5 to 12 membered heteroaryl;
X¹¹ and X²¹ are independently —F, —Cl, —Br, or —I;
z2 is an integer from 0 to 6;
z11 is an integer from 0 to 4;
z21 is an integer from 0 to 5;
L³ is —C(O)—, —CH₂—, —C(O)NH—, —NHC(O)—, —NHCH₂—, —CH₂CH₂NH—, —C(O)CH₂NH—, or —CH₂C(O)NH—;
L⁴ is a bond, —NH—, or —CH₂—; and
R⁵ is 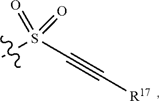 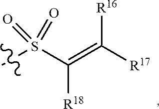 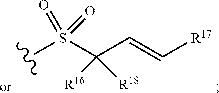
wherein R¹⁶, and R¹⁸ are independently hydrogen, —C(O)N(CH₃)₂, or unsubstituted C₁-C₃ alkyl; and
R¹⁷ is hydrogen, —C(O)N(CH₃)₂, unsubstituted C₁-C₃ alkyl, or substituted or unsubstituted 5 to 6 membered heteroaryl.

5. The compound of claim 4, wherein: R² is independently halogen, —OCX²₃, —OCH₂X², —OCHX²₂, unsubstituted C₁—C₃ alkyl, or unsubstituted 5 to 6 membered heteroaryl; R¹¹ is independently halogen, —OCX¹¹₃, —OCH₂X¹¹, -OCHX¹¹₂, unsubstituted C₁-C₃ alkyl, or unsubstituted 5 to 6 membered heteroaryl; and R²¹ is independently halogen, -OCX²¹₃, —OCH₂X²¹, —OCHX²¹₂, unsubstituted C₁-C₃ alkyl, or unsubstituted 5 to 6 membered heteroaryl.

6-9. (canceled)

10. The compound of claim 1, having the formula: 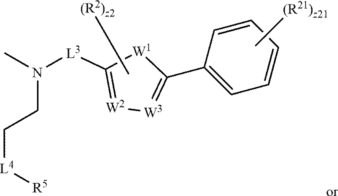 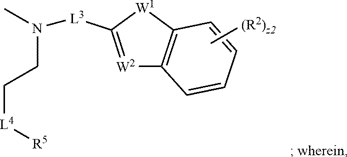
W¹ is O—, —NH—, or —NR²—;
W² and W³ are independently ═N—, ═CH—, or ═CR²—
R² and R²¹ are independently halogen, —OCX²₃, —OCH₂X², —OCHX²₂, unsubstituted C₁—C₃ alkyl, or unsubstituted 5 to 6 membered heteroaryl;
z2 is an integer from 0 to 6;
z21 is an integer from 0 to 5;
L³ is —C(O)— or —CH₂—;
L⁴ is a bond, —NH—, —NR⁴—, or —CH₂—; and
R⁵ is independently 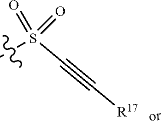 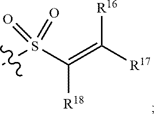
wherein R¹⁶, R¹⁷, and R¹⁸ are independently hydrogen, —C(O)N(CH₃)₂, or unsubstituted C₁-C₃ alkyl.

11. The compound of claim 10, having the formula: 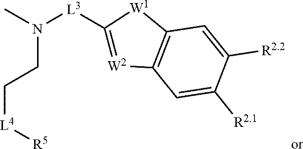 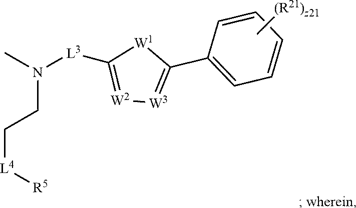
R^{2.1} is hydrogen, —OCX²₃, or unsubstituted 5 to 6 membered heteroaryl; and
R^{2.2} is hydrogen or halogen.

12. The compound of claim 1, wherein (Ring B)-(R²)_{z2} is 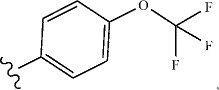 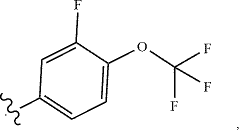 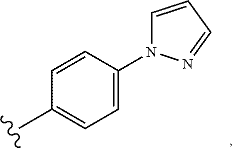 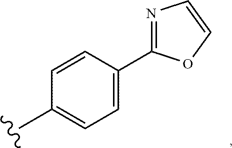 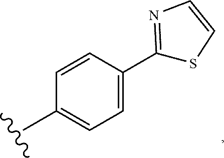 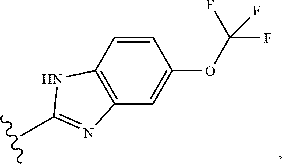 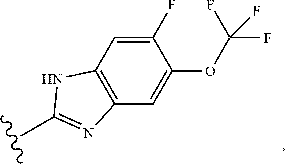 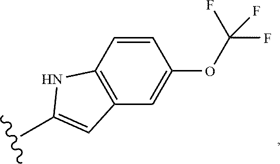 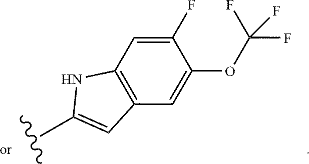
.

13. The compound of claim 1, wherein L⁴ is —NH—, —CH₂—, or —N(CH₃)—.

14. (canceled)

15. (canceled)

16. The compound of claim 1, wherein R⁵ is 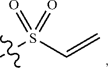 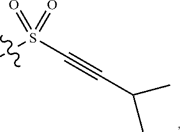 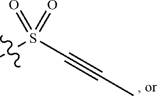 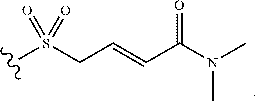
.

17. The compound of claim 1, wherein L³ is —C(O)—, —CH₂—, —C(O)NH—, —CH₂CH₂NH—, —C(O)CH₂NH—, or —CH₂C(O)NH.

18. (canceled)

19. (canceled)

20. The compound of claim 1 having the formula: 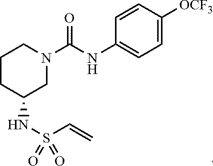 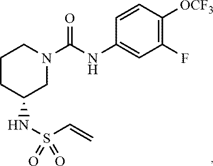 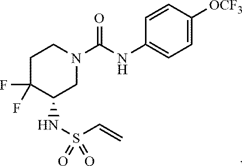 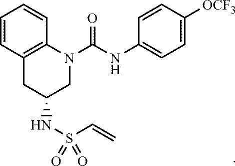 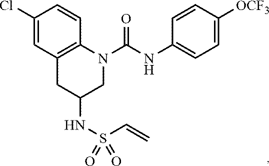 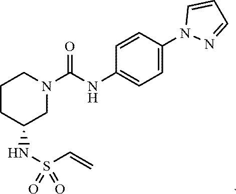 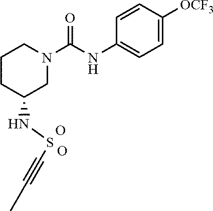 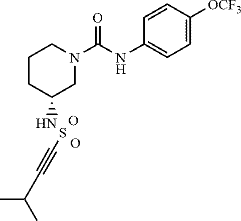 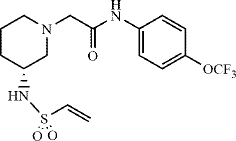 [SMILES: C=CS(=O)(=O)N[C@@H]1CCCN(CC(=O)Nc2ccc(OC(F)(F)F)cc2)C1] 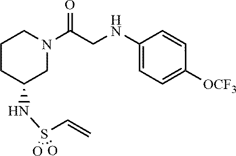 [SMILES: C=CS(=O)(=O)N[C@@H]1CCCN(C(=O)CNc2ccc(OC(F)(F)F)cc2)C1] 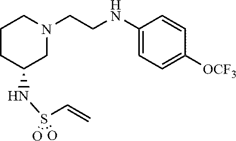 [SMILES: C=CS(=O)(=O)N[C@@H]1CCCN(CCNc2ccc(OC(F)(F)F)cc2)C1] 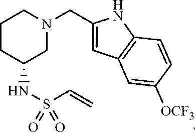 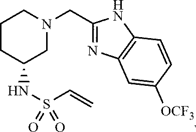 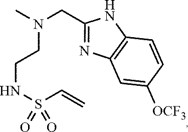 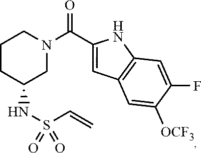 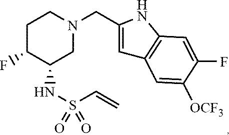 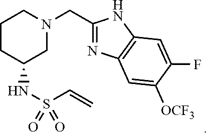 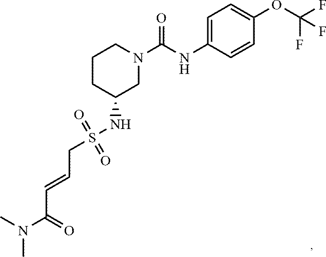 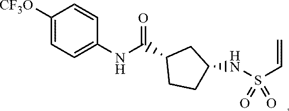 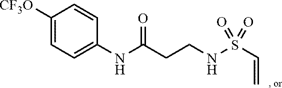 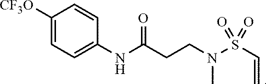
.

21. A pharmaceutical composition comprising the compound of claim 1 and a pharmaceutically acceptable excipient.

22. A method of inhibiting human Caspase 6 protein activity, said method comprising contacting the human Caspase 6 protein with a compound of claim 1.

23-25. (canceled)

26. A method of treating a neurodegenerative disease, said method comprising administering to a subject in need thereof an effective amount of a compound of claim 1.

27-29. (canceled)

30. A method of treating a memory loss, said method comprising administering to a subject in need thereof an effective amount of a compound of claim 1.

31. A method of treating axonal degradation, said method comprising administering to a subject in need thereof an effective amount of a compound of claim 1.

32. A method of treating neuroinflammation, said method comprising administering to a subject in need thereof an effective amount of a compound of claim 1.

33. A method of treating liver disease, said method comprising administering to a subject in need thereof an effective amount of a compound of claim 1.

34. A method of treating nonalcoholic steatohepatitis or nonalcoholic fatty liver disease, said method comprising administering to a subject in need thereof an effective amount of a compound of claim 1.

35-43. (canceled)


SEQUENCE LISTING (3 sequences)

SEQ ID NO: 1 - protein, 293 aa, Artificial Sequence
  Synthetic polypeptide
MSSASGLRRGHPAGGEENMTETDAFYKREMFDPAEKYKMDHRRRGIALIFNHERFFWHLTLPERRGTCADRDNLTRRFSDLGFEVKCFNDLKAEELLLKIHEVSTVSHADADCFVCVFLSHGEGNHIYAYDAKIEIQTLTGLFKGDKCHSLVGKPKIFIIQACRGNQHDVPVIPLDVVDNQTEKLDTNITEVDAASVYTLPAGADFLMCYSVAEGYYSHRETVNGSWYIQDLCEMLGKYGSSLEFTELLTLVNRKVSQRRVDFCKDPSAIGKKQVPCFASMLTKKLHFFPKSN

SEQ ID NO: 2 - protein, 277 aa, Artificial Sequence
  Synthetic polypeptide
MENTENSVDSKSIKNLEPKIIHGSESMDSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETDSGVDDDMACHKIPVEADFLYAYSTAPGYYSWRNSKDGSWFIQSLCAMLKQYADKLEFMHILTRVNRKVATEFESFSFDATFHAKKQIPCIVSMLTKELYFYH

SEQ ID NO: 3 - protein, 452 aa, Artificial Sequence
  Synthetic polypeptide
MAAPSAGSWSTFQHKELMAADRGRRILGVCGMHPHHQETLKKNRVVLAKQLLLSELLEHLLEKDIITLEMRELIQAKVGSFSQNVELLNLLPKRGPQAFDAFCEALRETKQGHLEDMLLTTLSGLQHVLPPLSCDYDLSLPFPVCESCPLYKKLRLSTDTVEHSLDNKDGPVCLQVKPCTPEFYQTHFQLAYRLQSRPRGLALVLSNVHFTGEKELEFRSGGDVDHSTLVTLFKLLGYDVHVLCDQTAQEMQEKLQNFAQLPAHRVTDSCIVALLSHGVEGAIYGVDGKLLQLQEVFQLFDNANCPSLQNKPKMFFIQACRGDETDRGVDQQDGKNHAGSPGCEESDAGKEKLPKMRLPTRSDMICGYACLKGTAAMRNTKRGSWYIEALAQVFSERACDMHVADMLVKVNALIKDREGYAPGTEFHRCKEMSEYCSTLCRHLYLFPGHPPT